(19) United States
(12) Patent Application Publication
(10) Pub. No.: US 2013/0029970 A1
(43) Pub. Date: Jan. 31, 2013

(54) CB Receptor Agonists

(71) Applicants: Kevin Sprott, Needham, MA (US); Jason Rohde, Andover, MA (US); Takashi Nakai, Newton, MA (US); Bo Peng, Arlington, MA (US); John Jeffrey Talley, Saint Louis, MO (US)

(72) Inventors: Kevin Sprott, Needham, MA (US); Jason Rohde, Andover, MA (US); Takashi Nakai, Newton, MA (US); Bo Peng, Arlington, MA (US); John Jeffrey Talley, Saint Louis, MO (US)

(73) Assignee: IRONWOOD PHARMACEUTICALS, INC, Cambridge, MA (US)

(21) Appl. No.: 13/383,241

(22) PCT Filed: Jul. 9, 2010
(86) PCT No.: PCT/US10/41523

§ 371 Date: Aug. 24, 2012

Related U.S. Application Data
(60) Provisional application No. 61/224,750, filed on Jul. 10, 2009.
(60) Provisional application No. 61/302,609, filed on Feb. 9, 2010.
(60) Provisional application No. 61/359,711, filed on Jun. 29, 2010.

(51) Int. Cl. A61K 31/5377; A61K 31/407; A61K 31/454; A61K 31/4439; A61K 31/4545; A61K 31/4178; A61K 31/422; A61K 31/496; A61K 31/541; A61K 31/553; A61P 25/00; A61P 37/00; A61P 29/00; A61P 25/30; A61P 25/18; A61P 25/28; A61P 27/02; C12N 5/07; C07D 495/04

(52) U.S. Cl. 514/211.15; 544/143; 514/233.8; 548/453; 544/131; 514/414; 546/198; 514/321; 544/122; 544/123; 514/412; 546/276.7; 514/338; 544/124; 546/193; 514/318; 544/144; 548/311.7; 514/392; 548/231; 514/376; 544/373; 514/254.08; 544/58.7; 514/228.2; 540/544; 435/375

(57) ABSTRACT

The present disclosure relates to compounds of formula I useful as agonists of cannabinoid receptors. The disclosure also provides pharmaceutically acceptable compositions comprising the compounds of the disclosure and methods of using the compositions in the treatment of various disorders.

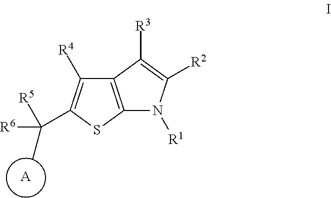

### PRIORITY CLAIM

This application claims priority to U.S. Ser. No. 61/224,750 which was filed on Jul. 10, 2009 and U.S. Ser. No. 61/302,609 which was filed on Feb. 9, 2010 and U.S. Ser. No. 61/359,711 which was filed Jun. 29, 2010. The entire contents of the aforementioned applications are incorporated herein.

### TECHNICAL FIELD

The present disclosure relates to compounds useful as agonists of cannabinoid receptors. The disclosure also provides pharmaceutically acceptable compositions comprising the compounds of the disclosure and methods of using the compositions in the treatment of various disorders.

### BACKGROUND

Cannabinoids are a group of compounds found in Cannabis sativa (also known as marijuana). Cannabis has been used in the treatment of various illnesses ranging from lack of appetite, emesis, cramps, menstrual pain, cancer pain and spasticity. Despite its clinical benefits, the therapeutic usage of cannabis is limited by its psychoactive effects including hallucination, addiction and dependence.

The physiological effects of cannabinoids are mediated by at least two G-protein coupled receptors, cannabinoid receptors 1 and 2 (CB1 and CB2). CB1 receptors are expressed primarily in the central nervous system and are thought to mediate many of the psychoactive effects of cannabis. CB2 receptors are predominantly found in the immune system. CB2 receptors are expressed on inflammatory cells (T cells, B cells, macrophages, mast cells) and mediate immune suppression through inhibition of cellular interaction/inflammatory mediator release. More recent data also suggests a role for CB2 receptor activation in the CNS. CB2 receptor expression appears to be induced by inflammatory pain in a rat spinal cord model, which coincided with the appearance of activated microglia. CB2 receptor agonists also have been shown to reduce mechanically-evoked responses and wind-up of wide dynamic range neurons in spinal cord dorsal horn in animal models of inflammatory pain. CB2 up regulation was also observed in lesioned areas of brains in an animal model mimicking Alzheimer's disease. In addition, hepatic expression of CB2 receptors has also been observed in patients with chronic liver disease, and activation of CB2 receptors can trigger potent growth inhibitory and apoptotic effects, two major antifibrogenic properties, in hepatic myofibroblasts.

Cannabinoid receptors (CB) agonists may be used for treating immune disorders, liver fibrosis, inflammation and disorders that have an inflammatory component, such as cardiovascular disease, osteoporosis and renal ischemia. In addition, CB agonists may also be used in the treatment of emesis and pain including acute, chronic, inflammatory, post-operative, cancer and neuropathic pain.

Thus, there is a need to develop compounds useful as selective agonists of the cannabinoid receptors.

### SUMMARY

The compounds disclosed herein, and pharmaceutically acceptable compositions thereof, are effective as CB agonists. These compounds have the general formula I:

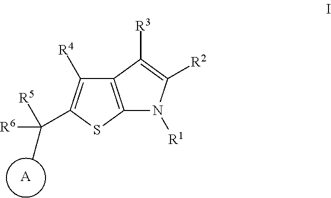

wherein

R¹ is V—R⁸;

V is a covalent bond between R⁸ and the nitrogen to which V is bonded, or is a divalent linker between R⁸ and the nitrogen to which V is bonded, wherein said linker is a saturated or unsaturated C₁₋₆ aliphatic which is optionally substituted with one or more (i.e. up to 6) instances of halogen, a C₁₋₄ aliphatic, —OR¹⁴, —CN, —SR¹⁴, —CO₂R¹⁴, —OC(O)R¹⁴, —C(O)N(R¹⁴H)₂, —N(R)C(O)R¹⁴, —N(R)C(O)OR¹⁴, —OC(O)N(R¹⁴H)₂, —N(R)C(O)NR¹⁴ or —N(R¹⁴H)₂; wherein said C₁₋₄ aliphatic is optionally substituted with one or more (i.e. up to 6) instances of halogen, —OR¹⁴, —CN or —N(R¹⁴H)₂; and wherein up to two saturated carbons of said C₁₋₆ aliphatic are replaced by —O—, —C(O)—, —C(S)—, —C(O)N(R)—, —N(R)C(O)—, —C(O)O—, —OC(O)—, —C(═N—N(R¹⁴H)₂)—, —C(═N—OR¹⁴H)—, —N(R)—, —N(R)S(O)₂—, —S(O)₂N(R)—, —N(R)S(O)₂N(R)—, —N(R)C(O)O—, —OC(O)N(R)—, —N(R)C(O)N(R)—, —OC(O)N(R)—, —S—, —S(O)—, —S(O)₂—, —C(O)S—, —SC(O)—, —C(S)S—, —SC(S)—, —OC(S)—, —C(S)O—, —C(S)N(R)—, —N(R)C(S)—, —N(R)C(S)S—, —SC(S)N(R)—, —N(R)C(S)O—, —N(R)C(O)S—, —OC(S)N(R)— or —SC(O)N(R)—;

each occurrence of R is independently selected from hydrogen, a C₁₋₄ aliphatic or a C₁₋₄ haloaliphatic;

each occurrence of R¹⁴ is independently selected from hydrogen, a C₁₋₄ aliphatic, a C₁₋₄ haloaliphatic, a C₃₋₇ cycloaliphatic or a 3-7 membered heterocyclyl; or two R¹⁴ attached to the same or different atom(s), together with the atom(s) to which they are attached, form a C₃₋₇ cycloaliphatic or 3-7 membered heterocycle; wherein said cycloaliphatic and heterocyclyl are optionally and independently substituted by one or more (i.e. up to 6) instances of halogen, —CN, oxo, C₁₋₄ alkyl, C₁₋₄ haloalkyl, C₁₋₄ alkoxy or C₁₋₄ haloalkoxy;

R⁸ is hydrogen, halogen, —NO₂, —CN, a C₁-C₆ aliphatic, a 5-14 membered aryl group, a 5-14 membered heteroaryl group, a C₃-C₁₂ cycloaliphatic group, or a 3-14 membered heterocyclyl group; wherein said aliphatic, aryl, heteroaryl, cycloaliphatic and heterocyclyl group are optionally and independently substituted with one or more (i.e. up to 6) instances of R¹⁵;

each occurrence of R¹⁵ is independently selected from halogen, oxo, —NO₂, —CN, —OR¹⁷, —SR¹⁷, —S(O)₂R¹⁷, —SO₂N(R¹⁷)₂, —S(O)R¹⁷, —N(R¹⁷)₂, —C(O)OR¹⁷, —C(O)R¹⁷, —C(O)C(O)R¹⁷, —C(═N—N(R¹⁷)₂)R¹⁷, —N(R′)C(O)R¹⁷, —N(R′)C(O)OR¹⁷, —N(R′)S(O)₂R¹⁷, —N(R′)S(O)₂N(R′)R¹⁷, —N(R′)C(O)N(R′)R¹⁷, —N(R′)N(R′)R¹⁷, —C(O)NOR¹⁷, —C(O)N(R¹⁷)₂, —OC(O)R¹⁷, —OC(O)N(R¹⁷)₂, —C(O)SR¹⁷, —SC(O)R¹⁷, —C(S)SR¹⁷, —SC(S)R¹⁷, —OC(S)R¹⁷, —C(S)OR¹⁷, —C(S)N(R′)R¹⁷, —N(R′)C(S)R¹⁷, —N(R′)C(S)SR¹⁷, —SC(S)N(R′)R¹⁷, —N(R′)C(S)OR¹⁷, —N(R′)C(O)SR¹⁷, —OC(S)N(R′)R¹⁷, —SC(O)N(R′)R¹⁷, or a C₁₋₄ aliphatic, wherein said aliphatic is optionally substituted with one or more (i.e. up to 6) instances of —R¹⁶;

each occurrence of R′ is independently selected from hydrogen, C₁₋₄ aliphatic or C₁₋₄ haloaliphatic;

each occurrence of R¹⁶ is independently selected from halogen, oxo, —OR¹⁸, —CN, —CO₂R¹⁸, —C(O)N(R¹⁸)₂, —N(R″)C(O)R¹⁸, —OC(O)N(R¹⁸)₂, —N(R″)C(O)OR¹⁸, —N(R¹⁸)C(O)N(R¹⁸)₂, —N(R¹⁸)₂, a C₃₋₇ cycloaliphatic or a 3-7 membered heterocycle; wherein said cycloaliphatic and heterocycle can be optionally and independently substituted by one or more (i.e. up to 6) instances of halogen, —CN, oxo, C₁₋₄ alkyl, C₁₋₄ haloalkyl, C₁₋₄ alkoxy or C₁₋₄ haloalkoxy;

each occurrence of R¹⁷ is independently selected from hydrogen, a C₁-C₄ aliphatic, a C₁-C₄ haloaliphatic, a C₃₋₇ cycloaliphatic or a 3-7 membered heterocyclyl; or two R¹⁷ attached to the same or different atom(s), together with the atom(s) to which they are attached, form a 3-7 membered heterocycle; wherein said cycloaliphatic and heterocyclyl are optionally and independently substituted by one or more (i.e. up to 6) instances of halogen, —CN, oxo, C₁₋₄ alkyl, C₁₋₄ haloalkyl, C₁₋₄ alkoxy or C₁₋₄ haloalkoxy;

each occurrence of R″ is independently selected from hydrogen, C₁₋₄ aliphatic or C₁₋₄ haloaliphatic;

each occurrence of R¹⁸ is independently selected from hydrogen, alkyl aryl, a C₁₋₄ aliphatic or a C₁-C₄ haloaliphatic; or two R¹⁸ attached to the same or different atom(s), together with the atom(s) to which they are attached, form a 3-7 membered heterocycle; wherein said 3-7 membered heterocyclyl ring is optionally substituted by one or more (i.e. up to 6) instances of halogen, —CN, oxo, C₁₋₄ alkyl, C₁₋₄ haloalkyl, C₁₋₄ alkoxy or C₁₋₄ haloalkoxy;

R² is hydrogen, halogen, —CN, —NO₂, a C₁₋₄ aliphatic or a C₃₋₆ cycloaliphatic, wherein said aliphatic and cycloaliphatic are optionally and independently substituted with one or more (i.e. up to 6) instances of halogen, —CN, —OH, —O(C₁₋₂ alkyl), —O(C₁₋₂ haloalkyl), C₁₋₂ alkyl or C₁₋₂ haloalkyl;

R³ is hydrogen, halogen, —CN, —NO₂, —C(O)NR^{X}, —C(O)OR^{X}, a C₁₋₄ aliphatic or a C₃₋₆ cycloaliphatic, wherein said aliphatic and cycloaliphatic are optionally and independently substituted with one or more (i.e. up to 6) instances of halogen, —CN, —OH, —O(C₁₋₂ alkyl), —O(C₁₋₂ haloalkyl), C₁₋₂ alkyl or C₁₋₂ haloalkyl;

R⁴ is hydrogen, halogen, —CN, —NO₂, —C(O)NR^{X}, a C₁₋₄ aliphatic or a C₃₋₆ cycloaliphatic, wherein said aliphatic and cycloaliphatic are independently and optionally substituted with one or more (i.e. up to 6) instances of halogen, —CN, —OH, —O(C₁₋₂ alkyl), —O(C₁₋₂ haloalkyl), —C₁₋₂ alkyl or —C₁₋₂ haloalkyl.

R⁵ is chosen from hydrogen, halogen, —CN, —OH, —C(O)OR^{X}, C₁₋₄ aliphatic, a 3-7 membered heterocyclyl, C₃₋₆ cycloaliphatic, —O—(C₁₋₄ aliphatic) or —O—(C₃₋₆ cycloaliphatic); wherein said aliphatic, cycloaliphatic, —O—(C₁₋₄ aliphatic) and —O—(C₃₋₆ cycloaliphatic) are optionally and independently substituted with one or more (i.e. up to three) instances of halogen;

R⁶ is chosen from hydrogen, halogen, C₁₋₄ aliphatic, C₁₋₄ haloaliphatic, —O(C₁₋₄ aliphatic) or —O—(C₁₋₄ haloaliphatic); or

R⁵ and R⁶ are taken together to form ═O, ═S, ═NR^{W} or a 3-6 membered cycloaliphatic or heterocyclyl, wherein said cycloaliphatic or heterocyclyl is optionally and independently substituted with one or more (i.e. up to 6) instances of halogen, —CN, —OH, —O(C₁₋₄ aliphatic), —NO₂, —N(R^{Y})₂ or a C₁₋₄ aliphatic; wherein said aliphatic is optionally and independently substituted with one or more (i.e. up to 6) instances of —OR^{Y} or halogen;

R^{W} is selected from hydrogen, C₁₋₄ alkyl, C₁₋₄ haloalkyl, C₃₋₆ cycloalkyl, —O(C₁₋₄ alkyl), —O(C₁₋₄ haloalkyl), —N(C₁₋₄ alkyl)₂, —N(C₁₋₄ haloalkyl)₂ or —O(aryl); wherein said aryl is optionally substituted by up to 6 instances of halogen, —NO₂, C₁₋₄ alkyl, C₁₋₄ haloalkyl, —O(C₁₋₄ alkyl), or —O(C₁₋₄ haloalkyl);

each occurrence of R^{X} and R^{Y} are independently selected from hydrogen or a C₁₋₄ aliphatic, wherein said aliphatic is optionally and independently substituted with one or more (i.e. up to three) instances of halogen, OR^{iv} or N(R^{iv})₂;

each occurrence of R^{iv} is independently selected from hydrogen, C₁₋₄ aliphatic or C₁₋₄ haloaliphatic;

ring A is phenyl or a 5-6 membered monocyclic heteroaryl having 1-3 heteroatoms independently selected from N, O or S; wherein said phenyl and heteroaryl are optionally and independently substituted with one or more (i.e. up to five) instances of R¹²;

each occurrence of R¹² is independently selected from halogen, —NO₂, —CN, a C₁₋₄ aliphatic optionally substituted with one or more (i.e. up to four) instances of R¹⁹, —OR¹³, —SR¹³, —S(O)₂R¹³, —SO₂N(R¹³)₂, —S(O)R¹³, —N(R¹³)₂, —C(O)OR¹³, —C(O)R¹³, —C(O)C(O)R¹³, —C(═N—N(R¹³)₂)R¹³, —N(R¹³)C(O)R¹³, —N(R¹³)C(O)OR¹³, —N(R¹³)C(S)R¹³, —N(R¹³)C(S)OR¹³, —N(R¹³)S(O)₂R¹³, —N(R¹³)S(O)₂N(R¹³)R¹³, —N(R¹³)C(O)N(R¹³)R¹³, —N(R¹³)C(S)N(R¹³)R¹³, —N(R¹³)N(R¹³)R¹³, —C(O)NOR¹³, —C(O)N(R¹³)₂, —C(O)N(R¹³)N(R¹³)₂, —C(S)N(R¹³)₂, —C(S)(R¹³), —C(S)OR¹³, —C(S)N(R¹³)N(R¹³)₂, —OC(O)R¹³ or —OC(O)N(R¹³)₂; or two R¹² attached to different atom(s), together with the atom(s) to which they are attached, form a 3-7 membered heterocycle or C₃₋₇ cycloaliphatic; wherein said cycloaliphatic and heterocycle are independently and optionally substituted with one or more (i.e. up to 6) instances of halogen, —CN, —OH, C₁₋₂ alkyl, C₁₋₂ haloalkyl, C₁₋₂ alkoxy or C₁₋₂ haloalkoxy;

each R¹³ is independently selected from hydrogen, a C₁-C₄ aliphatic, a C₃₋₇ cycloaliphatic or a 3-7 membered heterocyclyl, wherein said aliphatic, cycloaliphatic and heterocyclyl are independently and optionally substituted with one or more (i.e. up to 6) instances of halogen; or two R¹³ attached to the same or different atom(s), together with the atom(s) to which they are attached, form a 3-7 membered heterocycle; wherein said heterocycle is optionally substituted with one or more (i.e. up to 6) instances of halogen, —CN, —OH, C₁₋₂ alkyl, C₁₋₂ haloalkyl, C₁₋₂ alkoxy or C₁₋₂ haloalkoxy;

each occurrence of R¹⁹ is independently selected from halogen, —OR²⁰, —NO₂, —CN, —CO₂R²⁰, —C(O)N(R²⁰)₂, —N(R²⁰)C(O)R²⁰, —N(R²⁰)C(O)OR²⁰, —N(R²⁰)₂, a C₃₋₇ cycloaliphatic, a 3-7 membered heterocyclyl, a C₁₋₄ aliphatic or a C₁₋₄ haloaliphatic; wherein said cycloaliphatic and heterocyclyl are optionally and independently substituted with one or more (i.e. up to 6) instances of halogen, —CN, —OH, C₁₋₂ alkyl, C₁₋₂ haloalkyl, C₁₋₂ alkoxy or C₁₋₂ haloalkoxy; and

each occurrence of R²⁰ is independently selected from hydrogen, a C₁₋₄ aliphatic or a C₁₋₄ haloaliphatic; or two R²⁰ attached to the same or different atom(s), together with the atom(s) to which they are attached, form a 3-7 membered heterocycle, wherein said heterocycle is optionally substituted with one or more (i.e. up to 6) instances of halogen, —CN, —OH, C₁₋₂ alkyl, C₁₋₂ haloalkyl, C₁₋₂ alkoxy or C₁₋₂ haloalkoxy.

In another aspect, this disclosure provides pharmaceutical compositions comprising a compound of Formula I, or a pharmaceutically acceptable salt, solvate, co-crystal or pro-drug thereof, and a pharmaceutically acceptable carrier, vehicle or adjuvant.

In a third aspect, these compounds, and pharmaceutically acceptable compositions thereof, are useful for treating or lessening the severity of a variety of disorders in a patient. These disorders include but are not limited to pain, including acute, chronic, inflammatory, post-operative, cancer and neuropathic pain; immune disorders, including autoimmune disorders; inflammation; disorders that have an inflammatory component; emesis; and liver fibrosis.

### DETAILED DESCRIPTION

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls.

### DESCRIPTION OF EXEMPLARY COMPOUNDS

### Definitions and General Terminology

For purposes of this disclosure, the chemical elements are identified in accordance with the Periodic Table of the Elements, CAS version, and the Handbook of Chemistry and Physics, 75.sup.th Ed. 1994. Additionally, general principles of organic chemistry are described in “Organic Chemistry”, Thomas Sorrell, University Science Books, Sausalito: 1999, and “March's Advanced Organic Chemistry”, 5.sup.th Ed., Smith, M. B. and March, J., eds. John Wiley & Sons, New York: 2001, which are herein incorporated by reference in their entirety.

As described herein, compounds of the invention may optionally be substituted with one or more substituents, such as illustrated generally below, or as exemplified by particular classes, subclasses, and species of the invention. The phrase “optionally substituted” is used interchangeably with the phrase “substituted or unsubstituted.” In general, the term “substituted”, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. If a substituent radical or structure is not identified or defined as “optionally substituted”, the substituent radical or structure is not substituted. As it will be apparent to one of ordinary skill in the art, groups such as —H, halogen, —NO₂, —CN, —OH, —NH₂ or —OCF₃ would not be substitutable groups.

The phrase “up to”, as used herein, refers to zero or any integer number that is equal or less than the number following the phrase. For example, “up to 3” means any one of 0, 1, 2, or 3. As described herein, a specified number range of atoms includes any integer therein. For example, a group having from 1-4 atoms could have 1, 2, 3 or 4 atoms. It will be understood by one of ordinary skill in the art that when a group is characterized as substituted (as opposed to optionally substituted) with, e.g., “up to 3” substituents, it can only be substituted with 1, 2 or 3 substituents.

When any variable occurs more than one time at any position, its definition on each occurrence is independent from every other occurrence.

Selection of substituents and combinations envisioned by this disclosure are only those that result in the formation of stable or chemically feasible compounds. Such choices and combinations will be apparent to those of ordinary sill in the art and may be determined without undue experimentation. The term “stable”, as used herein, refers to compounds that are not substantially altered when subjected to conditions to allow for their production, detection, and, in some embodiments, their recovery, purification, and use for one or more of the purposes disclosed herein. In some embodiments, a stable compound or chemically feasible compound is one that is not substantially altered when kept at a temperature of 25° C. or less, in the absence of moisture or other chemically reactive conditions, for at least a week.

A compound, such as the compounds of the invention or other compounds herein disclosed, may be present in its free form (e.g. an amorphous form, a crystalline form or polymorphs). Under certain conditions, compounds may also form salts, and/or other multi-component crystalline forms (e.g. solvates, hydrates and co-crystals). As used herein, the term co-form is synonymous with the term multi-component crystalline form. When one of the components in the co-form has clearly transferred a proton to the other component, the resulting co-form is referred to as a “salt”. When both compounds in a multi-component crystalline form are independently solids at room temperature, the resulting co-form is referred to as a “co-crystal”. In co-crystals no proton transfer takes place between the different components of the co-form. The formation of a salt or a co-crystal is determined by how large is the difference in the pKas between the partners that form the mixture. As used herein, a “solvate” refers to an association or complex of one or more solvent molecules and a compound disclosed herein (or its salts or co-crystals). A “hydrate” is a particular type of solvate in which the solvent is water. Examples of solvents that can form solvates include, but are not limited to: water, isopropanol, ethanol, methanol, DMSO (dimethyl sulfoxide), ethyl acetate, acetic acid, ethanolamine, tetrahydrofuran (THF), dichloromethane (DCM), N,N-dimethylformamide (DMF).

Unless only one of the isomers is drawn or named specifically, structures depicted herein are also meant to include all stereoisomeric (e.g., enantiomeric, diastereomeric, atropoisomeric and cis-trans isomeric) forms of the structure; for example, the R and S configurations for each asymmetric center, Ra and Sa configurations for each asymmetric axis, (Z) and (E) double bond configurations, and cis and trans conformational isomers. Therefore, single stereochemical isomers as well as racemates, and mixtures of enantiomers, diastereomers, and cis-trans isomers (double bond or conformational isomers) of the present compounds are within the scope of the present disclosure. Unless otherwise stated, all tautomeric forms of the compounds of the present disclosure are within the scope of the disclosure.

The present disclosure also embraces isotopically-labeled compounds which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses. Exemplary isotopes that can be incorporated into compounds of the invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S ¹⁸F, ³⁶Cl, ¹²³I, and ¹²⁵I, respectively. Certain isotopically-labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate receptor occupancy. Isotopically labeled compounds of the present disclosure can generally be prepared by following procedures analogous to those disclosed in the Schemes and/or in the Examples herein, by substituting an isotopically labeled reagent for a non-isotopically labeled reagent.

The term “aliphatic” or “aliphatic group”, as used herein, means a straight-chain (i.e., unbranched) or branched, substituted or unsubstituted hydrocarbon chain that is completely saturated or that contains one or more units of unsaturation. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms. In other embodiments, aliphatic groups contain 1-4 aliphatic carbon atoms and in yet other embodiments, aliphatic groups contain 1-3 aliphatic carbon atoms. Suitable aliphatic groups include, but are not limited to, linear or branched, substituted or unsubstituted alkyl, alkenyl, or alkynyl groups. Specific examples of aliphatic groups include, but are not limited to: methyl, ethyl, propyl, butyl, isopropyl, isobutyl, vinyl, sec-butyl, tert-butyl, butenyl, propargyl, acetylene and the like.

The term “alkyl”, as used herein, refers to a saturated linear or branched-chain monovalent hydrocarbon radical. Unless otherwise specified, an alkyl group contains 1-20 carbon atoms (e.g., 1-20 carbon atoms, 1-10 carbon atoms, 1-8 carbon atoms, 1-6 carbon atoms, 1-4 carbon atoms or 1-3 carbon atoms). Examples of alkyl groups include, but are not limited to, methyl, ethyl, n-propyl, isopropyl, n-butyl, isobutyl, s-butyl, t-butyl, pentyl, hexyl, heptyl, octyl and the like.

The term “alkenyl” refers to a linear or branched-chain monovalent hydrocarbon radical with at least one site of unsaturation, i.e., a carbon-carbon, sp² double bond, wherein the alkenyl radical includes radicals having “cis” and “trans” orientations, or alternatively, “E” and “Z” orientations. Unless otherwise specified, an alkenyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, vinyl, allyl and the like.

The term “alkynyl” refers to a linear or branched monovalent hydrocarbon radical with at least one site of unsaturation, i.e., a carbon-carbon sp triple bond. Unless otherwise specified, an alkynyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, ethynyl, propynyl, and the like.

The term “carbocyclic” refers to a ring system formed only by carbon and hydrogen atoms. Unless otherwise specified, throughout this disclosure, carbocycle is used as a synonym of “non-aromatic carbocycle” or “cycloaliphatic”. In some instances the term can be used in the phrase “aromatic carbocycle”, and in this case it refers to an “aryl group” as defined below.

The term “cycloaliphatic” (or “non-aromatic carbocycle”, “non-aromatic carbocyclyl”, “non-aromatic carbocyclic”) refers to a cyclic hydrocarbon that is completely saturated or that contains one or more units of unsaturation but which is not aromatic, and which has a single point of attachment to the rest of the molecule. Unless otherwise specified, a cycloaliphatic group may be monocyclic, bicyclic, tricyclic, fused, Spiro or bridged. In one embodiment, the term “cycloaliphatic” refers to a monocyclic C₃-C₁₂ hydrocarbon or a bicyclic C₇-C₁₂ hydrocarbon. In some embodiments, any individual ring in a bicyclic or tricyclic ring system has 3-7 members. Suitable cycloaliphatic groups include, but are not limited to, cycloalkyl, cycloalkenyl, and cycloalkynyl. Examples of aliphatic groups include cyclopropyl, cyclobutyl, cyclopentyl, cyclopentenyl, cyclohexyl, cyclohexenyl, cycloheptyl, cycloheptenyl, norbornyl, cyclooctyl, cyclononyl, cyclodecyl, cycloundecyl, cyclododecyl, and the like.

The term “cycloaliphatic” also includes polycyclic ring systems in which the non-aromatic carbocyclic ring can be “fused” to one or more aromatic or non-aromatic carbocyclic or heterocyclic rings or combinations thereof, as long as the radical or point of attachment is on the non-aromatic carbocyclic ring.

“Heterocycle” (or “heterocyclyl” or “heterocyclic), as used herein, refers to a ring system in which one or more ring members are an independently selected heteroatom, which is completely saturated or that contains one or more units of unsaturation but which is not aromatic, and which has a single point of attachment to the rest of the molecule. Unless otherwise specified, through this disclosure, heterocycle is used as a synonym of “non-aromatic heterocycle”. In some instances the term can be used in the phrase “aromatic heterocycle”, and in this case it refers to a “heteroaryl group” as defined below. The team heterocycle also includes fused, Spiro or bridged heterocyclic ring systems. Unless otherwise specified, a heterocycle may be monocyclic, bicyclic or tricyclic. In some embodiments, the heterocycle has 3-18 ring members in which one or more ring members is a heteroatom independently selected from oxygen, sulfur or nitrogen, and each ring in the system contains 3 to 7 ring members. In other embodiments, a heterocycle may be a monocycle having 3-7 ring members (2-6 carbon atoms and 1-4 heteroatoms) or a bicycle having 7-10 ring members (4-9 carbon atoms and 1-6 heteroatoms). Examples of bicyclic heterocyclic ring systems include, but are not limited to: adamantanyl, 2-oxa-bicyclo[2.2.2]octyl, 1-aza-bicyclo[2.2.2]octyl.

As used herein, the term “heterocycle” also includes polycyclic ring systems wherein the heterocyclic ring is fused with one or more aromatic or non-aromatic carbocyclic or heterocyclic rings, or with combinations thereof, as long as the radical or point of attachment is in the heterocyclic ring.

Examples of heterocyclic rings include, but are not limited to, the following monocycles: 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl; and the following bicycles: 3-1H-benzimidazol-2-one, 3-(1-alkyl)-benzimidazol-2-one, indolinyl, tetrahydroquinolinyl, tetrahydroisoquinolinyl, benzothiolane, benzodithiane, and 1,3-dihydro-imidazol-2-one.

As used herein, the term “aryl” (as in “aryl ring” or “aryl group”), used alone or as part of a larger moiety, as in “aralkyl”, “aralkoxy”, “aryloxyalkyl”, refers to a carbocyclic ring system wherein at least one ring in the system is aromatic and has a single point of attachment to the rest of the molecule. Unless otherwise specified, an aryl group may be monocyclic, bicyclic or tricyclic and contain 6-18 ring members. The term also includes polycyclic ring systems where the aryl ring is fused with one or more aromatic or non-aromatic carbocyclic or heterocyclic rings, or with combinations thereof, as long as the radical or point of attachment is in the aryl ring. Examples of aryl rings include, but are not limited to, phenyl, naphthyl, indanyl, indenyl, tetralin, fluorenyl, and anthracenyl.

The term “heteroaryl” (or “heteroaromatic” or “heteroaryl group” or “aromatic heterocycle”) used alone or as part of a larger moiety as in “heteroaralkyl” or “heteroarylalkoxy” refers to a ring system wherein at least one ring in the system is aromatic and contains one or more heteroatoms, wherein each ring in the system contains 3 to 7 ring members and which has a single point of attachment to the rest of the molecule. Unless otherwise specified, a heteroaryl ring system may be monocyclic, bicyclic or tricyclic and have a total of five to fourteen ring members. In one embodiment, all rings in a heteroaryl system are aromatic. Also included in this definition are heteroaryl radicals where the heteroaryl ring is fused with one or more aromatic or non-aromatic carbocyclic or heterocyclic rings, or combinations thereof, as long as the radical or point of attachment is in the heteroaryl ring. A bicyclic 6,5 heteroaromatic system, as used herein, for example, is a six membered heteroaromatic ring fused to a second five membered ring wherein the radical or point of attachment is on the six membered ring.

Heteroaryl rings include, but are not limited to the following monocycles: 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, pyrazinyl, 1,3,5-triazinyl, and the following bicycles: benzimidazolyl, benzofuryl, benzothiophenyl, benzopyrazinyl, benzopyranonyl, indolyl (e.g., 2-indolyl), purinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

As used herein, “cyclo” (or “cyclic”, or “cyclic moiety”) encompasses mono-, bi- and tri-cyclic ring systems including cycloaliphatic, heterocyclic, aryl or heteroaryl, each of which has been previously defined.

“Fused” bicyclic ring systems comprise two rings which share two adjoining ring atoms.

“Bridged” bicyclic ring systems comprise two rings which share three or four adjacent ring atoms. As used herein, the term “bridge” refers to a bond or an atom or a chain of atoms connecting two different parts of a molecule. The two atoms that are connected through the bridge (usually but not always, two tertiary carbon atoms) are referred to as “bridgeheads”. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl.

“Spiro” bicyclic ring systems share only one ring atom (usually a quaternary carbon atom).

The term “ring atom” refers to an atom such as C, N, O or S that is part of the ring of an aromatic group, a cycloaliphatic group or a heteroaryl ring. A “substitutable ring atom” is a ring carbon or nitrogen atom bonded to at least one hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to one or more moiety other than hydrogen and no hydrogens are available for substitution.

“Heteroatom” refers to one or more of oxygen, sulfur, nitrogen, phosphorus, or silicon, including any oxidized form of nitrogen, sulfur, phosphorus, or silicon, the quaternized form of any basic nitrogen, or a substitutable nitrogen of a heterocyclic or heteroaryl ring, for example N (as in 3,4-dihydro-2H-pyrrolyl), NH (as in pyrrolidinyl) or NR⁺ (as in N-substituted pyrrolidinyl).

In some embodiments, two independent occurrences of a variable may be taken together with the atom(s) to which each variable is bound to form a 5-8-membered, heterocyclyl, aryl, or heteroaryl ring or a 3-8-membered cycloalkyl ring. Exemplary rings that are formed when two independent occurrences of a substituent are taken together with the atom(s) to which each variable is bound include, but are not limited to the following: a) two independent occurrences of a substituent that are bound to the same atom and are taken together with that atom to form a ring, where both occurrences of the substituent are taken together with the atom to which they are bound to form a heterocyclyl, heteroaryl, carbocyclyl or aryl ring, wherein the group is attached to the rest of the molecule by a single point of attachment; and b) two independent occurrences of a substituent that are bound to different atoms and are taken together with both of those atoms to form a heterocyclyl, heteroaryl, carbocyclyl or aryl ring, wherein the ring that is formed has two points of attachment with the rest of the molecule. For example, where a phenyl group is substituted with two occurrences of R^{o} as in Formula D1:

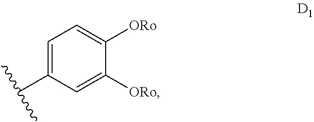

these two occurrences of R^{o} are taken together with the oxygen atoms to which they are bound to form a fused 6-membered oxygen containing ring as in Formula D2:

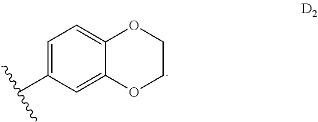 [SMILES: *c1ccc2c(c1)OCCO2]

It will be appreciated that a variety of other rings can be formed when two independent occurrences of a substituent are taken together with the atom(s) to which each substituent is bound and that the examples detailed above are not intended to be limiting.

In some embodiments, an alkyl or aliphatic chain can be optionally interrupted with another atom or group. This means that a methylene unit of the alkyl or aliphatic chain can optionally be replaced with said other atom or group. Unless otherwise specified, the optional replacements form a chemically stable compound. Optional interruptions can occur both within the chain and/or at either end of the chain; i.e. both at the point of attachment(s) to the rest of the molecule and/or at the terminal end. Two optional replacements can also be adjacent to each other within a chain so long as it results in a chemically stable compound. Unless otherwise specified, if the replacement or interruption occurs at a terminal end of the chain, the replacement atom is bound to an H on the terminal end. For example, if —CH₂CH₂CH₃ were optionally interrupted with —O—, the resulting compound could be —OCH₂CH₃, —CH₂OCH₃, or —CH₂CH₂OH. In another example, if the divalent linker —CH₂CH₂CH₂— were optionally interrupted with —O—, the resulting compound could be —OCH₂CH₂—, —CH₂OCH₂—, or —CH₂CH₂O—. The optional replacements can also completely replace all of the carbon atoms in a chain. For example, a C₃ aliphatic can be optionally replaced by —N(R^{$})—, —C(O)— and —N(R^{$})— to form —N(R^{$})C(O)N(R^{$})— (a urea).

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent at the end of the substituent bound to the rest of the chemical structure. Alkylcarboxy (e.g., alkyl-C(O)O— or alkyl-O(CO)—) and alkylcarboxyaryl (e.g., alkyl-C(O)O-aryl- or alkyl-O(CO)-aryl-) are examples of carboxy groups used internally.

As described herein, a bond drawn from a substituent to the center of one ring within a multiple-ring system (as shown below), represents substitution of the substituent at any substitutable position in any of the rings within the multiple ring system. For example, formula D3 represents possible substitution in any of the positions shown in formula D4:

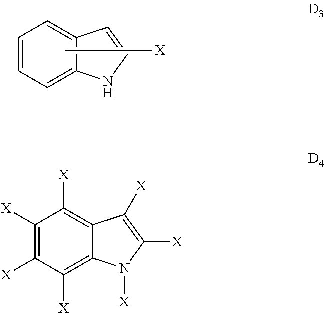

This also applies to multiple ring systems fused to optional ring systems (which would be represented by dotted lines). For example, in Formula D5, X is an optional substituent both for ring A and ring B.

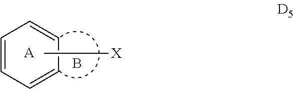

If, however, two rings in a multiple ring system each have different substituents drawn from the center of each ring, then, unless otherwise specified, each substituent only represents substitution on the ring to which it is attached. For example, in Formula D6, Y is an optional substituent for ring A only, and X is an optional substituent for ring B only.

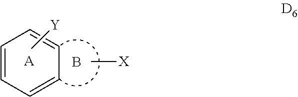

As used herein, the terms “alkoxy” or “alkylthio” refer to an alkyl group, as previously defined, attached to the molecule, or to another chain or ring, through an oxygen (“alkoxy” i.e., —O-alkyl) or a sulfur (“alkylthio” i.e., —S-alkyl) atom.

The terms Cₙ₋ₘ, “alkoxyalkyl”, Cₙ₋ₘ“alkoxyalkenyl”, Cₙ₋ₘ “alkoxyaliphatic”, and Cₙ₋ₘ “alkoxyalkoxy” mean alkyl, alkenyl, aliphatic or alkoxy, as the case may be, substituted with one or more alkoxy groups, wherein the combined total number of carbons of the alkyl and alkoxy groups, alkenyl and alkoxy groups, aliphatic and alkoxy groups or alkoxy and alkoxy groups, as the case may be, is between the values of n and m. For example, a C₄₋₆ alkoxyalkyl has a total of 4-6 carbons divided between the alkyl and alkoxy portion; e.g. it can be —CH₂OCH₂CH₂CH₃, —CH₂CH₂OCH₂CH₃ or —CH₂CH₂CH₂OCH₃.

When the moieties described in the preceding paragraph are optionally substituted, they can be substituted in either or both of the portions on either side of the oxygen or sulfur. For example, an optionally substituted C₄ alkoxyalkyl could be, for instance, —CH₂CH₂OCH₂(Me)CH₃ or —CH₂(OH)OCH₂CH₂CH₃; a C₅ alkoxyalkenyl could be, for instance, —CH═CHOCH₂CH₂CH₃ or —CH═CHCH₂OCH₂CH₃.

The terms aryloxy, arylthio, benzyloxy or benzylthio, refer to an aryl or benzyl group attached to the molecule, or to another chain or ring, through an oxygen (“aryloxy”, benzyloxy e.g., —O-Ph, —OCH₂Ph) or sulfur (“arylthio” e.g., —S-Ph, —S—CH₂Ph) atom. Further, the terms “aryloxyalkyl”, “benzyloxyalkyl” “aryloxyalkenyl” and “aryloxyaliphatic” mean alkyl, alkenyl or aliphatic, as the case may be, substituted with one or more aryloxy or benzyloxy groups, as the case may be. In this case, the number of atoms for each aryl, aryloxy, alkyl, alkenyl or aliphatic will be indicated separately. Thus, a 5-6-membered aryloxy(C₁₋₄ alkyl) is a 5-6 membered aryl ring, attached via an oxygen atom to a C₁₋₄ alkyl chain which, in turn, is attached to the rest of the molecule via the terminal carbon of the C₁₋₄ alkyl chain.

An “aralkyl” or “alkyl aryl” refers to an aryl ring attached to an alkyl chain, wherein the point of attachment is on the alkyl chain. Unless otherwise indicated, as used in this disclosure, an optionally substituted aralkyl is optionally substituted only in the aryl portion. The same principle applies to, for example, an optionally substituted aralkoxy (i.e. an aryl ring attached to an alkoxy), which would be attached to the rest of the molecule through the oxygen of the alkoxy and substituted on the aryl portion. A substituted aryloxyalkyl would be attached to the rest of the molecule through the alkyl chain and substituted on the aryl ring, and the aryl and alkyl would, in turn, be attached to each other through an oxygen atom. For example, an optionally substituted 6-membered aryloxy(C₃ alkyl) group could be, for instance, —(CH₃)CH₂-[p-(MeO)-Ph]; an optionally substituted 6-membered heteroaryloxy(C₄ alkyl) could, for instance, be —CH₂CH₂CH₂CH₂—O-(3-F-2-pyridyl) or —CH(CH₃)—O—CH₂CH₂-(5,6-dimethyl-1,3-pyrimidine).

If an alkyl chain on the “aralkyl” group is also optionally substituted that will be specifically indicated. For instance an optionally substituted 6-membered heteroaryloxy(C₄ alkyl) which is also optionally substituted on the alkyl, would be referred to as “an optionally substituted 6-membered heteroaryloxy(C₄ alkyl), wherein said C₄ alkyl chain is optionally substituted”. An example of this latter group could be —CH(OH)—CF(CH₃)—CH₂—O-(5,6-dimethyl-1,3-pyrimidine) wherein the alkyl chain is substituted with F and with —OH.

As used herein, the terms “halogen” or “halo” mean F, Cl, Br, or I.

The terms “haloalkyl”, “haloalkenyl”, “haloaliphatic”, and “haloalkoxy” mean alkyl, alkenyl, aliphatic or alkoxy, as the case may be, substituted with one or more halogen atoms. For example a C₁₋₃ haloalkyl could be —CFHCH₂CHF₂ and a C₁₋₂ haloalkoxy could be —OC(Br)HCHF₂. This term includes perfluorinated alkyl groups, such as —CF₃ and —CF₂CF₃.

As used herein, the term “cyano” refers to —CN or —C≡N.

The terms “cyanoalkyl”, “cyanoalkenyl”, “cyanoaliphatic”, and “cyanoalkoxy” mean alkyl, alkenyl, aliphatic or alkoxy, as the case may be, substituted with one or more cyano groups. For example a C₁₋₃ cyanoalkyl could be —C(CN)₂CH₂CH₃ and a C₁₋₂ cyanoalkenyl could be ═CHC(CN)H₂.

As used herein, an “amino” group refers to —NH₂.

The terms “aminoalkyl”, “aminoalkenyl”, “aminoaliphatic”, and “aminoalkoxy” mean alkyl, alkenyl, aliphatic or alkoxy, as the case may be, substituted with one or more amino groups. For example a C₁₋₃ aminoalkyl could be —CH(NH₂)CH₂CH₂NH₂ and a C₁₋₂ aminoalkoxy could be —OCH₂CH₂NH₂.

The term “hydroxyl” or “hydroxy” refers to —OH.

The terms “hydroxyalkyl”, “hydroxyalkenyl”, “hydroxyaliphatic”, and “hydroxyalkoxy” mean alkyl, alkenyl, aliphatic or alkoxy, as the case may be, substituted with one or more —OH groups. For example a C₁₋₃ hydroxyalkyl could be —CH₂(CH₂OH)CH₃ and a C₄ hydroxyalkoxy could be —OCH₂C(CH₃)(OH)CH₃.

As used herein, an “aroyl” or “heteroaroyl” refers to a —C(O)-aryl or a —C(O)-heteroaryl. The aryl and heteroaryl portion of the aroyl or heteroaroyl is optionally substituted as previously defined.

As used herein, a “carbonyl”, used alone or in connection with another group refers to —C(O)— or —C(O)H. For example, as used herein, an “alkoxycarbonyl,” refers to a group such as —C(O)O(alkyl).

As used herein, an “oxo” refers to ═O, wherein oxo is usually, but not always, attached to a carbon atom. An aliphatic chain can be optionally interrupted by a carbonyl group or can optionally be substituted by an oxo group, and both expressions refer to the same: e.g. —CH₂—C(O)—CH₃.

As used herein, in the context of resin chemistry (e.g. using solid resins or soluble resins or beads), the term “linker” refers to a bifunctional chemical moiety attaching a compound to a solid support or soluble support.

In all other situations, a “linker”, as used herein, refers to a divalent group in which the two free valences are on different atoms (e.g. carbon or heteroatom) or are on the same atom but can be substituted by two different substituents. For example, a methylene group can be C₁ alkyl linker (—CH₂—) which can be substituted by two different groups, one for each of the free valences (e.g. as in Ph-CH₂-Ph, wherein methylene acts as a linker between two phenyl rings). Ethylene can be C₂ alkyl linker (—CH₂CH₂—) wherein the two free valences are on different atoms. The amide group, for example, can act as a linker when placed in an internal position of a chain (e.g. —CONH—). A linker can be the result of interrupting an aliphatic chain by certain functional groups or of replacing methylene units on said chain by said functional groups. E.g. a linker can be a C₁₋₆ aliphatic chain in which up to two methylene units are substituted by —C(O)— or —NH—(as in —CH₂—NH—CH₂—C(O)—CH₂— or —CH₂—NH—C(O)—CH₂—). An alternative way to define the same —CH₂—NH—CH₂—C(O)—CH₂— and —CH₂—NH—C(O)—CH₂— groups is as a C₃ alkyl chain optionally interrupted by up to two —C(O)— or —NH— moieties. Cyclic groups can also form linkers: e.g. a 1,6-cyclohexanediyl can be a linker between two R groups, as in

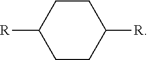

A linker can additionally be optionally substituted in any portion or position.

Divalent groups of the type R—CH═ or R₂C═, wherein both free valences are in the same atom and are attached the same substituent, are also possible. In this case, they will be referred to by their IUPAC accepted names. For instance an alkylidene (such as, for example, a methylidene (═CH₂) or an ethylidene (═CH—CH₃)) would not be encompassed by the definition of a linker in this disclosure.

The term “protecting group”, as used herein, refers to an agent used to temporarily block one or more desired reactive sites in a multifunctional compound. In certain embodiments, a protecting group has one or more, or preferably all, of the following characteristics: a) reacts selectively in good yield to give a protected substrate that is stable to the reactions occurring at one or more of the other reactive sites; and b) is selectively removable in good yield by reagents that do not attack the regenerated functional group. Exemplary protecting groups are detailed in Greene, T. W., Wuts, P. G in “Protective Groups in Organic Synthesis”, Third Edition, John Wiley & Sons, New York: 1999, the entire contents of which are hereby incorporated by reference. The term “nitrogen protecting group”, as used herein, refers to an agents used to temporarily block one or more desired nitrogen reactive sites in a multifunctional compound. Preferred nitrogen protecting groups also possess the characteristics exemplified above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

As used herein, the term “displaceable moiety” or “leaving group” refers to a group that is associated with an aliphatic or aromatic group as defined herein and is subject to being displaced by nucleophilic attack by a nucleophile.

As used herein, “amide coupling agent” or “amide coupling reagent” means a compound that reacts with the hydroxyl moiety of a carboxy moiety thereby rendering it susceptible to nucleophilic attack. Exemplary amide coupling agents include DIC (diisopropylcarbodiimide), EDCI (1-Ethyl-3-(3-dimethylaminopropyl)carbodiimide), DCC (dicyclohexylcarbodiimide), BOP (Benzotriazol-1-yloxy-tris(dimethylamino)-phosphonium hexafluorophosphate), pyBOP ((Benzotriazol-1-yloxy)tripyrrolidinophosphonium Hexafluorophosphate), etc.

The compounds of the invention are defined herein by their chemical structures and/or chemical names. Where a compound is referred to by both a chemical structure and a chemical name, and the chemical structure and chemical name conflict, the chemical structure is determinative of the compound's identity.

In one aspect, the invention is a compound of formula I

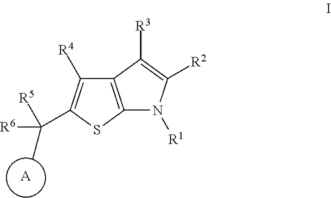

wherein

R¹ is V—R⁸;

V is a covalent bond between R⁸ and the nitrogen to which V is bonded, or is a divalent linker between R⁸ and the nitrogen to which V is bonded, wherein said linker is a saturated or unsaturated C₁₋₆ aliphatic which is optionally substituted with up to 6 instances of halogen, a C₁₋₄ aliphatic, —OR¹⁴, —CN, —SR¹⁴, —CO₂R¹⁴, —OC(O)R¹⁴, —C(O)N(R¹⁴H)₂, —N(R)C(O)R¹⁴, —N(R)C(O)OR¹⁴, —OC(O)N(R¹⁴H)₂, —N(R)C(O)NR¹⁴ or —N(R¹⁴H)₂; wherein said C₁₋₄ aliphatic is optionally substituted with up to 6 instances of halogen, —OR¹⁴, —CN or —N(R¹⁴H)₂; and wherein up to two saturated carbons of said C₁₋₆ aliphatic are replaced by —O—, —C(O)—, —C(S)—, —C(O)N(R)—, —N(R)C(O)—, —C(O)O—, —OC(O)—, —C(═N—N(R¹⁴H)₂)—, —C(═N—OR¹⁴H)—, —N(R)—, —N(R)S(O)₂—, —S(O)₂N(R)—, —N(R)S(O)₂N(R)—, —N(R)C(O)O—, —OC(O)N(R)—, —N(R)C(O)N(R)—, —OC(O)N(R)—, —S—, —S(O)—, —S(O)₂—, —C(O)S—, —SC(O)—, —C(S)S—, —SC(S)—, —OC(S)—, —C(S)O—, —C(S)N(R)—, —N(R)C(S)—, —N(R)C(S)S—, —SC(S)N(R)—, —N(R)C(S)O—, —N(R)C(O)S—, —OC(S)N(R)— or —SC(O)N(R)—;

each occurrence of R is independently selected from hydrogen, a C₁₋₄ aliphatic or a C₁₋₄ haloaliphatic;

each occurrence of R¹⁴ is independently selected from hydrogen, a C₁₋₄ aliphatic, a C₁₋₄ haloaliphatic, a C₃₋₇ cycloaliphatic or a 3-7 membered heterocyclyl; or two R¹⁴ attached to the same or different atom(s), together with the atom(s) to which they are attached, form a C₃₋₇ cycloaliphatic or 3-7 membered heterocycle; wherein said cycloaliphatic and heterocyclyl are optionally and independently substituted by up to 6 instances of halogen, —CN, oxo, C₁₋₄ alkyl, C₁₋₄ haloalkyl, C₁₋₄ alkoxy or C₁₋₄ haloalkoxy;

R⁸ is hydrogen, halogen, —NO₂, —CN, a C₁-C₆ aliphatic, a 5-14 membered aryl group, a 5-14 membered heteroaryl group, a C₃-C₁₂ cycloaliphatic group, or a 3-14 membered heterocyclyl group; wherein said aliphatic, aryl, heteroaryl, cycloaliphatic and heterocyclyl group are optionally and independently substituted with up to 6 instances of R¹⁵;

each occurrence of R¹⁵ is independently selected from halogen, oxo, —NO₂, —CN, —OR¹⁷, —SR¹⁷, —S(O)₂R¹⁷, —SO₂N(R¹⁷)₂, —S(O)R¹⁷, —N(R¹⁷)₂, —C(O)OR¹⁷, —C(O)R¹⁷, —C(O)C(O)R¹⁷, —C(═N—N(R¹⁷)₂)R¹⁷, —N(R′)C(O)R¹⁷, —N(R′)C(O)OR¹⁷, —N(R′)S(O)₂R¹⁷, —N(R′)S(O)₂N(R′)R¹⁷, —N(R′)C(O)N(R′)R¹⁷, —N(R′)N(R′)R¹⁷, —C(O)NOR¹⁷, —C(O)N(R¹⁷)₂, —OC(O)R¹⁷, —OC(O)N(R¹⁷)₂, —C(O)SR¹⁷, —SC(O)R¹⁷, —C(S)SR¹⁷, —SC(S)R¹⁷, —OC(S)R¹⁷, —C(S)OR¹⁷, —C(S)N(R′)R¹⁷, —N(R′)C(S)R¹⁷, —N(R′)C(S)SR¹⁷, —SC(S)N(R′)R¹⁷, —N(R′)C(S)OR¹⁷, —N(R′)C(O)SR¹⁷, —OC(S)N(R′)R¹⁷, —SC(O)N(R′)R¹⁷, or a C₁₋₄ aliphatic, wherein said aliphatic is optionally substituted with up to 6 instances of —R¹⁶;

each occurrence of R′ is independently selected from hydrogen, C₁₋₄ aliphatic or C₁₋₄ haloaliphatic;

each occurrence of R¹⁶ is independently selected from halogen, oxo, —OR¹⁸, —CN, —CO₂R¹⁸, —C(O)N(R¹⁸)₂, —N(R″)C(O)R¹⁸, —OC(O)N(R¹⁸)₂, —N(R″)C(O)OR¹⁸, —N(R¹⁸)C(O)N(R)₂, —N(R¹⁸)₂, a C₃₋₇ cycloaliphatic or a 3-7 membered heterocycle; wherein said cycloaliphatic and heterocycle can be optionally and independently substituted by up to 6 instances of halogen, —CN, oxo, C₁₋₄ alkyl, C₁₋₄ haloalkyl, C₁₋₄ alkoxy or C₁₋₄ haloalkoxy;

each occurrence of R¹⁷ is independently selected from hydrogen, a C₁-C₄ aliphatic, a C₁-C₄ haloaliphatic, a C₃₋₇ cycloaliphatic or a 3-7 membered heterocyclyl; or two R¹⁷ attached to the same or different atom(s), together with the atom(s) to which they are attached, form a 3-7 membered heterocycle; wherein said cycloaliphatic and heterocyclyl are optionally and independently substituted by up to 6 instances of halogen, —CN, oxo, C₁₋₄ alkyl, C₁₋₄ haloalkyl, C₁₋₄ alkoxy or C₁₋₄ haloalkoxy;

each occurrence of R″ is independently selected from hydrogen, C₁₋₄ aliphatic or C₁₋₄ haloaliphatic;

each occurrence of R¹⁸ is independently selected from hydrogen, alkyl aryl, a C₁₋₄ aliphatic or a C₁-C₄ haloaliphatic; or two R¹⁸ attached to the same or different atom(s), together with the atom(s) to which they are attached, form a 3-7 membered heterocycle; wherein said 3-7 membered heterocyclyl ring is optionally substituted by up to 6 instances of halogen, —CN, oxo, C₁₋₄ alkyl, C₁₋₄ haloalkyl, C₁₋₄ alkoxy or C₁₋₄ haloalkoxy;

R² is hydrogen, halogen, —CN, —NO₂, a C₁₋₄ aliphatic or a C₃₋₆ cycloaliphatic, wherein said aliphatic and cycloaliphatic are optionally and independently substituted with up to 6 instances of halogen, —CN, —OH, —O(C₁₋₂ alkyl), —O(C₁₋₂ haloalkyl), C₁₋₂ alkyl or C₁₋₂ haloalkyl;

R³ is hydrogen, halogen, —CN, —NO₂, —C(O)NR^{X}, —C(O)OR^{X}, a C₁₋₄ aliphatic or a C₃₋₆ cycloaliphatic, wherein said aliphatic and cycloaliphatic are optionally and independently substituted with up to 6 instances of halogen, —CN, —OH, —O(C₁₋₂ alkyl), —O(C₁₋₂ haloalkyl), C₁₋₂ alkyl or C₁₋₂ haloalkyl;

R⁴ is hydrogen, halogen, —CN, —NO₂, —C(O)NR^{X}, a C₁₋₄ aliphatic or a C₃₋₆ cycloaliphatic, wherein said aliphatic and cycloaliphatic are independently and optionally substituted with up to 6 instances of halogen, —CN, —OH, —O(C₁₋₂ alkyl), —O(C₁₋₂ haloalkyl), —C₁₋₂ alkyl or —C₁₋₂₂ haloalkyl.

R⁵ is chosen from hydrogen, halogen, —CN, —OH, —C(O)OR^{X}, C₁₋₄ aliphatic, a 3-7 membered heterocyclyl, C₃₋₆ cycloaliphatic, —O—(C₁₋₄ aliphatic) or —O—(C₃₋₆ cycloaliphatic); wherein said aliphatic, cycloaliphatic, —O—(C₁₋₄ aliphatic) and —O—(C₃₋₆ cycloaliphatic) are optionally and independently substituted with up to three instances of halogen;

R⁶ is chosen from hydrogen, halogen, C₁₋₄ aliphatic, C₁₋₄ haloaliphatic, —O(C₁₋₄ aliphatic) or —O—(C₁₋₄ haloaliphatic); or

R⁵ and R⁶ are taken together to form ═O, ═S, ═NR^{W} or a 3-6 membered cycloaliphatic or heterocyclyl, wherein said cycloaliphatic or heterocyclyl is optionally and independently substituted with up to 6 instances of halogen, —CN, —OH, —O(C₁₋₄ aliphatic), —NO₂, —N(R^{Y}), or a C₁₋₄ aliphatic; wherein said aliphatic is optionally and independently substituted with up to 6 instances of —OR^{Y} or halogen;

R^{W} is selected from hydrogen, C₁₋₄ alkyl C₁₋₄ haloalkyl, C₃₋₆ cycloalkyl, —O(C₁₋₄ alkyl), —O(C₁₋₄ haloalkyl), —N(C₁₋₄ alkyl)₂, —N(C₁₋₄ haloalkyl)₂ or —O(aryl); wherein said aryl is optionally substituted by up to 6 instances of halogen, —NO₂, C₁₋₄ alkyl, C₁₋₄ haloalkyl, —O(C₁₋₄ alkyl), or —O(C₁₋₄ haloalkyl);

each occurrence of R^{X} and R^{Y} are independently selected from hydrogen or a C₁₋₄ aliphatic, wherein said aliphatic is optionally and independently substituted with up to three instances of halogen, OR^{iv} or N(R^{iv})₂;

each occurrence of R^{iv} is independently selected from hydrogen, C₁₋₄ aliphatic or C₁₋₄ haloaliphatic;

ring A is phenyl or a 5-6 membered monocyclic heteroaryl having 1-3 heteroatoms independently selected from N, O or S; wherein said phenyl and heteroaryl are optionally and independently substituted with up to five instances of R¹²;

each occurrence of R¹² is independently selected from halogen, —NO₂, —CN, a C₁₋₄ aliphatic optionally substituted with up to four instances of R¹⁹, —OR¹³, —SR¹³, —S(O)₂R¹³, —SO₂N(R¹³)₂, —S(O)R¹³, —N(R¹³)₂, —C(O)OR¹³, —C(O)R¹³, —C(O)C(O)R¹³, —C(═N—N(R¹³)₂)R¹³, —N(R¹³)C(O)R¹³, —N(R¹³)C(O)OR¹³, —N(R¹³)C(S)R¹³, —N(R¹³)C(S)OR¹³, —N(R¹³)S(O)₂R¹³, —N(R¹³)S(O)₂N(R¹³)R¹³, —N(R¹³)C(O)N(R¹³)R¹³, —N(R¹³)C(S)N(R¹³)R¹³, —N(R¹³)N(R¹³)R¹³, —C(O)NOR¹³, —C(O)NR¹³)₂, —C(O)N(R¹³)N(R¹³)₂, —C(S)N(R¹³)₂, —C(S)(R¹³), —C(S)OR¹³, —C(S)N(R¹³)N(R¹³)₂, —OC(O)R¹³ or —OC(O)N(R¹³)₂; or two R¹² attached to different atom(s), together with the atom(s) to which they are attached, form a 3-7 membered heterocycle or C₃₋₇ cycloaliphatic; wherein said cycloaliphatic and heterocycle are independently and optionally substituted with up to 6 instances of halogen, —CN, —OH, C₁₋₂ alkyl, C₁₋₂ haloalkyl, C₁₋₂ alkoxy or C₁₋₂ haloalkoxy;

each R¹³ is independently selected from hydrogen, a C₁-C₄ aliphatic, a C₃₋₇ cycloaliphatic or a 3-7 membered heterocyclyl, wherein said aliphatic, cycloaliphatic and heterocyclyl are independently and optionally substituted with up to 6 instances of halogen; or two R¹³ attached to the same or different atom(s), together with the atom(s) to which they are attached, form a 3-7 membered heterocycle; wherein said heterocycle is optionally substituted with up to 6 instances of halogen, —CN, —OH, C₁₋₂ alkyl, C₁₋₂ haloalkyl, C₁₋₂ alkoxy or C₁₋₂ haloalkoxy;

each occurrence of R¹⁹ is independently selected from halogen, —OR²⁰, —NO₂, —CN, —CO₂R²⁰, —C(O)N(R²⁰)₂, —N(R²⁰)C(O)R²⁰, —N(R²OC(O)OR²⁰, —N(R²⁰)₂, a C₃₋₇ cycloaliphatic, a 3-7 membered heterocyclyl, a C₁₋₄ aliphatic or a C₁₋₄ haloaliphatic; wherein said cycloaliphatic and heterocyclyl are optionally and independently substituted with up to 6 instances of halogen, —CN, —OH, C₁₋₂ alkyl, C₁₋₂ haloalkyl, C₁₋₂ alkoxy or C₁₋₂ haloalkoxy; and

each occurrence of R²⁰ is independently selected from hydrogen, a C₁₋₄ aliphatic or a C₁₋₄ haloaliphatic; or two R²⁰ attached to the same or different atom(s), together with the atom(s) to which they are attached, form a 3-7 membered heterocycle, wherein said heterocycle is optionally substituted with up to 6 instances of halogen, —CN, —OH, C₁₋₂ alkyl, C₁₋₂ haloalkyl, C₁₋₂ alkoxy or C₁₋₂ haloalkoxy.

In one embodiment of this aspect, V is a bond or a C₁₋₆ aliphatic, wherein up to two saturated carbons of said C₁₋₆ aliphatic are replaced by —O—, —C(O)—, —C(O)N(R)—, —OC(O)—, —C(O)O—, —N(R)—, —N(R)C(O)—, —N(R)S(O)₂—, —N(R)S(O)₂N(R)—, —S(O)₂— or —S(O)₂N(R)—.

In another embodiment, V is a bond, methylene, ethylene, propylene, butylene or pentylene, wherein up to two carbons of said methylene, ethylene, propylene, butylene or pentylene are replaced by —O—, —C(O)—, —C(O)N(R)—, —C(O)O—, —N(R)—, —N(R)C(O)—, —N(R)S(O)₂—, —N(R)S(O)₂N(R)—, —S(O)₂— or —S(O)₂N(R)—.

In another embodiment, V is a bond.

In one embodiment, V is methylene, ethylene, propylene, butylene or pentylene.

In another embodiment, —V—R⁸ is selected from the group consisting of:

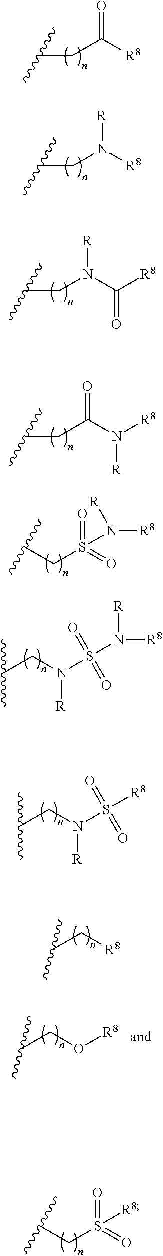

wherein n is an integer selected from 0, 1, 2, 3 or 4.

In one embodiment, R⁸ is independently selected from hydrogen, halogen, —NO₂, —CN, a C₁-C₆ aliphatic, a 5-14 membered aryl group, a 5-14 membered heteroaryl group, a C₃₋₁₂ cycloaliphatic group, or a 3-14 membered heterocyclyl group, wherein said C₁-C₆ aliphatic or said aryl, heteroaryl, cycloaliphatic or heterocyclyl is optionally and independently substituted with up to four instances of halogen, oxo, —NO₂, —CN, C₁₋₄ aliphatic, —OR¹⁷, —C(O)R¹⁷, —C(O)OR¹⁷ or —C(O)N(R′)R¹⁷; wherein said C₁₋₄ aliphatic is optionally substituted with up to four instances of —R¹⁶.

In another embodiment, R⁸ is independently selected from hydrogen, halogen, —NO₂, —CN or a C₁-C₆ aliphatic, wherein said C₁-C₆ aliphatic is optionally and independently substituted with up to four instances of halogen, oxo, —CN or C₁₋₄ aliphatic, C₁₋₄ haloaliphatic, C₁₋₄ alkoxy, C₁₋₄ haloalkoxy or —C(O)OR¹⁷.

In one embodiment, R⁸ is independently selected from hydrogen, fluoride, chloride, methyl, ethyl, propyl, butyl, vinyl, isopropyl, t-butyl, methoxymethyl, methoxy, isopropoxy, ethoxy, —C(OH)(CH₃)₂, trifluoromethyl, trifluoromethoxy or —CO₂H.

In one embodiment, R⁸ is independently selected from a 5-14 membered aryl group, or a 5-14 membered heteroaryl group, wherein said aryl or heteroaryl is optionally and independently substituted with up to four instances of halogen, oxo, —NO₂, —CN, —OR¹⁷, —C(O)OR¹⁷ or a C₁₋₆ alkyl, said alkyl being optionally substituted with up to 6 instances of —R¹⁶.

In a further embodiment, R⁸ is independently selected from the group consisting of:

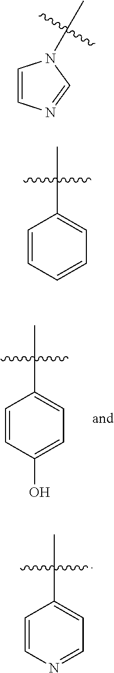 [SMILES: *n1ccnc1 ; *c1ccccc1 ; *c1ccc(O)cc1 ; *c1ccncc1]

In one embodiment, R⁸ is independently selected from a 3 to 12-membered cycloaliphatic group or a 3-14 membered heterocyclyl group, wherein each of said cycloaliphatic or heterocyclic ring is optionally and independently substituted with up to four instances of halogen, oxo, —CN, a C₁₋₄ aliphatic, a C₁₋₄ haloaliphatic, C₁₋₄ alkoxy or C₁₋₄ haloalkoxy.

In another embodiment, R⁸ is independently selected from the group consisting of

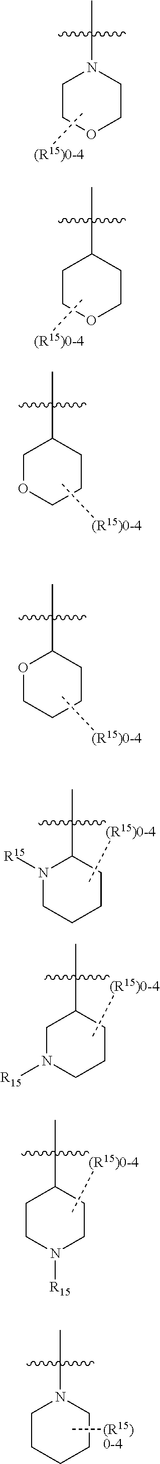

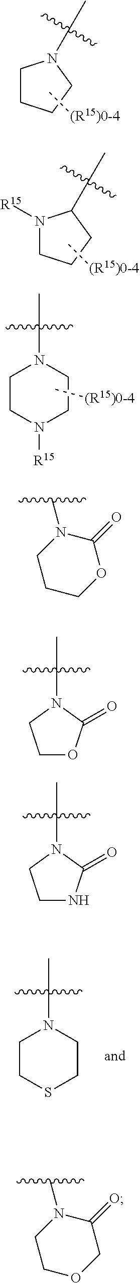

wherein R¹⁵ on a carbon atom is an optional substituent selected from halogen, —CN, C₁₋₄ alkyl, C₁₋₄ haloalkyl, C₁₋₄ alkoxy, C₁₋₄ haloalkoxy, —SO₂(C₁₋₄ alkyl) or —C(O)OR¹⁷; and
R¹⁵ on a nitrogen atom is an optional substituent selected from C₁₋₄ alkyl, C₁₋₄ haloalkyl, C₁₋₄ alkoxy, C₁₋₄ haloalkoxy, —SO₂(C₁₋₄ alkyl) or —C(O)OR¹⁷.

In another embodiment, R⁸ is independently selected from the group consisting of

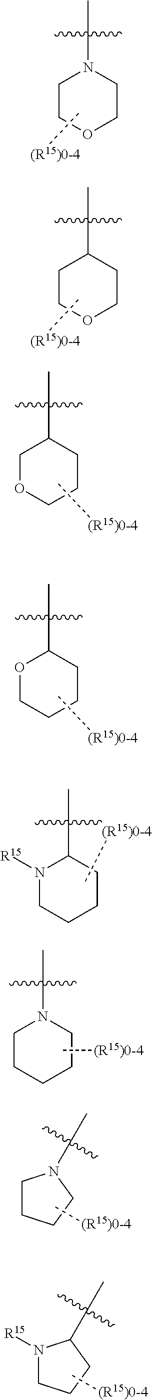

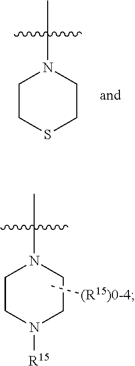

wherein R¹⁵ on a carbon atom is an optional substituent selected from halogen, C₁₋₄ alkyl, C₁₋₄ haloalkyl, C₁₋₄ alkoxy, C₁₋₄ haloalkoxy, —SO₂(C₁₋₄ alkyl) or —C(O)OR¹⁷; and
R¹⁵ on a nitrogen atom is an optional substituent selected from C₁₋₄ alkyl, C₁₋₄ haloalkyl, C₁₋₄ alkoxy, C₁₋₄ haloalkoxy, —SO₂(C₁₋₄ alkyl) or —C(O)OR¹⁷.

In further embodiment, R⁸ is independently selected from the group consisting of:

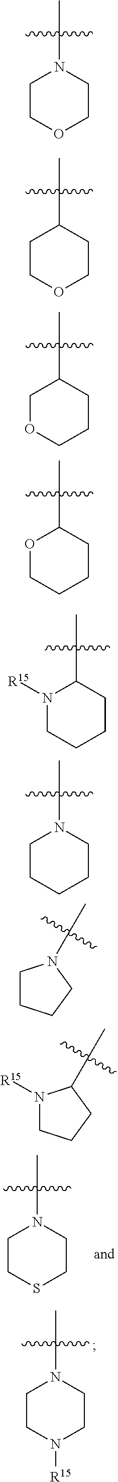

wherein R¹⁵ on a N atom is an optional substituent selected from C₁₋₄-alkyl or C₁₋₄ haloalkyl.

In one embodiment, R² is hydrogen, halogen or a C₁₋₄ aliphatic; wherein said C₁₋₄ aliphatic is optionally substituted with up to six instances of halogen, —O(C₁₋₂ alkyl), —O(C₁₋₂ haloalkyl), C₁₋₂ alkyl or C₁₋₂ haloalkyl.

In another embodiment, R² is a C₁₋₄ aliphatic.

In a further embodiment, R² is methyl.

In one embodiment, R³ is hydrogen, halogen, —C(O)OR^{X} or a C₁₋₄ aliphatic; said aliphatic being optionally substituted with up to six instances of halogen, —O(C₁₋₂ alkyl), —O(C₁₋₂ haloalkyl), C₁₋₂ alkyl or C₁₋₂ haloalkyl.

In another embodiment, R³ is hydrogen or halogen.

In another embodiment, R³ is chloro or fluoro.

In a further embodiment, R³ is chloro.

In another further embodiment, R³ is hydrogen.

In one embodiment, R⁴ is hydrogen, halogen or a C₁₋₄ aliphatic optionally substituted with up to six instances of halogen, —O(C₁₋₂ alkyl), —O(C₁₋₂ haloalkyl), C₁₋₂ alkyl or C₁₋₂ haloalkyl.

In another embodiment, R⁴ is hydrogen or halogen.

In a further embodiment, R⁴ is hydrogen.

In one embodiment, ring A is phenyl or a 5 or 6-membered monocyclic heteroaryl having 1-3 heteroatoms independently selected from N, O or S, wherein said heteroaryl is optionally substituted with up to five instances of R¹².

In one embodiment, ring A is phenyl or a 6-membered heteroaryl ring containing up to two nitrogen atoms, wherein said phenyl or said heteroaryl ring is optionally substituted with up to three instances of chloro, fluoro, —CN, —NO₂, C₁₋₄ alkyl, C₁₋₄ haloalkyl, C₁₋₄ haloalkoxy, C₁₋₄ alkoxy, C₁₋₄ alkylthio, hydroxy, or amino; or wherein said phenyl or heteroaryl ring is fused with a 5 membered heterocycle or cycloaliphatic.

In a further embodiment, ring A is benzo[d][1,3]dioxole-5-yl, 2,3-dihydrobenzofuran-7-yl or phenyl, wherein the phenyl ring is optionally substituted with up to 3 instances of chloro, fluoro, hydroxy, methyl, ethyl, methoxy, ethoxy, trifluoromethyl, trifluoromethoxy, thiomethoxy, isopropoxy, —N(C₁₋₄ alkyl)₂, or amino.

In one embodiment, ring A is phenyl, optionally substituted with up to 2 instances of chloro, fluoro, hydroxy, methyl, ethyl, methoxy, ethoxy, trifluoromethyl, trifluoromethoxy, thiomethoxy, isopropoxy, —N(C₁₋₄ alkyl)₂, or amino.

In one embodiment, R⁵ is chosen from hydrogen, halogen, —CN, —OH, —O(C₁₋₄ aliphatic), a C₁₋₄ aliphatic or a 3-7 membered heterocyclyl, wherein said C₁₋₄ aliphatic and said —O(C₁₋₄ aliphatic) of R⁵ are optionally and independently substituted with up to three instances of halogen; and R⁶ is chosen from hydrogen, halogen, C₁₋₄ aliphatic or —O(C₁₋₄ aliphatic), wherein said C₁₋₄ aliphatic of R⁶ is optionally substituted with up to three instances of halogen.

In another embodiment, R⁵ is chosen from hydrogen, halogen, —OH, —O(C₁₋₄ aliphatic) or —O(C₁₋₄ haloaliphatic); and R⁶ is chosen from hydrogen, halogen or a C₁₋₄ aliphatic, optionally substituted with up to three instances of halogen.

In a further embodiment, R⁵ is hydrogen or fluoro and R⁶ is hydrogen or fluoro.

In another further embodiment, R⁵ is —OH and R⁶ is hydrogen.

In one embodiment, R⁵ is hydrogen, —OH, —O(C₁₋₄ alkyl) or —O(C₁₋₄ haloalkyl) and R⁶ is C₁₋₄ alkyl or C₁₋₄ haloalkyl.

In another aspect, the invention includes a compound having the formula I-A, or is a pharmaceutically acceptable salt thereof:

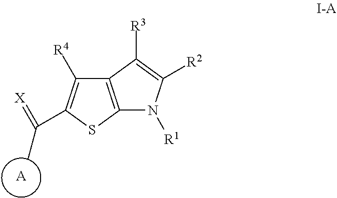

wherein X is O, S or NH; and Ring A, R¹, R², R³ and R⁴ are defined as above.

In one embodiment of this aspect, X is O.

In another aspect, the invention includes a compound having the formula I-B, or is a pharmaceutically acceptable salt thereof:

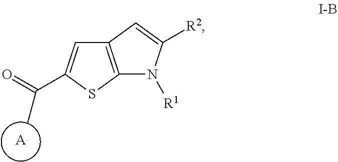

wherein Ring A, R¹ and R² are defined as above.

In another aspect, the invention includes a compound having the formula I-C, or is a pharmaceutically acceptable salt thereof:

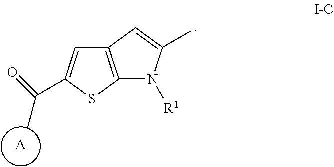

wherein Ring A and R¹ are defined as above.

In another aspect, the invention includes a compound having the formula I-D:

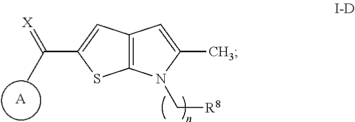

or is a pharmaceutically acceptable salt thereof, wherein

n is selected from 0, 1, 2 or 3; and R⁸ is defined as above.

In one embodiment of this aspect, n is selected from 2 or 3.

In another embodiment, X is O.

In one embodiment, R⁸ is a 5-8 membered heterocyclyl, optionally substituted with up to 6 instances of R¹⁵.

In one embodiment, R⁸ is tetrahydropyran, morpholinyl, thiomorpholinyl, piperidinyl, pyrrolidinyl, piperazinyl or oxazolidinyl; wherein each is optionally substituted with up to 6 instances of oxo, C₁₋₂ alkyl or C₁₋₂ haloalkyl.

In another embodiment, R⁸ is selected from the group consisting of:

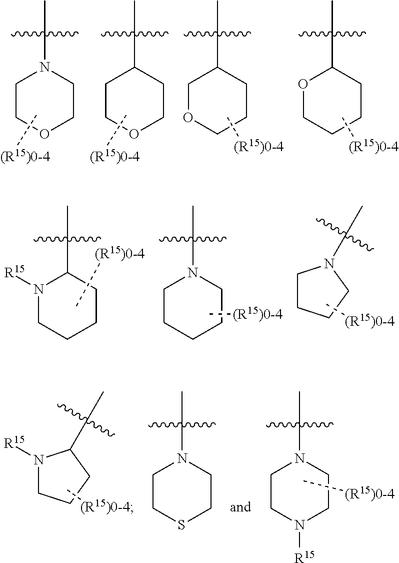

wherein R¹⁵ on a carbon atom is an optional substituent selected from halogen, C₁₋₄ alkyl, C₁₋₄ haloalkyl, C₁₋₄ alkoxy, C₁₋₄ haloalkoxy, —SO₂(C₁₋₄ alkyl) or —C(O)OR¹⁷; and
R¹⁵ on a nitrogen atom is an optional substituent selected from C₁₋₄ alkyl, C₁₋₄ haloalkyl, C₁₋₄ alkoxy, C₁₋₄ haloalkoxy, —SO₂(C₁₋₄ alkyl) or —C(O)OR¹⁷.

In a further embodiment, R⁸ is selected from the group consisting of:

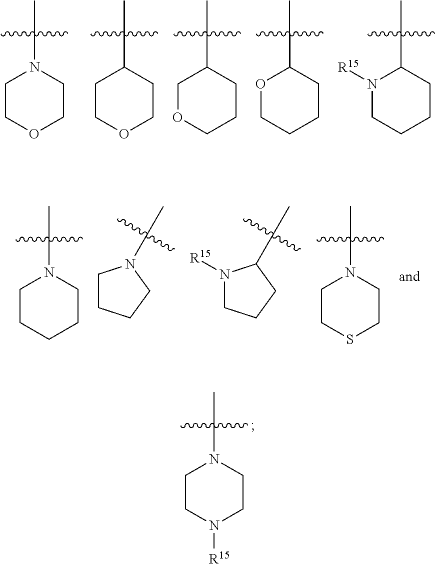

wherein R¹⁵ is C₁₋₄-alkyl or C₁₋₄ haloalkyl.

In another embodiment, R⁸ is a C₁-C₆ aliphatic, wherein said aliphatic is optionally and independently substituted with up to four instances of R¹⁵.

In one embodiment, the compound is selected from the group consisting of:

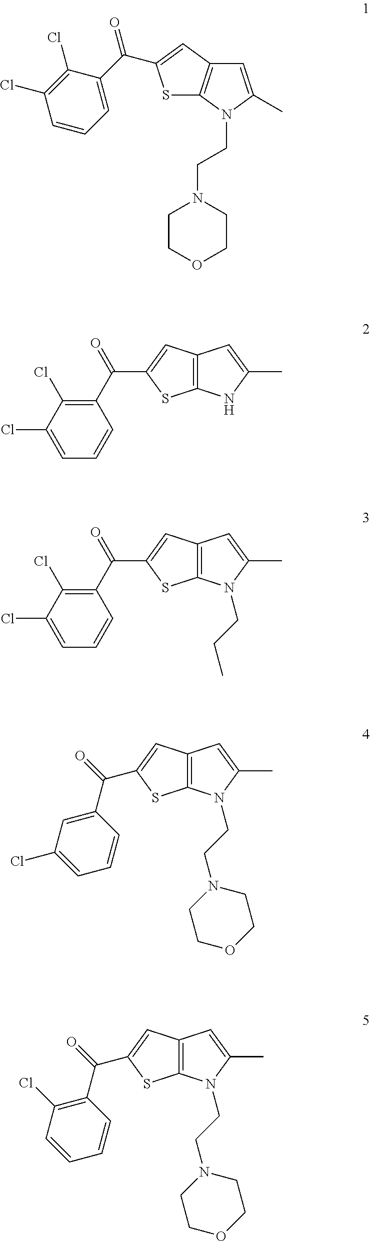 [SMILES: Cc1cc2cc(C(=O)c3cccc(Cl)c3Cl)sc2n1CCN1CCOCC1 ; Cc1cc2cc(C(=O)c3cccc(Cl)c3Cl)sc2[nH]1 ; CCCn1c(C)cc2cc(C(=O)c3cccc(Cl)c3Cl)sc21 ; Cc1cc2cc(C(=O)c3cccc(Cl)c3)sc2n1CCN1CCOCC1 ; Cc1cc2cc(C(=O)c3ccccc3Cl)sc2n1CCN1CCOCC1]

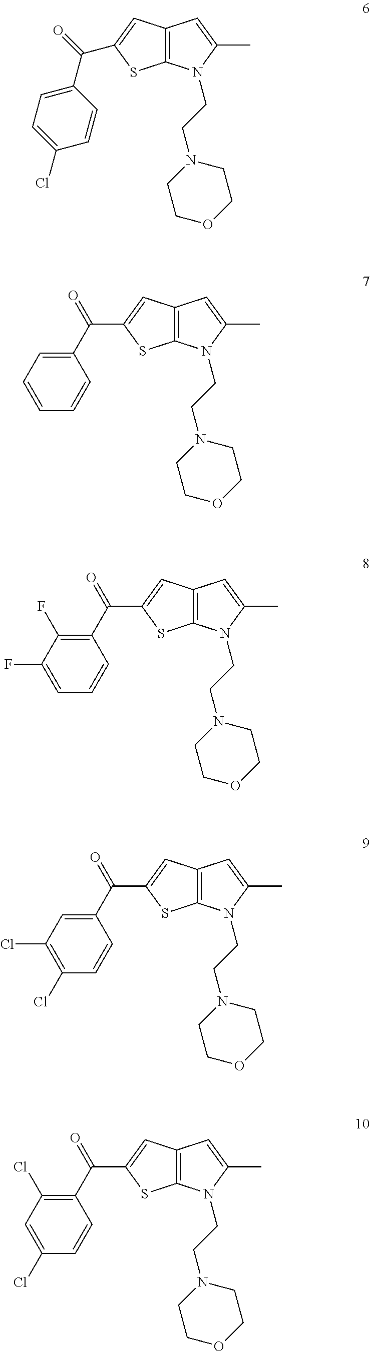 [SMILES: Cc1cc2cc(C(=O)c3ccc(Cl)cc3)sc2n1CCN1CCOCC1 ; Cc1cc2cc(C(=O)c3ccccc3)sc2n1CCN1CCOCC1 ; Cc1cc2cc(C(=O)c3cccc(F)c3F)sc2n1CCN1CCOCC1 ; Cc1cc2cc(C(=O)c3ccc(Cl)c(Cl)c3)sc2n1CCN1CCOCC1 ; Cc1cc2cc(C(=O)c3ccc(Cl)cc3Cl)sc2n1CCN1CCOCC1]

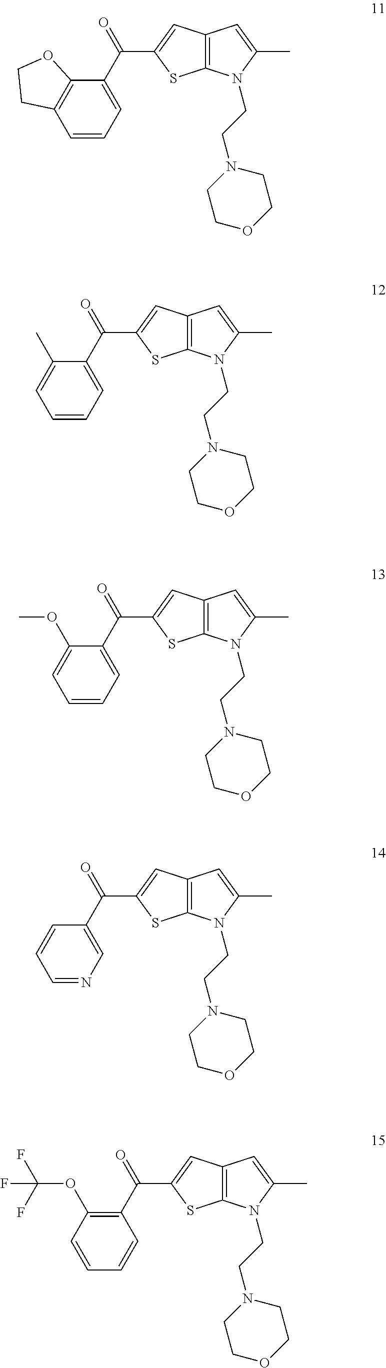

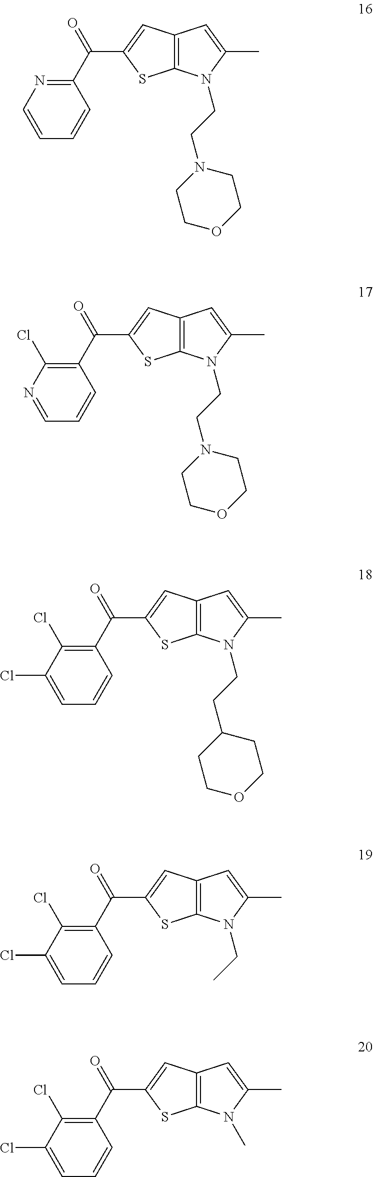 [SMILES: Cc1cc2cc(C(=O)c3ccccn3)sc2n1CCN1CCOCC1 ; Cc1cc2cc(C(=O)c3cccnc3Cl)sc2n1CCN1CCOCC1 ; Cc1cc2cc(C(=O)c3cccc(Cl)c3Cl)sc2n1CCC1CCOCC1 ; CCn1c(C)cc2cc(C(=O)c3cccc(Cl)c3Cl)sc21 ; Cc1cc2cc(C(=O)c3cccc(Cl)c3Cl)sc2n1C]

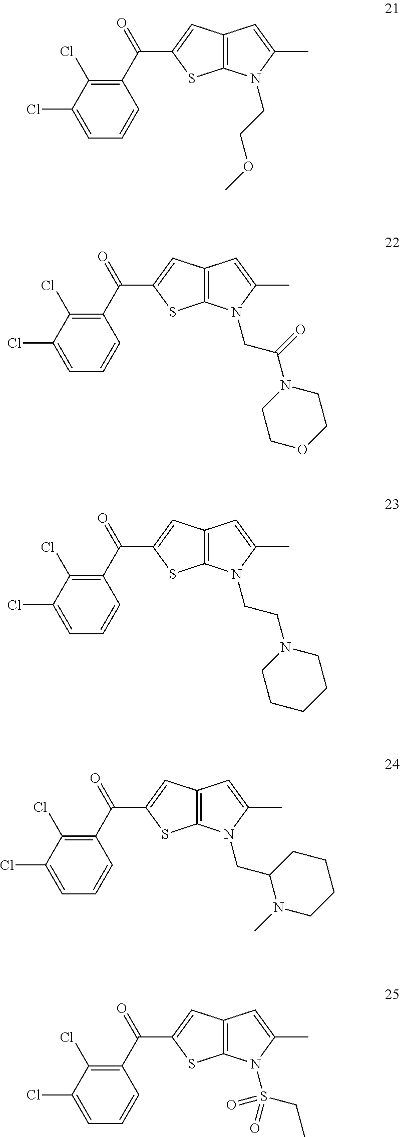 [SMILES: COCCn1c(C)cc2cc(C(=O)c3cccc(Cl)c3Cl)sc21 ; Cc1cc2cc(C(=O)c3cccc(Cl)c3Cl)sc2n1CC(=O)N1CCOCC1 ; Cc1cc2cc(C(=O)c3cccc(Cl)c3Cl)sc2n1CCN1CCCCC1 ; Cc1cc2cc(C(=O)c3cccc(Cl)c3Cl)sc2n1CC1CCCCN1C ; CCS(=O)(=O)n1c(C)cc2cc(C(=O)c3cccc(Cl)c3Cl)sc21]

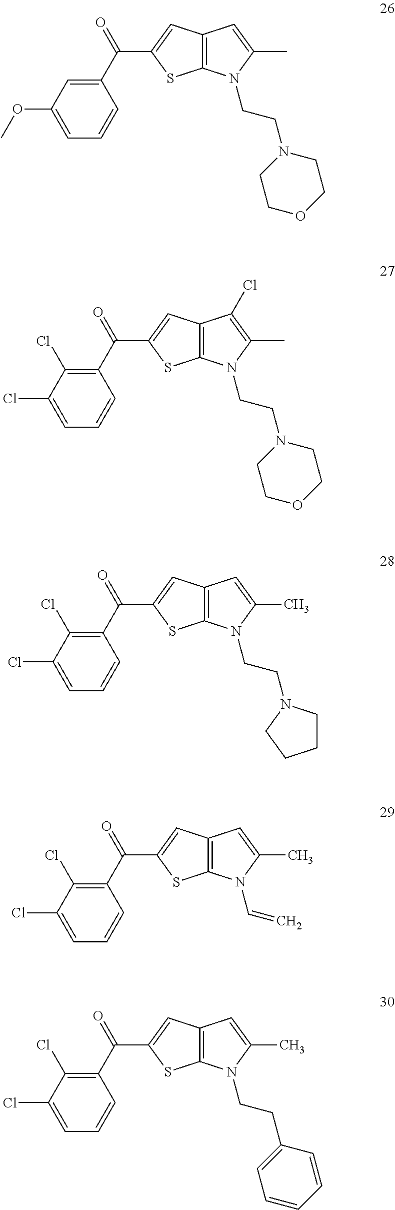 [SMILES: COc1cccc(C(=O)c2cc3cc(C)n(CCN4CCOCC4)c3s2)c1 ; Cc1c(Cl)c2cc(C(=O)c3cccc(Cl)c3Cl)sc2n1CCN1CCOCC1 ; Cc1cc2cc(C(=O)c3cccc(Cl)c3Cl)sc2n1CCN1CCCC1 ; C=Cn1c(C)cc2cc(C(=O)c3cccc(Cl)c3Cl)sc21 ; Cc1cc2cc(C(=O)c3cccc(Cl)c3Cl)sc2n1CCc1ccccc1]

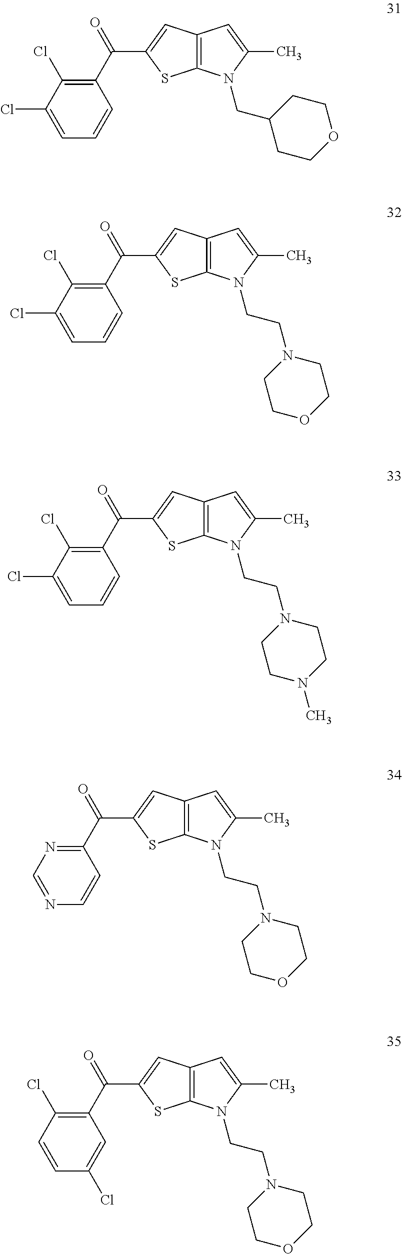 [SMILES: Cc1cc2cc(C(=O)c3cccc(Cl)c3Cl)sc2n1CC1CCOCC1 ; Cc1cc2cc(C(=O)c3cccc(Cl)c3Cl)sc2n1CCN1CCOCC1 ; Cc1cc2cc(C(=O)c3cccc(Cl)c3Cl)sc2n1CCN1CCN(C)CC1 ; Cc1cc2cc(C(=O)c3ccncn3)sc2n1CCN1CCOCC1 ; Cc1cc2cc(C(=O)c3cc(Cl)ccc3Cl)sc2n1CCN1CCOCC1]

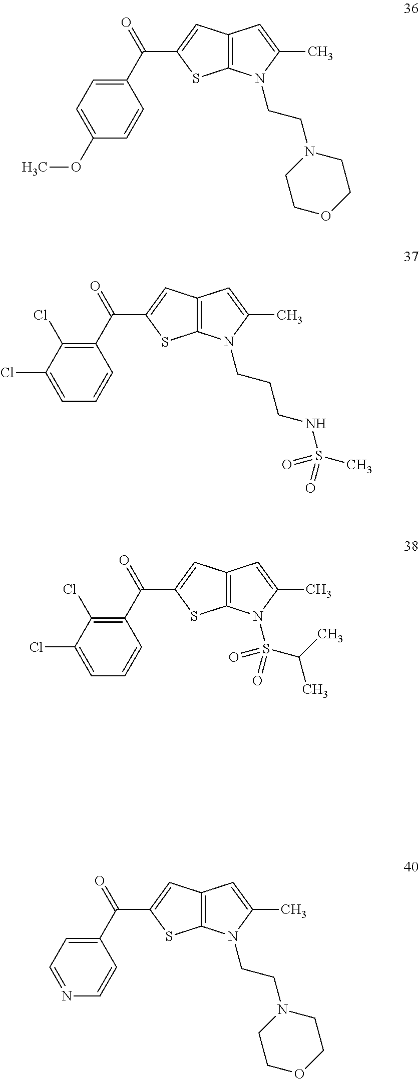 [SMILES: COc1ccc(C(=O)c2cc3cc(C)n(CCN4CCOCC4)c3s2)cc1 ; Cc1cc2cc(C(=O)c3cccc(Cl)c3Cl)sc2n1CCCNS(C)(=O)=O ; Cc1cc2cc(C(=O)c3cccc(Cl)c3Cl)sc2n1S(=O)(=O)C(C)C ; Cc1cc2cc(C(=O)c3ccncc3)sc2n1CCN1CCOCC1]

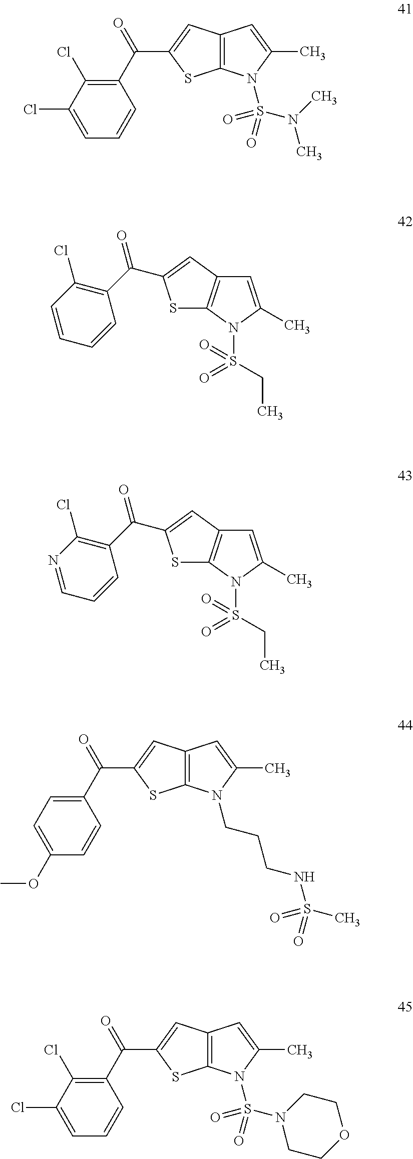

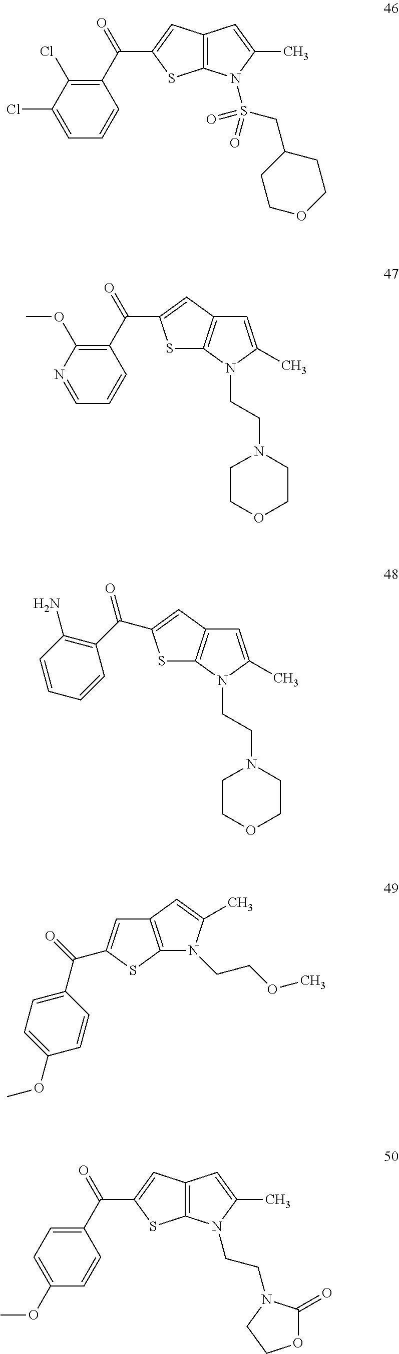

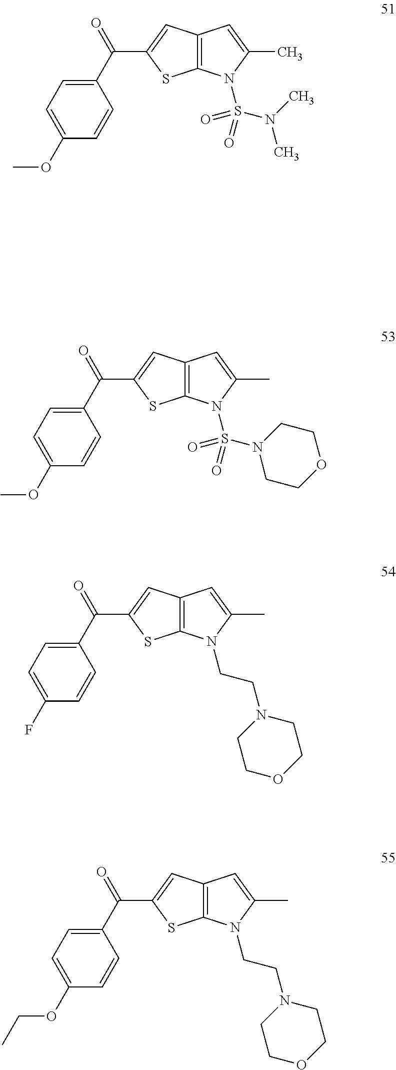

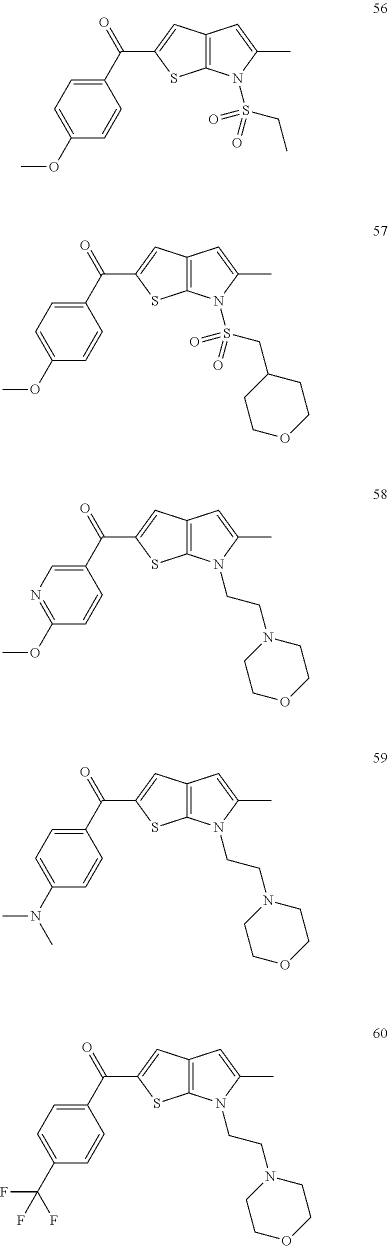

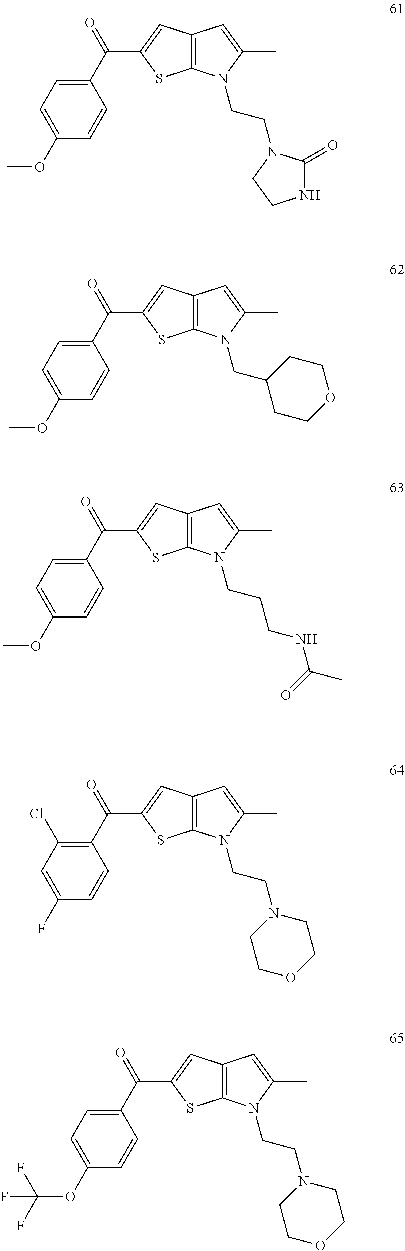 [SMILES: COc1ccc(C(=O)c2cc3cc(C)n(CCN4CCNC4=O)c3s2)cc1 ; COc1ccc(C(=O)c2cc3cc(C)n(CC4CCOCC4)c3s2)cc1 ; COc1ccc(C(=O)c2cc3cc(C)n(CCCNC(C)=O)c3s2)cc1 ; Cc1cc2cc(C(=O)c3ccc(F)cc3Cl)sc2n1CCN1CCOCC1 ; Cc1cc2cc(C(=O)c3ccc(OC(F)(F)F)cc3)sc2n1CCN1CCOCC1]

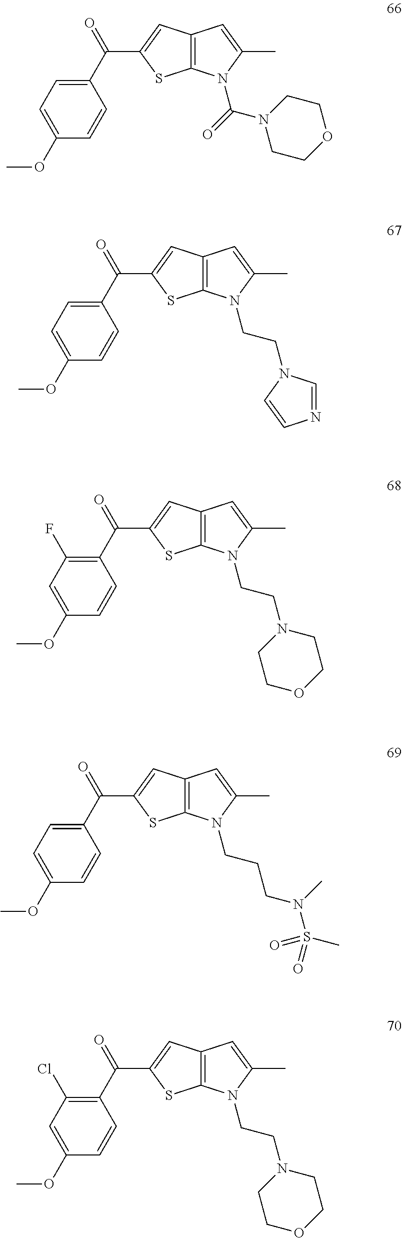

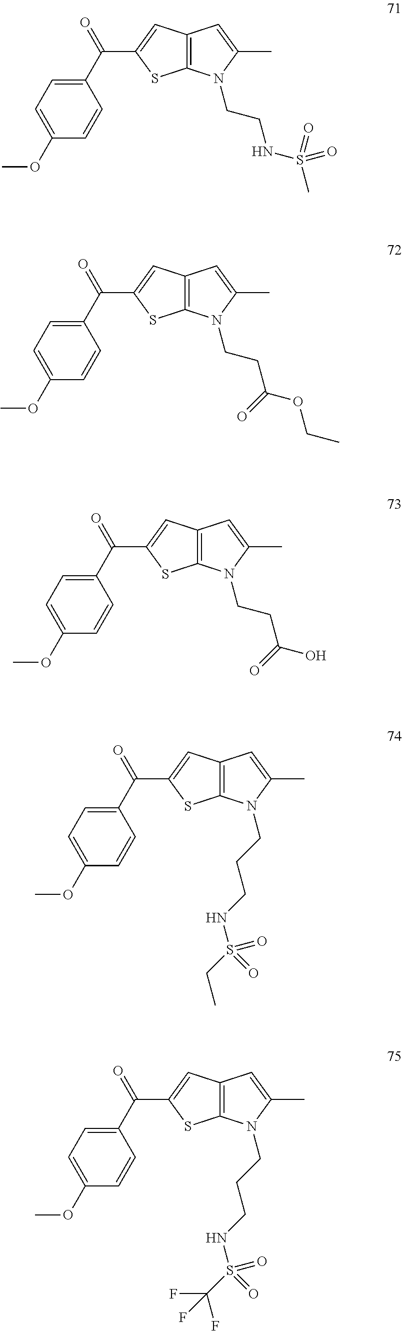

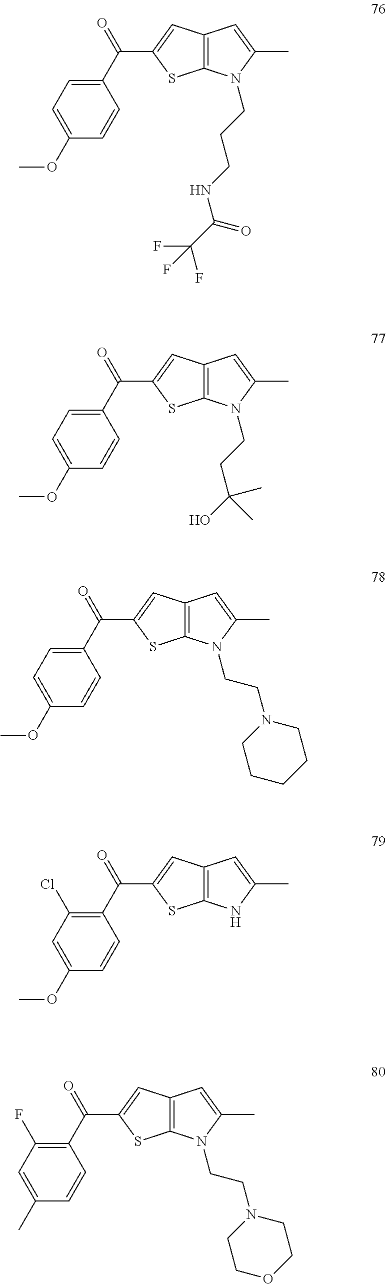

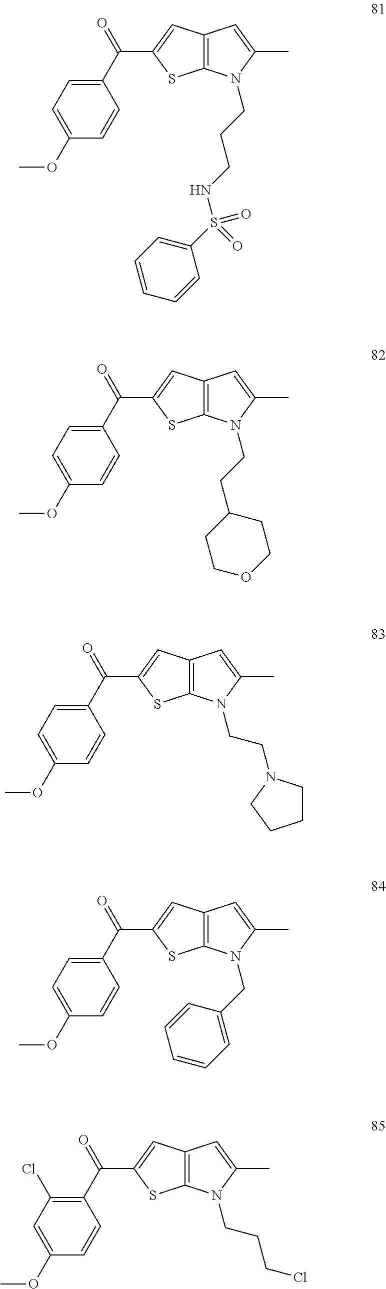

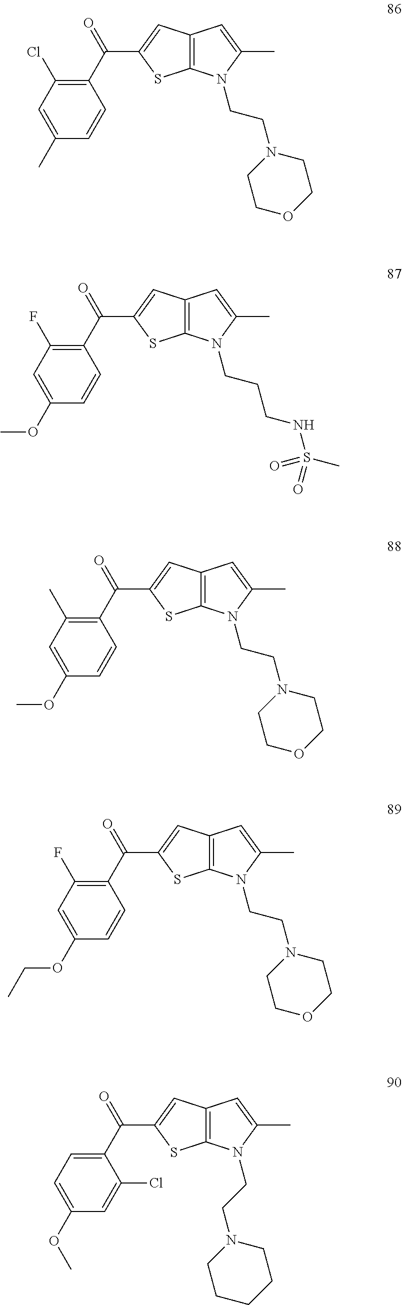

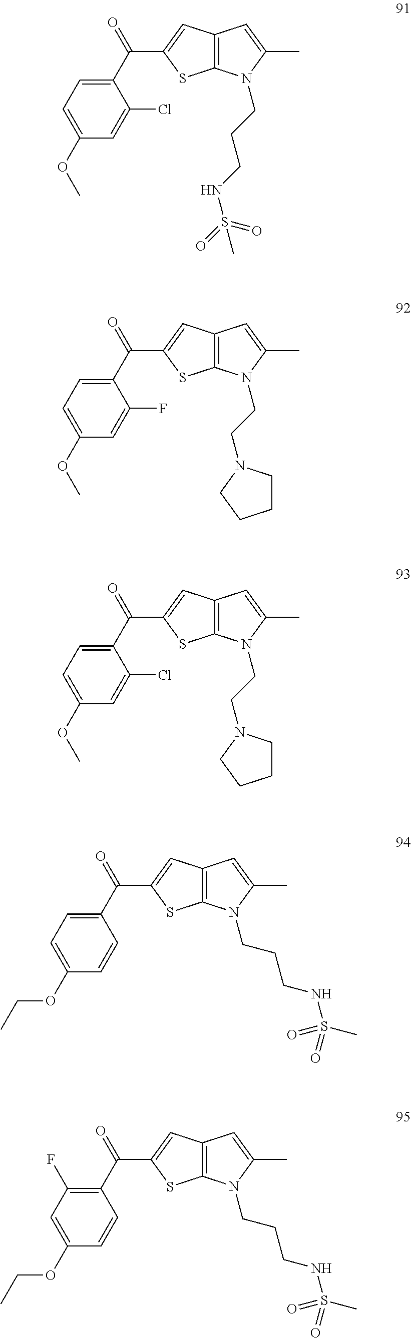

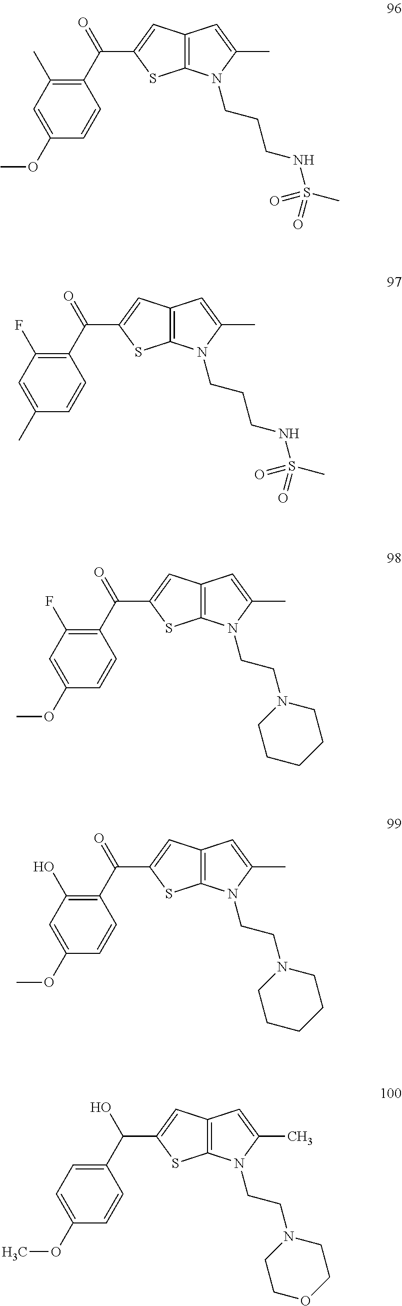

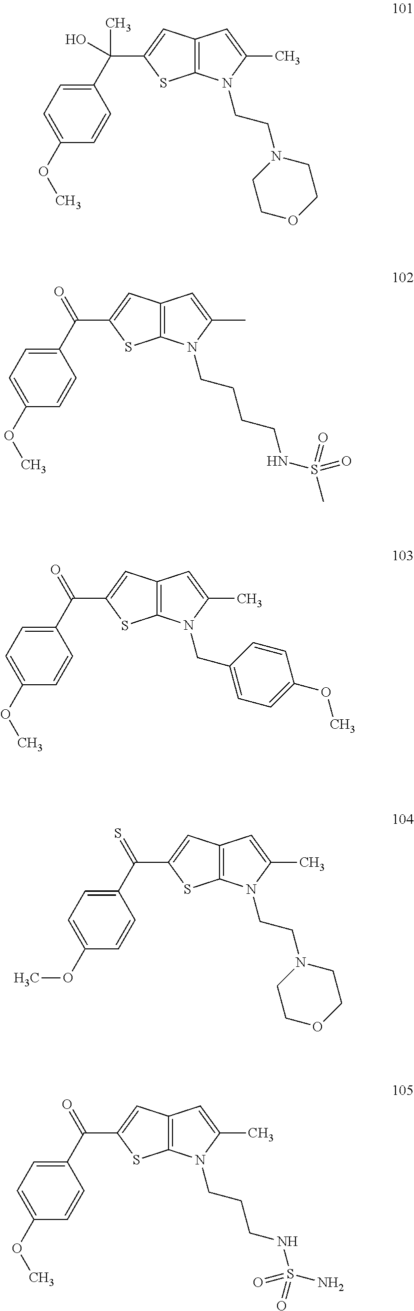

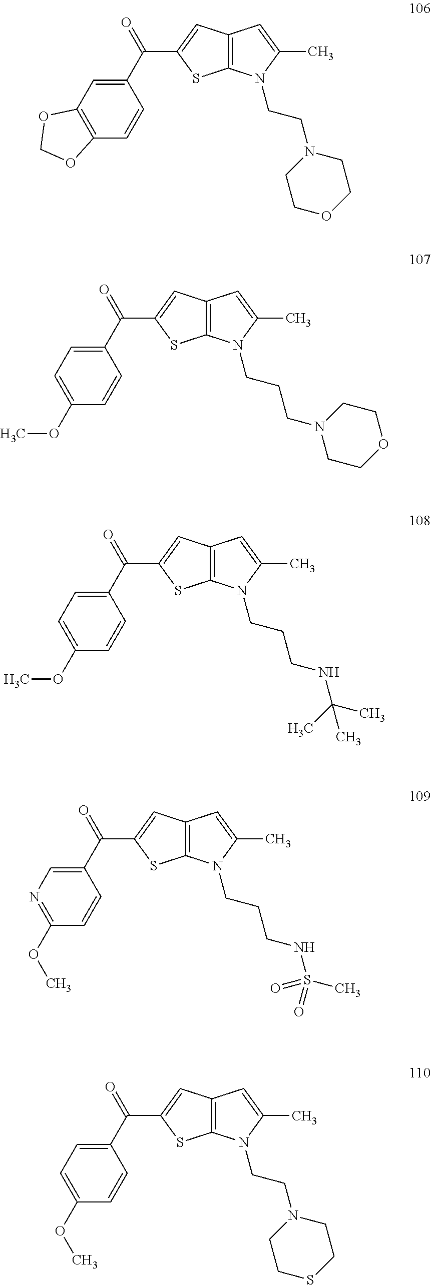 [SMILES: Cc1cc2cc(C(=O)c3ccc4c(c3)OCO4)sc2n1CCN1CCOCC1 ; COc1ccc(C(=O)c2cc3cc(C)n(CCCN4CCOCC4)c3s2)cc1 ; COc1ccc(C(=O)c2cc3cc(C)n(CCCNC(C)(C)C)c3s2)cc1 ; COc1ccc(C(=O)c2cc3cc(C)n(CCCNS(C)(=O)=O)c3s2)cn1 ; COc1ccc(C(=O)c2cc3cc(C)n(CCN4CCSCC4)c3s2)cc1]

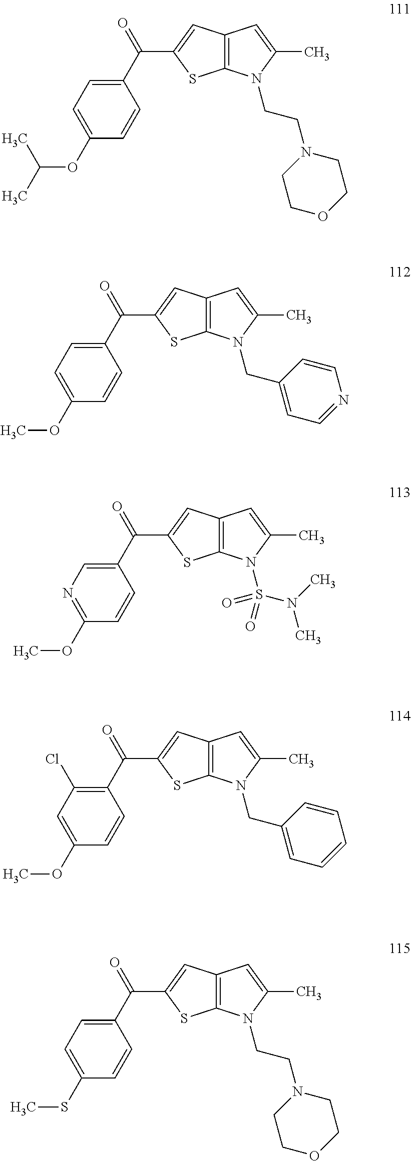

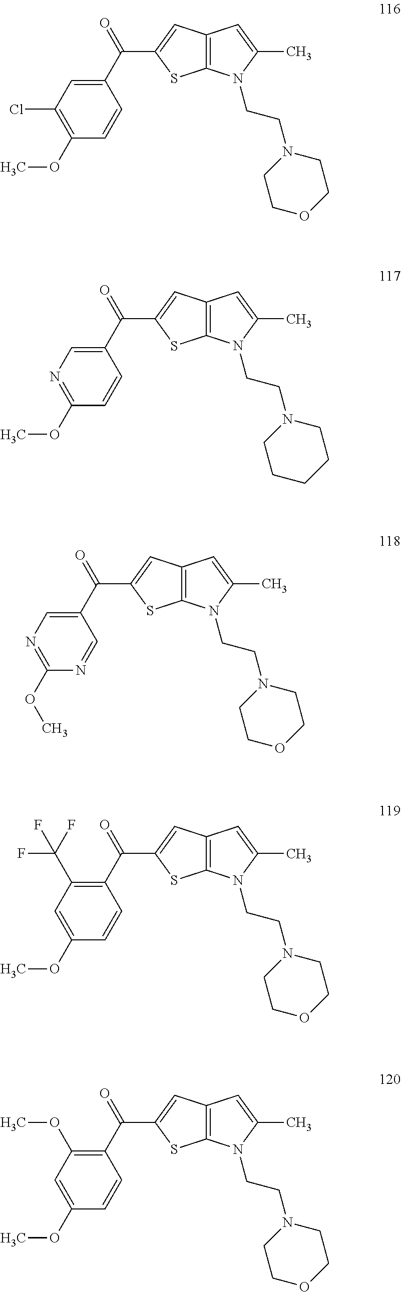 [SMILES: COc1ccc(C(=O)c2cc3cc(C)n(CCN4CCOCC4)c3s2)cc1Cl ; COc1ccc(C(=O)c2cc3cc(C)n(CCN4CCCCC4)c3s2)cn1 ; COc1ncc(C(=O)c2cc3cc(C)n(CCN4CCOCC4)c3s2)cn1 ; COc1ccc(C(=O)c2cc3cc(C)n(CCN4CCOCC4)c3s2)c(C(F)(F)F)c1 ; COc1ccc(C(=O)c2cc3cc(C)n(CCN4CCOCC4)c3s2)c(OC)c1]

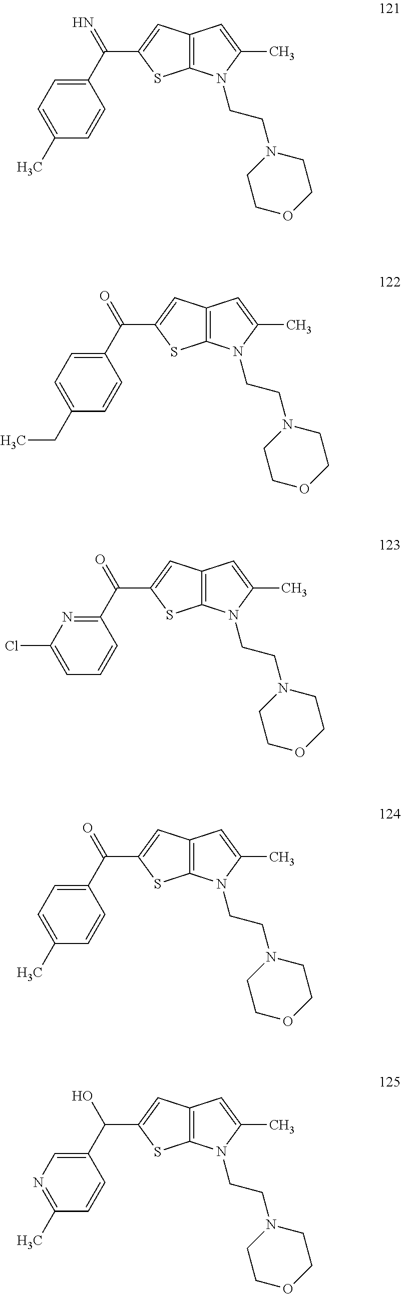 [SMILES: Cc1ccc(C(=N)c2cc3cc(C)n(CCN4CCOCC4)c3s2)cc1 ; CCc1ccc(C(=O)c2cc3cc(C)n(CCN4CCOCC4)c3s2)cc1 ; Cc1cc2cc(C(=O)c3cccc(Cl)n3)sc2n1CCN1CCOCC1 ; Cc1ccc(C(=O)c2cc3cc(C)n(CCN4CCOCC4)c3s2)cc1 ; Cc1ccc(C(O)c2cc3cc(C)n(CCN4CCOCC4)c3s2)cn1]

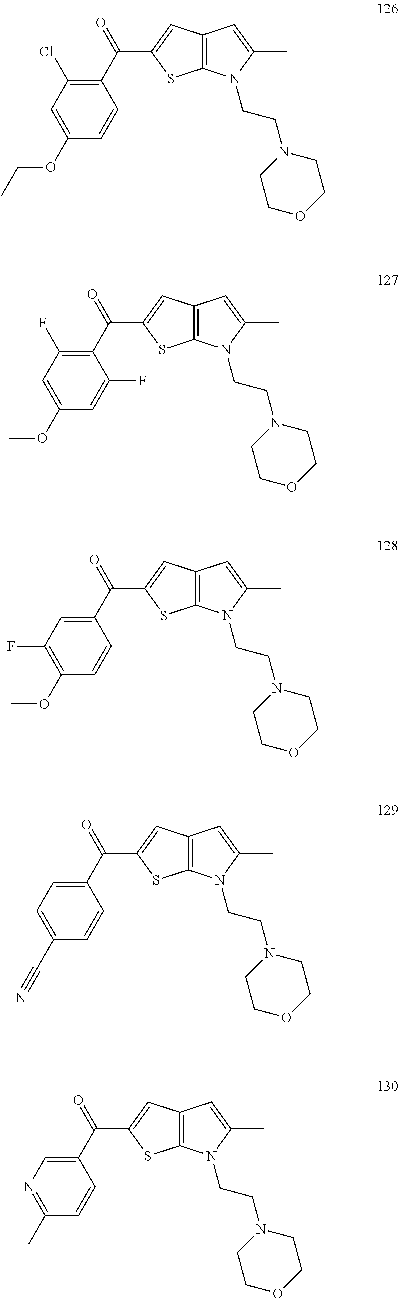 [SMILES: CCOc1ccc(C(=O)c2cc3cc(C)n(CCN4CCOCC4)c3s2)c(Cl)c1 ; COc1cc(F)c(C(=O)c2cc3cc(C)n(CCN4CCOCC4)c3s2)c(F)c1 ; COc1ccc(C(=O)c2cc3cc(C)n(CCN4CCOCC4)c3s2)cc1F ; Cc1cc2cc(C(=O)c3ccc(C#N)cc3)sc2n1CCN1CCOCC1 ; Cc1ccc(C(=O)c2cc3cc(C)n(CCN4CCOCC4)c3s2)cn1]

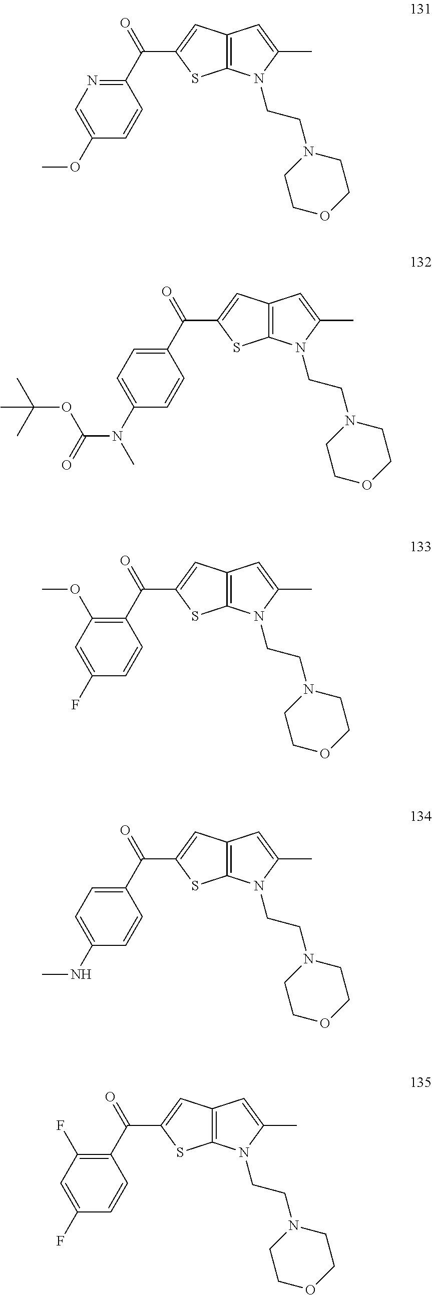 [SMILES: COc1ccc(C(=O)c2cc3cc(C)n(CCN4CCOCC4)c3s2)nc1 ; Cc1cc2cc(C(=O)c3ccc(N(C)C(=O)OC(C)(C)C)cc3)sc2n1CCN1CCOCC1 ; COc1cc(F)ccc1C(=O)c1cc2cc(C)n(CCN3CCOCC3)c2s1 ; CNc1ccc(C(=O)c2cc3cc(C)n(CCN4CCOCC4)c3s2)cc1 ; Cc1cc2cc(C(=O)c3ccc(F)cc3F)sc2n1CCN1CCOCC1]

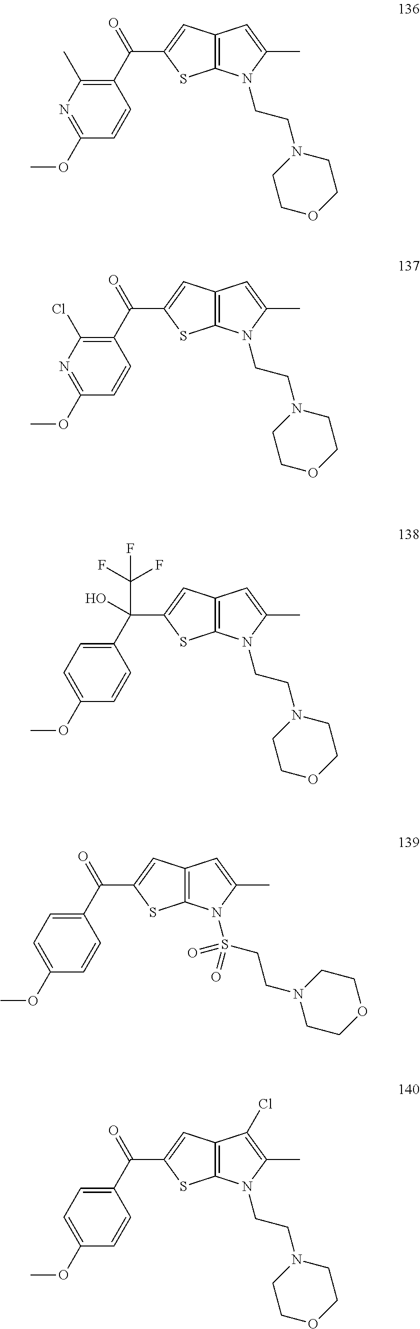 [SMILES: COc1ccc(C(=O)c2cc3cc(C)n(CCN4CCOCC4)c3s2)c(C)n1 ; COc1ccc(C(=O)c2cc3cc(C)n(CCN4CCOCC4)c3s2)c(Cl)n1 ; COc1ccc(C(O)(c2cc3cc(C)n(CCN4CCOCC4)c3s2)C(F)(F)F)cc1 ; COc1ccc(C(=O)c2cc3cc(C)n(S(=O)(=O)CCN4CCOCC4)c3s2)cc1 ; COc1ccc(C(=O)c2cc3c(Cl)c(C)n(CCN4CCOCC4)c3s2)cc1]

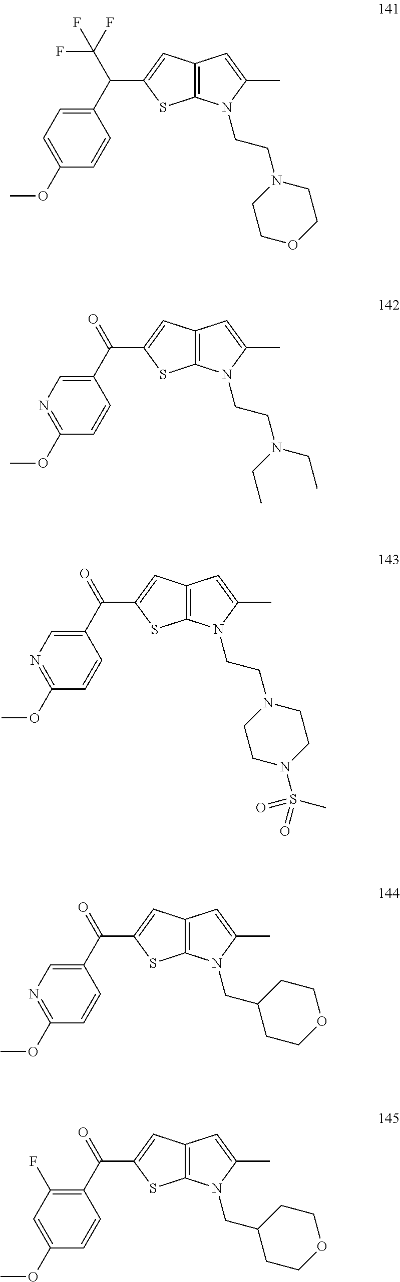

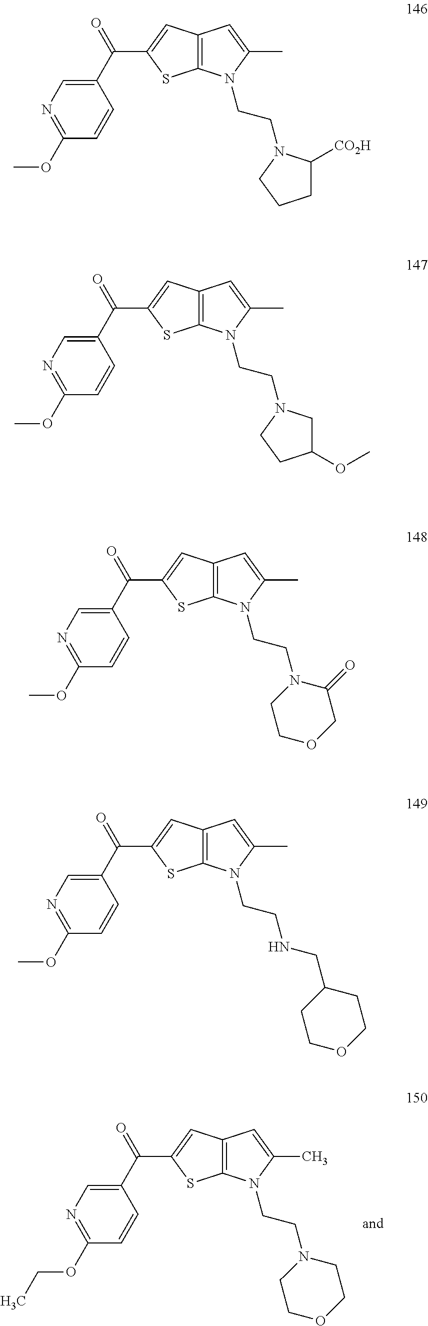

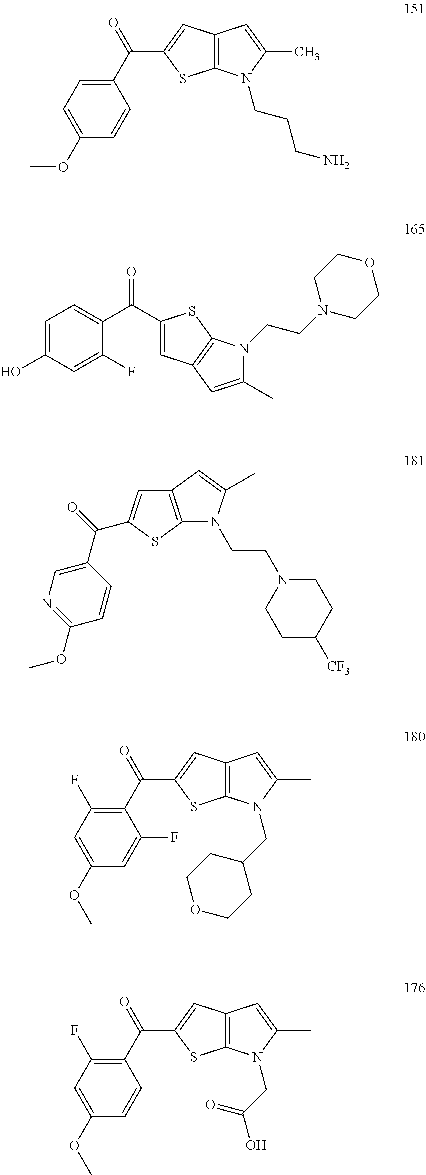 [SMILES: COc1ccc(C(=O)c2cc3cc(C)n(CCCN)c3s2)cc1 ; Cc1cc2cc(C(=O)c3ccc(O)cc3F)sc2n1CCN1CCOCC1 ; COc1ccc(C(=O)c2cc3cc(C)n(CCN4CCC(C(F)(F)F)CC4)c3s2)cn1 ; COc1cc(F)c(C(=O)c2cc3cc(C)n(CC4CCOCC4)c3s2)c(F)c1 ; COc1ccc(C(=O)c2cc3cc(C)n(CC(=O)O)c3s2)c(F)c1]

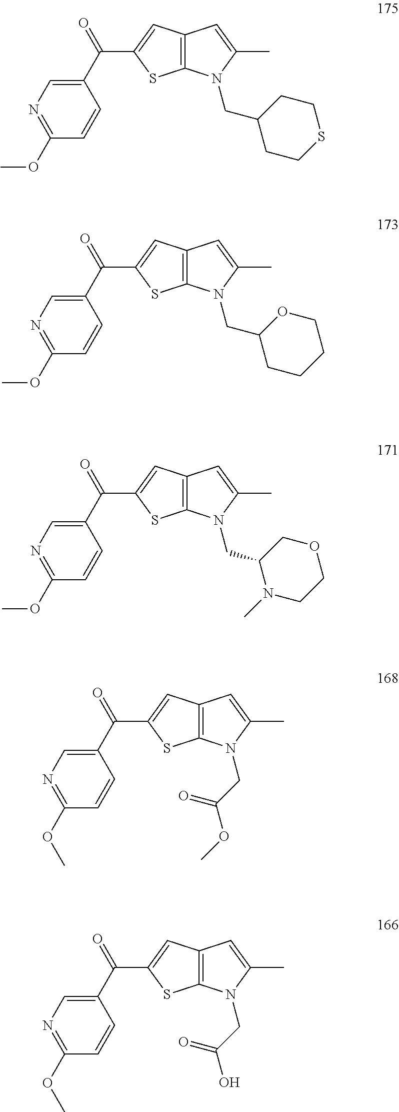 [SMILES: COc1ccc(C(=O)c2cc3cc(C)n(CC4CCSCC4)c3s2)cn1 ; COc1ccc(C(=O)c2cc3cc(C)n(CC4CCCCO4)c3s2)cn1 ; COc1ccc(C(=O)c2cc3cc(C)n(C[C@@H]4COCCN4C)c3s2)cn1 ; COC(=O)Cn1c(C)cc2cc(C(=O)c3ccc(OC)nc3)sc21 ; COc1ccc(C(=O)c2cc3cc(C)n(CC(=O)O)c3s2)cn1]

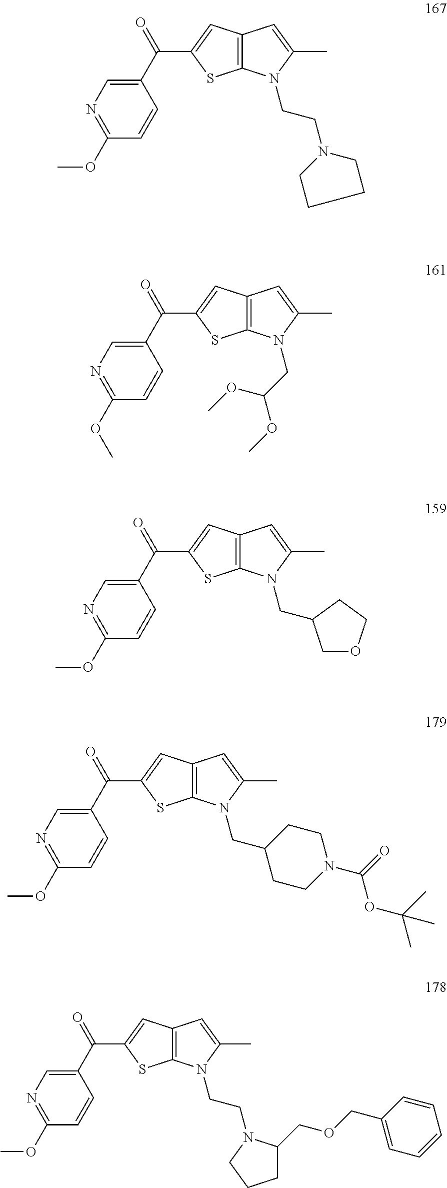

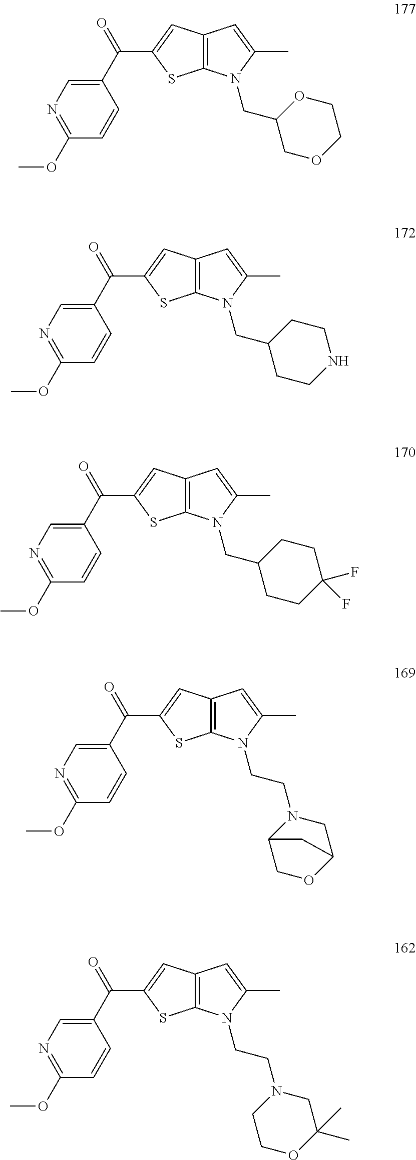

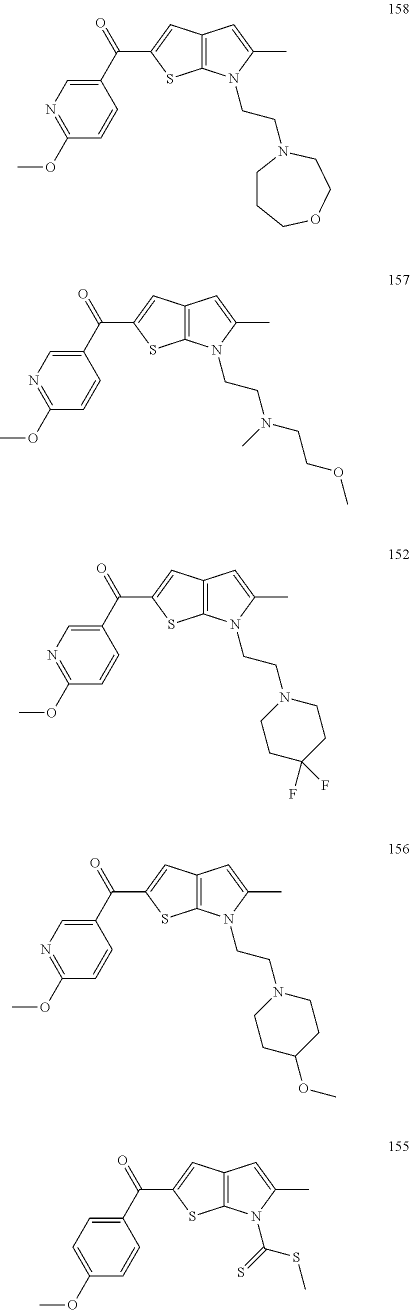

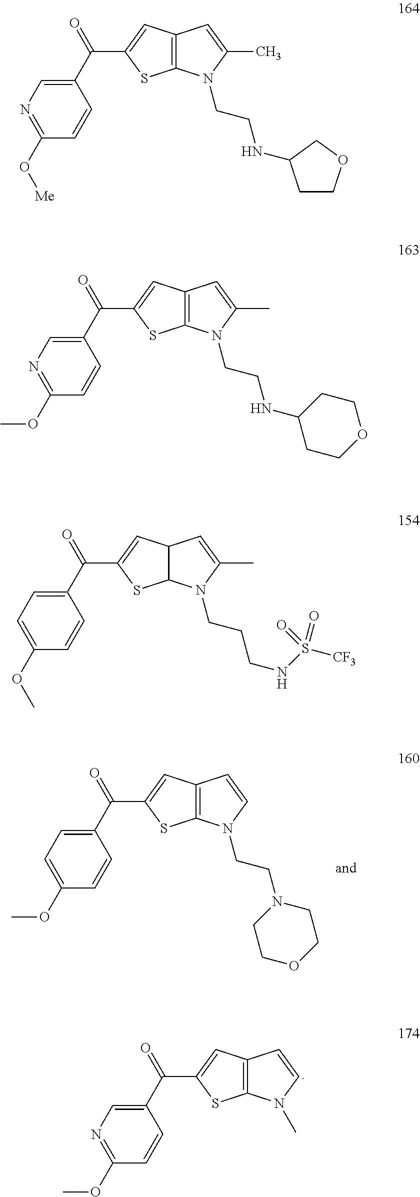 [SMILES: COc1ccc(C(=O)c2cc3cc(C)n(CCNC4CCOC4)c3s2)cn1 ; COc1ccc(C(=O)c2cc3cc(C)n(CCNC4CCOCC4)c3s2)cn1 ; COc1ccc(C(=O)C2=CC3C=C(C)N(CCCNS(=O)(=O)C(F)(F)F)C3S2)cc1 ; COc1ccc(C(=O)c2cc3ccn(CCN4CCOCC4)c3s2)cc1 ; COc1ccc(C(=O)c2cc3ccn(C)c3s2)cn1]

In another aspect, the invention is a pharmaceutical composition comprising a compound of formula I, or a pharmaceutically acceptable salt thereof, and a pharmaceutically acceptable carrier, vehicle or adjuvant.

In one embodiment of this aspect, the pharmaceutical composition further comprises at least one additional therapeutic agent.

In one embodiment, the additional therapeutic agent is chosen from the group consisting of pain relieving agents, non-steroidal anti-inflammatory drugs (NSAIDs), cannabinoid receptor agonists, opiate receptor agonists, sodium channel blockers, N-type calcium channel blockers, local anesthetics, VR1 agonists and antagonists, agents used for migraine, anti-inflammatory and/or immunosuppressive agents, agents designed to treat tobacco abuse (e.g., nicotine receptor partial agonists and nicotine replacement therapies), ADD/ADHD agents, agents to treat alcoholism, such as opioid antagonists, agents for reducing alcohol withdrawal symptoms such as benzodiazepines and beta-blockers, antihypertensive agents such as ACE inhibitors and Angiotensin II Receptor blockers, Renin inhibitors, vasodilators, agents used to treat glaucoma such as direct-acting Miotics (cholinergic agonists), indirect acting Miotics (cholinesterase inhibitors), Carbonic anhydrase inhibitors, selective adrenergic agonists, Osmotic diuretics, antidepressants such as SSRIs, tricyclic antidepressants, and dopaminergic antidepressants, cognitive improvement agents, acetyl cholinesterase inhibitors, anti-emetic agents (e.g., 5HT3 antagonists), neuroprotective agents, neuroprotective agents currently under investigation, antipsychotic medications, agents used for multiple sclerosis, disease-modifying antirheumatic drugs (DMARDS), biological response modifiers (BRMs), COX-2 selective inhibitors, COX-1 inhibitors, immunosuppressives, PDE4 inhibitors, corticosteroids, histamine H1 receptor antagonists, histamine H2 receptor antagonists, proton pump inhibitors, leukotriene antagonists, 5-lipoxygenase inhibitors, nicotinic acetylcholine receptor agonists, P2X3 receptor antagonists, NGF agonists and antagonists, NK1 and NK2 antagonists, NMDA antagonist, potassium channel modulators, GABA modulators, and serotonergic and noradrenergic modulators.

In another aspect, the invention is a method for the treatment or prevention of pain comprising administering, alone or in combination therapy, to a patient in need thereof a therapeutically or prophylactically acceptable dose of a pharmaceutical composition comprising a compound of formula I, or a pharmaceutically acceptable salt thereof, and a pharmaceutically acceptable carrier, vehicle or adjuvant.

In one embodiment of this aspect, the pain is chronic pain, acute pain, perioperative pain (e.g., associated with surgery), postoperative pain, visceral pain, inflammatory pain, cancer pain, headache pain, neuropathic pain, dental pain (such as odontalgia), bone pain, joint pain (e.g., osteoarthritis or rheumatoid arthritis), myofascial pain (e.g., muscular injury, fibromyalgia), labor pain, pain associated with injuries, pain resulting from trauma, pain resulting from allergies, pain resulting from dermatitis, pain resulting from immunodeficiency, pain resulting from Hodgkin's disease, pain resulting from Myasthenia gravis, pain resulting from nephrotic syndrome, pain resulting from scleroderma, pain resulting from thyroiditis, central and peripheral pathway mediated pain, or pain associated with or the result of injury or age.

In one aspect, the invention is a method for the treatment or prevention of autoimmune disorders comprising administering, alone or in combination therapy, to a patient in need thereof a therapeutically or prophylactically acceptable dose of a pharmaceutical composition comprising a compound of formula I, or a pharmaceutically acceptable salt thereof, and a pharmaceutically acceptable carrier, vehicle or adjuvant.

In one embodiment of this aspect, the autoimmune disorder is selected from the group consisting of alopecia areata (also known as systemic sclerosis (SS)), amyloses, amyotrophic lateral sclerosis, ankylosing spondylarthritis, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, connective tissue diseases, crest syndrome, Crohn's disease, Degos' disease, dermatomyositis-juvenile, discoid lupus, essential mixed cryoglobulinemia, fibromyalgia-fibromyositis, graft vs. host disease, transplantation rejection, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, lupus erythrematosus, Meniere's disease, multiple sclerosis, myasthenia gravis, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Reynaud's phenomena, reactional arthritis, Reiter's syndrome, rheumatic fever, rheumatoid arthritis, sarcoidosis, scleroderma (progressive systemic sclerosis (PSS), Sjögren's syndrome, stiff-man syndrome, systemic lupus erythematosus, Takayasu arteritis, temporal arteritis/giant cell arteritis, ulcerative colitis, undifferentiated spondylarthritis, uveitis, vitiligo, and Wegener's granulomatosis.

In one aspect, the invention is a method for the treatment or prevention of disease-states or indications that are accompanied by inflammatory processes comprising administering, alone or in combination therapy, to a patient in need thereof a therapeutically or prophylactically acceptable dose of a pharmaceutical composition comprising a compound of formula I, or a pharmaceutically acceptable salt thereof, and a pharmaceutically acceptable carrier, vehicle or adjuvant.

In one embodiment of this aspect, the disease-states or indications that are accompanied by inflammatory processes are chosen from the group consisting of:
lung diseases such as asthma, bronchitis, allergic rhinitis, emphysema, adult respiratory distress syndrome (ARDS), pigeon fancier's disease, farmer's lung, chronic obstructive pulmonary disease (COPD), asthma including allergic asthma (atopic or non-atopic) as well as exercise-induced bronchoconstriction, occupational asthma, viral- or bacterial exacerbation of asthma, other non-allergic asthmas and “wheezy-infant syndrome”, pneumoconiosis, including aluminosis, anthracnosis, asbestosis, chalicosis, ptilosis, siderosis, silicosis, tabacosis and byssinosis;
rheumatic diseases or autoimmune diseases or musculoskeletal diseases such as all forms of rheumatic diseases, especially rheumatoid arthritis, acute rheumatic fever, and polymyalgia rheumatica; reactive arthritis; rheumatic soft tissue diseases; inflammatory soft tissue diseases of other genesis; arthritic symptoms in degenerative joint diseases (arthroses); tendinitis, bursitis, osteoarthritis, traumatic arthritis, gout (metabolic arthritis); collagenoses of any genesis, e.g., systemic lupus erythematosus, scleroderma, polymyositis, dermatomyositis, Sjögren syndrome, Still disease, Felty syndrome; and osteoporosis and other bone resorption diseases;
allergic diseases including all forms of allergic reactions, e.g., allergic rhinitis, allergic conjunctivitis infectious parasitic, angioneurotic edema, hay fever, insect bites, allergic reactions to drugs, blood derivatives, contrast agents, etc., anaphylactic shock (anaphylaxis), urticaria, angioneurotic edema, delayed or immediate hypersensitivity, and contact dermatitis;
vascular diseases such as panarteritis nodosa, polyarteritis nodosa, periarteritis nodosa, arteritis temporalis, Wegner granulomatosis, giant cell arthritis, atherosclerosis, reperfusion injury and erythema nodosum;
dermatological diseases such as dermatitis, psoriasis, sunburn, burns, and eczema;
renal, urinary and pancreatic diseases such as nephrotic syndrome and all types of nephritis (such as glomerulonephritis); pancreatitis; bladder hyperreflexia following bladder inflammation;
hepatic diseases such as acute liver cell disintegration; acute hepatitis of various genesis (such as viral, toxic, drug-induced) and chronically aggressive and/or chronically intermittent hepatitis, liver fibrosis associated with liver injury or disease, including fibrosis caused or exacerbated by alcoholic liver cirrhosis, chronic viral hepatitis, non alcoholic steatohepatitis and primary liver cancer;
gastrointestinal diseases such as inflammatory bowel diseases, irritable bowel syndrome, regional enteritis (Crohns' disease), colitis ulcerosa, gastritis, aphthous ulcer, celiac disease, regional ileitis, and gastroesophageal reflux disease;
neurodegenerative diseases such as in the treatment of neurodegeneration following stroke, cardiac arrest, pulmonary bypass, traumatic brain injury, spinal cord injury or the like;
eye diseases such as allergic keratitis, uveitis, or iritis, conjunctivitis, blepharitis, neuritis nervi optici, choroiditis, glaucoma and sympathetic ophthalmia;
diseases of the ear, nose, and throat (ENT) area such as tinnitus, allergic rhinitis or hay fever, otitis externa, caused by contact eczema, infection, etc., and otitis media;
neurological diseases such as brain edema, particularly tumor-related brain edema, multiple sclerosis, acute encephalomyelitis, meningitis, acute spinal cord injury, trauma, dementia, particularly degenerative dementia (including senile dementia, Alzheimer's disease, Parkinson's disease and Creutzfeldt-Jacob disease, Huntington's chorea, Pick's disease, motor neuron disease), vascular dementia (including multi-infarct dementia and dementia associated with intracranial space occupying lesions, infections and related conditions such as HIV infection), Guillain-Barre syndrome, myasthenia gravis, stroke, and various forms of seizures (such as nodding spasms);
blood diseases such as acquired hemolytic anemia, aplastic anemia, and idiopathic thrombocytopenia;
tumor diseases such as acute lymphatic leukemia, Hodgkin's disease, malignant lymphoma, lymphogranulomatoses, lymphosarcoma, solid malignant tumors, and extensive metastases;
endocrine diseases such as endocrine opthalmopathy, endocrine orbitopathia, thyrotoxic crisis, Thyroiditis de Quervain, Hashimoto thyroiditis, Morbus Basedow, granulomatous thyroiditis, struma lymphomatosa, Graves disease, type I diabetes (such as insulin-dependent diabetes); Organ and tissue transplantations and graft-versus-host diseases; and
severe states of shock such as septic shock, anaphylactic shock, and systemic inflammatory response syndrome (SIRS); and various other disease-states or conditions including, restenosis following percutaneous transluminal coronary angioplasty, acute and chronic pain, atherosclerosis, reperfusion injury, congestive heart failure, myocardial infarction, thermal injury, multiple organ injury secondary to trauma, necrotizing enterocolitis and syndromes associated with hemodialysis, leukopheresis, granulocyte transfusion, sarcoidosis, gingivitis, pyrexia. Edema resulting from trauma associated with burns, sprains or fracture, cerebral edema and angioedema, and diabetes (such as diabetic vasculopathy, diabetic neuropathy, diabetic retinopathy, post capillary resistance and diabetic symptoms associated with insulitis (e.g. Hyperglycemia, diuresis, proteinuria and increased nitrite and kallikrein urinary excretion)).

In one aspect, the invention is a method for the treatment or prevention of substance abuse related syndromes, disorders, diseases or withdrawal symptoms comprising administering, alone or in combination therapy, to a patient in need thereof a therapeutically or prophylactically acceptable dose of a pharmaceutical composition comprising a compound of formula I, or a pharmaceutically acceptable salt thereof, and a pharmaceutically acceptable carrier, vehicle or adjuvant.

In one embodiment of this aspect, the substance abuse related syndromes, disorders, diseases or withdrawal symptoms are chosen from the group consisting of drug abuse and drug withdrawal, wherein the abused substances include alcohol, amphetamines, amphetamine like substances, caffeine, cannabis, cocaine, hallucinogens, inhalants, opioids, nicotine (and/or tobacco products), heroin abuse, barbiturates, phencyclidine (or phencyclidine-like compounds), sedative-hypnotics, benzodiazepines, or combinations of any of the foregoing; and the withdrawal symptoms include tobacco craving or nicotine dependency, addiction, or withdrawal.

In one aspect, the invention is a method for the treatment or prevention of psychiatric disorders comprising administering, alone or in combination therapy, to a patient in need thereof a therapeutically or prophylactically acceptable dose of a pharmaceutical composition comprising a compound of formula I, or a pharmaceutically acceptable salt thereof, and a pharmaceutically acceptable carrier, vehicle or adjuvant.

In one embodiment of this aspect, the psychiatric disorders are chosen from the group consisting of depressions (including major depressive disorder, bipolar depression, unipolar depression, single or recurrent major depressive episodes (e.g., with or without psychotic features, catatonic features, and/or melancholic features), postpartum onset, seasonal affective disorder, dysthymic disorders (e.g., with early or late onset and with or without atypical features), neurotic depression and social phobia, depression accompanying dementia, anxiety, psychosis, social affective disorders, and/or cognitive disorders), manic-depressive psychoses, bipolar disorders, extreme psychotic states (such as mania, schizophrenia, and excessive mood swings where behavioral stabilization is desired), attention disorders such as ADHD (attention deficit hyperactivity disorders), autism, anxiety states, generalized anxiety, agoraphobia, as well as those behavioral states characterized by social withdrawal.

In one aspect, the invention is a method for the treatment or prevention of neurological or neurodegenerative disorders comprising administering, alone or in combination therapy, to a patient in need thereof a therapeutically or prophylactically acceptable dose of a pharmaceutical composition comprising a compound of formula I, or a pharmaceutically acceptable salt thereof, and a pharmaceutically acceptable carrier, vehicle or adjuvant.

In one embodiment of this aspect, the neurological or neurodegenerative disorders are chosen from the group consisting of dementia, particularly degenerative dementia (including senile dementia, Alzheimer's disease, Pick's disease, Huntington's chorea, Parkinson's disease and Creutzfeldt-Jakob disease, motor neuron disease); vascular dementia (including multi-infarct dementia); as well as dementia associated with intracranial space occupying lesions; trauma; infections and related conditions (including HIV infection); dementia in Parkinson's disease, metabolism; toxins; anoxia and vitamin deficiency; and mild cognitive impairment associated with ageing, particularly Age Associated Memory Impairment; amyotrophic lateral sclerosis (ALS), multiple sclerosis, epilepsy, ischemia, traumatic head or brain injury, brain inflammation, eye injury, stroke and neuroinflammation.

In one aspect, the invention is a method for the treatment or prevention of ocular disorders comprising administering, alone or in combination therapy, to a patient in need thereof a therapeutically or prophylactically acceptable dose of a pharmaceutical composition comprising a compound of formula I, or a pharmaceutically acceptable salt thereof, and a pharmaceutically acceptable carrier, vehicle or adjuvant.

In one embodiment of this aspect, the ocular disorders are chosen from the group consisting of glaucoma (such as normal tension glaucoma), glaucoma-associated intraocular pressure retinitis, retinopathies, uveitis, acute injury to the eye tissue (e.g. conjunctivitis), high intraocular pressure, family history of glaucoma, glaucoma in the contralateral eye and high myopia.

In one embodiment, the patient is a human.

In one embodiment, the patient is a companion animal, exotic animal or a farm animal such as a dog, cat, mouse, rat, hamster, gerbil, guinea pig, rabbit, horse, pig or cow.

In one aspect, the invention is a method of increasing CB receptor activity in a biological sample, comprising contacting said biological sample with a composition comprising a compound of formula I, or a pharmaceutically acceptable salt thereof, and a pharmaceutically acceptable carrier, vehicle or adjuvant.

### Pharmaceutically Acceptable Salts, Co-Forms and Pro-Drugs of the Invention.

The phrase “pharmaceutically acceptable salt,” as used herein, refers to pharmaceutically acceptable organic or inorganic salts of a compound of Formula I. For use in medicine, the salts of the compounds of Formula I will be pharmaceutically acceptable salts. Other salts may, however, be useful in the preparation of the compounds of Formula I or of their pharmaceutically acceptable salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more (i.e. up to 6) charged atoms and/or one or more (i.e. up to 6) counter ion.

Pharmaceutically acceptable salts of the compounds described herein include those derived from suitable inorganic and organic acids and bases. In some embodiments, the salts can be prepared in situ during the final isolation and purification of the compounds. In other embodiments the salts can be prepared from the free form of the compound in a separate synthetic step.

When the compound of Formula I is acidic or contains a sufficiently acidic bioisostere, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous salts, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine, caffeine, choline, N,N.sup.1-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound of Formula I is basic or contains a sufficiently basic bioisostere, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particular embodiments include citric, hydrobromic, hydrochloric, maleic, phosphoric, sulfuric and tartaric acids. Other exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The preparation of the pharmaceutically acceptable salts described above and other typical pharmaceutically acceptable salts is more fully described by Berg et al., “Pharmaceutical Salts,” J. Pharm. Sci., 1977:66:1-19, incorporated here by reference in its entirety.

In addition to the compounds described herein and their pharmaceutically acceptable salts, pharmaceutically acceptable solvates (e.g., hydrates) and co-crystals of these compounds and salts may also be employed in compositions to treat or prevent the herein identified disorders.

As used herein, the term “pharmaceutically acceptable solvate,” is a solvate formed from the association of one or more (i.e. up to 6) pharmaceutically acceptable solvent molecules to one of the compounds described herein. As used herein, the term “hydrate” means a compound described herein or a salt thereof that further includes a stoichiometric or non-stoichiometric amount of water bound by non-covalent intermolecular forces. The term solvate includes hydrates (e.g., hemi hydrate, monohydrate, dihydrate, trihydrate, tetrahydrate, and the like).

“Pharmaceutically acceptable co-crystals” result when a pharmaceutically active compound crystallizes with another material (e.g. a carboxylic acid, a 4,4′-bipyridine or an excipient) that is also a solid at room temperature. Some pharmaceutically acceptable excipients are described in the next section. Other pharmaceutically acceptable substances that can be used to form co-crystals are exemplified by the GRAS (Generally regarded as safe) list of the US FDA.

In addition to the compounds described herein, pharmaceutically acceptable pro-drugs of these compounds may also be employed in compositions to treat or prevent the herein identified disorders.

A “pharmaceutically acceptable pro-drug” includes any pharmaceutically acceptable ester, salt of an ester or other derivative or salt thereof of a compound described herein which, upon administration to a recipient, is capable of providing, either directly or indirectly, a compound described herein. Particularly favoured pro-drugs are those that increase the bioavailability of the compounds when such compounds are administered to a patient (e.g., by allowing an orally administered compound to be more readily absorbed into the blood) or which enhance delivery of the parent compound to a biological compartment (e.g., the brain or lymphatic system) relative to the parent species. The term “pro-drug” encompasses a derivative of a compound that can hydrolyze, oxidize, or otherwise react under biological conditions (in vitro or in vivo) to provide a compound described herein. Examples of pro-drugs contemplated in this invention include, but are not limited to, analogs or derivatives of compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of pro-drugs include derivatives of compounds described herein that comprise —NO, —NO₂, —ONO, or —ONO₂ moieties. Pro-drugs can typically be prepared using well-known methods, such as those described by Burger's Medicinal Chemistry and Drug Discovery, 1995,172-178, 949-982 (Manfred E. Wolff ed., 5th ed.).

### Pharmaceutical Compositions and Methods of Administration.

The compounds herein disclosed, and their pharmaceutically acceptable salts, solvates, co-crystals and pro-drugs thereof may be formulated as pharmaceutical compositions or “formulations”.

A typical formulation is prepared by mixing a compound of Formula I, or a pharmaceutically acceptable salt, solvate, co-crystal or pro-drug thereof, and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of Formula I is being formulated. Solvents are generally selected based on solvents recognized by persons skilled in the art as safe (GRAS-Generally Regarded as Safe) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include other types of excipients such as one or more (i.e. up to 6) buffers, stabilizing agents, antiadherents, surfactants, wetting agents, lubricating agents, emulsifiers, binders, suspending agents, disintegrants, fillers, sorbents, coatings (e.g. enteric or slow release) preservatives, antioxidants, opaquing agents, glidants, processing aids, colorants, sweeteners, perfuming agents, flavoring agents and other known additives to provide an elegant presentation of the drug (i.e., a compound of Formula I or pharmaceutical composition thereof) or aid in the manufacturing of the pharmaceutical product (i.e., medicament).

The formulations may be prepared using conventional dissolution and mixing procedures. For example, the bulk drug substance (i.e., compound of Formula I, a pharmaceutically acceptable salt, solvate, co-crystal or pro-drug thereof, or a stabilized form of the compound, such as a complex with a cyclodextrin derivative or other known complexation agent) is dissolved in a suitable solvent in the presence of one or more (i.e. up to 6) of the excipients described above. A compound having the desired degree of purity is optionally mixed with pharmaceutically acceptable diluents, carriers, excipients or stabilizers, in the form of a lyophilized formulation, milled powder, or an aqueous solution. Formulation may be conducted by mixing at ambient temperature at the appropriate pH, and at the desired degree of purity, with physiologically acceptable carriers. The pH of the formulation depends mainly on the particular use and the concentration of compound, but may range from about 3 to about 8.

The compound of Formula I or a pharmaceutically acceptable salt, solvate, co-crystal or pro-drug thereof is typically formulated into pharmaceutical dosage forms to provide an easily controllable dosage of the drug and to enable patient compliance with the prescribed regimen. Pharmaceutical formulations of compounds of Formula I, or a pharmaceutically acceptable salt, solvate, co-crystal or pro-drug thereof, may be prepared for various routes and types of administration. Various dosage forms may exist for the same compound, since different medical conditions may warrant different routes of administration. The amount of active ingredient that may be combined with the carrier material to produce a single dosage form will vary depending upon the subject treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur. As a general proposition, the initial pharmaceutically effective amount of the inhibitor administered will be in the range of about 0.01-100 mg/kg per dose, namely about 0.1 to 20 mg/kg of patient body weight per day, with the typical initial range of compound used being 0.3 to 15 mg/kg/day.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically or pharmaceutically effective amount” of the compound to be administered will be governed by such considerations, and is the minimum amount necessary to ameliorate, cure or treat the disease or disorder or one or more (i.e. up to 6) of its symptoms.

The pharmaceutical compositions of Formula I will be formulated, dosed, and administered in a fashion, i.e., amounts, concentrations, schedules, course, vehicles, and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners, such as the age, weight, and response of the individual patient.

The term “prophylactically effective amount” refers to an amount effective in preventing or substantially lessening the chances of acquiring a disease or disorder or in reducing the severity of the disease or disorder or one or more (i.e. up to 6) of its symptoms before it is acquired or before the symptoms develop. Roughly, prophylactic measures are divided between primary prophylaxis (to prevent the development of a disease) and secondary prophylaxis (whereby the disease has already developed and the patient is protected against worsening of this process).

Acceptable diluents, carriers, excipients, and stabilizers are those that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulin; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, e.g., hydroxymethylcellulose or gelatin-microcapsules and poly-(methylmetacrylate) microcapsules, respectively, in colloidal drug delivery systems (for example, liposomes, albumin microspheres, micro emulsions, nano-particles and nanocapsules) or in macroemulsions. Such techniques are disclosed in Remington's: The Science and Practice of Pharmacy, 21^{st} Edition, University of the Sciences in Philadelphia, Eds., 2005 (hereafter “Remington's”).

“Controlled drug delivery systems” supply the drug to the body in a manner precisely controlled to suit the drug and the conditions being treated. The primary aim is to achieve a therapeutic drug concentration at the site of action for the desired duration of time. The term “controlled release” is often used to refer to a variety of methods that modify release of drug from a dosage form. This term includes preparations labeled as “extended release”, “delayed release”, “modified release” or “sustained release”. In general, one can provide for controlled release of the agents described herein through the use of a wide variety of polymeric carriers and controlled release systems including erodible and non-erodible matrices, osmotic control devices, various reservoir devices, enteric coatings and multiparticulate control devices.

“Sustained-release preparations” are the most common applications of controlled release. Suitable examples of sustained-release preparations include semipermeable matrices of solid hydrophobic polymers containing the compound, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers and poly-D-(−)-3-hydroxybutyric acid.

“Immediate-release preparations” may also be prepared. The objective of these formulations is to get the drug into the bloodstream and to the site of action as rapidly as possible. For instance, for rapid dissolution, most tablets are designed to undergo rapid disintegration to granules and subsequent deaggregation to fine particles. This provides a larger surface area exposed to the dissolution medium, resulting in a faster dissolution rate.

Agents described herein can be incorporated into an erodible or non-erodible polymeric matrix controlled release device. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous environment of use, the erodible polymeric matrix imbibes water and forms an aqueous-swollen gel or matrix that entraps the agent described herein. The aqueous-swollen matrix gradually erodes, swells, disintegrates or dissolves in the environment of use, thereby controlling the release of a compound described herein to the environment of use. One ingredient of this water-swollen matrix is the water-swellable, erodible, or soluble polymer, which may generally be described as an osmopolymer, hydrogel or water-swellable polymer. Such polymers may be linear, branched, or cross linked. The polymers may be homopolymers or copolymers. In certain embodiments, they may be synthetic polymers derived from vinyl, acrylate, methacrylate, urethane, ester and oxide monomers. In other embodiments, they can be derivatives of naturally occurring polymers such as polysaccharides (e.g. chitin, chitosan, dextran and pullulan; gum agar, gum arabic, gum karaya, locust bean gum, gum tragacanth, carrageenans, gum ghatti, guar gum, xanthan gum and scleroglucan), starches (e.g. dextrin and maltodextrin), hydrophilic colloids (e.g. pectin), phosphatides (e.g. lecithin), alginates (e.g. ammonium alginate, sodium, potassium or calcium alginate, propylene glycol alginate), gelatin, collagen, and cellulosics. Cellulosics are cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent. In certain embodiments, the cellulosics for the erodible matrix comprises aqueous-soluble and aqueous-erodible cellulosics can include, for example, ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC). In certain embodiments, the cellulosics comprises various grades of low viscosity (MW less than or equal to 50,000 Daltons, for example, the Dow Methocel™ series E5, E15LV, E50LV and K100LY) and high viscosity (MW greater than 50,000 Daltons, for example, E4MCR, E10MCR, K4M, K15M and K100M and the Methocel™ K series) HPMC. Other commercially available types of HPMC include the Shin Etsu Metolose 90SH series.

Other materials useful as the erodible matrix material include, but are not limited to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGITO, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

Alternatively, the agents of the present invention may be administered by or incorporated into a non-erodible matrix device. In such devices, an agent described herein is distributed in an inert matrix. The agent is released by diffusion through the inert matrix. Examples of materials suitable for the inert matrix include insoluble plastics (e.g. methyl acrylate-methyl methacrylate copolymers, polyvinyl chloride, polyethylene), hydrophilic polymers (e.g. ethyl cellulose, cellulose acetate, cross linked polyvinylpyrrolidone (also known as crospovidone)), and fatty compounds (e.g. carnauba wax, microcrystalline wax, and triglycerides). Such devices are described further in Remington: The Science and Practice of Pharmacy, 20th edition (2000).

As noted above, the agents described herein may also be incorporated into an osmotic control device. Such devices generally include a core containing one or more (i.e. up to 6) agents as described herein and a water-permeable, non-dissolving and non-eroding coating surrounding the core which controls the influx of water into the core from an aqueous environment of use so as to cause drug release by extrusion of some or the entire core to the environment of use. In certain embodiments, the coating is polymeric, aqueous-permeable, and has at least one delivery port. The core of the osmotic device optionally includes an osmotic agent which acts to imbibe water from the surrounding environment via such a semi-permeable membrane. The osmotic agent contained in the core of this device may be an aqueous-swellable hydrophilic polymer or it may be an osmogen, also known as an osmagent. Pressure is generated within the device which forces the agent(s) out of the device via an orifice (of a size designed to minimize solute diffusion while preventing the build-up of a hydrostatic pressure head). Non-limiting examples of osmotic control devices are disclosed in U.S. patent application Ser. No. 09/495,061.

The amount of water-swellable hydrophilic polymers present in the core may range from about 5 to about 80 wt % (including for example, 10 to 50 wt %). Non limiting examples of core materials include hydrophilic vinyl and acrylic polymers, polysaccharides such as calcium alginate, polyethylene oxide (PEO), polyethylene glycol (PEG), polypropylene glycol (PPG), poly (2-hydroxyethyl methacrylate), poly (acrylic) acid, poly (methacrylic) acid, polyvinylpyrrolidone (PVP) and cross linked PVP, polyvinyl alcohol (PVA), PVA/PVP copolymers and PVA/PVP copolymers with hydrophobic monomers such as methyl methacrylate, vinyl acetate, and the like, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl cellulose (CEC), sodium alginate, polycarbophil, gelatin, xanthan gum, and sodium starch glycolat. Other materials include hydrogels comprising interpenetrating networks of polymers that may be formed by addition or by condensation polymerization, the components of which may comprise hydrophilic and hydrophobic monomers such as those just mentioned. Water-swellable hydrophilic polymers include but are not limited to PEO, PEG, PVP, sodium croscarmellose, HPMC, sodium starch glycolate, polyacrylic acid and cross linked versions or mixtures thereof.

The core may also include an osmogen (or osmagent). The amount of osmogen present in the core may range from about 2 to about 70 wt % (including, for example, from 10 to 50 wt %). Typical classes of suitable osmogens are water-soluble organic acids, salts and sugars that are capable of imbibing water to, thereby, effect an osmotic pressure gradient across the barrier of the surrounding coating. Typical useful osmogens include but are not limited to magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, inositol, raffinose, sucrose, glucose, fructose, lactose, citric acid, succinic acid, tartaric acid, and mixtures thereof. In certain embodiments, the osmogen is glucose, lactose, sucrose, mannitol, xylitol, sodium chloride, including combinations thereof.

The rate of drug delivery is controlled by such factors as the permeability and thickness of the coating, the osmotic pressure of the drug-containing layer, the degree of hydrophilicity of the hydrogel layer, and the surface area of the device. Those skilled in the art will appreciate that increasing the thickness of the coating will reduce the release rate, while any of the following will increase the release rate: increasing the permeability of the coating; increasing the hydrophilicity of the hydrogel layer; increasing the osmotic pressure of the drug-containing layer; or increasing the device's surface area.

In certain embodiments, entrainment of particles of agents described herein in the extruding fluid during operation of such osmotic device is desirable. For the particles to be well entrained, the agent drug form is dispersed in the fluid before the particles have an opportunity to settle in the tablet core. One means of accomplishing this is by adding a disintegrant that serves to break up the compressed core into its particulate components. Non-limiting examples of standard disintegrants include materials such as sodium starch glycolate (e.g., Explotab™ CLV), microcrystalline cellulose (e.g., Avicel™) microcrystalline silicified cellulose (e.g., ProSoIv™) and croscarmellose sodium (e.g., Ac-Di-Sol™), and other disintegrants known to those skilled in the art. Depending upon the particular formulation, some disintegrants work better than others. Several disintegrants tend to form gels as they swell with water, thus hindering drug delivery from the device. Non-gelling, non-swelling disintegrants provide a more rapid dispersion of the drug particles within the core as water enters the core. In certain embodiments, non-gelling, non-swelling disintegrants are resins, for example, ion-exchange resins. In one embodiment, the resin is Amberlite™ IRP 88 (available from Rohm and Haas, Philadelphia, Pa.). When used, the disintegrant is present in amounts ranging from about 1-25% of the core agent.

Another example of an osmotic device is an osmotic capsule. The capsule shell or portion of the capsule shell can be semipermeable. The capsule can be filled either by a powder or liquid consisting of an agent described herein, excipients that imbibe water to provide osmotic potential, and/or a water-swellable polymer, or optionally solubilizing excipients. The capsule core can also be made such that it has a bilayer or multilayer agent analogous to the bilayer, trilayer or concentric geometries described above.

Another class of osmotic device useful in this invention comprises coated swellable tablets, for example, as described in EP378404. Coated swellable tablets comprise a tablet core comprising an agent described herein and a swelling material, preferably a hydrophilic polymer, coated with a membrane, which contains holes, or pores through which, in the aqueous use environment, the hydrophilic polymer can extrude and carry out the agent. Alternatively, the membrane may contain polymeric or low molecular weight water-soluble porosigens. Porosigens dissolve in the aqueous use environment, providing pores through which the hydrophilic polymer and agent may extrude. Examples of porosigens are water-soluble polymers such as HPMC, PEG, and low molecular weight compounds such as glycerol, sucrose, glucose, and sodium chloride. In addition, pores may be formed in the coating by drilling holes in the coating using a laser or other mechanical means. In this class of osmotic devices, the membrane material may comprise any film-forming polymer, including polymers which are water permeable or impermeable, providing that the membrane deposited on the tablet core is porous or contains water-soluble porosigens or possesses a macroscopic hole for water ingress and drug release. Embodiments of this class of sustained release devices may also be multilayered, as described, for example, in EP378404.

When an agent described herein is a liquid or oil, such as a lipid vehicle formulation, for example as described in WO05/011634, the osmotic controlled-release device may comprise a soft-gel or gelatin capsule formed with a composite wall and comprising the liquid formulation where the wall comprises a barrier layer formed over the external surface of the capsule, an expandable layer formed over the barrier layer, and a semipermeable layer formed over the expandable layer. A delivery port connects the liquid formulation with the aqueous use environment. Such devices are described, for example, in U.S. Pat. No. 6,419,952, U.S. Pat. No. 6,342,249, U.S. Pat. No. 5,324,280, U.S. Pat. No. 4,672,850, U.S. Pat. No. 4,627,850, U.S. Pat. No. 4,203,440, and U.S. Pat. No. 3,995,631.

As further noted above, the agents described herein may be provided in the form of microparticulates, generally ranging in size from about 10 μm to about 2 mm (including, for example, from about 100 μm to 1 mm in diameter). Such multiparticulates may be packaged, for example, in a capsule such as a gelatin capsule or a capsule formed from an aqueous-soluble polymer such as HPMCAS, HPMC or starch; dosed as a suspension or slurry in a liquid; or they may be formed into a tablet, caplet, or pill by compression or other processes known in the art. Such multiparticulates may be made by any known process, such as wet- and dry-granulation processes, extrusion/spheronization, roller-compaction, melt-congealing, or by spray-coating seed cores. For example, in wet- and dry-granulation processes, the agent described herein and optional excipients may be granulated to form multiparticulates of the desired size.

The agents can be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifier”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

The compounds described herein can be incorporated into pharmaceutically-acceptable nanoparticle, nanosphere, and nanocapsule formulations (Delie and Blanco-Prieto 2005 Molecule 10:65-80). Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, ultrafine particles (sized around 0.1 μm) can be designed using polymers able to be degraded in vivo (e.g. biodegradable polyalkyl-cyanoacrylate nanoparticles). Such particles are described in the prior art (Couvreur et al, 1980; 1988; zur Muhlen et al., 1998; Zambaux et al. 1998; Pinto-Alphandry et al., 1995 and U.S. Pat. No. 5,145,684).

Implantable devices coated with a compound of this invention are another embodiment of the present invention. The compounds may also be coated on implantable medical devices, such as beads, or co-formulated with a polymer or other molecule, to provide a “drug depot”, thus permitting the drug to be released over a longer time period than administration of an aqueous solution of the drug. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

The formulations include those suitable for the administration routes detailed herein. The formulations may conveniently be presented in unit dosage form and may be prepared by any of the methods well known in the art of pharmacy. Techniques and formulations generally are found in Remington's. Such methods include the step of bringing into association the active ingredient with the carrier which constitutes one or more (i.e. up to 6) accessory ingredients. In general the formulations are prepared by uniformly and intimately bringing into association the active ingredient with liquid carriers or finely divided solid carriers or both, and then, if necessary, shaping the product.

The terms “administer”, “administering” or “administration” in reference to a compound, composition or formulation of the invention means introducing the compound into the system of the animal in need of treatment. When a compound of the invention is provided in combination with one or more (i.e. up to 6) other active agents, “administration” and its variants are each understood to include concurrent and/or sequential introduction of the compound and the other active agents.

The compositions described herein may be administered systemically or locally, e.g.: orally (e.g. using capsules, powders, solutions, suspensions, tablets, sublingual tablets and the like), by inhalation (e.g. with an aerosol, gas, inhaler, nebulizer or the like), to the ear (e.g. using ear drops), topically (e.g. using creams, gels, liniments, lotions, ointments, pastes, transdermal patches, etc), ophthalmically (e.g. with eye drops, ophthalmic gels, ophthalmic ointments), rectally (e.g. using enemas or suppositories), nasally, buccally, vaginally (e.g. using douches, intrauterine devices, vaginal suppositories, vaginal rings or tablets, etc), via an implanted reservoir or the like, or parenterally depending on the severity and type of the disease being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

The pharmaceutical compositions described herein may be orally administered in any orally acceptable dosage form including, but not limited to, capsules, tablets, aqueous suspensions or solutions. Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. Tablets may be uncoated or may be coated by known techniques including microencapsulation to mask an unpleasant taste or to delay disintegration and adsorption in the gastrointestinal tract and thereby provide a sustained action over a longer period. For example, a time delay material such as glyceryl monostearate or glyceryl distearate alone or with a wax may be employed. A water soluble taste masking material such as hydroxypropyl-methylcellulose or hydroxypropyl-cellulose may be employed.

Formulations of a compound of Formula I that are suitable for oral administration may be prepared as discrete units such as tablets, pills, troches, lozenges, aqueous or oil suspensions, dispersible powders or granules, emulsions, hard or soft capsules, e.g. gelatin capsules, syrups or elixirs. Formulations of a compound intended for oral use may be prepared according to any method known to the art for the manufacture of pharmaceutical compositions.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

The active compounds can also be in microencapsulated form with one or more (i.e. up to 6) excipients as noted above.

When aqueous suspensions are required for oral use, the active ingredient is combined with emulsifying and suspending agents. If desired, certain sweetening and/or flavoring agents may be added. Syrups and elixirs may be formulated with sweetening agents, for example glycerol, propylene glycol, sorbitol or sucrose. Such formulations may also contain a demulcent, a preservative, flavoring and coloring agents and antioxidant.

Sterile injectable forms of the compositions described herein (e.g. for parenteral administration) may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage fauns including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of injectable formulations.

Oily suspensions may be formulated by suspending the compound of Formula I in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Aqueous suspensions of compounds of Formula I contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more (i.e. up to 6) preservatives such as ethyl or n-propyl p-hydroxy-benzoate, one or more (i.e. up to 6) coloring agents, one or more (i.e. up to 6) flavoring agents and one or more (i.e. up to 6) sweetening agents, such as sucrose or saccharin.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsulated matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

The injectable solutions or microemulsions may be introduced into a patient's bloodstream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS model 5400 intravenous pump.

Compositions for rectal or vaginal administration are preferably suppositories which can be prepared by mixing the compounds described herein with suitable non-irritating excipients or carriers such as cocoa butter, beeswax, polyethylene glycol or a suppository wax which are solid at ambient temperature but liquid at body temperature and therefore melt in the rectum or vaginal cavity and release the active compound. Other formulations suitable for vaginal administration may be presented as pessaries, tampons, creams, gels, pastes, foams or sprays.

The pharmaceutical compositions described herein may also be administered topically, especially when the target of treatment includes areas or organs readily accessible by topical application, including diseases of the eye, the ear, the skin, or the lower intestinal tract. Suitable topical formulations are readily prepared for each of these areas or organs.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more (i.e. up to 6) carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more (i.e. up to 6) pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum. For treatment of the eye or other external tissues, e.g., mouth and skin, the formulations may be applied as a topical ointment or cream containing the active ingredient(s) in an amount of, for example, 0.075 to 20% w/w. When formulated in an ointment, the active ingredients may be employed with either an oil-based, paraffinic or a water-miscible ointment base.

Alternatively, the active ingredients may be formulated in a cream with an oil-in-water cream base. If desired, the aqueous phase of the cream base may include a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of emulsions prepared using compounds of Formula I may be constituted from known ingredients in a known manner. While the phase may comprise merely an emulsifier (otherwise known as an emulgent), it desirably comprises a mixture of at least one emulsifier with a fat or an oil or with both a fat and an oil. A hydrophilic emulsifier may be included together with a lipophilic emulsifier which acts as a stabilizer. In some embodiments, the emulsifier includes both oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulgents and emulsion stabilizers suitable for use in the formulation of compounds of Formula I include Tween™-60, Span™-80, cetostearyl alcohol, benzyl alcohol, myristyl alcohol, glyceryl mono-stearate and sodium lauryl sulfate.

The pharmaceutical compositions may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. Formulations suitable for intrapulmonary or nasal administration have a particle size for example in the range of 0.1 to 500 micros (including particles in a range between 0.1 and 500 microns in increments microns such as 0.5, 1, 30, 35 microns, etc) which is administered by rapid inhalation through the nasal passage or by inhalation through the mouth so as to reach the alveolar sacs.

The pharmaceutical composition (or formulation) for use may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

In another aspect, a compound of Formula I or a pharmaceutically acceptable salt, co-crystal, solvate or pro-drug thereof may be formulated in a veterinary composition comprising a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Therapeutic Methods

The terms, “disease”, “disorder”, and “condition” may be used interchangeably here to refer to a CB receptor mediated medical or pathological condition.

As used herein, the terms “subject” and “patient” are used interchangeably. The terms “subject” and “patient” refer to an animal (e.g., a bird such as a chicken, quail or turkey, or a mammal), preferably a “mammal” including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

The term “biological sample”, as used herein, refers to an in vitro or ex vivo sample, and includes, without limitation, cell cultures or extracts thereof; biopsied material obtained from a mammal or extracts thereof; blood, saliva, urine, faeces, semen, tears, lymphatic fluid, ocular fluid, vitreous humour, or other body fluids or extracts thereof.

“Treat”, “treating” or “treatment” with regard to a disorder or disease refers to alleviating or abrogating the cause and/or the effects of the disorder or disease. As used herein, the terms “treat”, “treatment” and “treating” refer to the reduction or amelioration of the progression, severity and/or duration of a CB receptor mediated condition, or the amelioration of one or more (i.e. up to 6) symptoms (preferably, one or more (i.e. up to 6) discernible symptoms) of said condition, resulting from the administration of one or more (i.e. up to 6) therapies (e.g., one or more (i.e. up to 6) therapeutic agents such as a compound or composition of the invention). In specific embodiments, the terms “treat”, “treatment” and “treating” refer to the amelioration of at least one measurable physical parameter of a CB receptor mediated condition. In other embodiments the terms “treat”, “treatment” and “treating” refer to the inhibition of the progression of a CB receptor mediated condition, either physically by, e.g., stabilization of a discernible symptom, physiologically by, e.g., stabilization of a physical parameter, or both.

As used herein, the terms “prevent”, “preventing” and “prevention” with regard to a disorder or disease refer to averting the cause and/or effects of a disease or disorder prior to the disease or disorder manifesting itself. The terms “prophylaxis” or “prophylactic use”, as used herein, refer to any medical or public health procedure whose purpose is to prevent, rather than treat or cure a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a person with the disease.

The term “chemotherapy” refers to the use of medications, e.g. small molecule drugs (rather than “vaccines”) for treating a disorder or disease.

The term “chemoprophylaxis” refers to the use of medications, e.g. small molecule drugs (rather than “vaccines”) for the prevention of a disorder or disease.

In one embodiment, the methods of the invention are a preventative or “pre-emptive” measure to a patient, preferably a human; having a predisposition to developing a CB receptor related disease or symptom.

The compounds and pharmaceutical compositions described herein can be used alone or in combination therapy for the treatment or prevention of pain. The pain can be chronic pain, acute pain, perioperative pain (e.g., associated with surgery), postoperative pain, visceral pain, inflammatory pain, cancer pain, headache pain, neuropathic pain, dental pain (such as odontalgia), bone pain, joint pain (e.g., osteoarthritis or rheumatoid arthritis), myofascial pain (e.g., muscular injury, fibromyalgia), labor pain, pain associated with injuries, trauma, allergies, dermatitis, immunodeficiency, Hodgkin's disease, Myasthenia gravis, nephrotic syndrome, scleroderma, or thyroiditis, central and peripheral pathway mediated pain, or pain associated with or the result of injury or age.

Neuropathic pain can be associated with neuronal lesions such as those induced by diabetes, HIV, herpes infection, or stroke. Chronic pain can result from injury and/or inflammation and includes chronic lower back pain, as well as pain from osteoarthritis or rheumatoid arthritis. Acute pain includes, for example, traumatic pain (e.g., bony fracture pain, sprains, strains and soft tissue damage), muscle pain, burn pain, and sun burn pain. Neuropathic pain can be associated with, for example, nerve injury, head trauma, hyperalgesia, allodynia, sciatica, amputation, trigeminal neuralgia, chemotherapeutic neuropathy, AIDS-related neuropathy, diabetic neuropathy, painful traumatic mononeuropathy, painful polyneuropathy, multiple sclerosis, root avulsions, postthoracotomy syndrome, central nervous system injury, non-herpetic neuralgia and post herpetic neuralgia. Neuropathic pain also includes lower back pain, toxin induced pain, chemotherapy induced pain, phantom limb pain, thalamic pain syndrome, post-stroke pain, stump pain, repetitive motion pain, pain induced by post mastectomy syndrome.

Visceral pain includes, for example, pain associated with pancreatitis, peptic ulcer, interstitial cystitis, renal colic, angina, dysmenorrhea, menstruation, irritable bowel syndrome (IBS), myocardial ischemia, and non-ulcer dyspepsia. Visceral pain also includes gynecological pain, non-cardiac chest pain, and chronic pelvic pain.

Inflammatory pain includes, for example, pain induced by or associated with disorders such as osteoarthritis, rheumatic fever, rheumatoid arthritis, rheumatic disease, tendonitis, juvenile arthritis, spondylitis, gouty arthritis, psoriatic arthritis, interstitial cystitis, peripheral neuritis, mucositis, fibromyalgia, pancreatitis, enteritis, cellulites, bony fractures, post-operative ileus, irritable bowel syndrome, Crohn's Disease, ulcerative colitis, cholecystitis, teno-synovitis, gout, vulvodynia, fibromyalgia, sprains and strains, systemic lupus erythematosus, myositis, and influenza and other viral infections such as the common cold. Inflammatory pain also includes sympathetically maintained pain, pain due to venomous and non-venomous snake bite, spider bite or insect sting, sports injury pain, myofascial pain (muscular injury, fibromyalgia), musculoskeletal pain, and pain due to inflammatory bowel diseases.

Cancer pain can be induced by or associated with tumors such as lymphatic leukemia, Hodgkin's disease, malignant lymphoma, lymphogranulomatoses, lymphosarcoma, solid malignant tumors, and extensive metastases.

Headache pain includes cluster headache, migraine with and without aura, tension type headache, headaches caused by injury or infection, hangovers, and headaches with unknown origins.

The compounds and pharmaceutical compositions described herein can be used alone or in combination therapy for the treatment or prevention of autoimmune disorders including, for example, alopecia areata (also known as systemic sclerosis (SS)), amyloses, amyotrophic lateral sclerosis, ankylosing spondylarthritis, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, connective tissue diseases, crest syndrome, Crohn's disease, Degos' disease, dermatomyositis-juvenile, discoid lupus, essential mixed cryoglobulinemia, fibromyalgia-fibromyositis, graft vs. host disease, transplantation rejection, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, lupus erythematosus, Meniere's disease, multiple sclerosis, myasthenia gravis, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Reynaud's phenomena, reactional arthritis, Reiter's syndrome, rheumatic fever, rheumatoid arthritis, sarcoidosis, scleroderma (progressive systemic sclerosis (PSS), Sjögren's syndrome, stiff-man syndrome, systemic lupus erythematosus, Takayasu arteritis, temporal arteritis/giant cell arteritis, ulcerative colitis, undifferentiated spondylarthritis, uveitis, vitiligo, and Wegener's granulomatosis.

The compounds and pharmaceutical compositions described herein can be used alone or in combination therapy for the treatment or prevention of inflammatory disorders, including, for example, chronic and acute inflammatory disorders. Examples of disorders with inflammatory components include asthma, atopic allergy, allergy, atherosclerosis, bronchial asthma, eczema, glomerulonephritis, graft vs. host disease, hemolytic anemia, osteoarthritis, sepsis, septic shock (e.g. as antihypovolemic and/or antihypotensive agents), stroke, transplantation of tissue and organs, vasculitis, diabetic retinopathy and ventilator induced lung injury. The compounds and pharmaceutical compositions described herein can also be used alone or in combination therapy for the treatment or prevention of disease-states or indications that are accompanied by inflammatory processes such as:

(1) Lung diseases: e.g. asthma, bronchitis, allergic rhinitis, emphysema, adult respiratory distress syndrome (ARDS), pigeon fancier's disease, farmer's lung, chronic obstructive pulmonary disease (COPD), asthma including allergic asthma (atopic or non-atopic) as well as exercise-induced bronchoconstriction, occupational asthma, viral- or bacterial exacerbation of asthma, other non-allergic asthmas and “wheezy-infant syndrome”, pneumoconiosis, including aluminosis, anthracosis, asbestosis, chalicosis, ptilosis, siderosis, silicosis, tabacosis and byssinosis;

(2) Rheumatic diseases or autoimmune diseases or musculoskeletal diseases: e.g., all fauns of rheumatic diseases, especially rheumatoid arthritis, acute rheumatic fever, and polymyalgia rheumatica; reactive arthritis; rheumatic soft tissue diseases; inflammatory soft tissue diseases of other genesis; arthritic symptoms in degenerative joint diseases (arthroses); tendinitis, bursitis, osteoarthritis, traumatic arthritis, gout (metabolic arthritis); collagenoses of any genesis, e.g., systemic lupus erythematosus, scleroderma, polymyositis, dermatomyositis, Sjögren syndrome, Still disease, Felty syndrome; and osteoporosis and other bone resorption diseases;

(3) Allergic diseases including all forms of allergic reactions, e.g., allergic rhinitis, allergic conjunctivitis infectious parasitic, angioneurotic edema, hay fever, insect bites, allergic reactions to drugs, blood derivatives, contrast agents, etc., anaphylactic shock (anaphylaxis), urticaria, angioneurotic edema, delayed or immediate hypersensitivity, and contact dermatitis;

(4) Vascular diseases: e.g., panarteritis nodosa, polyarteritis nodosa, periarteritis nodosa, arteritis temporalis, Wegner granulomatosis, giant cell arthritis, atherosclerosis, reperfusion injury and erythema nodosum;

(5) Dermatological diseases: e.g., dermatitis, psoriasis, sunburn, burns, and eczema;

(6) Renal, urinary and pancreatic diseases: e.g., nephrotic syndrome and all types of nephritis (such as glomerulonephritis); pancreatitis; bladder hyperreflexia following bladder inflammation;

(7) Hepatic diseases: e.g., acute liver cell disintegration; acute hepatitis of various genesis (such as viral, toxic, drug-induced) and chronically aggressive and/or chronically intermittent hepatitis, liver fibrosis associated with liver injury or disease, including fibrosis caused or exacerbated by alcoholic liver cirrhosis, chronic viral hepatitis, non alcoholic steatohepatitis and primary liver cancer;

(8) Gastrointestinal diseases: e.g., inflammatory bowel diseases, irritable bowel syndrome, regional enteritis (Crohns disease), colitis ulcerosa, gastritis, aphthous ulcer, celiac disease, regional ileitis, and gastroesophageal reflux disease;

(9) Neurodegenerative diseases: e.g. in the treatment of neurodegeneration following stroke, cardiac arrest, pulmonary bypass, traumatic brain injury, spinal cord injury or the like;

(10) Eye diseases: e.g., allergic keratitis, uveitis, or iritis, conjunctivitis, blepharitis, neuritis nervi optici, choroiditis, glaucoma and sympathetic ophthalmia;

(11) Diseases of the ear, nose, and throat (ENT) area: e.g., tinnitus, allergic rhinitis or hay fever, otitis externa, caused by contact eczema, infection, etc., and otitis media;

(12) Neurological diseases: e.g. brain edema, particularly tumor-related brain edema, multiple sclerosis, acute encephalomyelitis, meningitis, acute spinal cord injury, trauma, dementia, particularly degenerative dementia (including senile dementia, Alzheimer's disease, Parkinson's disease and Creutzfeldt-Jacob disease, Huntington's chorea, Pick's disease, motor neuron disease), vascular dementia (including multi-infarct dementia and dementia associated with intracranial space occupying lesions, infections and related conditions such as HIV infection), Guillain-Barre syndrome, myasthenia gravis, stroke, and various forms of seizures (such as nodding spasms);

(13) Blood diseases: e.g., acquired hemolytic anemia, aplastic anemia, and idiopathic thrombocytopenia;

(14) Tumor diseases: e.g., acute lymphatic leukemia, Hodgkin's disease, malignant lymphoma, lymphogranulomatoses, lymphosarcoma, solid malignant tumors, and extensive metastases;

(15) Endocrine diseases: e.g., endocrine opthalmopathy, endocrine orbitopathia, thyrotoxic crisis, Thyroiditis de Quervain, Hashimoto thyroiditis, Morbus Basedow, granulomatous thyroiditis, struma lymphomatosa, Graves disease, type I diabetes (such as insulin-dependent diabetes); Organ and tissue transplantations and graft-versus-host diseases;

(16) Severe states of shock: e.g., septic shock, anaphylactic shock, and systemic inflammatory response syndrome (SIRS); and various other disease-states or conditions including, restenosis following percutaneous transluminal coronary angioplasty, acute and chronic pain, atherosclerosis, reperfusion injury, congestive heart failure, myocardial infarction, thermal injury, multiple organ injury secondary to trauma, necrotizing enterocolitis and syndromes associated with hemodialysis, leukopheresis, granulocyte transfusion, sarcoidosis, gingivitis, pyrexia. edema resulting from trauma associated with burns, sprains or fracture, cerebral edema and angioedema, and diabetes (such as diabetic vasculopathy, diabetic neuropathy, diabetic retinopathy, post capillary resistance and diabetic symptoms associated with insulitis (e.g. hyperglycemia, diuresis, proteinuria and increased nitrite and kallikrein urinary excretion)).

The compounds and pharmaceutical compositions described herein can be used alone or in combination therapy for the treatment or prevention of substance abuse related syndromes, disorders or diseases including, for example, drug abuse and drug withdrawal. Abused substances can include alcohol, amphetamines, amphetamine like substances, caffeine, cannabis, cocaine, hallucinogens, inhalants, opioids, nicotine (and/or tobacco products), heroin abuse, barbiturates, phencyclidine (or phencyclidine-like compounds), sedative-hypnotics, benzodiazepines, or combinations of any of the foregoing. The compounds and pharmaceutical compositions can also be used to treat withdrawal symptoms and substance-induced anxiety or mood disorder. In addition, they can be used to reduce tobacco craving; treat nicotine dependency, addiction, or withdrawal; or aid in the cessation or lessening of tobacco in a subject in need thereof.

The compounds and pharmaceutical compositions described herein can be used alone or in combination therapy for the treatment or prevention of psychiatric disorders, such as depressions (including major depressive disorder, bipolar depression, unipolar depression, single or recurrent major depressive episodes (e.g., with or without psychotic features, catatonic features, and/or melancholic features), postpartum onset, seasonal affective disorder, dysthymic disorders (e.g., with early or late onset and with or without atypical features), neurotic depression and social phobia, depression accompanying dementia, anxiety, psychosis, social affective disorders, and/or cognitive disorders), manic-depressive psychoses, bipolar disorders, extreme psychotic states (such as mania, schizophrenia, and excessive mood swings where behavioral stabilization is desired). The compounds and pharmaceutical compositions described herein can also be used alone or in combination therapy for the treatment or prevention of attention disorders such as ADHD (attention deficit hyperactivity disorders), autism, anxiety states, generalized anxiety, agoraphobia, as well as those behavioral states characterized by social withdrawal.

The compounds and pharmaceutical compositions described herein can be used alone or in combination therapy for the treatment or prevention of neurological or neurodegenerative disorders. Examples of neurodegenerative diseases include dementia, particularly degenerative dementia (including senile dementia, Alzheimer's disease, Pick's disease, Huntington's chorea, Parkinson's disease and Creutzfeldt-Jakob disease, motor neuron disease); vascular dementia (including multi-infarct dementia); as well as dementia associated with intracranial space occupying lesions; trauma; infections and related conditions (including HIV infection); dementia in Parkinson's disease, metabolism; toxins; anoxia and vitamin deficiency; and mild cognitive impairment associated with ageing, particularly Age Associated Memory Impairment. Examples of neurological disorders include amyotrophic lateral sclerosis (ALS), multiple sclerosis, epilepsy, ischemia, traumatic head or brain injury, brain inflammation, eye injury, stroke and neuroinflammation.

The compounds and pharmaceutical compositions described herein can be used alone or in combination therapy for the treatment or prevention of ocular disorders including, for example, glaucoma (such as normal tension glaucoma), glaucoma-associated intraocular pressure retinitis, retinopathies, uveitis, and acute injury to the eye tissue (e.g. conjunctivitis). Ocular disorders also include neurodegenerative diseases conditions of the retina and the optic nerve, for example, in patients presenting risk factors for glaucoma, such as high intraocular pressure, family history of glaucoma, glaucoma in the contralateral eye and high myopia.

Compounds and compositions of the invention are also useful for veterinary treatment of companion animals, exotic animals and farm animals, including, without limitation, dogs, cats, mice, rats, hamsters, gerbils, guinea pigs, rabbits, horses, pigs and cattle.

In another embodiment, the invention provides a method of increasing CB receptor activity in a biological sample, comprising contacting said biological sample with a compound or composition of the invention. Use of a CB receptor agonist in a biological sample is useful for a variety of purposes known to one of skill in the art. Examples of such purposes include, without limitation, biological assays and biological specimen storage.

### Combination Therapies

The compounds and pharmaceutical compositions described herein can be used in combination therapy with one or more (i.e. up to 6) additional therapeutic agents. For combination treatment with more than one active agent, where the active agents are in separate dosage formulations, the active agents may be administered separately or in conjunction. In addition, the administration of one element may be prior to, concurrent to, or subsequent to the administration of the other agent.

When co-administered with other agents, e.g., when co-administered with another pain medication, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, compounds described herein can be administered to a subject in a dosage range from between about 0.01 to about 10,000 mg/kg body weight/day, about 0.01 to about 5000 mg/kg body weight/day, about 0.01 to about 3000 mg/kg body weight/day, about 0.01 to about 1000 mg/kg body weight/day, about 0.01 to about 500 mg/kg body weight/day, about 0.01 to about 300 mg/kg body weight/day, about 0.01 to about 100 mg/kg body weight/day.

When “combination therapy” is employed, an effective amount can be achieved using a first amount of a compound of Formula I or a pharmaceutically acceptable salt, solvate (e.g., hydrate), co-crystal or pro-drug thereof and a second amount of an additional suitable therapeutic agent (e.g. an agent to treat pain).

In one embodiment of this invention, the compound of Formula I and the additional therapeutic agent are each administered in an effective amount (i.e., each in an amount which would be therapeutically effective if administered alone). In another embodiment, the compound of Structural Formula I and the additional therapeutic agent are each administered in an amount which alone does not provide a therapeutic effect (a sub-therapeutic dose). In yet another embodiment, the compound of Structural Formula I can be administered in an effective amount, while the additional therapeutic agent is administered in a sub-therapeutic dose. In still another embodiment, the compound of Structural Formula I can be administered in a sub-therapeutic dose, while the additional therapeutic agent, for example, a suitable cancer-therapeutic agent is administered in an effective amount.

As used herein, the terms “in combination” or “co-administration” can be used interchangeably to refer to the use of more than one therapy (e.g., one or more (i.e. up to 6) prophylactic and/or therapeutic agents). The use of the terms does not restrict the order in which therapies (e.g., prophylactic and/or therapeutic agents) are administered to a subject.

Co-administration encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, such co-administration also encompasses use of each compound in a sequential manner in either order. When co-administration involves the separate administration of the first amount of a compound of Structural Formulae I and a second amount of an additional therapeutic agent, the compounds are administered sufficiently close in time to have the desired therapeutic effect. For example, the period of time between each administration which can result in the desired therapeutic effect, can range from minutes to hours and can be deter pined taking into account the properties of each compound such as potency, solubility, bioavailability, plasma half-life and kinetic profile. For example, a compound of Formula I and the second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as a compound described herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as an anti-cancer agent) to a subject.

Additional therapeutic agents include, without limitation:

Pain relieving agents such as acetaminophen or paracetamol;

non-steroidal anti-inflammatory drugs (NSAIDs) such as propionic acid derivatives (alminoprofen, benoxaprofen, bucloxic acid, carprofen, fenhufen, fenoprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (meclofenamic acid, mefe-namic acid, and tolfenamic acid), biphenyl-carboxylic acid derivatives, oxicams (isoxicam, meloxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone), and COX-2 inhibitors, such as the coxibs (celecoxib, deracoxib, valdecoxib, rofecoxib, parecoxib and etoricoxib);

other pain relieving agents such as gabapentin, topical capsaicin, tanezumab, esreboxetine;

cannabinoid receptor agonists such as Dronabinol, 09-THC, CP-55940, WIN-55212-2, HU-210;

opiate receptor agonists such as morphine, propoxyphene (Darvon), tramadol, buprenorphin;

sodium channel blockers such as carbamazepine, mexiletine, lamotrigine, pregabaline, tectin, NW-1029, CGX-1002;

N-type calcium channel blockers such as Ziconotide, NMED-160, SPI-860; serotonergic and noradrenergic modulators such as SR-57746, paroxetine, duloxetine, clonidine, amitriptyline, citalopram;

local anesthetics such as ambroxol, lidocaine;

VR1 agonists and antagonists such as NGX-4010, WL-1002, ALGR^{X}-4975, WL-10001, AMG-517;

agents used for migraine, such as sumatriptan, zolmitriptan, naratriptan, eletriptan, rauwolscine, yohimbine, metoclopramide;

anti-inflammatory and/or immunosuppressive agents such as methotrexate, cyclosporin A (including, for example, cyclosporin microemulsion), tacrolimus, corticosteroids, statins, interferon beta, Remicade (Infliximab), Enbrel (Etanercept) and Humira (Adalimumab);

agents designed to treat tobacco abuse (e.g., nicotine receptor partial agonists, bupropion hypochloride (also known under the tradename Zyban™) and nicotine replacement therapies);

ADD/ADHD agents (e.g., Ritalin™ (methylphenidate hydrochloride), Strattera™ (atomoxetine hydrochloride), Concerta™ (methylphenidate hydrochloride) and Adderall™ (amphetamine aspartate; amphetamine sulfate; dextroamphetamine saccharate; and dextroamphetamine sulfate);

agents to treat alcoholism, such as opioid antagonists (e.g., naltrexone (also known under the tradename ReVia M) and nalmefene), disulfuram (also known under the tradename Antabuse™), and acamprosate (also known under the tradename Campral™);

agents for reducing alcohol withdrawal symptoms such as benzodiazepines, beta-blockers, clonidine, carbamazepine, pregabalin, and gabapentin (Neurontin™);

antihypertensive agents such as ACE inhibitors and Angiotensin II Receptor blockers such as benazepril, captopril, enalapril, fosinopril, lisinopril, candesartan, eprosartan, Irbesartan, losartan, olmesartan, telmisartan, valsartan, Renin inhibitors such as aliskiren, vasodilators such as minoxidil;

agents used to treat glaucoma such as direct-acting Miotics (cholinergic agonists), indirect acting Miotics (cholinesterase inhibitors), Carbonic anhydrase inhibitors (e.g. Acetazolamide, Methazolamide, Brinzolamide, Dorzolamide, Selective adrenergic agonists (e.g. Apraclonidine, Brimonidine), Beta-blockers (Timolol, Betaxolol, Carteolol, Levobetaxolol, Levobunolol, Metipranolol), Osmotic diuretics (e.g. Glycerin, Mannitol);

antidepressants, such as SSRIs (e.g., fluoxetine, citalopram, femoxetine, fluvoxamine, paroxetine, indalpine, sertraline, zimeldine), tricyclic antidepressants (e.g., imipramine, amitriptiline, chlomipramine and nortriptiline), dopaminergic antidepressants (e.g., bupropion and amineptine), SNRIs (e.g., venlafaxine and reboxetine);

cognitive improvement agents (e.g., donepezil hydrochloride (Aircept™) and other acetylcholinesterase inhibitors);

anti-emetic agents (e.g., 5HT3 antagonists) such as ondansetron, granisetron, metoclopramide;

neuroprotective agents such as memantine, L-dopa, bromocriptine, pergolide, talipexol, pramipexol, cabergoline, neuroprotective agents currently under investigation including anti-apoptotic drugs (CEP 1347 and CTCT346), lazaroids, bioenergetics, antiglutamatergic agents and dopamine receptors. Other clinically evaluated neuroprotective agents are the monoamine oxidase B inhibitors selegiline and rasagiline, dopamine agonists, and the complex I mitochondrial fortifier coenzyme Q10;

antipsychotic medications (e.g., ziprasidone (Geodon™), risperidone (Risperdal™), and olanzapine (Zyprexa™);

agents used for multiple sclerosis such as beta-interferon (e.g., Avonex™, Betaseron™) and Copaxone.

disease-modifying antirheumatic drugs (DMARDS) such as methotrexate, azathioptrine, leflunomide, pencillinamine, gold salts, mycophenolate mofetil, cyclophosphamide; biological response modifiers (BRMs) such as Enbrel, Remicade, IL-1 antagonists; NSAIDS such as piroxicam, naproxen, indomethacin, ibuprofen and the like; COX-2 selective inhibitors such as Celebrex™; COX-1 inhibitors such as Feldene; immunosuppressives such as steroids, cyclosporine, Tacrolimus, rapamycin and the like;

PDE4 inhibitors such as theophylline, drotaverine hydrochloride, cilomilast, roflumilast, denbufylline, rolipram, tetomilast, enprofylline, arofylline, cipamfylline, tofimilast, filaminast, piclamilast, (R)-(+)-4-[2-(3-cyclopentyloxy-4-methoxyphenyl)-2-phenylethyl]pyridine, mesopram, N-(3,5-dichloro-4-pyridinyl)-2-[1-(4-fluorobenzyl)-5-hydroxy-1H-indol-3-yl]-2-oxoacetamide, CDC-801 (Celgene), CC-1088 (Celgene), Lirimilast, ONO-6126 (Ono), CC-10004 (Celgene) and MN-001 (Kyorin), ibudilast and pentoxifylline, for use in treating inflammation, lung disorders and as bronchodilators;

corticosteroids such as betamethasone, budesonide, cortisone, dexamethasone, hydrocortisone, methylprednisolone, prednisolone, prednisone and triamcinolone;

histamine H1 receptor antagonists such as bromopheniramine, chlorpheniramine, dexchlorpheniramine, triprolidine, clemastine, diphenhydramine, diphenylpyraline, tripelennamine, hydroxyzine, methdiazine, promethazine, trimeprazine, azatadine, cyproheptadine, antazoline, pheniramine pyrilamine, astemizole, terfenadine, loratadine, cetirizine, desloratadine, fexofenadine and levocetirizine;

histamine H2 receptor antagonists such as cimetidine, famotidine and ranitidine;

proton pump inhibitors such as omeprazole, pantoprazole and esomeprazole;

leukotriene antagonists and 5-lipoxygenase inhibitors such as zafirlukast, montelukast, pranlukast and zileuton;

nicotinic acetylcholine receptor agonists such as ABT-202, A-366833, ABT-594; BTG-102, A-85380, CGX1204;

P2X3 receptor antagonists such as A-317491, ISIS-13920, AZD-9056;

NGF agonists and antagonists such as RI-724, RI-1024, AMG-819, AMG-403, PPH 207;

NK1 and NK2 antagonists such as DA-5018, R-116301; CP-728663, ZD-2249;

NMDA antagonist such as NER-MD-11, CNS-5161, EAA-090, AZ-756, CNP-3381; potassium channel modulators such as CL-888, ICA-69673, retigabine;

GABA modulators such as lacosamide; and serotonergic and noradrenergic modulators such as SR-57746, paroxetine, duloxetine, clonidine, amitriptyline, citalopram, flibanserin.

### Methods of Preparing the Compounds

The compounds of Formula I may be prepared according to the schemes and examples depicted and described below. The starting materials and various intermediates may be obtained from commercial sources, prepared from commercially available compounds or prepared using well-known synthetic methods.

### Synthesis

General synthetic procedures for the compounds of this invention are described below. These synthetic schemes are presented as examples of synthetic methods and do not limit the scope of this invention in any way.

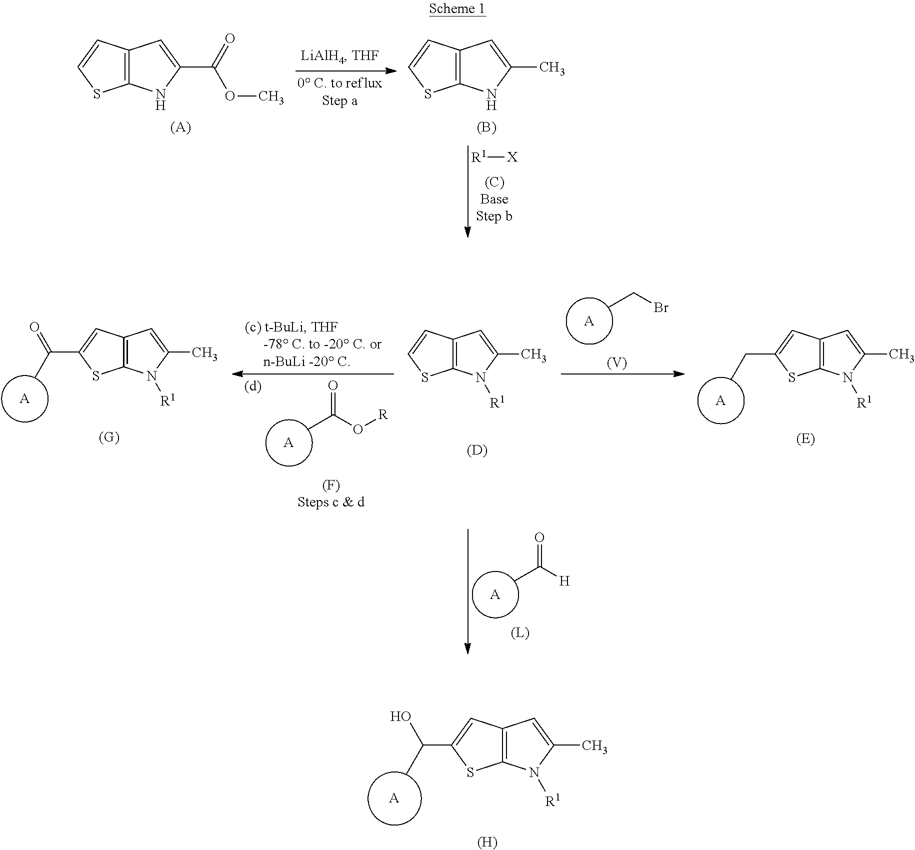

Scheme 1 depicts the synthesis of invention compounds of Formula D, E, G, and H, wherein R² is methyl. Thus, 5-methyl-6H-thieno[2,3-b]pyrrole (B), can be synthesized from methyl 6H-thieno[2,3-b]pyrrole-5-carboxylate (A) via complete reduction of the ester functionality with a reducing agent, such as lithium aluminum hydride. Compound B can then be diversified to compounds of Formula D via alkylation of the ring nitrogen using an alkyl bearing a leaving group X (e.g. alkyl halides) of Formula C in the presence of a strong base. Compounds of Formula D can be further diversified to compounds of Formula E via alkylation using alkylating agents of Formula V, or to ketones of Formula G through deprotonation (e.g. with an alkyl lithium reagent) followed by acylation with esters of Formula F (Scheme 1). Similarly, alcohols of Formula H can be prepared by deprotonation of the thienopyrrole and subsequent reaction with an aldehyde of Formula L.

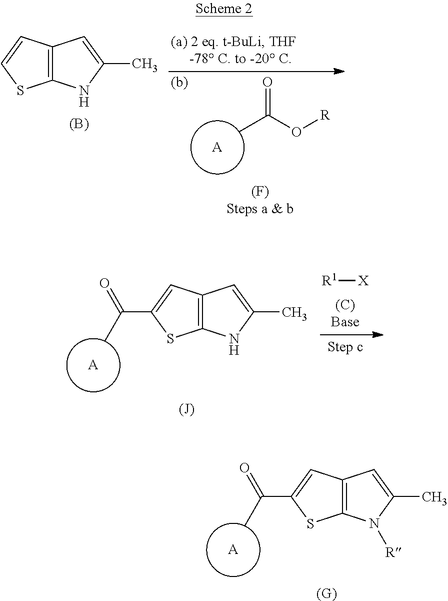

Scheme 2 depicts an alternative approach to the synthesis of ketones of Formula G using compound B. Thus, treatment of B with two equivalents of a strong base (e.g. tert-butyllithium) to create the dianion, followed by acylation with an ester of Formula F wherein OR is a suitable leaving group (e.g. an alkoxy or aryloxy moiety) to give ketones of Formula J. These ketones can then be functionalized further using alkylating agents of Formula C.

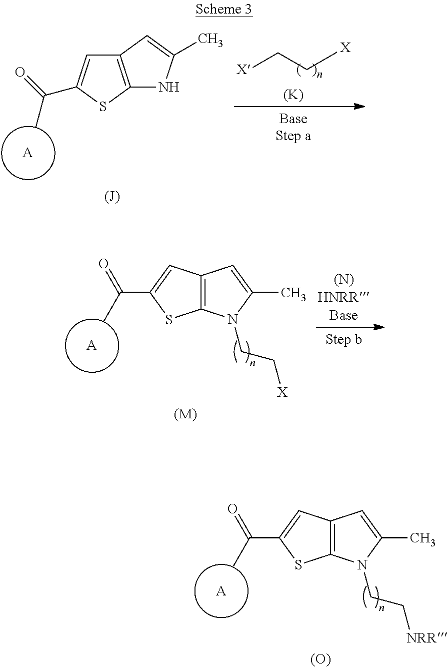

Scheme 3 depicts the conversion of compounds of Formula J to compounds of Formula O, which is a variant of a compound of Formula G, wherein R¹ is

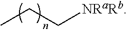

Thus, treatment of J with a bis-alkylating agent K wherein X and X′ are leaving groups such as Cl, Br, or I, OTs or the like, provides a compound of Formula M. The resulting compounds can then be further functionalized to compounds of Formula O with nucleophilic species such as the amine compounds of Formula N. The nature of the base and solvent used in step b depends on the nature of the nucleophile 14. For instance, for primary or secondary amines, the following potassium carbonate/dioxane has also been used and for sulfur nucleophiles the reaction has been carried out in the absence of base. Other bases and solvents that could be used include: sodium carbonate or Et₃N in acetonitrile, DMF, THF or DMSO. For generating sulfonamides (wherein R or R″′ is SO₂R), NaH in DMF has been used. Other solvents and bases that could be used include, for instance, KOH, NaOH in DMSO, H₂O, THF or dioxane.

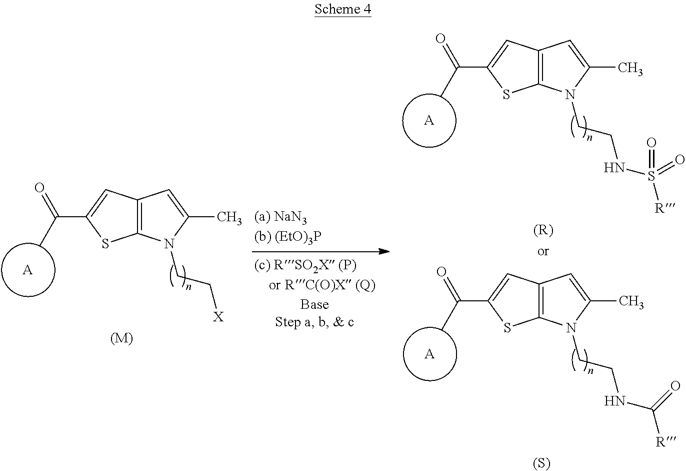

In Scheme 4, a compound of Formula M wherein X is defined as in Scheme 3 can also be converted to a compound of Formula R or S, which are further variants of a compound of Formula G, wherein R¹ is

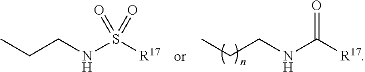

Thus, conversion of M azide, followed by reduction using a phosphene reagent such as triethylphosphite provides the primary amine. The amine can then be functionalized to the corresponding sulfonamide of Formula R using a sulfonating reagent of Formula P, or to the corresponding amide of Formula S using an acylating reagent of Formula Q, wherein R¹⁷ is defined as above. The nature of the base used in step c depends on the nature of the electrophile. For instance, for sulfonating agents such as sulfonyl chlorides and acylating agents such as acyl chlorides, Et₃N/DMAP has been used. Other bases that could be used include, for instance, pyridine or Hünig's base.

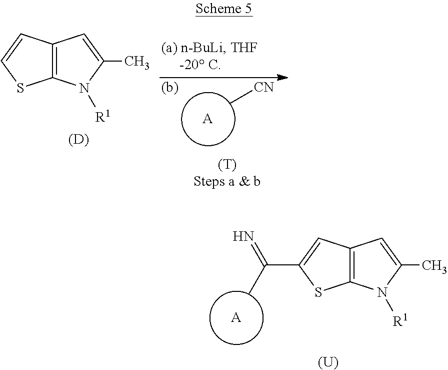

Scheme 5 depicts the conversion of a compound of Formula D to an imine of Formula U, which is a further variant of a compound of Formula G. Thus, deprotonation of D using n-butyl lithium or the like, followed by subsequent addition to nitrile compound of Formula T provides the target imine U.

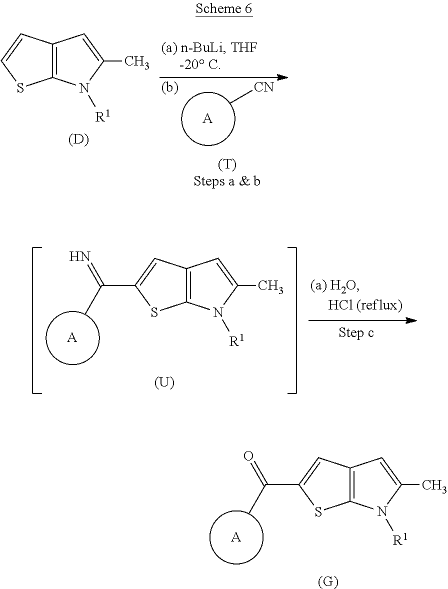

Similar to Scheme 5, in Scheme 6, the imine of Formula U is converted to the ketone of Formula G upon acid hydrolysis either with or without isolation of the imine U.

### EXAMPLES

All references provided in the Examples are herein incorporated by reference in their entirety. As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997, herein incorporated in its entirety by reference.

Many of the following compounds may be prepared by more than one method.

### Example 1

### Synthesis of 5-methyl-6H-thieno[2,3-b]pyrrole (B)

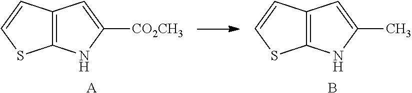

A 2.0 M solution of lithium aluminum hydride (308 ml, 616 mmol) in THF was added slowly to a 0° C. solution of methyl 6H-thieno[2,3-b]pyrrole-5-carboxylate (40.0 g, 221 mmol) in 400 mL of THF. A significant amount of hydrogen gas evolved. The mixture was heated at reflux for 5 h. The reaction mixture was then cooled to 0° C. and quenched by the slow addition of H₂O (23 mL), 15% NaOH solution (23 mL), and H₂O (70 mL), respectively. The mixture was stirred vigorously throughout the quenching process. The precipitate generated was filtered and discarded. The filtrate was diluted with Et₂O (200 mL) and washed with H₂O (50 mL). The organic layer was dried with MgSO₄ and concentrated in vacuo to afford 25.1 g (83%) of the title compound as a tan solid (azeotroped 2 times with benzene prior to use in subsequent reactions); ¹H NMR (CDCl₃/400 MHz) 7.95 (br s, 1H), 6.91 (dd, J=5.2, 0.4 Hz, 1H), 6.77 (d, J=5.2 Hz, 1H), 6.13 (dd, J=2.0, 0.8 Hz, 1H), 2.39 (d, J=0.8 Hz, 3H).

### Example 2

### Synthesis of 4-(2-(5-methyl-6H-thieno[2,3-b]pyrrol-6-yl)ethyl)morpholine (D-1)

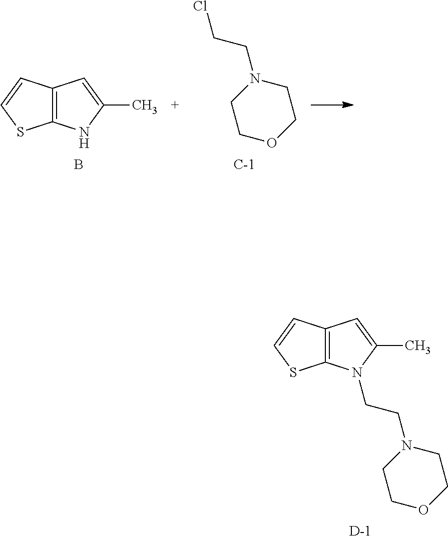

Potassium hydroxide (4.09 g, 72.9 mmol) and H₂O (110 μL) (about 10 drops) were added to a solution of 4-(2-chloroethyl)morpholine hydrochloride (3.26 g, 17.50 mmol) and compound B (1.0 equiv) in 31 mL of DMSO. The reaction mixture was heated at 75° C. with stirring for 1.5 hours during which time the reaction mixture darkened to a deep brown. The reaction mixture was diluted with H₂O and extracted with EtOAc (3 times). The combined organic layers were washed with H₂O (2 times). The residue was purified by flash silica gel chromatography (10%→70% EtOAc in hexanes) to afford 2.95 g (81%) of 4-(2-(5-methyl-6H-thieno[2,3-b]pyrrol-6-yl)ethyl)morpholine as a light yellow crystalline solid (azeotroped 2× with benzene prior to use in subsequent reactions); ¹H NMR (CDCl₃/400 MHz) 6.92 (d, J=4.8 Hz, 1H), 6.76 (d, J=5.2 Hz, 1H), 6.12 (d, J=0.8 Hz, 1H), 4.04 (t, J=6.8 Hz, 2H), 3.71 (t, J=4.8 Hz, 4H), 2.71 (t, J=6.8 Hz, 2H), 2.49 (t, J=4.8 Hz, 4H), 2.37 (d, J=0.8 Hz, 3H); MS m/z: 251.14 (M+1).

### Example 3

### Synthesis of (2,3-dichlorophenyl)(5-methyl-6-(2-morpholinoethyl)-6H-thieno[2,3-b]pyrrol-2-yl)methanone (1)

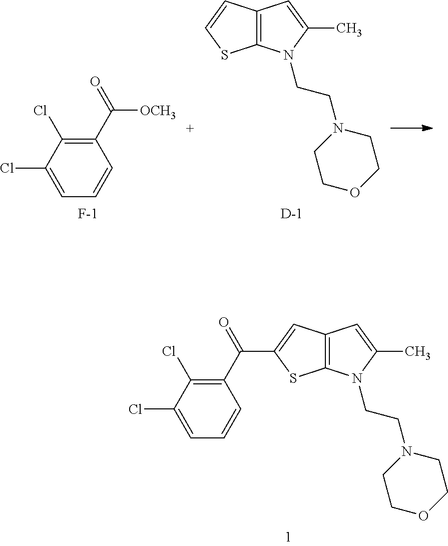

A 1.7 M solution of t-butyllithium (1.05 mL, 1.79 mmol) in pentane was slowly added to a −78° C. solution of 4-(2-(5-methyl-6H-thieno[2,3-b]pyrrol-6-yl)ethyl)morpholine (410 mg, 1.64 mmol) in 6 mL of THF. The reaction mixture turned yellow and stirred at this temperature for 1 h. A solution of methyl 2,3-dichlorobenzoate (520 mg, 2.54 mmol) in 2 mL of THF was added relatively quickly to the reaction. The reaction mixture darkened and was slowly allowed to warm to 0° C. over a period of 2 h. The reaction was then quenched by the addition of H₂O and dichloromethane. The layers were separated and the aqueous layer was extracted with dichloromethane (3 times). The organic layers were combined, dried with MgSO₄, and concentrated in vacuo to afford a brown residue. The residue was purified by flash silica gel chromatography (30% to 100% EtOAc in hexanes) to afford 386 mg (56%) of the title compound as a yellow foam; ¹H NMR (CDCl₃/400 MHz) 7.54 (dd, J=7.6, 1.6 Hz, 1H), 7.34 (dd, J=7.6, 1.6 Hz, 1H), 7.29 (d, J=7.6 Hz, 1H), 7.20 (s, 1H), 6.12 (d, J=0.8 Hz, 1H), 4.04 (t, J=6.4 Hz, 2H), 3.69 (t, J=4.4 Hz, 4H), 2.72 (t, J=6.4 Hz, 2H), 2.48 (t, J=4.4 Hz, 4H), 2.35 (d, J=0.8 Hz, 3H).

### Example 4

### Synthesis of (2,3-dichlorophenyl)(5-methyl-6H-thieno[2,3-b]pyrrol-2-yl)methanone (2)

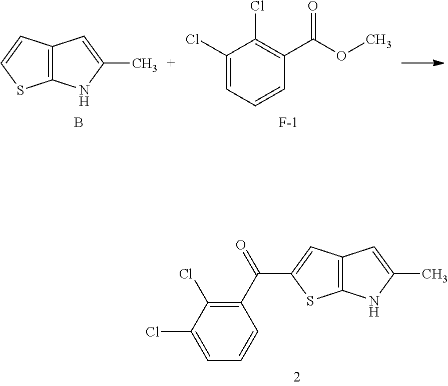

A 1.7 M solution of t-butyllithium (9.93 mL, 16.87 mmol) in pentane was slowly added to a −78° C. solution of 5-methyl-6H-thieno[2,3-b]pyrrole (1.0289 g, 7.50 mmol) in 24 mL of THF. The reaction mixture turned deep yellow and a precipitate formed after 20 min at −78° C. The cooling bath was replaced with a bath maintained at −20° C., and the reaction mixture stirred at this temperature for 1 h. Within minutes, the precipitate dissolved and the reaction became a homogeneous solution. The reaction color darkened a bit to an orange color. The reaction mixture was then cooled back to −78° C. A solution of methyl 2,3-dichlorobenzoate (2.5676 g, 12.15 mmol) in 12 mL of THF was added relatively quickly to the reaction. The reaction mixture darkened to a deep brown color and was stirred at this temperature for 1.75 h. The reaction was then quenched by the addition of H₂O and dichloromethane. The layers were separated and the aqueous layer was extracted with dichloromethane (3 times). The organic layers were combined, dried with MgSO₄, and concentrated in vacuo to afford a brown solid. The residue was triturated in a mixture of Et₂O and hexanes to afford 1.15 g (50%) of the title compound as a yellow solid; ¹H NMR (acetone-d₆/400 MHz) 10.69 (br s, 1H), 7.73 (dd, J=6.8, 2.4 Hz, 1H), 7.52-7.46 (m, 2H), 7.24 (s, 1H), 6.14 (br s, 1H), 2.38 (s, 3H).

### Example 5

### Synthesis of (4-methoxyphenyl)(5-methyl-6-(2-morpholinoethyl)-6H-thieno[2,3-b]pyrrol-2-yl)methanone (36)

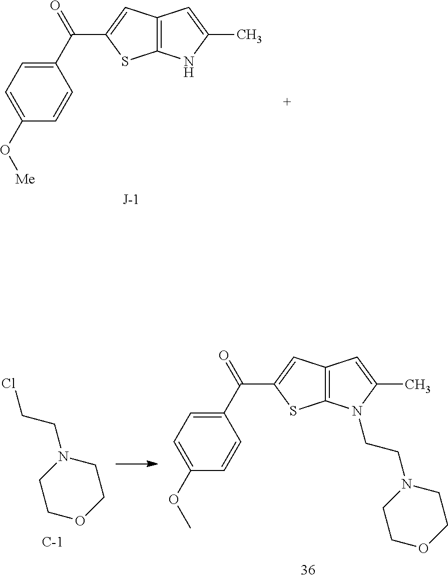

A mixture of (4-methoxyphenyl)(5-methyl-6H-thieno[2,3-b]pyrrol-2-yl)methanone (1.1 g, 4.05 mmol), 4-(2-chloroethyl)morpholine hydrochloride (3.02 g, 16.22 mmol), and potassium carbonate (3.9 g, 28.2 mmol) in 20 mL of DMF was heated at 110° C. for 24 h. The mixture was cooled to room temperature (rt). It was diluted with EtOAc (100 mL) and washed with H₂O (50 mL×3). The organic layer was dried with MgSO₄ and concentrated in vacuo to afford an oil. The residue was purified by flash silica gel chromatography (0% to 50% EtOAc in hexanes) to afford 1.18 g (76%) of the title compound as a pale yellow solid; ¹H NMR (CDCl₃/400 MHz) 7.88-7.84 (m, 2H), 7.51 (s, 1H), 7.00-6.96 (m, 2H), 6.16 (d, J=0.8 Hz, 1H), 4.06 (t, J=6.8 Hz, 2H), 3.88 (s, 3H), 3.70 (t, J=4.8 Hz, 4H), 2.74 (t, J=6.8 Hz, 2H), 2.50 (t, J=4.8 Hz, 4H), 2.38 (d, J=0.4 Hz, 3H).

The following compounds below were prepared according to Scheme 1:

(2,3-Dichlorophenyl)(5-methyl-6-propyl-6H-thieno[2,3-b]pyrrol-2-yl)methanone (3)

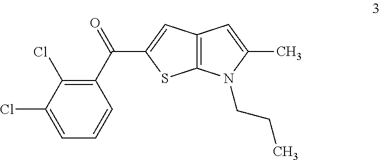 [SMILES: CCCn1c(C)cc2cc(C(=O)c3cccc(Cl)c3Cl)sc21]

¹H NMR (CDCl₃/400 MHz) δ 7.54 (dd, J=7.6, 1.6 Hz, 1H), 7.34 (dd, J=7.6, 1.6 Hz, 1H), 7.29 (d, J=8.0 Hz, 1H), 7.20 (br s, 1H), 6.12 (s, 1H), 3.90 (t, J=7.2 Hz, 2H), 2.33 (s, 3H), 1.87 (m, 2H), 0.96 (t, J=7.2 Hz, 3H);

### (3-Chlorophenyl)(5-methyl-6-(2-morpholinoethyl)-6H-thieno[2,3-b]pyrrol-2-yl)methanone (4)

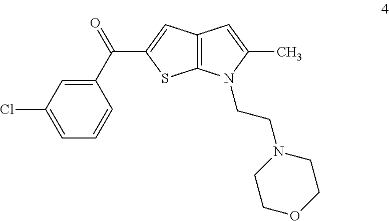 [SMILES: Cc1cc2cc(C(=O)c3cccc(Cl)c3)sc2n1CCN1CCOCC1]

¹H NMR (CDCl₃/400 MHz) δ 7.78 (t, J=1.6 Hz, 1H), 7.68 (dt, J=7.6, 1.6 Hz, 1H), 7.50 (dq, J=8.4, 1.2 Hz, 1H), 7.47 (s, 1H), 7.41 (d, J=8.0 Hz, 1H), 6.17 (d, J=0.8 Hz, 1H), 4.05 (t, J=6.8 Hz, 2H), 3.69 (t, J=4.8 Hz, 4H), 2.73 (t, J=6.8 Hz, 2H), 2.49 (t, J=4.8 Hz, 4H), 2.37 (d, J=0.8 Hz, 3H).

### (2-Chlorophenyl)(5-methyl-6-(2-morpholinoethyl)-61-1-thieno[2,3-b]pyrrol-2-yl)methanone (5)

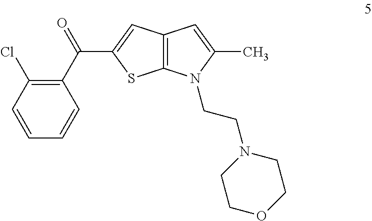 [SMILES: Cc1cc2cc(C(=O)c3ccccc3Cl)sc2n1CCN1CCOCC1]

¹H NMR (CDCl₃/400 MHz) δ 7.45-7.30 (m, 4H), 7.20 (s, 1H), 6.11 (d, J=1.2 Hz, 1H), 4.02 (t, J=6.8 Hz, 2H), 3.68 (t, J=4.4 Hz, 4H), 2.71 (t, J=6.8 Hz, 2H), 2.48 (t, J=4.4 Hz, 4H), 2.34 (d, J=1.2 Hz, 3H).

### (4-Chlorophenyl)(5-methyl-6-(2-morpholinoethyl)-6H-thieno[2,3-b]pyrrol-2-yl)methanone (6)

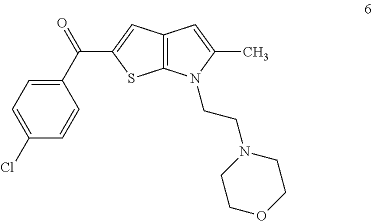 [SMILES: Cc1cc2cc(C(=O)c3ccc(Cl)cc3)sc2n1CCN1CCOCC1]

¹H NMR (CDCl₃/400 MHz) δ 7.72-7.69 (m, 2H), 7.41-7.37 (m, 3H), 6.10 (d, J=1.2 Hz, 1H), 3.99 (t, J=6.8 Hz, 2H), 3.64 (t, J=4.4 Hz, 4H), 2.67 (t, J=6.8 Hz, 2H), 2.43 (t, J=4.4 Hz, 4H), 2.31 (d, J=0.8 Hz, 3H).

### (5-Methyl-6-(2-morpholinoethyl)-6H-thieno[2,3-b]pyrrol-2-yl)(phenyl)methanone (7)

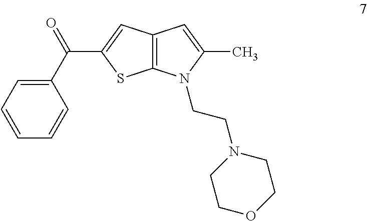 [SMILES: Cc1cc2cc(C(=O)c3ccccc3)sc2n1CCN1CCOCC1]

¹H NMR (CDCl₃/400 MHz) δ 7.83-7.81 (m, 2H), 7.56-7.45 (m, 4H), 6.16 (d, J=0.8 Hz, 1H), 4.06 (t, J=6.8 Hz, 2H), 3.70 (t, J=4.4 Hz, 4H), 2.74 (t, J=6.8 Hz, 2H), 2.50 (t, J=4.4 Hz, 4H), 2.37 (d, J=0.8 Hz, 3H).

### (2,3-Difluorophenyl)(5-methyl-6-(2-morpholinoethyl)-6H-thieno[2,3-b]pyrrol-2-yl)methanone (8)

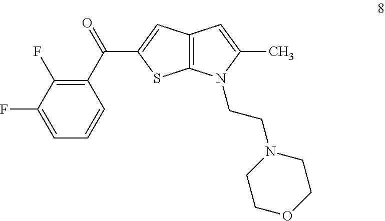 [SMILES: Cc1cc2cc(C(=O)c3cccc(F)c3F)sc2n1CCN1CCOCC1]

¹H NMR (CDCl₃/400 MHz) δ 7.35 (d, J=2.0 Hz, 1H), 7.32-7.27 (m, 2H), 7.19-7.14 (m, 1H), 6.13 (d, J=1.2 Hz, 1H), 4.03 (t, J=6.8 Hz, 2H), 3.69 (t, J=4.8 Hz, 4H), 2.72 (t, J=6.8 Hz, 2H), 2.48 (t, J=4.8 Hz, 4H), 2.36 (s, 3H).

### (3,4-Dichlorophenyl)(5-methyl-6-(2-morpholinoethyl)-6H-thieno[2,3-b]pyrrol-2-yl)methanone (9)

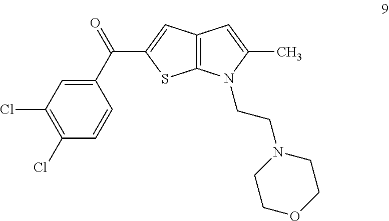 [SMILES: Cc1cc2cc(C(=O)c3ccc(Cl)c(Cl)c3)sc2n1CCN1CCOCC1]

¹H NMR (CDCl₃/400 MHz) δ 7.84 (d, J=1.6 Hz, 1H), 7.59 (dd, J=8.0, 2.0 Hz, 1H), 7.49 (d, J=8.4 Hz, 1H), 7.39 (s, 1H), 6.12 (d, J=1.2 Hz, 1H), 3.99 (t, J=6.8 Hz, 2H), 3.63 (t, J=4.4 Hz, 4H), 2.67 (t, J=6.8 Hz, 2H), 2.43 (t, J=4.4 Hz, 4H), 2.32 (d, J=0.8 Hz, 3H).

### (2,4-Dichlorophenyl)(5-methyl-6-(2-morpholinoethyl)-6H-thieno[2,3-b]pyrrol-2-yl)methanone (10)

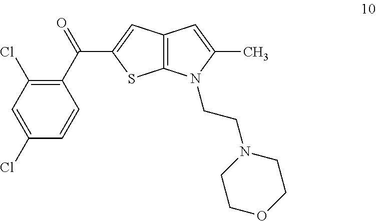 [SMILES: Cc1cc2cc(C(=O)c3ccc(Cl)cc3Cl)sc2n1CCN1CCOCC1]

¹H NMR (CDCl₃/400 MHz) δ 7.40 (d, J=2.0 Hz, 1H), 7.32 (d, J=8.4 Hz, 1H), 7.25 (dd, J=8.0, 2.0 Hz, 1H), 7.14 (s, 1H), 6.06 (d, J=1.2 Hz, 1H), 3.97 (t, J=6.8 Hz, 2H), 3.63 (t, J=4.8 Hz, 4H), 2.65 (t, J=6.8 Hz, 2H), 2.42 (t, J=4.8 Hz, 4H), 2.29 (d, J=0.8 Hz, 3H).

### (2,3-Dihydrobenzofuran-7-yl)(5-methyl-6-(2-morpholinoethyl)-6H-thieno[2,3-b]pyrrol-2-yl)methanone (11)

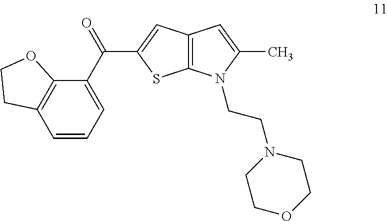 [SMILES: Cc1cc2cc(C(=O)c3cccc4c3OCC4)sc2n1CCN1CCOCC1]

¹H NMR (CDCl₃/400 MHz) δ 7.46 (s, 1H), 7.39 (dd, J=7.6, 0.8 Hz, 1H), 7.30 (dd, J=7.6, 0.8 Hz, 1H), 6.89 (t, J=7.6 Hz, 1H), 6.13 (s, 1H), 4.62 (t, J=8.8 Hz, 2H), 4.03 (t, J=6.8 Hz, 2H), 3.69 (t, J=4.4 Hz, 4H), 3.24 (t, J=8.8 Hz, 2H), 2.71 (t, J=6.8 Hz, 2H), 2.48 (t, J=4.4 Hz, 4H), 2.36 (s, 3H).

### (5-Methyl-6-(2-morpholinoethyl)-6H-thieno[2,3-b]pyrrol-2-yl)(o-tolyl)methanone (12)

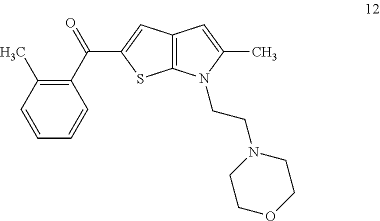 [SMILES: Cc1ccccc1C(=O)c1cc2cc(C)n(CCN3CCOCC3)c2s1]

¹H NMR (CDCl₃/400 MHz) δ 7.43-7.41 (m, 1H), 7.37-7.33 (m, 1H), 7.27-7.21 (m, 3H), 6.11 (d, J=1.2 Hz, 1H), 4.04 (t, J=6.8 Hz, 2H), 3.70 (t, J=4.8 Hz, 4H), 2.73 (t, J=6.8 Hz, 2H), 2.49 (t, J=4.8 Hz, 4H), 2.38 (s, 3H), 2.36 (s, 3H).

### (2-Methoxyphenyl)(5-methyl-6-(2-morpholinoethyl)-6H-thieno[2,3-b]pyrrol-2-yl)methanone (13)

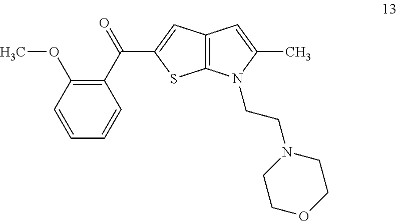 [SMILES: COc1ccccc1C(=O)c1cc2cc(C)n(CCN3CCOCC3)c2s1]

¹H NMR (CDCl₃/400 MHz) δ 7.43-7.37 (m, 2H), 7.27 (s, 1H), 7.01-6.97 (m, 2H), 6.10 (d, J=0.8 Hz, 1H), 4.03 (t, J=6.8 Hz, 2H), 3.79 (s, 3H), 3.69 (t, J=4.8 Hz, 4H), 2.71 (t, J=6.8 Hz, 2H), 2.48 (t, J=4.8 Hz, 4H), 2.35 (d, J=0.8 Hz, 3H).

### (5-Methyl-6-(2-morpholinoethyl)-6H-thieno[2,3-b]pyrrol-2-yl)(pyridin-3-yl)methanone (14)

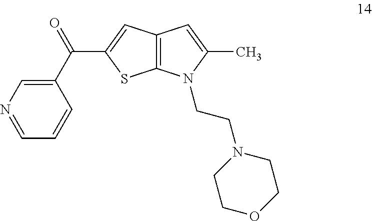 [SMILES: Cc1cc2cc(C(=O)c3cccnc3)sc2n1CCN1CCOCC1]

¹H NMR (CDCl₃/400 MHz) δ 9.05-9.04, (m, 1H), 8.77 (dd, J=4.8, 1.6 Hz, 1H), 8.10 (dt, J=8.0, 2.0 Hz, 1H), 7.48 (s, 1H), 7.43 (ddd, J=8.0, 2.0, 1.0 Hz, 1H), 6.18 (d, J=0.8 Hz, 1H), 4.06 (t, J=6.8 Hz, 2H), 3.70 (t, J=4.8 Hz, 4H), 2.74 (t, J=6.8 Hz, 2H), 2.50 (t, J=4.8 Hz, 4H), 2.38 (d, J=0.8 Hz, 3H).

### (5-Methyl-6-(2-morpholinoethyl)-6H-thieno[2,3-b]pyrrol-2-yl)(2-(trifluoromethoxy)phenyl)methanone (15)

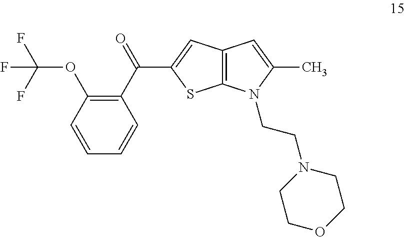 [SMILES: Cc1cc2cc(C(=O)c3ccccc3OC(F)(F)F)sc2n1CCN1CCOCC1]

¹H NMR (CDCl₃/400 MHz) δ 7.56-7.49 (m, 2H), 7.39-7.35 (m, 2H), 7.24 (s, 1H), 6.12 (d, J=0.8 Hz, 1H), 4.04 (t, J=6.8 Hz, 2H), 3.69 (t, J=4.8 Hz, 4H), 2.73 (t, J=6.8 Hz, 2H), 2.49 (t, J=4.8 Hz, 4H), 2.36 (d, J=0.8 Hz, 3H).

### (5-Methyl-6-(2-morpholinoethyl)-6H-thieno[2,3-b]pyrrol-2-yl)(pyridin-2-yflmethanone (16)

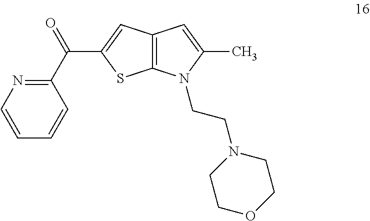 [SMILES: Cc1cc2cc(C(=O)c3ccccn3)sc2n1CCN1CCOCC1]

¹H NMR (CDCl₃/400 MHz) δ 8.71 (ddd, J=4.8, 2.0, 0.8 Hz, 1H), 8.37 (s, 1H), 8.12 (dt, J=8.0, 0.8 Hz, 1H), 7.85 (td, J=7.6, 2.0 Hz, 1H), 7.44 (ddd, J=7.6, 4.8, 1.2 Hz, 1H), 6.19 (d, J=1.2 Hz, 1H), 4.04 (t, J=6.8 Hz, 2H), 3.69 (t, J=4.8 Hz, 4H), 2.73 (t, J=6.8 Hz, 2H), 2.48 (t, J=4.8 Hz, 4H), 2.36 (d, J=0.8 Hz, 3H).

### (2-Chloropyridin-3-yl)(5-methyl-6-(2-morpholinoethyl)-6H-thieno[2,3-b]pyrrol-2-yl)methanone (17)

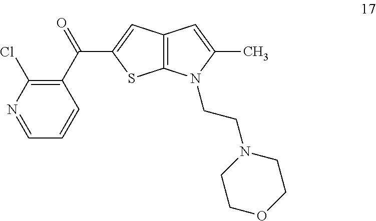 [SMILES: Cc1cc2cc(C(=O)c3cccnc3Cl)sc2n1CCN1CCOCC1]

¹H NMR (CDCl₃/400 MHz) δ 8.49 (dd, J=4.8, 2.0 Hz, 1H), 7.78 (dd, J=7.6, 2.0 Hz, 1H), 7.34 (dd, J=7.6, 4.8 Hz, 1H), 7.20 (s, 1H), 6.13 (d, J=1.2 Hz, 1H), 4.04 (t, J=6.8 Hz, 2H), 3.68 (t, J=4.8 Hz, 4H), 2.72 (t, J=6.8 Hz, 2H), 2.48 (t, J=4.8 Hz, 4H), 2.35 (d, J=0.8 Hz, 3H).

### (2,3-Dichlorophenyl)(5-methyl-6-(2-(tetrahydro-2H-pyran-4-yl)ethyl)-6H-thieno[2,3-b]pyrrol-2-yl)methanone (18)

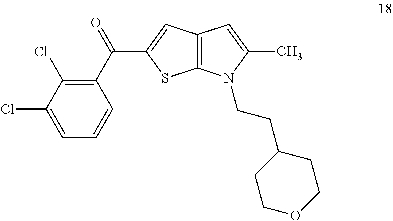 [SMILES: Cc1cc2cc(C(=O)c3cccc(Cl)c3Cl)sc2n1CCC1CCOCC1]

¹H NMR (CDCl₃/400 MHz) δ 7.56 (dd, J=7.6, 1.6 Hz, 1H), 7.35 (dd, J=7.6, 1.6 Hz, 1H), 7.29 (t, J=7.6 Hz, 1H), 7.21 (s, 1H), 6.14 (d, J=1.2 Hz, 1H), 3.98 (t, J=8.0 Hz, 4H), 3.38 (td, J=8.0, 2.0 Hz, 2H), 2.34 (d, J=0.8 Hz, 3H), 1.79 (dd, J=14.8, 6.8 Hz, 2H), 1.68-1.64 (m, 2H), 1.64-1.53 (m, 1H), 1.37 (ddd, J=24.8, 12.8, 0.4 Hz, 2H).

### (2,3-Dichlorophenyl)(6-ethyl-5-methyl-6H-thieno[2,3-b]pyrrol-2-yl)methanone (19)

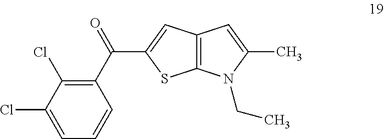 [SMILES: CCn1c(C)cc2cc(C(=O)c3cccc(Cl)c3Cl)sc21]

¹H NMR (CDCl₃/400 MHz) δ 7.56 (dd, J=7.6, 1.6 Hz, 1H), 7.35 (dd, J=7.6, 1.6 Hz, 1H), 7.29 (t, J=7.6 Hz, 1H), 7.22 (br s, 1H), 6.13 (d, J=0.8 Hz, 1H), 4.01 (q, J=7.2 Hz, 2H), 2.35 (d, J=0.8 Hz, 3H), 1.45 (t, J=7.2 Hz, 3H).

### (2,3-Dichlorophenyl)(5,6-dimethyl-6H-thieno[2,3-b]pyrrol-2-yl)methanone (20)

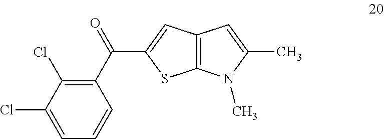 [SMILES: Cc1cc2cc(C(=O)c3cccc(Cl)c3Cl)sc2n1C]

¹H NMR (CDCl₃/400 MHz) δ 7.56 (dd, J=7.6, 1.6 Hz, 1H), 7.35 (dd, J=7.6, 1.6 Hz, 1H), 7.29 (t, J=7.6 Hz, 1H) 7.21 (br s, 1H), 6.14 (d, J=0.8 Hz, 1H), 3.64 (s, 3H), 2.34 (d, J=1.2 Hz, 3H).

### (2,3-Dichlorophenyl)(6-(2-methoxyethyl)-5-methyl-6H-thieno[2,3-b]pyrrol-2-yl)methanone (21)

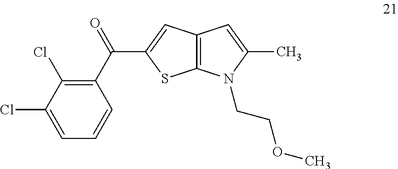 [SMILES: COCCn1c(C)cc2cc(C(=O)c3cccc(Cl)c3Cl)sc21]

¹H NMR (CDCl₃/400 MHz) δ 7.55 (dd, J=7.6, 1.6 Hz, 1H), 7.34 (dd, J=7.6, 1.6 Hz, 1H), 7.29 (t, J=7.6 Hz, 1H), 7.20 (br s, 1H), 6.12 (d, J=0.8 Hz, 1H), 4.11 (t, J=5.2 Hz, 2H), 3.72 (t, J=5.2 Hz, 2H), 3.33 (s, 3H), 2.35 (d, J=0.8 Hz, 3H).

### (2,3-Dichlorophenyl)(5-methyl-6-(2-(piperidin-1-yl)ethyl)-6H-thieno[2,3-b]pyrrol-2-yl)methanone (23)

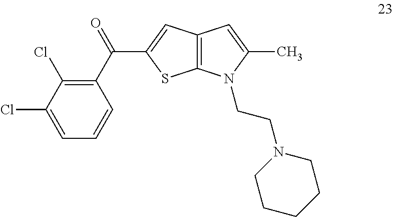 [SMILES: Cc1cc2cc(C(=O)c3cccc(Cl)c3Cl)sc2n1CCN1CCCCC1]

¹H NMR (CDCl₃/400 MHz) δ 7.55 (dd, J=8.0, 1.6 Hz, 1H), 7.35 (dd, J=7.6, 1.6 Hz, 1H), 7.29 (t, J=7.6 Hz, 1H), 7.20 (br s, 1H), 6.12 (s, 1H), 4.05 (t, J=7.2 Hz, 2H), 2.68 (t, J=7.2 Hz, 2H), 2.46 (br s, 4H), 2.36 (s, 3H), 1.59 (m, 4H), 1.49-1.40 (m, 2H).

### (5-Methyl-6-(2-morpholinoethyl)-6H-thieno[2,3-b]pyrrol-2-yl)(pyrimidin-4-yl)methanone (34)

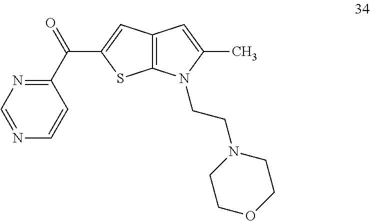 [SMILES: Cc1cc2cc(C(=O)c3ccncn3)sc2n1CCN1CCOCC1]

¹H NMR (CDCl₃/400 MHz) δ 9.37 (d, J=1.2 Hz, 1H), 8.97 (d, J=0.8 Hz, 1H), 8.44 (s, 1H), 8.01 (dd, J=5.2, 1.2 Hz, 1H), 6.21 (d, J=0.8 Hz, 1H), 4.04 (t, J=6.8 Hz, 2H), 3.69 (t, J=4.8 Hz, 4H), 2.73 (t, J=6.8 Hz, 2H), 2.49 (t, J=4.8 Hz, 4H), 2.37 (d, J=0.8 Hz, 3H).

### (2,5-Dichlorophenyl)(5-methyl-6-(2-morpholinoethyl)-6H-thieno[2,3-b]pyrrol-2-yl)methanone (35)

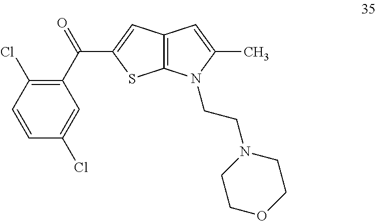 [SMILES: Cc1cc2cc(C(=O)c3cc(Cl)ccc3Cl)sc2n1CCN1CCOCC1]

¹H NMR (CDCl₃/400 MHz) δ 7.43 (d, J=2.4 Hz, 1H), 7.38 (s, 1H), 7.36 (d, J=2.4 Hz, 1H) 7.23 (br s, 1H), 6.14 (s, 1H), 4.04 (t, J=6.8 Hz, 2H), 3.70 (t, J=4.8 Hz, 4H), 2.72 (t, J=6.8 Hz, 2H), 2.49 (t, J=4.8 Hz, 4H), 2.36 (s, 3H).

### (3-Methoxyphenyl)(5-methyl-6-(2-morpholinoethyl)-6H-thieno[2,3-b]pyrrol-2-yl)methanone (26)

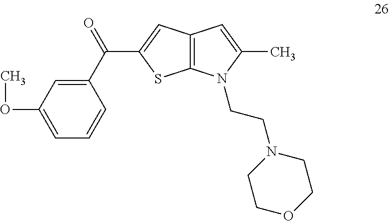 [SMILES: COc1cccc(C(=O)c2cc3cc(C)n(CCN4CCOCC4)c3s2)c1]

¹H NMR (CDCl₃/400 MHz) δ 7.53 (s, 1H), 7.43-7.33 (m, 3H), 7.09 (ddd, J=7.6, 2.8, 1.6 Hz, 1H), 6.16 (d, J=0.8 Hz, 1H), 4.06 (t, J=6.8 Hz 2H), 3.86 (s, 3H), 3.71 (t, J=4.8 Hz 4H), 2.74 (t, J=6.8 Hz, 2H), 2.50 (t, J=4.8 Hz, 4H), 2.38 (d, J=0.4 Hz, 3H).

### (5-Methyl-6-(2-morpholinoethyl)-6H-thieno[2,3-b]pyrrol-2-yl)(pyridin-4-yl)methanone (40)

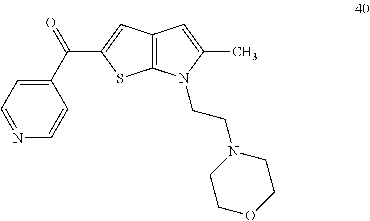 [SMILES: Cc1cc2cc(C(=O)c3ccncc3)sc2n1CCN1CCOCC1]

¹H NMR (CDCl₃/400 MHz) δ 8.77 (d, J=5.6 Hz, 2H), 7.62 (d, J=6.0 Hz, 2H), 7.45 (s, 1H), 6.18 (s, 1H), 4.06 (t, J=6.4 Hz, 2H), 3.70 (t, J=4.4 Hz, 4H), 2.74 (t, J=6.4 Hz, 2H), 2.49 (t, J=4.4 Hz, 4H), 2.38 (s, 3H).

### (6-Chloropyridin-2-yl)(5-methyl-6-(2-morpholinoethyl)-6H-thieno[2,3-b]pyrrol-2-yl)methanone (123)

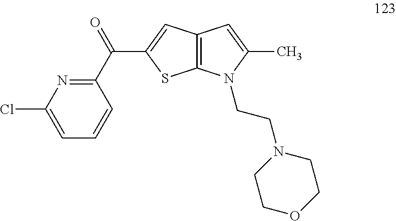 [SMILES: Cc1cc2cc(C(=O)c3cccc(Cl)n3)sc2n1CCN1CCOCC1]

¹H NMR (CDCl₃/400 MHz) δ 8.44 (s, 1H), 8.07 (dd, J=7.6, 0.8 Hz, 1H), 7.83 (t, J=7.6 Hz, 1H), 7.49 (dd, J=7.6, 0.8 Hz, 1H), 6.23 (d, J=0.8 Hz, 1H), 4.06 (t, J=6.8 Hz, 2H), 3.71 (t, J=4.4 Hz, 4H), 2.75 (t, J=6.8 Hz, 2H), 2.50 (t, J=4.4 Hz, 4H), 2.38 (d, J=0.8 Hz, 3H).

(2-Methoxypyridin-3-yl)(5-methyl-6-(2-morpholinoethyl)-6H-thieno[2,3-b]pyrrol-2-yl)methanone (47).

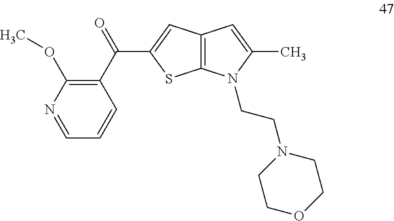 [SMILES: COc1ncccc1C(=O)c1cc2cc(C)n(CCN3CCOCC3)c2s1]

¹H NMR (CDCl₃/400 MHz) δ 8.27 (dd, J=5.2, 2.0 Hz, 1H), 7.71 (dd, J=7.6, 2.0 Hz, 1H), 7.28 (s, 1H), 6.98-6.95 (m. 1H), 6.12 (d, J=0.8 Hz, 1H), 4.04 (t, J=6.8 Hz, 2H), 3.94 (s, 3H), 3.69 (t, J=4.4 Hz, 4H), 2.72 (t, J=6.4 Hz, 2H), 2.49 (t, J=4.4 Hz, 4H), 2.36 (d, J=0.8 Hz, 3H); MS m/z: 386 (M+1).

### (5-Methyl-6-(2-morpholinoethyl)-6H-thieno[2,3-b]pyrrol-2-yl)(p-tolyl)methanone (124)

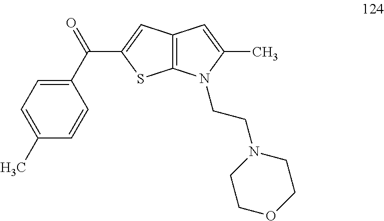 [SMILES: Cc1ccc(C(=O)c2cc3cc(C)n(CCN4CCOCC4)c3s2)cc1]

¹H NMR (CDCl₃/400 MHz) δ 7.74 (d, J=8.4 Hz, 2H), 7.50 (s, 1H), 7.28 (d, J=7.6 Hz, 2H), 6.16 (d, J=0.8 Hz, 1H), 4.06 (t, J=6.4 Hz, 2H), 3.72-3.70 (m, 4H), 2.74 (t, J=6.4 Hz, 2H), 2.51-2.49 (m, 4H), 2.44 (s, 3H), 2.38 (d, J=0.8 Hz, 3H).

### (4-Fluorophenyl)(5-methyl-6-(2-morpholinoethyl)-6H-thieno[2,3-b]pyrrol-2-yl)methanone (54)

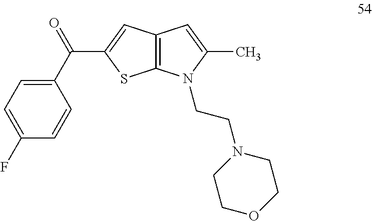 [SMILES: Cc1cc2cc(C(=O)c3ccc(F)cc3)sc2n1CCN1CCOCC1]

¹H NMR (CDCl₃/400 MHz) δ 7.88-7.84 (m, 2H), 4.74 (s, 1H), 7.16 (t, J=8.8 Hz, 2H), 6.18 (br s, 1H), 4.07 (t, J=6.4 Hz, 2H), 3.72-3.69 (m, 4H), 2.75 (t, J=6.8 Hz, 2H), 2.51-2.49 (m, 4H), 2.39 (s, 3H).

### (4-Ethoxyphenyl)(5-methyl-6-(2-morpholinoethyl)-6H-thieno[2,3-b]pyrrol-2-yl)methanone (55)

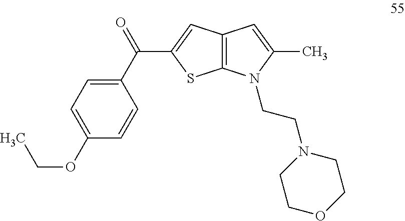 [SMILES: CCOc1ccc(C(=O)c2cc3cc(C)n(CCN4CCOCC4)c3s2)cc1]

¹H NMR (CDCl₃/400 MHz) δ 7.84 (d, J=8.8 Hz, 2H), 7.51 (s, 1H), 6.96 (d, J=8.8 Hz, 2H), 6.17 (s, 1H), 4.11 (q, J=7.2 Hz, 2H), 4.06 (t, J=6.4 Hz, 2H), 3.71 (t, J=4.8 Hz, 4H), 2.74 (t, J=6.4 Hz, 2H), 2.54-2.49 (m, 4H), 2.39 (s, 3H), 1.46 (t, J=7.2 Hz, 3H).

### (4-(Dimethylamino)phenyl)(5-methyl-6-(2-morpholinoethyl)-6H-thieno[2,3-b]pyrrol-2-yl)methanone (59)

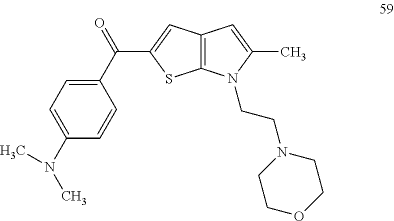 [SMILES: Cc1cc2cc(C(=O)c3ccc(N(C)C)cc3)sc2n1CCN1CCOCC1]

¹H NMR (CDCl₃/400 MHz) δ 7.85 (d, J=8.4 Hz, 2H), 7.54 (s, 1H), 6.71 (d, J=8.4 Hz, 2H), 6.16 (s, 1H), 4.13 (t, J=6.4 Hz, 2H), 3.64-3.77 (m, 4H), 3.07 (s, 6H), 2.75 (t, J=6.8 Hz, 2H), 2.48-2.55 (m, 4H), 2.40 (s, 3H).

### (5-Methyl-6-(2-morpholinoethyl)-6H-thieno[2,3-b]pyrrol-2-yl)(4-(trifluoromethyl)phenyl)methanone (60)

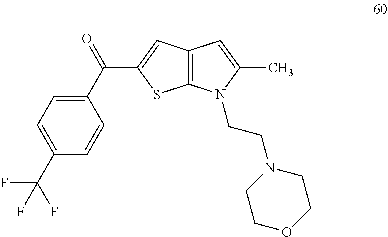 [SMILES: Cc1cc2cc(C(=O)c3ccc(C(F)(F)F)cc3)sc2n1CCN1CCOCC1]

¹H NMR (CDCl₃/400 MHz) δ 7.87 (d, J=8.8 Hz, 2H), 7.48 (s, 1H), 7.32 (d, J=8.8 Hz, 2H), 6.18 (s, 1H), 4.07 (t, J=6.4 Hz, 2H), 3.72-3.69 (m, 4H), 2.75 (t, J=6.8 Hz, 2H), 2.52-2.49 (m, 4H), 2.39 (s, 3H).

### (2-Chloro-4-fluorophenyl)(5-methyl-6-(2-morpholinoethyl)-6H-thieno[2,3-b]pyrrol-2-yl)methanone (64)

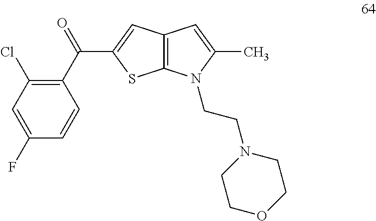 [SMILES: Cc1cc2cc(C(=O)c3ccc(F)cc3Cl)sc2n1CCN1CCOCC1]

¹H NMR (CDCl₃/400 MHz) δ 7.46 (dd, J=8.4, 2.0 Hz, 1H), 7.23-7.20 (m, 2H), 7.07 (ddd, J=10.8, 8.4, 2.4 Hz, 1H), 6.13 (s, 1H), 4.07-4.02 (m, 2H), 3.71-3.69 (m, 4H), 2.75-2.71 (m, 2H), 2.55-2.50 (m, 4H), 2.38 (s, 3H).

### (5-Methyl-6-(2-morpholinoethyl)-6H-thieno[2,3-b]pyrrol-2-yl)(4-(trifluoromethoxy)phenyl)methanone (65)

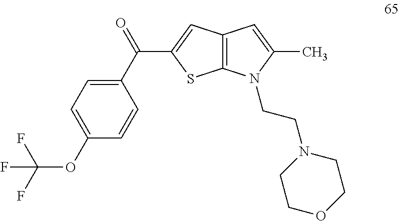 [SMILES: Cc1cc2cc(C(=O)c3ccc(OC(F)(F)F)cc3)sc2n1CCN1CCOCC1]

MS m/z: 439.17 (M+1).

### (2-Fluoro-4-methoxyphenyl)(5-methyl-6-(2-morpholinoethyl)-6H-thieno[2,3-b]pyrrol-2-yl)methanone (68)

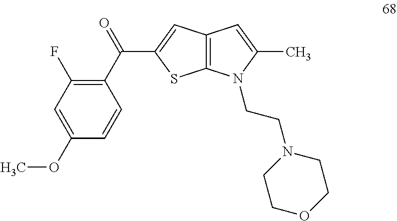 [SMILES: COc1ccc(C(=O)c2cc3cc(C)n(CCN4CCOCC4)c3s2)c(F)c1]

¹H NMR (CDCl₃/400 MHz) δ 7.55 (t, J=8.4 Hz, 1H), 7.40 (d, J=2.4 Hz, 1H), 6.77 (dd, J=8.8, 2.4 Hz, 1H), 6.69 (dd, J=12.0, 2.4 Hz, 1H), 6.15 (d, J=1.2 Hz, 1H), 4.05 (t, J=6.8 Hz, 2H), 3.86 (s, 3H), 3.71 (t, J=4.8 Hz, 4H), 2.74 (t, J=6.8 Hz, 2H), 2.50 (t, J=4.8 Hz, 4H), 2.37 (d, J=0.8 Hz, 3H); MS m/z: 403 (M+1).

### (2-fluoro-4-hydroxyphenyl)(5-methyl-6-(2-morpholinoethyl)-6H-thieno[2,3-b]pyrrol-2-yl)methanone (165)

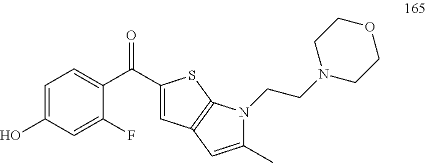 [SMILES: Cc1cc2cc(C(=O)c3ccc(O)cc3F)sc2n1CCN1CCOCC1]

This compound was made according to Scheme 1 with the ether of (2-fluoro-4-methoxyphenyl)(5-methyl-6-(2-morpholinoethyl)-6H-thieno[2,3-b]pyrrol-2-yl)methanone (68). At room temperature, to a solution of (2-fluoro-4-methoxyphenyl)(5-methyl-6-(2-morpholinoethyl)-6H-thieno[2,3-b]pyrrol-2-yl)methanone (40 mg, 0.1 mmol) in DCM (2 mL) in small scintillation vial was added 8 drops BBr3. Solution darkens straight away. After 30 min at RT, add more DCM (2 mL), 5 more drops BBr3. After 10 mins, quench carefully with 1 drop aqueous bicarbonate, followed by 1 ml bicarbonate. The mixture was capped and shaken to get a heterogeneous mixture. EtOAc (1 mL) was added and the mixture was shaken again. (Not everything dissolves in the layers). The reaction mixture was poured into a separatory funnel and workup between EtOAc and bicarbonate. Filter EtOAc layer thru sodium sulfate. The organic layers were concentrated in vacuo. The residue was purified by flash silica gel chromatography (solvent A (1:1 Hexane/EtOAc); Solvent B (MeOH); 1-12% solvent B in solvent A) to afford 35% of the title compound as a yellow solid. 1H NMR (CDCl3/400 MHz) 7.48 (t, J=8.0 Hz, 1H), 7.43 (d, J=1.6 Hz, 1H), 6.66 (m, 2H), 6.16 (s, 1H), 5.30 (s, 1H), 4.07 (t, J=6.4 Hz, 2H), 3.72 (t, J=4.0 Hz, 4H), 2.76 (t, J=6.4 Hz, 2H), 2.52 (m, 4H), 2.38 (s, 3H);

### (2-Chloro-4-methoxyphenyl)(5-methyl-6-(2-morpholinoethyl)-6H-thieno[2,3-b]pyrrol-2-yl)methanone (70)

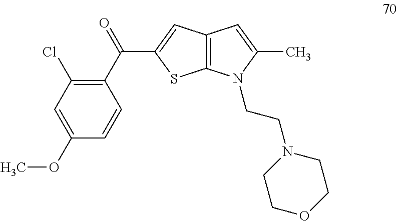 [SMILES: COc1ccc(C(=O)c2cc3cc(C)n(CCN4CCOCC4)c3s2)c(Cl)c1]

¹H NMR (CDCl₃/400 MHz) δ 7.42 (d, J=8.4 Hz, 1H), 7.26 (s, 1H), 7.00 (d, J=2.4 Hz, 1H), 6.86 (dd, J=8.4, 2.4 Hz, 1H), 6.13 (d, J=0.8 Hz, 1H), 4.05 (t, J=6.8 Hz, 2H), 3.86 (s, 3H), 3.71 (t, J=4.8 Hz, 4H), 2.74 (t, J=6.8 Hz, 2H), 2.50 (t, J=4.8 Hz, 4H), 2.37 (d, J=0.8 Hz, 3H); MS m/z: 419 (M+1).

### (2-Fluoro-4-methylphenyl)(5-methyl-6-(2-morpholinoethyl)-6H-thieno[2,3-b]pyrrol-2-yl)methanone (80)

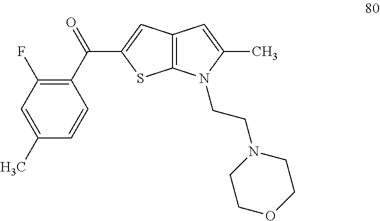 [SMILES: Cc1ccc(C(=O)c2cc3cc(C)n(CCN4CCOCC4)c3s2)c(F)c1]

¹H NMR (CDCl₃/400 MHz) δ 7.46 (t, J=7.2 Hz, 1H), 7.38 (d, J=2.0 Hz, 1H), 7.27 (s, 1H), 7.03 (d, J=7.6 Hz, 1H), 6.98 (d, J=11.2 Hz, 1H), 6.14 (d, J=1.2 Hz, 1H), 4.05 (t, J=6.8 Hz, 2H), 3.71 (t, J=4.8 Hz, 4H), 2.73 (t, J=6.8 Hz, 2H), 2.50 (t, J=4.8 Hz, 4H), 2.37 (s, 3H), 2.36 (s, 3H); MS m/z: 387 (M+1).

### (2-Chloro-4-methylphenyl)(5-methyl-6-(2-morpholinoethyl)-6H-thieno[2,3-b]pyrrol-2-yl)methanone (86)

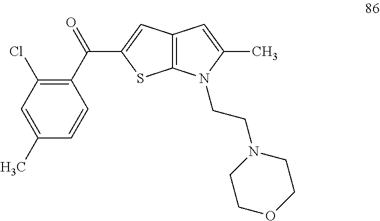 [SMILES: Cc1ccc(C(=O)c2cc3cc(C)n(CCN4CCOCC4)c3s2)c(Cl)c1]

¹H NMR (CDCl₃/400 MHz) δ 7.35 (d, J=8.0 Hz, 1H), 7.28 (s, 1H), 7.23 (s, 1H), 7.15-7.12 (m, 1H), 6.12 (d, J=0.8 Hz, 1H), 4.05 (t, J=6.8 Hz, 2H), 3.70 (t, J=4.8 Hz, 4H), 2.73 (t, J=4.8 Hz, 2H), 2.50 (t, J=6.8 Hz, 4H), 2.40 (m, 4H), 2.37 (d, J=1.2 Hz, 3H); MS m/z: 403 (M+1).

### (4-Methoxy-2-methylphenyl)(5-methyl-6-(2-morpholinoethyl)-6H-thieno[2,3-b]pyrrol-2-yl)methanone (88)

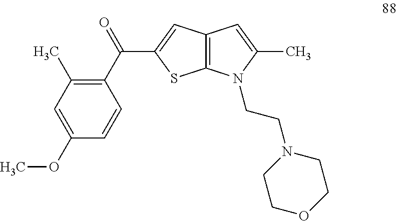 [SMILES: COc1ccc(C(=O)c2cc3cc(C)n(CCN4CCOCC4)c3s2)c(C)c1]

¹H NMR (CDCl₃/400 MHz) δ 7.45 (d, J=8.4 Hz, 1H), 7.27 (s, 1H), 6.81 (d, J=2.4 Hz, 1H), 6.75 (dd, J=8.0, 2.4 Hz, 1H), 6.17 (d, J=0.8 Hz, 1H), 4.05 (t, J=6.8 Hz, 2H), 3.85 (s, 3H), 3.72-3.70 (m, 4H), 2.74 (t, J=6.4 Hz, 2H), 2.51-2.49 (m, 4H), 2.41 (s, 3H), 2.37 (d, J=0.8 Hz, 3H).

### (4-Ethoxy-2-fluorophenyl)(5-methyl-6-(2-morpholinoethyl)-6H-thieno[2,3-b]pyrrol-2-yl)methanone (89)

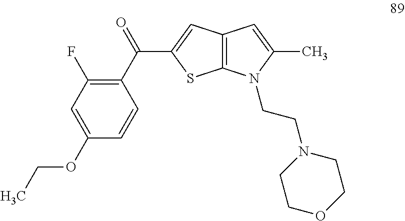 [SMILES: CCOc1ccc(C(=O)c2cc3cc(C)n(CCN4CCOCC4)c3s2)c(F)c1]

¹H NMR (CDCl₃/400 MHz) δ 7.53 (t, J=8.4 Hz, 1H), 7.40 (d, J=2.4 Hz, 1H), 6.74 (dd, J=8.8, 2.4 Hz, 1H), 6.67 (dd, J=12.0, 2.4 Hz, 1H), 6.15 (d, J=0.8 Hz, 1H), 4.10-4.03 (m, 4H), 3.70 (t, J=2.4 Hz, 4H), 2.73 (t, J=6.8 Hz, 2H), 2.51-2.49 (m, 4H), 2.37 (d, J=0.8 Hz, 3H), 1.45 (t, J=7.2 Hz, 3H).

### Benzo[d][1,3]dioxol-5-yl(5-methyl-6-(2-morpholinoethyl)-6H-thieno[2,3-b]pyrrol-2-yl)methanone (106)

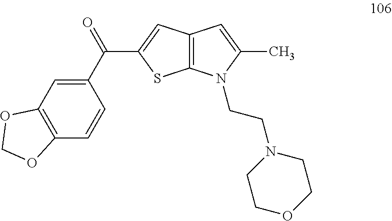 [SMILES: Cc1cc2cc(C(=O)c3ccc4c(c3)OCO4)sc2n1CCN1CCOCC1]

¹H NMR (CDCl₃/400 MHz) δ 7.52 (s, 1H), 7.44 (dd, J=8.0, 1.6 Hz, 1H), 7.34 (d, J=1.6 Hz, 1H), 6.88 (d, J=8.0 Hz, 1H), 6.16 (d, J=1.2 Hz, 1H), 6.05 (s, 2H), 4.05 (t, J=6.8 Hz, 2H), 3.70 (t, J=4.8 Hz, 4H), 2.73 (t, J=6.8 Hz, 2H), 2.50 (t, J=4.8 Hz, 4H), 2.38 (d, J=0.8 Hz, 3H).

### (4-Isopropoxyphenyl)(5-methyl-6-(2-morpholinoethyl)-6H-thieno[2,3-b]pyrrol-2-yl)methanone (111)

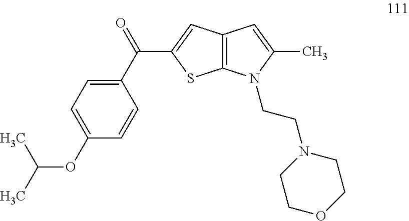 [SMILES: Cc1cc2cc(C(=O)c3ccc(OC(C)C)cc3)sc2n1CCN1CCOCC1]

¹H NMR (CDCl₃/400 MHz) δ 7.84 (d, J=8.8 Hz, 2H), 7.52 (s, 1H), 6.95 (d, J=8.8 Hz, 2H), 6.17 (d, J=1.2 Hz, 1H), 4.69-4.63 (m, 1H), 4.06 (t, J=6.8 Hz, 4H), 3.71 (t, J=4.4 Hz, 4H), 2.74 (t, J=6.8 Hz, 2H), 2.50 (t, J=4.4 Hz, 4H), 2.38 (d, J=0.8 Hz, 3H), 1.38 (d, J=6.4 Hz, 6H); MS m/z: 413(M+1).

### (5-Methyl-6-(2-morpholinoethyl)-6H-thieno[2,3-b]pyrrol-2-yl)(4-(methylthio)phenyl)methanone (115)

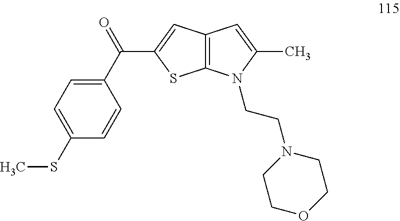 [SMILES: CSc1ccc(C(=O)c2cc3cc(C)n(CCN4CCOCC4)c3s2)cc1]

¹H NMR (CDCl₃/400 MHz) δ 7.78 (d, J=8.4 Hz, 2H), 7.50 (s, 1H), 7.31 (d, J=8.4 Hz, 2H), 6.17 (d, J=0.8 Hz, 1H), 4.06 (t, J=6.8 Hz, 2H), 3.71 (t, J=4.8 Hz, 4H), 2.74 (t, J=6.8 Hz, 4H), 2.54 (s, 3H), 2.50 (t, J=4.8 Hz, 4H), 2.38 (d, J=1.2 Hz, 3H); MS m/z: 401 (M+1).

### (3-Chloro-4-methoxyphenyl)(5-methyl-6-(2-morpholinoethyl)-6H-thieno[2,3-b]pyrrol-2-yl)methanone (116)

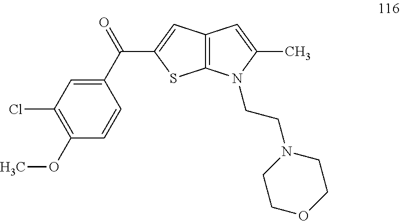 [SMILES: COc1ccc(C(=O)c2cc3cc(C)n(CCN4CCOCC4)c3s2)cc1Cl]

¹H NMR (CDCl₃/400 MHz) δ 7.91 (d, J=2.0 Hz, 1H), 7.77 (dd, J=8.4, 2.4 Hz, 1H), 7.50 (s, 1H), 7.00 (d, J=8.8 Hz, 1H), 6.18 (d, J=1.2 Hz, 1H), 4.05 (t, J=6.8 Hz, 2H), 3.98 (s, 3H), 3.70 (t, J=4.8 Hz, 4H), 2.73 (t, J=6.8 Hz, 2H), 2.49 (t, J=4.8 Hz, 4H), 2.38 (d, J=0.8 Hz, 3H).

### (2-Methoxypyrimidin-5-yl)(5-methyl-6-(2-morpholinoethyl)-6H-thieno[2,3-b]pyrrol-2-yl)methanone (118)

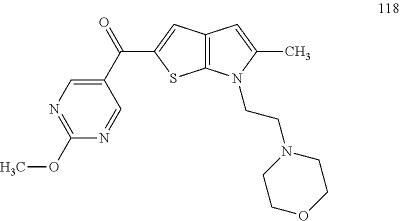 [SMILES: COc1ncc(C(=O)c2cc3cc(C)n(CCN4CCOCC4)c3s2)cn1]

¹H NMR (CDCl₃/400 MHz) δ 8.98 (s, 2H), 7.49 (s, 1H), 6.20 (d, J=0.8 Hz, 1H), 4.11 (s, 3H), 4.06 (t, J=4.8 Hz, 2H), 3.70 (t, J=4.4 Hz, 4H), 2.74 (s, J=6.8 Hz, 2H), 2.50 (t, J=4.8 Hz, 4H), 2.39 (d, J=0.8 Hz, 3H); MS m/z: 387 (M+1).

### (4-Methoxy-2-(trifluoromethyl)phenyl)(5-methyl-6-(2-morpholinoethyl)-6H-thieno[2,3-b]pyrrol-2-yl)methanone (119)

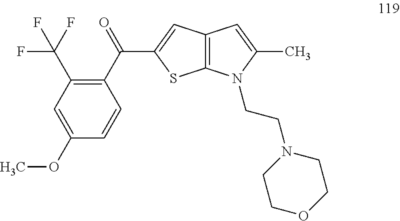 [SMILES: COc1ccc(C(=O)c2cc3cc(C)n(CCN4CCOCC4)c3s2)c(C(F)(F)F)c1]

¹H NMR (CD₃OD/400 MHz) δ 7.54 (d, J=8.4 Hz, 1H), 7.32 (d, J=2.8 Hz, 1H), 7.25 (dd, J=8.4, 2.8 Hz, 1H), 7.20 (s, 1H), 6.17 (d, J=0.8 Hz, 1H), 4.13 (t, J=5.2 Hz, 2H), 3.93 (s, 3H), 3.66 (t, J=4.4 Hz, 4H), 3.73 (t, J=6.4 Hz, 2H), 2.90 (t, J=4.8 Hz, 4H), 2.38 (d, J=1.2 Hz, 3H); MS m/z: 453 (M+1).

### (2,4-Dimethoxyphenyl)(5-methyl-6-(2-morpholinoethyl)-6H-thieno[2,3-b]pyrrol-2-yl)methanone (120)

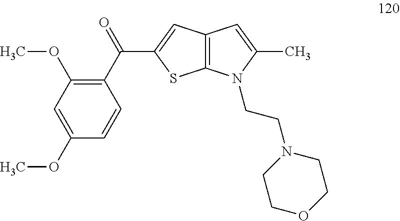 [SMILES: COc1ccc(C(=O)c2cc3cc(C)n(CCN4CCOCC4)c3s2)c(OC)c1]

¹H NMR (CDCl₃/400 MHz) δ 7.40 (d, J=8.8 Hz, 1H), 7.31 (s, 1H), 6.54-6.50 (m, 2H), 6.12 (s, 1H), 4.04 (t, J=6.8 Hz, 2H), 3.86 (s, 3H), 3.79 (s, 3H), 3.72-3.67 (m, 4H), 2.73 (t, J=6.8 Hz, 2H), 2.52-2.45 (m, 4H), 2.36 (d, J=0.8 Hz, 3H); MS m/z: 415.40 (M+1).

### (2,3-Dichlorophenyl)(5-methyl-6((1-methylpiperidin-2-yl)methyl)-6H-thieno[2,3-b]pyrrol-2-yl)methanone (24)

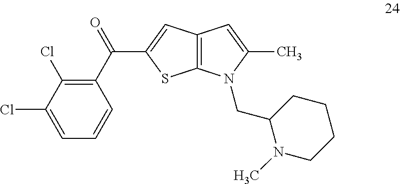 [SMILES: Cc1cc2cc(C(=O)c3cccc(Cl)c3Cl)sc2n1CC1CCCCN1C]

¹H NMR (CDCl₃/400 MHz) δ 7.55 (dd, J=8.0, 1.6 Hz, 1H), 7.35 (dd, J=7.6, 1.6 Hz, 1H), 7.29 (t, J=7.6 Hz, 1H), 7.19 (br s, 1H), 6.13 (s, 1H), 4.35 (dd, J=14.0, 4.0 Hz, 1H), 3.76 (dd, J=14.0, 10.0 Hz, 1H), 2.92-2.86 (m, 1H), 2.54-2.44 (m, 1H), 2.45 (s, 3H), 2.35 (s, 3H), 2.17 (td, J=11.6, 4.4 Hz, 1H), 1.74-1.52 (m, 3H), 1.46-1.38 (m, 1H), 1.32-1.12 (m, 3H).

### (2,3-Dichlorophenyl)(5-methyl-6-((tetrahydro-2H-pyran-4-yl)methylsulfonyl)-6H-thieno[2,3-b]pyrrol-2-yl)methanone (46)

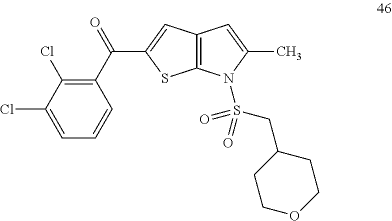 [SMILES: Cc1cc2cc(C(=O)c3cccc(Cl)c3Cl)sc2n1S(=O)(=O)CC1CCOCC1]

¹H NMR (CDCl₃/400 MHz) δ 7.61-7.56 (m, 1H), 7.36-7.28 (m, 2H), 7.23 (s, 1H), 6.31 (d, J=1.2 Hz, 1H), 4.00-3.90 (m, 2H), 3.38 (t, J=12.0 Hz, 2H), 3.17 (d, J=6.4 Hz, 2H), 2.53 (d, J=1.2 Hz, 3H), 2.29 (br s, 1H), 1.77 (d, J=12.8 Hz, 2H), 1.50-1.38 (m, 2H).

### 2-(2,3-Dichlorobenzoyl)-N,N,5-trimethyl-6H-thieno[2,3-b]pyrrole-6-sulfonamide (41)

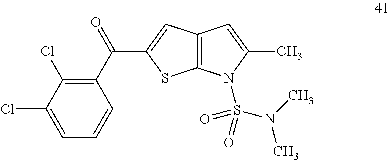 [SMILES: Cc1cc2cc(C(=O)c3cccc(Cl)c3Cl)sc2n1S(=O)(=O)N(C)C]

MS m/z: 417.03 (M+1).

### 2-(4-Methoxybenzoyl)-N,N,5-trimethyl-6H-thieno[2,3-b]pyrrole-6-sulfonamide (51)

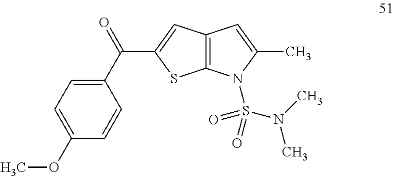

¹H NMR (CDCl₃/400 MHz) δ 7.89-7.83 (m, 2H), 7.46 (s, 1H), 7.00-6.95 (m, 2H), 6.30 (d, J=0.8 Hz, 1H), 3.88 (s, 3H), 2.93 (s, 6H), 2.52 (d, J=1.2 Hz, 3H).

### 2-(6-Methoxynicotinoyl)-N,N,5-trimethyl-6H-thieno[2,3-b]pyrrole-6-sulfonamide (113)

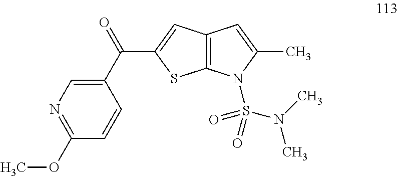

¹H NMR (CDCl₃/400 MHz) δ 8.72 (dd, J=2.4, 0.8 Hz, 1H), 8.07 (dd, J=8.4, 2.4 Hz, 1H), 7.50 (s, 1H), 6.85 (dd, J=8.4, 0.8 Hz, 1H), 6.32 (m, 1H), 4.03 (s, 3H), 2.95 (s, 6H), 2.54 (d, J=1.2 Hz, 3H).

### (5-Methyl-6-(2-morpholinoethyl)-6H-thieno[2,3-b]pyrrol-2-yl)(6-methylpyridin-3-yl)methanol (125)

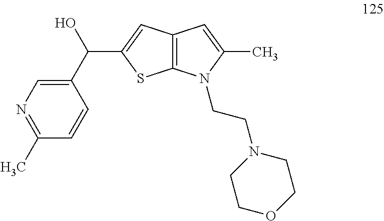 [SMILES: Cc1ccc(C(O)c2cc3cc(C)n(CCN4CCOCC4)c3s2)cn1]

This compound was prepared according to Scheme 1, starting from 2-methyl-5-carboxyaldehydepyridine, and using the same conditions that were used in the examples provided with esters. ¹H NMR (CDCl₃/400 MHz) δ 8.52 (d, J=2.0 Hz, 1H), 7.69 (dd, J=8.0, 2.4 Hz, 1H), 7.13 (d, J=8.0 Hz, 1H), 6.73 (s, 1H), 6.02 (s, 1H) 6.01 (d, J=2.4 Hz, 1H), 3.98 (t, J=6.8 Hz, 2H), 3.69-3.67 (m, 4H), 2.66 (t, J=6.8 Hz, 2H), 2.54 (s, 3H), 2.47-2.44 (m, 4H), 2.33 (s, 3H); MS m/z: 372.50 (M+1).

### 4-(2-(2-(2,3-Dichlorobenzyl)-5-methyl-6H-thieno[2,3-b]pyrrol-6-yl)ethyl)morpholine (32)

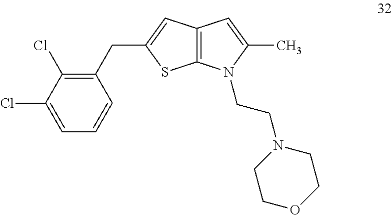 [SMILES: Cc1cc2cc(Cc3cccc(Cl)c3Cl)sc2n1CCN1CCOCC1]

This compound was prepared according to Scheme 1, starting from the benzyl bromide instead of the ester, all other reagents and conditions as indicated in the ester examples. (¹H NMR (CDCl₃/400 MHz) δ 7.26 (dd, J=8.0, 1.6 Hz, 1H), 7.10 (dd, J=8.0, 1.6 Hz, 1H), 7.04 (t, J=8.0 Hz, 1H), 6.62 (s, 1H), 5.95 (s, 1H), 4.20 (s, 2H), 3.90 (t, J=6.8 Hz, 2H), 3.61 (t, J=4.8 Hz, 4H), 2.60 (t, J=6.8 Hz, 2H), 2.40 (t, J=4.8 Hz, 4H), 2.26 (s, 3H).

### (6-(3-Hydroxy-3-methylbutyl)-5-methyl-6H-thieno[2,3-b]pyrrol-2-yl)(4-methoxyphenyl)methanone (77)

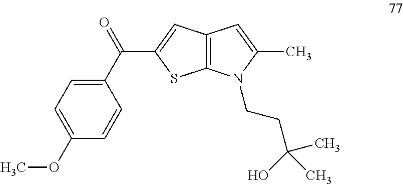 [SMILES: COc1ccc(C(=O)c2cc3cc(C)n(CCC(C)(C)O)c3s2)cc1]

Additional standard alcohol protection and deprotection using tert-butyl silicon ether intermediate were used for the preparation of this compound. ¹H NMR (CDCl₃/400 MHz) δ 7.87-7.81 (m, 2H), 7.51 (s, 1H), 6.99-6.92 (m, 2H), 6.16 (d, J=0.8 Hz, 1H), 4.13 (t, J=8.0 Hz, 2H), 3.88 (s, 3H), 2.37 (d, J=0.8 Hz, 3H), 1.97 (t, J=8.0 Hz, 2H), 1.32 (s, 6H).

### (2-chloro-4-ethoxyphenyl)(5-methyl-6-(2-morpholinoethyl)-6H-thieno[2,3-b]pyrrol-2-yl)methanone (126)

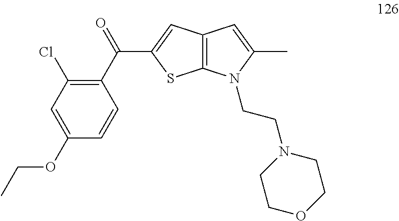 [SMILES: CCOc1ccc(C(=O)c2cc3cc(C)n(CCN4CCOCC4)c3s2)c(Cl)c1]

¹H NMR (CDCl₃/400 MHz) 7.40 (d, J=8.0 Hz, 1H), 7.26 (s, 1H), 6.98 (d, J=4.0 Hz, 1H), 6.84 (dd, J=8.0, 4.0 Hz, 1H), 6.13 (d, J=0.8 Hz, 1H), 6.01 (d, J=2.4 Hz, 1H), 4.07 (q, J=6.8 Hz, 2H), 4.08-4.29 (m, 2H), 3.75-3.64 (m, 4H), 2.77-2.69 (m, 2H), 2.56-2.43 (m, 4H), 2.37 (s, 3H), 1.45 (t, J=6.8 Hz, 3H); MS m/z: 433.12 (M+1).

### (2,6-difluoro-4-methoxyphenyl)(5-methyl-6-(2-morpholinoethyl)-6H-thieno[2,3-b]pyrrol-2-yl)methanone (127)

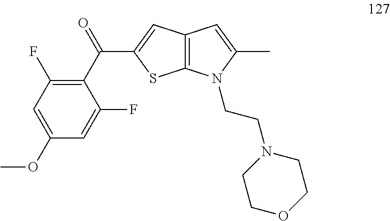 [SMILES: COc1cc(F)c(C(=O)c2cc3cc(C)n(CCN4CCOCC4)c3s2)c(F)c1]

¹H NMR (CDCl₃/400 MHz) 7.35 (s, 1H), 6.52 (dd, J=11.6, 2.4 Hz, 2H), 6.14 (d, J=1.2 Hz, 1H), 4.04 (t, J=6.8 Hz, 2H), 3.84 (s, 3H), 3.70 (t, J=4.4 Hz, 4H), 2.73 (t, J=6.8 Hz, 2H), 2.50 (t, J=4.4 Hz, 4H), 2.36 (d, J=0.8 Hz, 3H); MS m/z: 421.14 (M+1).

### (3-fluoro-4-methoxyphenyl)(5-methyl-6-(2-morpholinoethyl)-6H-thieno[2,3-b]pyrrol-2-yl)methanone (128)

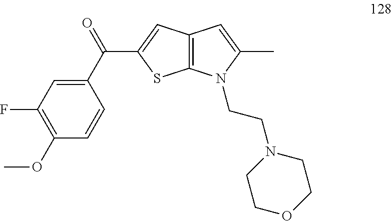 [SMILES: COc1ccc(C(=O)c2cc3cc(C)n(CCN4CCOCC4)c3s2)cc1F]

¹H NMR (CDCl₃/400 MHz) 7.61-7.55 (m, 2H), 7.45 (s, 1H), 6.97 (t, J=8.4 Hz, 1H), 6.11 (d, J=1.2 Hz, 1H), 4.00 (t, J=6.8 Hz, 2H), 3.90 (s, 3H), 3.64 (t, J=4.4 Hz, 4H), 2.68 (t, J=6.8 Hz, 2H), 2.34 (t, J=4.4 Hz, 4H), 2.32 (d, J=0.8 Hz, 3H); MS m/z: 403.17 (M+1).

### 4-(5-methyl-6-(2-morpholinoethyl)-6H-thieno[2,3-b]pyrrole-2-carbonyl)benzonitrile (129)

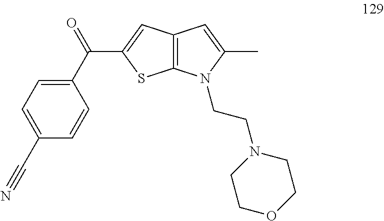 [SMILES: Cc1cc2cc(C(=O)c3ccc(C#N)cc3)sc2n1CCN1CCOCC1]

¹H NMR (CDCl₃/400 MHz) 7.92-7.86 (m, 2H), 7.80-7.75 (m, 2H), 7.42 (s, 1H), 6.17 (d, J=1.2 Hz, 1H), 4.15-3.98 (m, 2H), 3.78-3.55 (m, 4H), 2.80-2.68 (m, 2H), 2.51-2.42 (m, 2H), 2.38 (s, 3H); MS m/z: 380.10 (M+1).

### (5-methyl-6-(2-morpholinoethyl)-6H-thieno[2,3-b]pyrrol-2-yl)(6-methylpyridin-3-yl)methanone (130)

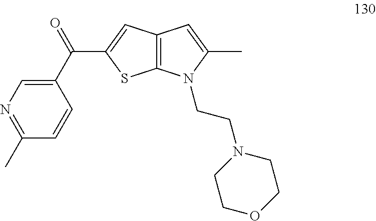 [SMILES: Cc1ccc(C(=O)c2cc3cc(C)n(CCN4CCOCC4)c3s2)cn1]

¹H NMR (CDCl₃/400 MHz) 8.02 (dd, J=8.0, 2.4 Hz, 1H), 7.49 (s, 1H), 7.28 (d, J=8.0 Hz, 1H), 6.18 (d, J=0.8 Hz, 1H), 4.06 (t, J=6.8 Hz, 2H), 3.76-3.64 (m, 4H), 2.74 (t, J=6.4 Hz, 2H), 2.65 (s, 3H), 2.53-2.45 (m, 4H), 2.38 (d, J=0.8 Hz, 3H); MS m/z: 370.19 (M+1).

### (5-methoxypyridin-2-yl)(5-methyl-6-(2-morpholinoethyl)-6H-thieno[2,3-b]pyrrol-2-yl)methanone (131)

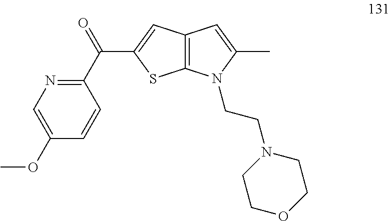 [SMILES: COc1ccc(C(=O)c2cc3cc(C)n(CCN4CCOCC4)c3s2)nc1]

¹H NMR (CDCl₃/400 MHz) 8.34 (m, 2H), 8.17 (d, J=8.8 Hz, 1H), 8.31 (dd, J=8.8, 2.8 Hz, 1H), 6.20 (s, 1H), 4.06 (t, J=6.8 Hz, 2H), 3.94 (t, J=4.0 Hz, 4H), 2.75 (t, J=6.8 Hz, 2H), 2.50 (t, J=4.0 Hz, 4H), 2.38 (s, 3H); MS m/z: 386.15 (M+1).

### tert-butylmethyl(4-(5-methyl-6-(2-morpholinoethyl)-6H-thieno[2,3-b]pyrrole-2-carbonyl)phenyl)carbamate (132)

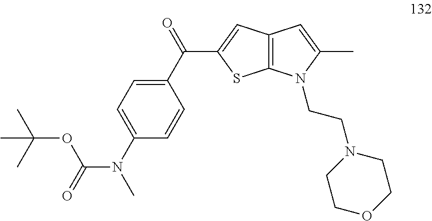 [SMILES: Cc1cc2cc(C(=O)c3ccc(N(C)C(=O)OC(C)(C)C)cc3)sc2n1CCN1CCOCC1]

¹H NMR (CDCl₃/400 MHz) 7.79 (d, J=8.4 Hz, 2H), 7.49 (s, 1H), 7.35 (d, J=8.4 Hz, 2H), 6.15 (s, 1H), 4.04 (t, J=6.4 Hz, 2H), 3.69 (t, J=4.4 Hz, 4H), 3.30 (s, 1H), 2.72 (t, J=6.4 Hz, 2H), 2.48 (t, J=4.4 Hz, 4H), 2.37 (s, 3H), 1.47 (s, 9H); MS m/z: 484.24 (M+1).

### (4-fluoro-2-methoxyphenyl)(5-methyl-6-(2-morpholinoethyl)-6H-thieno[2,3-b]pyrrol-2-yl)methanone (133)

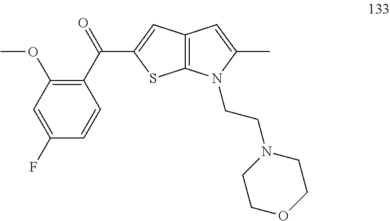 [SMILES: COc1cc(F)ccc1C(=O)c1cc2cc(C)n(CCN3CCOCC3)c2s1]

¹H NMR (CDCl₃/400 MHz) 7.34-7.31 (m, 1H), 7.20 (s, 1H), 6.66-6.63 (m, 2H), 6.05 (s, 1H), 3.98 (t, J=6.8 Hz, 2H), 3.73 (s, 3H), 3.64 (t, J=4.0 Hz, 4H), 2.66 (t, J=6.8 Hz, 2H), 2.43 (t, J=4.4 Hz, 4H), 2.30 (s, 3H); MS m/z: 403.17 (M+1).

### (5-methyl-6-(2-morpholinoethyl)-6H-thieno[2,3-b]pyrrol-2-yl)(4-(methylamino)phenyl)methanone (134)

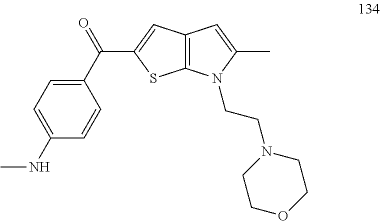 [SMILES: CNc1ccc(C(=O)c2cc3cc(C)n(CCN4CCOCC4)c3s2)cc1]

¹H NMR (CDCl₃/400 MHz) 7.75 (d, J=8.4 Hz, 2H), 7.46 (d, J=1.2 Hz, 1H), 6.55 (d, J=8.4 Hz, 2H), 6.10 (s, 1H), 4.11 (br s, 1H), 3.99 (t, J=6.8 Hz, 2H), 3.64 (t, J=4.4 Hz, 4H), 2.85 (s, 3H), 2.68 (t, J=6.8 Hz, 2H), 2.44 (t, J=4.4 Hz, 4H), 2.32 (s, 3H); MS m/z: 384.18 (M+1).

### (2,4-difluorophenyl)(5-methyl-6-(2-morpholinoethyl)-6H-thieno[2,3-b]pyrrol-2-yl)methanone (135)

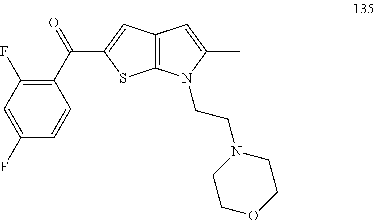 [SMILES: Cc1cc2cc(C(=O)c3ccc(F)cc3F)sc2n1CCN1CCOCC1]

¹H NMR (CDCl₃/400 MHz) 7.59-7.53 (m, 1H), 7.33 (s, 1H), 6.97-6.87 (m, 2H), 6.13 (s, 1H), 4.04 (t, J=6.8 Hz, 2H), 3.68 (t, J=4.4 Hz, 4H), 2.71 (t, J=6.8 Hz, 2H), 2.47 (t, J=4.4 Hz, 4H), 2.35 (s, 3H); MS m/z: 391.15 (M+1).

### (6-methoxy-2-methylpyridin-3-yl)(5-methyl-6-(2-morpholinoethyl)-6H-thieno[2,3-b]pyrrol-2-yl)methanone (136)

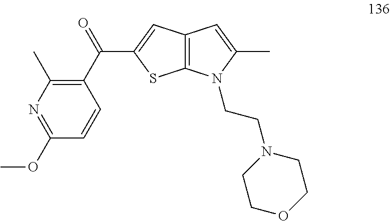 [SMILES: COc1ccc(C(=O)c2cc3cc(C)n(CCN4CCOCC4)c3s2)c(C)n1]

¹H NMR (CDCl₃/400 MHz) 7.65 (d, J=8.4 Hz, 1H), 7.21 (d, J=2.0 Hz, 1H), 6.58 (d, J=8.4 Hz, 1H), 6.11 (d, J=0.8 Hz, 1H), 4.03 (t, J=6.4 Hz, 2H), 3.95 (d, J=1.6 Hz, 3H), 3.68 (t, J=4.0 Hz, 4H), 2.71 (t, J=6.8 Hz, 2H), 2.51 (s, 3H), 2.47 (t, J=4.4 Hz, 4H), 2.37 (s, 31-1); MS m/z: 400.18 (M+1).

### (2-chloro-6-methoxypyridinyl-3-yl)(5-methyl-6-(2-morpholinoethyl)-6H-thieno[2,3-b]pyrrol-2-yl)methanone (137)

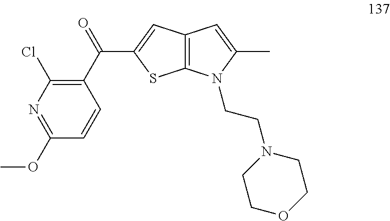 [SMILES: COc1ccc(C(=O)c2cc3cc(C)n(CCN4CCOCC4)c3s2)c(Cl)n1]

¹H NMR (CDCl₃/400 MHz) 7.69 (d, J=8.0 Hz, 1H), 7.27 (s, 1H), 6.74 (d, J=8.0 Hz, 1H), 6.14 (d, J=0.8 Hz, 1H), 4.04 (t, J=6.4 Hz, 2H), 4.06 (s, 3H), 3.73-3.67 (m, 4H), 2.73 (t, J=6.4 Hz, 2H), 2.53-2.47 (m, 4H), 2.37 (d, J=0.8 Hz, 3H); MS m/z: 420.14 (M+1).

### 2,2,2-trifluoro-1-(4-methoxyphenyl)-1-(5-methyl-6-(2-morpholinoethyl)-6H-thieno[2,3-b]pyrrol-2-yl)ethanol (138)

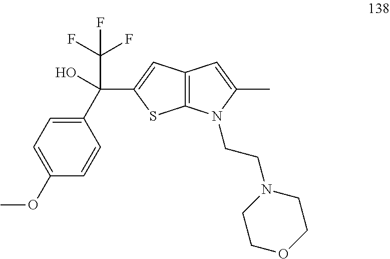 [SMILES: COc1ccc(C(O)(c2cc3cc(C)n(CCN4CCOCC4)c3s2)C(F)(F)F)cc1]

¹H NMR (CDCl₃/400 MHz) 7.66-7.53 (m, 2H), 7.07 (s, 1H), 6.95-6.86 (m, 2H), 6.07 (s, 1H), 4.01-3.94 (m, 2H), 3.84 (s, 3H), 2.69-2.60 (m, 2H), 2.49-2.40 (m, 4H), 2.35 (s, 3H); MS m/z: 455.21 (M+1).

The compounds below were prepared according to Scheme 2:

### Ethyl 3-(2-(4-methoxybenzoyl)-5-methyl-6H-thieno[2,3-b]pyrrol-6-yl)propanoate (72)

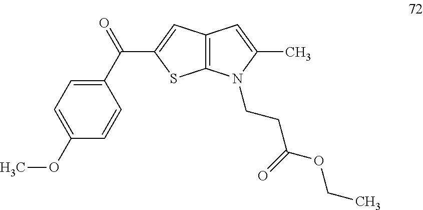 [SMILES: CCOC(=O)CCn1c(C)cc2cc(C(=O)c3ccc(OC)cc3)sc21]

¹H NMR (CDCl₃/400 MHz) δ 7.87-7.82 (m, 2H), 7.50 (s, 1H), 6.99-6.94 (m, 2H), 6.15 (d, J=0.8 Hz, 1H), 4.26 (t, J=7.2 Hz, 2H), 4.13 (q, J=6.8 Hz, 2H), 3.87 (s, 3H), 2.84 (t, J=7.2 Hz, 2H), 2.38 (d, J=0.8 Hz, 3H), 1.23 (t, J=6.8 Hz, 3H).

### 3-(2-(4-Methoxybenzoyl)-5-methyl-6H-thieno[2,3-b]pyrrol-6-yl)propanoic acid (73)

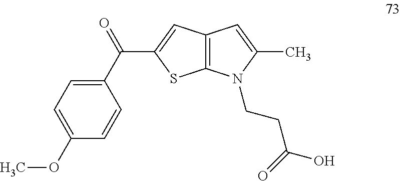 [SMILES: COc1ccc(C(=O)c2cc3cc(C)n(CCC(=O)O)c3s2)cc1]

The title compound was prepared by the hydrolysis of ethyl 3-(2-(4-methoxybenzoyl)-5-methyl-6H-thieno[2,3-b]pyrrol-6-yl)propanoate (72). ¹H NMR (CDCl₃/400 MHz) δ 7.88-7.83 (m, 2H), 7.52 (s, 1H), 7.00-6.96 (m, 2H), 6.18 (d, J=0.8 Hz, 1H), 4.30 (t, J=6.8 Hz, 2H), 3.89 (s, 3H), 2.93 (t, J=6.8 Hz, 2H), 2.40 (d, J=1.2 Hz, 3H).

### 2-(2-(2,3-Dichlorobenzoyl)-5-methyl-6H-thieno[2,3-b]pyrrol-6-yl)-1-morpholinoethanone (22)

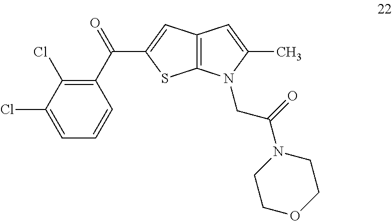 [SMILES: Cc1cc2cc(C(=O)c3cccc(Cl)c3Cl)sc2n1CC(=O)N1CCOCC1]

¹H NMR (CDCl₃/400 MHz) δ 7.55 (dd, J=7.6, 1.6 Hz, 1H), 7.33 (dd, J=7.6, 2.0 Hz, 1H), 7.29 (t, J=7.6 Hz, 1H), 7.22 (br s, 1H), 6.20 (s, 1H), 4.73 (s, 2H), 3.74 (m, 4H), 3.66 (m, 2H), 3.55 (m, 2H), 2.31 (s, 3H).

### (6-(2-Methoxyethyl)-5-methyl-6H-thieno[2,3-b]pyrrol-2-yl)(4-methoxyphenyl)methanone (49)

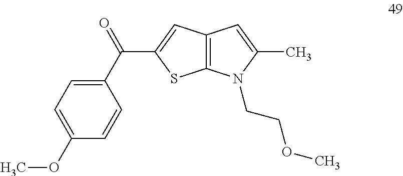

MS m/z: 330.16 (M+1).

### (6-Methoxypyridin-3-yl)(5-methyl-6-(2-(piperidin-1-yl)ethyl)-6H-thieno[2,3-b]pyrrol-2-yl)methanone (117)

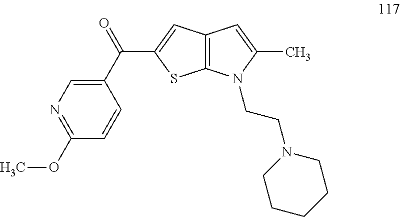 [SMILES: COc1ccc(C(=O)c2cc3cc(C)n(CCN4CCCCC4)c3s2)cn1]

¹H NMR (MeOD/400 MHz) δ 8.61 (dd, J=2.4, 0.4 Hz, 1H), 8.07 (dd, J=8.8, 2.4 Hz, 1H), 7.58 (s, 1H), 6.90 (dd, J=8.8, 0.8 Hz, 1H), 6.21 (d, J=0.8 Hz, 1H), 4.13 (t, J=6.8 Hz, 2H), 3.99 (s, 3H), 2.69 (d, J=6.8 Hz, 2H), 2.54-2.44 (m, 4H), 2.38 (d, J=0.8 Hz, 3H), 1.60 (m, 4H), 1.51-1.43 (m, 2H); MS m/z: 384.14 (M+1).

### (2,3-Dichlorophenyl)(6-(ethylsulfonyl)-5-methyl-6H-thieno[2,3-b]pyrrol-2-yl)methanone (25)

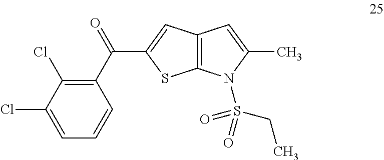 [SMILES: CCS(=O)(=O)n1c(C)cc2cc(C(=O)c3cccc(Cl)c3Cl)sc21]

¹H NMR (CDCl₃/400 MHz) δ 7.58 (dd, J=7.6, 2.0 Hz, 1H), 7.35-7.29 (m, 2H), 7.23 (s, 1H), 6.31 (d, J=0.8 Hz, 1H), 3.37 (q, J=7.6 Hz, 2H), 2.53 (d, J=0.8 Hz, 3H), 1.32 (t, J=7.6 Hz, 3H).

### (2,3-Dichlorophenyl)(5-methyl-6-phenethyl-6H-thieno[2,3-b]pyrrol-2-yl)methanone (30)

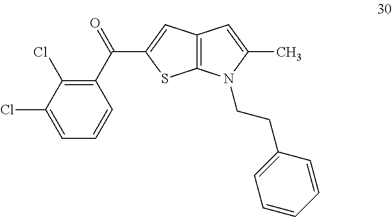 [SMILES: Cc1cc2cc(C(=O)c3cccc(Cl)c3Cl)sc2n1CCc1ccccc1]

¹H NMR (CDCl₃/400 MHz) δ 7.56 (dd, J=7.6, 1.6 Hz, 1H), 7.37 (dd, J=7.6, 1.6 Hz, 1H), 7.32-7.20 (m, 5H), 7.06-7.04 (m, 2H), 6.07 (d, J=0.8 Hz, 1H), 4.15 (t, J=7.2 Hz, 2H), 3.10 (t, J=7.2 Hz, 2H), 2.05 (d, J=0.8 Hz, 31-1).

### (2,3-Dichlorophenyl)(5-methyl-6-((tetrahydro-2H-pyran-4-yl)methyl)-6H-thieno[2,3-b]pyrrol-2-yl)methanone (31)

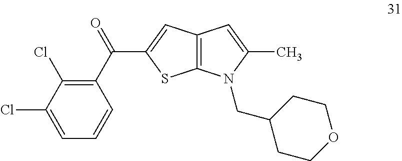 [SMILES: Cc1cc2cc(C(=O)c3cccc(Cl)c3Cl)sc2n1CC1CCOCC1]

¹H NMR (CDCl₃/400 MHz) δ 7.55 (dd, J=8.0, 1.6 Hz, 1H), 7.34 (dd, J=7.6, 1.6 Hz, 1H), 7.29 (t, J=8.0 Hz, 1H), 7.20 (br s, 1H), 6.13 (d, J=1.2 Hz), 3.98 (dd, J=7.6, 3.2 Hz, 2H), 3.81 (d, J=7.6, 2.0 Hz, 2H), 3.35 (td, J=12.0, 2.4 Hz, 2H), 2.34 (d, J=0.8 Hz, 3H), 2.28-2.14 (m, 1H), 1.58-1.52 (m, 2H), 1.44 (td, J=12.0, 4.4 Hz, 2H).

### (2,3-Dichlorophenyl)(6-(isopropylsulfonyl)-5-methyl-6H-thieno[2,3-b]pyrrol-2-yl)methanone (38)

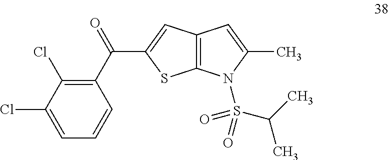 [SMILES: Cc1cc2cc(C(=O)c3cccc(Cl)c3Cl)sc2n1S(=O)(=O)C(C)C]

¹H NMR (CDCl₃/400 MHz) δ 7.57 (dd, J=7.6, 2.0 Hz, 1H), 7.36-7.28 (m, 2H), 7.21 (s, 1H), 6.29 (d, J=1.2 Hz, 1H), 3.60-3.50 (m, 1H), 2.52 (d, J=0.8 Hz, 3H), 1.37 (s, 3H), 1.35 (s, 3H).

### (2-Chlorophenyl)(6-(ethylsulfonyl)-5-methyl-6H-thieno[2,3-b]pyrrol-2-yl)methanone (42)

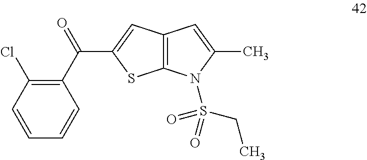 [SMILES: CCS(=O)(=O)n1c(C)cc2cc(C(=O)c3ccccc3Cl)sc21]

¹H NMR (CDCl₃/400 MHz) δ 7.42-7.27 (m, 4H), 7.18 (s, 1H) 6.23 (d, 1.6 Hz, 1H), 3.30 (q, J=7.2 Hz, 2H), 2.47 (d, J=1.2 Hz, 3H), 1.26 (t, J=7.2 Hz, 3H).

### (2-Chloropyridin-3-yl)(6-(ethylsulfonyl)-5-methyl-6H-thieno[2,3-b]pyrrol-2-yl)methanone (43)

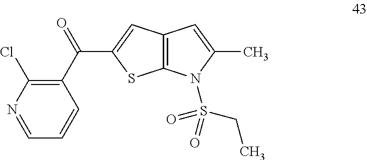 [SMILES: CCS(=O)(=O)n1c(C)cc2cc(C(=O)c3cccnc3Cl)sc21]

¹H NMR (CDCl₃/400 MHz) δ 8.55 (dd, J=4.8, 2.0 Hz, 1H), 7.80 (dd, J=7.6 Hz, 1H),

7.37 (dd, J=7.6, 2.0 Hz, 1H), 7.25 (s, 1H) 6.33 (d, 1.2 Hz, 1H), 3.38 (q, J=7.2 Hz, 2H), 2.55 (d, J=0.8 Hz, 3H), 1.34 (t, J=7.2 Hz, 3H).

### (2,3-Dichlorophenyl)(5-methyl-6-(morpholinosulfonyl)-6H-thieno[2,3-b]pyrrol-2-yl)methanone (45)

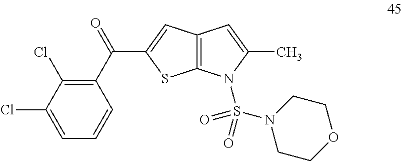 [SMILES: Cc1cc2cc(C(=O)c3cccc(Cl)c3Cl)sc2n1S(=O)(=O)N1CCOCC1]

¹H NMR (CDCl₃/400 MHz) δ 7.60-7.58 (m, 1H), 7.35-7.29 (m, 2H), 7.21 (s, 1H), 6.28 (d, J=1.2 Hz, 1H), 3.74-3.71 (m, 4H), 3.32-3.30 (m, 4H), 2.52 (d, J=0.8 Hz, 3H).

### (4-methoxyphenyl)(5-methyl-6-(morpholinosulfonyl)-6H-thieno[2,3-b]pyrrol-2-yl)methanone (53)

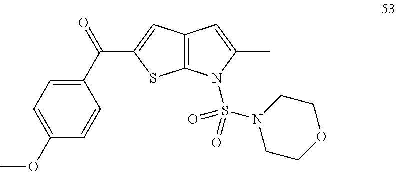 [SMILES: COc1ccc(C(=O)c2cc3cc(C)n(S(=O)(=O)N4CCOCC4)c3s2)cc1]

¹H NMR (CDCl₃/400 MHz) 7.89-7.83 (m, 2H), 7.47 (s, 1H), 7.01-6.95 (m, 2H), 6.33 (d, J=1.2 Hz, 1H), 3.89 (s, 3H), 3.70 (t, J=4.8 Hz, 4H), 3.29 (t, J=4.8 Hz, 4H), 2.53 (d, J=1.2 Hz, 3H).

### (6-(Ethylsulfonyl)-5-methyl-6H-thieno[2,3-b]pyrrol-2-yl)(4-methoxyphenyl)methanone (56)

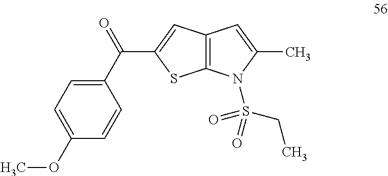

¹H NMR (CDCl₃/400 MHz) δ 7.90-7.85 (m, 2H), 7.49 (s, 1H), 7.01-6.96 (m, 2H), 6.35 (d, J=0.8 Hz, 1H), 3.89 (s, 3H), 3.36 (q, J=7.6 Hz, 2H), 2.55 (s, 3H), 1.31 (t, J=7.6 Hz, 3H).

### 3-(2-(2-(4-Methoxybenzoyl)-5-methyl-6H-thieno[2,3-b]pyrrol-6-yl)ethyl)oxazolidin-2-one (50)

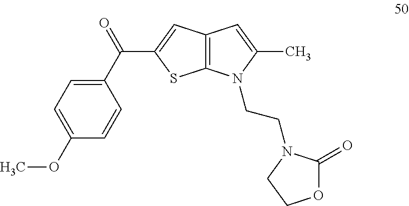 [SMILES: COc1ccc(C(=O)c2cc3cc(C)n(CCN4CCOC4=O)c3s2)cc1]

¹H NMR (CDCl₃/400 MHz) δ 7.86-7.80 (m, 2H), 7.50 (s, 1H), 7.00-6.94 (m, 2H), 6.19 (d, J=1.2 Hz, 1H), 4.20-4.14 (m, 4H), 3.86 (s, 3H), 3.65 (t, J=6.0 Hz, 2H), 3.16 (t, J=8.0 Hz, 2H), 2.36 (d, J=0.8 Hz, 3H).

### 1-(2-(2-(4-Methoxybenzoyl)-5-methyl-6H-thieno[2,3-b]pyrrol-6-yl)ethypimidazolidin-2-one (61)

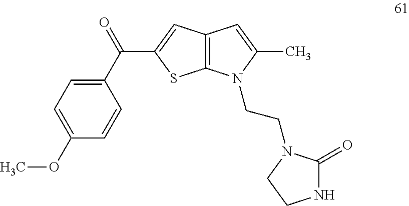 [SMILES: COc1ccc(C(=O)c2cc3cc(C)n(CCN4CCNC4=O)c3s2)cc1]

¹H NMR (CDCl₃/400 MHz) δ 7.87-7.82 (m, 2H), 7.50 (s, 1H), 7.00-6.94 (m, 2H), 6.18 (s, 1H), 4.84 (s, 1H), 4.14 (t, J=6.0 Hz, 2H), 3.87 (s, 3H), 3.56 (t, J=6.0 Hz, 2H), 3.28 (t, J=8.4 Hz, 2H), 3.04 (t, J=8.4 Hz, 3H), 2.37 (s, 3H).

### (6-(2-(1H-Imidazol-1-yl)ethyl)-5-methyl-6H-thieno[2,3-b]pyrrol-2-yl)(4-methoxyphenyl)methanone (67)

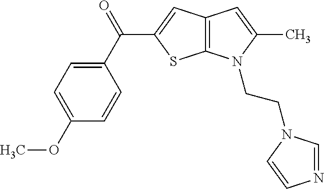 [SMILES: COc1ccc(C(=O)c2cc3cc(C)n(CCn4ccnc4)c3s2)cc1]

¹H NMR (CDCl₃/400 MHz) δ 7.88-7.83 (m, 2H), 7.53 (s, 1H), 7.10 (s, 1H), 7.00 (s, 1H), 7.00-6.95 (m, 2H), 6.59 (d, J=1.2 Hz, 1H), 6.10 (d, J=1.2 Hz, 1H), 4.40-4.35 (m, 2H), 4.26-4.21 (m, 2H), 3.89 (s, 3H), 1.84 (d, J=0.8 Hz, 3H).

### (6-Methoxypyridin-3-yl)(5-methyl-6-(2-morpholinoethyl)-6H-thieno[2,3-b]pyrrol-2-yl)methanone (58)

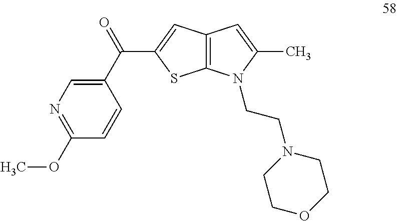 [SMILES: COc1ccc(C(=O)c2cc3cc(C)n(CCN4CCOCC4)c3s2)cn1]

¹H NMR (CD₃OD/400 MHz) δ 8.62 (d, J=2.0 Hz, 1H), 8.09 (dd, J=8.4, 2.4 Hz, 1H), 7.59 (s, 1H), 6.91 (d, J=8.4 Hz, 1H), 6.23 (d, J=0.8 Hz, 1H), 4.14 (t, J=6.4 Hz, 2H), 4.00 (s, 3H), 3.67 (t, J=4.4 Hz, 4H), 2.74 (t, J=6.4 Hz, 2H), 2.51-2.49 (m, 4H), 2.39 (d, J=0.8 Hz, 3H).

### 4-(2-(2-(6-methoxynicotinoyl)-5-methyl-6H-thieno[2,3-b]pyrrol-6-yl)ethyl)morpholin-4-ium p-toluene sulfonate (58*TsOH)

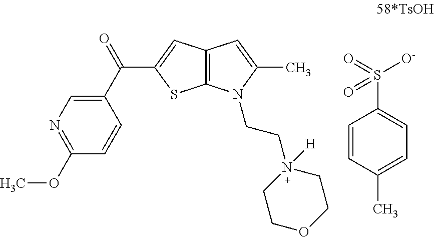

In order to prepare the salt, compound 58 was dissolved in Et₂O and 1.0 equivalents of TsOH dissolved in Et₂O. The resulting white solid was filtered and washed with copious amounts of Et₂O to afford the salt after drying. ¹H NMR (D₂O/400 MHz) δ 8.36 (s, 1H), 7.97 (d, J=9.6 Hz, 1H), 7.57-7.55 (m, 3H), 7.25 (d, J=7.6 Hz, 2H), 6.87 (d, J=8.4 Hz, 1H), 6.23 (s, 1H), 4.38 (t, J=6.8 Hz, 2H), 3.87 (br s, 5H), 3.49 (t, J=6.8 Hz, 2H), 3.32 (br s, 3H), 2.27-2.26 (m, 8H).

### (4-Methoxyphenyl)(5-methyl-6-((tetrahydro-2H-pyran-4-yl)methyl)-6H-thieno[2,3-b]pyrrol-2-yl)methanone (62)

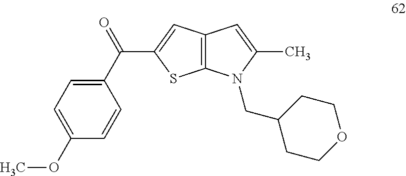 [SMILES: COc1ccc(C(=O)c2cc3cc(C)n(CC4CCOCC4)c3s2)cc1]

MS m/z: 355.15 (M+1).

### (4-Methoxyphenyl)(5-methyl-6-(2-(piperidin-1-yl)ethyl)-6H-thieno[2,3-b]pyrrol-2-yl)methanone (78)

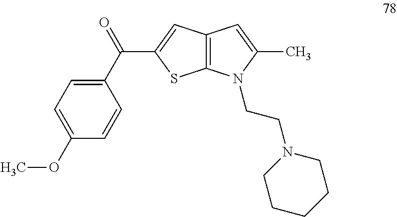 [SMILES: COc1ccc(C(=O)c2cc3cc(C)n(CCN4CCCCC4)c3s2)cc1]

¹H NMR (CDCl₃/400 MHz) δ 7.87-7.82 (m, 2H), 7.49 (s, 1H), 6.99-6.94 (m, 2H), 6.14 (d, J=1.2 Hz, 1H), 4.05 (t, J=7.2 Hz, 2H), 3.87 (s, 3H), 2.68 (t, J=7.6 Hz, 2H), 2.50-2.40 (m, 4H), 2.36 (d, J=0.8 Hz, 3H), 1.62-1.54 (m, 4H), 1.48-1.39 (m, 2H).

### (2-Chloro-4-methoxyphenyl)(5-methyl-6H-thieno[2,3-b]pyrrol-2-yl)methanone (79)

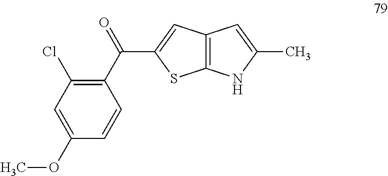

¹H NMR (CDCl₃/400 MHz) δ 8.26 (br s, 1H), 7.43 (d, J=8.4 Hz, 1H), 7.26 (d, J=2.0 Hz, 1H), 7.00 (d, J=2.4 Hz, 1H), 6.87 (dd, J=8.8, 2.4 Hz, 1H), 6.11 (d, J=1.2 Hz, 1H), 3.86 (s, 3H), 2.40 (d, J=1.2 Hz, 3H); MS m/z: 306 (M+1).

### (4-Methoxyphenyl)(5-methyl-6-(2-(tetrahydro-2H-pyran-4-yl)ethyl)-6H-thieno[2,3-b]pyrrol-2-yl)methanone (82)

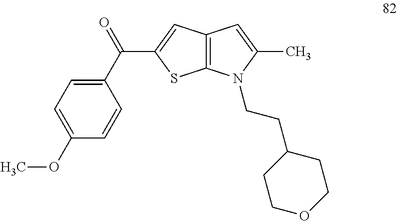 [SMILES: COc1ccc(C(=O)c2cc3cc(C)n(CCC4CCOCC4)c3s2)cc1]

MS m/z: 384.21 (M+1).

### (4-Methoxyphenyl)(5-methyl-6-(2-(pyrrolidin-1-yl)ethyl)-6H-thieno[2,3-b]pyrrol-2-yl)methanone (83)

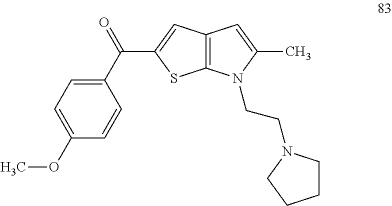 [SMILES: COc1ccc(C(=O)c2cc3cc(C)n(CCN4CCCC4)c3s2)cc1]

¹H NMR (CDCl₃/400 MHz) δ 7.87-7.82 (m, 2H), 7.51 (s, 1H), 6.99-6.94 (m, 2H), 6.16 (d, J=0.8 Hz, 1H), 4.09 (t, J=7.2 Hz, 2H), 3.89 (s, 3H), 2.89 (t, J=7.2 Hz, 2H), 2.61-2.57 (m, 4H), 2.38 (d, J=0.8 Hz, 3H), 1.82-1.77 (m, 4H).

### (6-Benzyl-5-methyl-6H-thieno[2,3-b]pyrrol-2-yl)(4-methoxyphenyl)methanone (84)

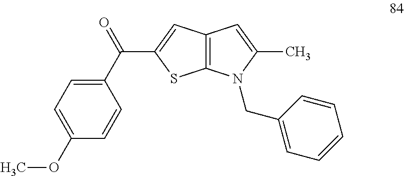 [SMILES: COc1ccc(C(=O)c2cc3cc(C)n(Cc4ccccc4)c3s2)cc1]

¹H NMR (CDCl₃/400 MHz) δ 7.86-7.82 (m, 2H), 7.52 (s, 1H), 7.37-7.24 (m, 2H), 7.00-6.95 (m, 2H), 6.23 (d, J=1.2 Hz, 1H), 5.14 (s, 2H), 3.88 (s, 3H), 2.35 (d, J=0.8 Hz, 3H).

### (2-Chloro-4-methoxyphenyl)(5-methyl-6-(2-(piperidin-1-yl)ethyl)-6H-thieno[2,3-b]pyrrol-2-yl)methanone (90)

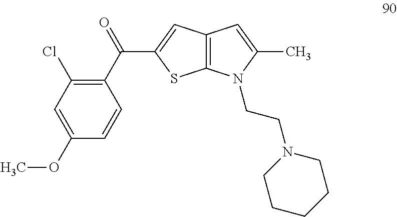 [SMILES: COc1ccc(C(=O)c2cc3cc(C)n(CCN4CCCCC4)c3s2)c(Cl)c1]

¹H NMR (CD₃OD/400 MHz) δ 7.43 (d, J=8.8 Hz, 1H), 7.26 (s, 1H), 7.09 (d, J=2.4 Hz, 1H), 6.99 (dd, J=8.8, 2.4 Hz, 1H), 6.18 (d, J=1.2 Hz, 1H), 4.14 (t, J=7.2 Hz, 2H), 3.87 (s, 3H), 2.70 (t, J=7.2 Hz, 2H), 2.50 (br s, 4H), 2.38 (d, J=0.8 Hz, 3H), 1.64-1.58 (m, 4H), 1.50-1.47 (m, 2H); MS m/z: 417 (M+1).

### (2-Fluoro-4-methoxyphenyl)(5-methyl-6-(2-(pyrrolidin-1-yl)ethyl)-6H-thieno[2,3-b]pyrrol-2-yl)methanone (92)

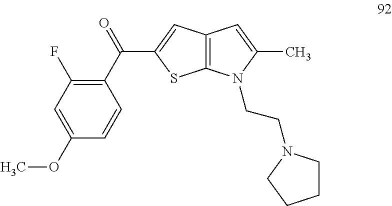 [SMILES: COc1ccc(C(=O)c2cc3cc(C)n(CCN4CCCC4)c3s2)c(F)c1]

¹H NMR (CD₃OD/400 MHz) δ 7.53 (t, J=8.4 Hz, 1H), 7.42 (d, J=1.6 Hz, 1H), 6.89-6.82 (m, 2H), 6.21 (d, J=1.2 Hz, 1H), 4.16 (t, J=7.2 Hz, 2H), 3.88 (s, 3H), 2.90 (t, J=7.2 Hz, 2H), 2.64-2.60 (m, 4H), 2.39 (d, J=1.2 Hz, 3H), 1.84-1.80 (m, 4H); MS m/z: 387 (M+1).

### (2-Chloro-4-methoxyphenyl)(5-methyl-6-(2-(pyrrolidin-1-yl)ethyl)-6H-thieno[2,3-b]pyrrol-2-yl)methanone (93)

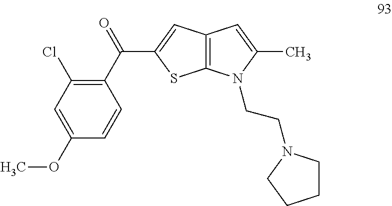 [SMILES: COc1ccc(C(=O)c2cc3cc(C)n(CCN4CCCC4)c3s2)c(Cl)c1]

¹H NMR (CD₃OD/400 MHz) δ 7.44 (d, J=8.4 Hz, 1H), 7.27 (s, 1H), 7.09 (d, J=2.8 Hz, 1H), 6.99 (dd, J=8.4, 2.4 Hz, 1H), 6.20 (d, J=1.2 Hz, 1H), 4.16 (t, J=7.2 Hz, 2H), 3.87 (s, 3H), 2.90 (t, J=6.8 Hz, 2H), 2.64-2.60 (m, 4H), 2.39 (d, J=1.2 Hz, 3H), 1.83-1.78 (m, 4H); MS m/z: 403 (M+1).

### (2-Fluoro-4-methoxyphenyl)(5-methyl-6-(2-(piperidin-1-yl)ethyl)-6H-thieno[2,3-b]pyrrol-2-yl)methanone (98)

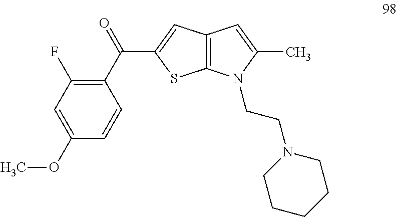 [SMILES: COc1ccc(C(=O)c2cc3cc(C)n(CCN4CCCCC4)c3s2)c(F)c1]

¹H NMR (CD₃OD/400 MHz) δ 7.92 (d, J=8.8 Hz, 1H), 7.72 (s, 1H), 6.56 (dd, J=8.8, 2.4 Hz, 1H), 6.52 (d, J=2.4 Hz, 1H), 6.33 (d, J=0.8 Hz, 1H), 4.49 (t, J=7.6 Hz, 2H), 3.86 (s, 3H), 3.66-3.63 (m, 2H), 3.50 (t, J=8.0 Hz, 2H), 3.11-3.05 (m, 2H), 2.45 (d, J=0.8 Hz, 3H), 2.01-1.98 (m, 2H), 1.88-1.78 (m, 2H), 1.56-1.52 (m, 2H); MS m/z: 399 (M+1).

### (2-Hydroxy-4-methoxyphenyl)(5-methyl-6-(2-(piperidin-1-yl)ethyl)-6H-thieno[2,3-b]pyrrol-2-yl)methanone (99)

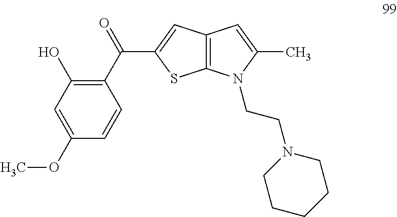 [SMILES: COc1ccc(C(=O)c2cc3cc(C)n(CCN4CCCCC4)c3s2)c(O)c1]

This compound was isolated as a side product en route to the preparation of compound 98 above. ¹H NMR (CD₃OD/400 MHz) δ 7.52 (t, J=8.0 Hz, 1H), 7.45 (d, J=2.0 Hz, 1H), 6.88-6.82 (m, 2H), 6.28 (d, J=0.8 Hz, 1H), 4.49 (t, J=8.0 Hz, 2H), 3.88 (s, 3H), 3.69-3.62 (m, 2H), 3.48 (t, J=7.6 Hz, 2H), 3.10-3.04 (m, 2H), 2.42 (d, J=0.8 Hz, 3H), 1.99-1.93 (m, 2H), 1.83-1.76 (m, 2H), 1.55-1.52 (m, 2H); MS m/z: 401 (M+1).

### (6-(4-Methoxybenzyl)-5-methyl-6H-thieno[2,3-b]pyrrol-2-yl)(4-methoxyphenyl)methanone (103)

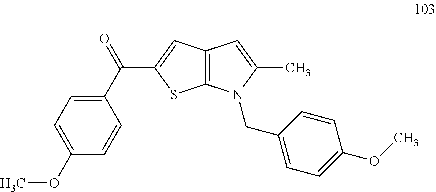

¹H NMR (CDCl₃/400 MHz) δ 7.85-7.80 (m, 2H), 7.50 (s, 1H), 7.14-7.12 (m, 2H), 6.98-6.94 (m, 2H), 6.88-6.84 (m, 2H), 6.21 (d, J=1.2 Hz, 1H), 5.07 (s, 2H), 3.88 (s, 3H), 3.81 (s, 3H), 2.37 (d, J=1.2 Hz, 3H).

### (4-Methoxyphenyl)(5-methyl-6-(2-thiomorpholinoethyl)-6H-thieno[2,3-b]pyrrol-2-yl)methanone (110)

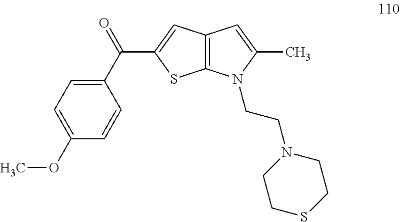 [SMILES: COc1ccc(C(=O)c2cc3cc(C)n(CCN4CCSCC4)c3s2)cc1]

¹H NMR (CDCl₃/400 MHz) δ 7.88-7.83 (m, 2H), 7.51 (s, 1H), 7.00-6.96 (m, 2H), 6.16 (d, J=1.2 Hz, 1H), 4.03 (t, J=6.4 Hz, 2H), 3.89 (s, 3H), 2.80-2.72 (m, 4H), 2.77 (d, J=6.4 Hz, 2H), 2.70-2.62 (m, 4H), 2.37 (d, J=1.2 Hz, 31-1); MS m/z: 401.40 (M+1).

### (4-Methoxyphenyl)(5-methyl-6-(pyridin-4-ylmethyl)-6H-thieno[2,3-b]pyrrol-2-yl)methanone (112)

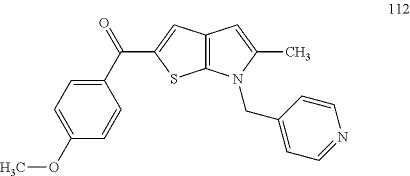 [SMILES: COc1ccc(C(=O)c2cc3cc(C)n(Cc4ccncc4)c3s2)cc1]

¹H NMR (CDCl₃/400 MHz) δ 8.57-8.54 (m, 2H), 7.86-7.82 (m, 2H), 6.99-6.95 (m, 4H), 6.27 (d, J=0.8 Hz, 1H), 5.16 (s, 2H), 3.88 (s, 3H), 2.30 (d, J=0.8 Hz, 3H).

### (6-Benzyl-5-methyl-6H-thieno[2,3-b]pyrrol-2-yl)(2-chloro-4-methoxyphenyl)methanone (114)

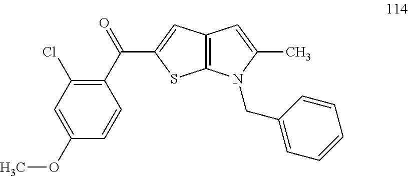 [SMILES: COc1ccc(C(=O)c2cc3cc(C)n(Cc4ccccc4)c3s2)c(Cl)c1]

¹H NMR (CDCl₃/400 MHz) δ 7.40 (d, J=8.4 Hz, 1H), 7.35-7.32 (m, 4H), 7.18-7.16 (m, 1H), 6.99 (d, J=2.4 Hz, 1H), 6.85 (dd, J=8.8, 2.4 Hz, 1H), 6.19 (d, J=1.2 Hz, 1H), 5.13 (s, 2H), 3.85 (s, 3H), 2.35 (d, J=1.2 Hz, 3H); MS m/z: 396 (M+1).

### (4-Methoxyphenyl)(5-methyl-6-((tetrahydro-2H-pyran-4-yl)methylsulfonyl)-6H-thieno[2,3-b]pyrrol-2-yl)methanone (57)

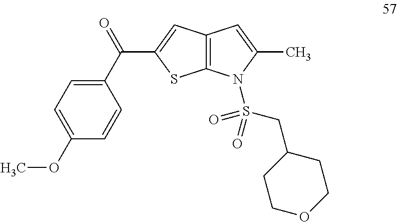 [SMILES: COc1ccc(C(=O)c2cc3cc(C)n(S(=O)(=O)CC4CCOCC4)c3s2)cc1]

¹H NMR (CDCl₃/400 MHz) δ 7.90-7.84 (m, 2H), 7.49 (s, 1H), 7.01-6.96 (m, 2H), 6.35 (d, J=1.2 Hz, 1H), 3.92 (dd, J=11.6, 3.2 Hz, 2H), 3.89 (s, 3H), 3.37 (td, J=12.0, 1.6 Hz, 2H), 3.16 (d, J=6.4 Hz, 2H), 2.54 (s, 3H), 2.35-2.22 (m, 1H), 1.77 (dd, J=12.8, 1.6 Hz, 2H), 1.49-1.36 (m, 2H).

### (2-(4-Methoxybenzoyl)-5-methyl-6H-thieno[2,3-b]pyrrol-6-yl)(morpholino)methanone (66)

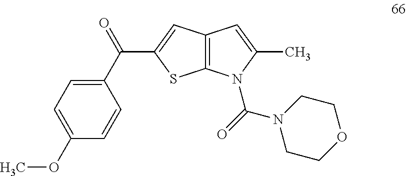 [SMILES: COc1ccc(C(=O)c2cc3cc(C)n(C(=O)N4CCOCC4)c3s2)cc1]

¹H NMR (CDCl₃/400 MHz) δ 7.88-7.83 (m, 2H), 7.49 (s, 1H), 7.01-6.96 (m, 2H), 6.27 (d, J=1.2 Hz, 1H), 3.93-3.72 (br m, 4H), 3.89 (s, 3H), 3.70-3.44 (br m, 4H), 2.47 (d, J=0.8 Hz, 3H).

### (6-methoxypyridin-3-yl)(5-methyl-6-(2-(4-(trifluoromethyl)piperidin-1-yl)ethyl)-6H-thieno[2,3-b]pyrrol-2-yl)methanone (181)

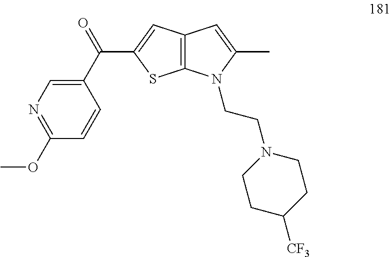 [SMILES: COc1ccc(C(=O)c2cc3cc(C)n(CCN4CCC(C(F)(F)F)CC4)c3s2)cn1]

HNMR (CDCl₃/400 MHz) 8.71 (d, J=2.0 Hz, 1H), 8.06 (dd, J=8.8, 2.8 Hz, 1H), 7.53 (s, 1H), 6.84 (dd, J=8.8, 0.4 Hz, 1H), 6.17 (d, J=0.8 Hz, 1H), 4.06 (t, J=6.8 Hz, 2H), 4.02 (s, 3H), 2.97 (d, J=11.6 Hz, 2H), 2.74 (t, J=6.8 Hz, 2H), 2.38 (d, J=0.8 Hz, 3H), 2.08 (td, J=12.0, 2.0 Hz, 2H), 1.99 (m, 1H), 1.84 (d, J=12.8 Hz, 2H), 1.67-1.59 (m, 2H); MS m/z: 452.27 (M+1).

### (2,6-difluoro-4-methoxyphenyl)(5-methyl-6-((tetrahydro-2H-pyran-4-yl)methyl)-6H-thieno[2,3-b]pyrrol-2-yl)methanone (180)

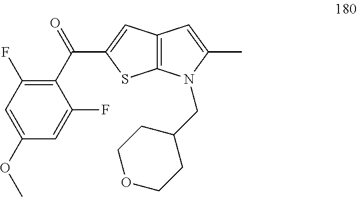 [SMILES: COc1cc(F)c(C(=O)c2cc3cc(C)n(CC4CCOCC4)c3s2)c(F)c1]

¹H NMR (CDCl₃/400 MHz) 6.80 (d, J=5.2 Hz, 1H), 6.53 (d, J=9.2 Hz, 1H), 6.25 (d, J=4.8 Hz, 2H), 3.98 (dd, J=10.8, 4.0 Hz, 2H), 3.88 (d, J=7.6 Hz, 2H), 3.85 (s, 3H), 3.38-3.32 (m, 2H), 2.73 (s, 3H), 2.23-2.16 (m, 1H), 1.57-1.55 (m, 2H), 1.48-1.39 (m, 2H);

MS m/z: 406.23 (M+1).

### 2-(2-(2-fluoro-4-methoxybenzoyl)-5-methyl-6H-thieno[2,3-b]pyrrol-6-yl)acetic acid (176)

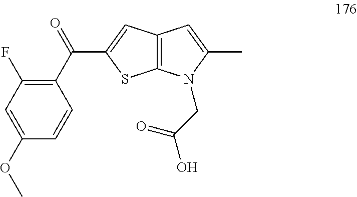 [SMILES: COc1ccc(C(=O)c2cc3cc(C)n(CC(=O)O)c3s2)c(F)c1]

The title compound was prepared by the hydrolysis of methyl 2-(2-(2-fluoro-4-methoxybenzoyl)-5-methyl-6H-thieno[2,3-b]pyrrol-6-yl)acetate. ¹H NMR (DMSO/400 MHz): 7.57 (t, J=8.4 Hz, 1H), 7.43 (d, J=1.6 Hz, 1H), 6.99 (dd, J=12.4, 2.4 Hz, 1H), 6.91 (dd, J=8.4, 2.4 Hz, 1H), 6.24 (s, 1H), 4.90 (s, 2H), 3.86 (s, 3H), 2.26 (d, J=0.4 Hz, 3H); MS m/z: 348.18 (M+1)

### (6-methoxypyridin-3-yl)(5-methyl-6-((tetrahydro-2H-thiopyran-4-yl)methyl)-6H-thieno[2,3-b]pyrrol-2-yl)methanone (175)

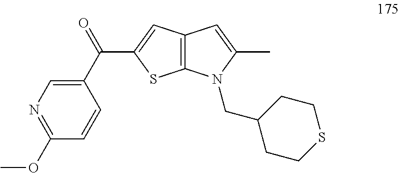 [SMILES: COc1ccc(C(=O)c2cc3cc(C)n(CC4CCSCC4)c3s2)cn1]

¹H NMR (CDCl3/400 MHz): 8.71 (d, J=2.4 Hz, 1H), 8.06 (dd, J=8.8, 2.4 Hz, 1H), 7.53 (s, 1H), 6.84 (d, J=8.8 Hz, 1H), 6.19 (d, J=0.8 Hz, 1H), 4.03-3.99 (m, 4H), 2.97-2.93 (m, 1H), 2.88-2.85 (m, 1H), 2.65-2.62 (m, 1H), 2.55-2.51 (m, 2H), 2.37 (s, 3H), 2.21-2.16 (m, 2H), 2.02-1.93 (m, 2H), 1.68-1.62 (m, 1H); MS m/z: 387.20 (M+1).

### (6-methoxypyridin-3-yl)(5-methyl-6-((tetrahydro-2H-pyran-2-yl)methyl)-6H-thieno[2,3-b]pyrrol-2-yl)methanone (173)

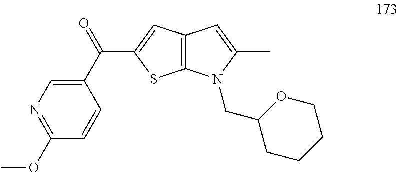 [SMILES: COc1ccc(C(=O)c2cc3cc(C)n(CC4CCCCO4)c3s2)cn1]

¹H NMR (CDCl3/400 MHz): 8.71 (d, J=2.4 Hz, 1H), 8.01 (dd, J=8.4, 2.4 Hz, 1H), 7.52 (s, 1H), 6.84 (dd, J=8.8, 0.8 Hz, 1H), 6.17 (s, 1H), 4.03-3.95 (m, 5H), 3.90-3.85 (m, 1H), 3.76-3.70 (m, 1H), 3.39-3.35 (m, 1H), 2.37 (s, 3H), 1.87 (d, J=11.6 Hz, 1H), 1.63-1.44 (m, 4H), 1.40-1.30 (m, 1H); MS m/z: 371.24 (M+1).

### (R)-(6-methoxypyridin-3-yl)(5-methyl-6-((4-methylmorpholin-3-yl)methyl)-6H-thieno[2,3-b]pyrrol-2-yl)methanone (171)

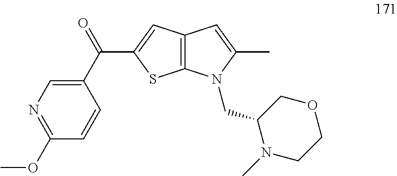 [SMILES: COc1ccc(C(=O)c2cc3cc(C)n(C[C@@H]4COCCN4C)c3s2)cn1]

¹H NMR (CDCl3/400 MHz): 8.71 (dd, J=2.4, 0.8 Hz, 1H), 8.06 (dd, J=8.4, 2.4 Hz, 1H), 7.52 (s, 1H), 6.84 (dd, J=8.8, 0.8 Hz, 1H), 6.19 (d, J=0.8 Hz, 1H), 4.26 (dd, J=14.4, 4.0 Hz, 1H), 4.02 (s, 3H), 3.98-3.92 (m, 1H), 3.80-3.68 (m, 2H), 3.54 (dd, J=11.6, 2.8 Hz, 1H), 3.39-3.35 (m, 1H), 2.87-2.76 (m, 2H), 2.52 (s, 3H), 2.47-2.41 (m, 1H), 2.38 (d, J=0.8 Hz, 3H); MS m/z: 386.24 (M+1).

### Methyl 2-(2-(6-methoxynicotinoyl)-5-methyl-6H-thieno[2,3-b]pyrrol-6-yl)acetate (168)

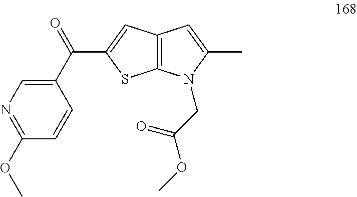 [SMILES: COC(=O)Cn1c(C)cc2cc(C(=O)c3ccc(OC)nc3)sc21]

¹H NMR (CDCl3/400 MHz): 8.71 (d, J=2.4 Hz, 1H), 8.06 (dd, J=8.4, 2.0 Hz, 1H), 7.54 (s, 1H), 6.84 (d, J=8.4 Hz, 1H), 6.25 (s, 1H), 4.70 (s, 2H), 4.02 (s, 3H), 3.80 (s, 3H), 2.33 (s, 3H); MS m/z: 345.15 (M+1).

### 2-(2-(6-methoxynicotinoyl)-5-methyl-6H-thieno[2,3-b]pyrrol-6-yl)acetic acid (166)

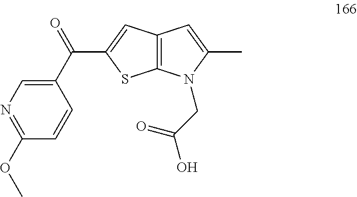 [SMILES: COc1ccc(C(=O)c2cc3cc(C)n(CC(=O)O)c3s2)cn1]

The title compound was prepared by the hydrolysis of methyl 24246-methoxynicotinoyl)-5-methyl-6H-thieno[2,3-b]pyrrol-6-yl)acetate (168). ¹H NMR (Acetonitrile/400 MHz): 8.68 (d, J=2.0 Hz, 1H), 8.09 (dd, J=8.4, 2.4 Hz, 1H), 7.60 (s, 1H), 6.91 (d, J=8.8 Hz, 1H), 6.28 (s, 1H), 4.79 (s, 2H), 4.00 (s, 3H), 2.31 (s, 3H); MS m/z: 331.12 (M+1)

### (6-methoxypyridin-3-yl)(5-methyl-6-(2-(pyrrolidin-1-yl)ethyl)-6H-thieno[2,3-b]pyrrol-2-yl)methanone (167)

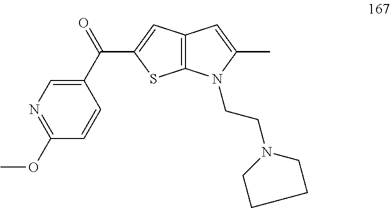 [SMILES: COc1ccc(C(=O)c2cc3cc(C)n(CCN4CCCC4)c3s2)cn1]

¹H NMR (CD3OD/400 MHz): 8.63 (d, J=2.4 Hz, 1H), 8.09 (dd, J=8.4, 2.4 Hz, 1H), 7.61 (s, 1H), 6.92 (d, J=8.4 Hz, 1H), 6.25 (d, J=0.8 Hz, 1H), 4.17 (t, J=6.8 Hz, 2H), 4.01 (s, 3H), 2.91 (t, J=6.8 Hz, 2H), 2.62-2.60 (m, 4H), 2.40 (d, J=0.4 Hz, 3H), 1.83-1.80 (m, 4H); MS m/z: 370.23 (M+1).

### (6-(2,2-dimethoxyethyl)-5-methyl-6H-thieno[2,3-b]pyrrol-2-yl)(6-methoxypyridin-3-yl)methanone (161)

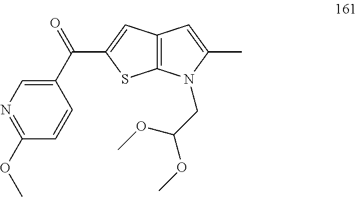 [SMILES: COc1ccc(C(=O)c2cc3cc(C)n(CC(OC)OC)c3s2)cn1]

¹H NMR (CDCl3/400 MHz) 8.71 (dd, J=1.2, 0.4 Hz, 1H), 8.08-8.05 (m, 1H), 7.52 (d, J=1.6 Hz, 1H), 6.84 (dd, J=8.4, 0.4 Hz, 1H), 6.18 (s, 1H), 4.61 (td, J=4.8, 0.8 Hz, 1H), 4.04 (dd, J=5.2, 0.8 Hz, 2H), 4.02 (d, J=1.2 Hz, 3H), 3.40 (d, J=1.2 Hz, 6H), 2.38 (s, 3H); MS m/z: 361.16 (M+1).

### (6-methoxypyridin-3-yl)(5-methyl-6-((tetrahydrofuran-3-yl)methyl)-6H-thieno[2,3-b]pyrrol-2-yl)methanone (159)

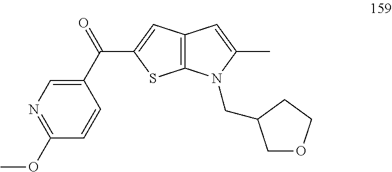 [SMILES: COc1ccc(C(=O)c2cc3cc(C)n(CC4CCOC4)c3s2)cn1]

¹H NMR (CDCl3/400 MHz): 8.67 (d, J=2.4 Hz, 1H), 8.07 (dd, J=8.8, 2.4 Hz, 1H), 7.52 (s, 1H), 6.83 (d, J=8.4 Hz, 1H), 6.19 (s, J=0.8 Hz, 1H), 4.41-4.37 (m, 1H), 4.32-4.27 (m, 1H), 3.96-3.89 (m, 2H), 3.83-3.77 (m, 1H), 3.73-3.70 (m, 1H), 3.65 (s, 3H), 2.80-2.75 (m, 1H), 2.36 (d, J=0.8 Hz, 3H), 2.15-2.11 (m, 1H), 1.80-1.72 (m, 1H); MS m/z: 357.21 (M+1).

### tert-butyl 4((2-(6-methoxynicotinoyl)-5-methyl-6H-thieno[2,3-b]pyrrol-6-yl)methyl)piperidine-1-carboxylate (179)

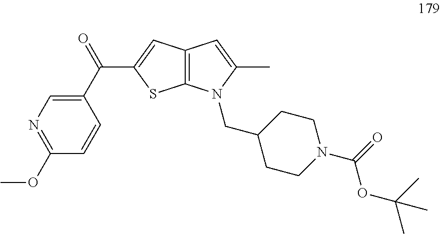 [SMILES: COc1ccc(C(=O)c2cc3cc(C)n(CC4CCN(C(=O)OC(C)(C)C)CC4)c3s2)cn1]

The title compound was prepared with tert-butyl 4-(tosyloxymethyl)piperidine-1-carboxylate for the N-alkylation. ¹H NMR (CDCl₃/400 MHz) 8.72 (s, 1H), 8.07 (d, J=8.8 Hz, 1H), 7.54 (s, 1H), 6.85 (d, J=8.4 Hz, 1H), 6.19 (s, 1H), 4.21-4.10 (m, 2H), 4.04 (s, 3H), 3.84-3.83 (m, 2H), 2.65-2.63 (m, 2H), 2.36 (s, 3H), 2.17-2.09 (m, 1H), 1.53-1.51 (m, 2H), 1.46 (s, 9H), 1.30-1.14 (m, 2H); MS m/z: 470.33 (M+1).

### (6-(2-(2-((benzyloxy)methyl)pyrrolidin-1-yl)ethyl)-5-methyl-6H-thieno[2,3-b]pyrrol-2-yl)(6-methoxypyridin-3-yl)methanone (178)

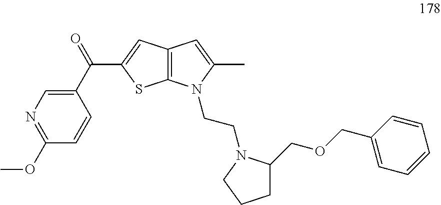 [SMILES: COc1ccc(C(=O)c2cc3cc(C)n(CCN4CCCC4COCc4ccccc4)c3s2)cn1]

HNMR (CDCl₃/400 MHz) 8.46 (t, J=0.8 Hz, 1H), 7.81 (dd, J=8.4, 2.4 Hz, 1H), 7.28 (s, 1H), 7.08-7.02 (m, 5H), 6.59 (d, J=8.4 Hz, 1H), 5.91 (s, 1H), 4.19 (s, 2H), 3.80-3.77 (m, 6H), 3.10-3.04 (m, 3H), 2.92 (br s, 1H), 2.55-2.49 (m, 2H), 2.06 (s, 3H), 1.68-1.61 (m, 1H), 1.57-1.54 (m, 31-1); MS m/z: 490.33 (M+1).

### (6((1,4-dioxan-2-yl)methyl)-5-methyl-6H-thieno[2,3-b]pyrrol-2-yl)(6-methoxypyridin-3-yl)methanone (177)

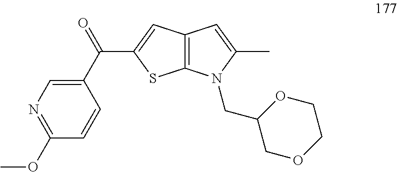 [SMILES: COc1ccc(C(=O)c2cc3cc(C)n(CC4COCCO4)c3s2)cn1]

HNMR (CD₃OD/400 MHz) 8.70 (t, J=0.80 Hz, 1H), 8.05 (dt, J=8.8, 1.2 Hz, 1H), 7.52 (s, 1H), 6.83 (d, J=8.8 Hz, 1H), 6.19 (s, 1H), 4.02 (s, 3H), 3.98-3.97 (m, 2H), 3.94-3.89 (m, 1H), 3.83-3.78 (m, 2H), 3.72-3.66 (m, 2H), 3.64-3.56 (m, 1H), 3.41-3.36 (m, 1H), 2.36 (s, 3H); MS m/z: 373.21 (M+1).

### (6-methoxypyridin-3-yl)(5-methyl-6-(piperidin-4-ylmethyl)-6H-thieno[2,3-b]pyrrol-2-yl)methanone (172)

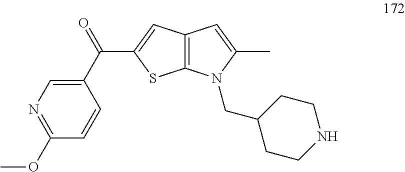 [SMILES: COc1ccc(C(=O)c2cc3cc(C)n(CC4CCNCC4)c3s2)cn1]

The title compound was made from a Boc-protected precursor using Scheme 2 and HCl was used to remove Boc group. HNMR (CD₃OD/400 MHz) 8.66 (d, J=2.0 Hz, 1H), 8.11 (dd, J=8.8, 2.4 Hz, 1H), 7.66 (s, 1H), 6.95 (d, J=8.4 Hz, 1H), 6.32 (s, 1H), 4.04 (s, 3H), 4.02 (s, 2H), 3.43 (d, J=12.8 Hz, 2H), 3.01 (td, J=13.2, 3.2 Hz, 2H), 2.43 (s, 3H), 2.39-2.34 (m, 1H), 1.88 (d, J=12.4 Hz, 2H), 1.56 (d, J=16.4 Hz, 2H); MS m/z: 370.27 (M+1).

### (6-((4,4-difluorocyclohexyl)methyl)-5-methyl-6H-thieno[2,3-b]pyrrol-2-yl)(6-methoxypyridin-3-yl)methanone (170)

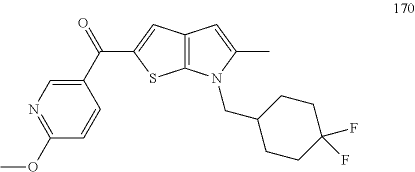 [SMILES: COc1ccc(C(=O)c2cc3cc(C)n(CC4CCC(F)(F)CC4)c3s2)cn1]

HNMR (CDCl₃/400 MHz) 8.72 (S, 1H), 8.09-8.06 (m, 1H), 7.55 (d, J=1.6 Hz, 1H), 6.85 (dd, J=8.8, 0.8 Hz, 1H), 6.20 (s, 1H), 4.04 (d, J=1.2 Hz, 3H), 3.85 (d, J=6.8 Hz, 2H), 2.37 (s, 3H), 2.07 (br s, 3H), 1.74 (d, J=11.2 Hz, 2H), 1.60 (d, J=1.2 Hz, 2H), 1.47-1.41 (m, 2H); MS m/z: 405.27 (M+1).

### (6-(2-(2-oxa-5-azabicyclo[2.2.1]heptan-5-yl)ethyl)-5-methyl-6H-thieno[2,3-b]pyrrol-2-yl)(6-methoxypyridin-3-yl)methanone (169)

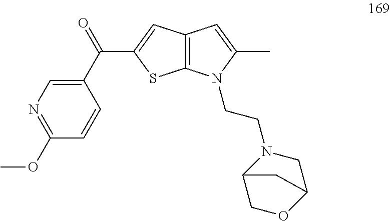 [SMILES: COc1ccc(C(=O)c2cc3cc(C)n(CCN4CC5CC4CO5)c3s2)cn1]

HNMR (CDCl₃/400 MHz) 8.71 (d, J=2.0 Hz, 1H), 8.08-8.05 (m, 1H), 7.53 (d, J=2.0 Hz, 1H), 6.84 (dd, J=8.8, 1.2 Hz, 1H), 6.18 (s, 1H), 4.40 (s, 1H), 4.02 (d, J=2.0 Hz, 3H), 3.97 (d, J=8.0 Hz, 1H), 3.61 (d, J=8.0 Hz, 1H), 3.41 (s, 1H), 3.03-2.91 (m, 2H), 2.93 (d, J=9.6 Hz, 1H), 2.52 (d, J=10.0 Hz, 1H), 2.39 (s, 3H), 1.83 (d, J=10.0 Hz, 2H), 1.73 (d, J=9.2 Hz, 2H); MS m/z: 398.25 (M+1).

### (6-(2-(2,2-dimethylmorpholino)ethyl)-5-methyl-6H-thieno[2,3-b]pyrrol-2-yl)(6-methoxypyridin-3-yl)methanone (162)

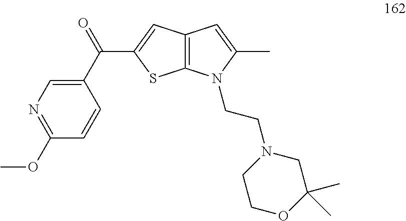

HNMR (CDCl₃/400 MHz) 8.68 (d, J=2.0 Hz, 1H), 8.04 (dd, J=8.4, 2.4 Hz, 1H).

7.51 (s, 1H), 6.81 (d, J=8.8 Hz, 1H), 6.16 (s, 1H), 4.04-4.02 (m, 2H), 4.00 (s, 3H), 3.70 (t, J=4.4 Hz, 2H), 2.67 (t, J=6.4 Hz, 2H), 2.40-2.39 (m, 2H), 2.37 (s, 3H), 2.21 (s, 2H), 1.17 (s, 6H); MS m/z: 414.26 (M+1).

### (6-(2-(1,4-oxazepan-4-yl)ethyl)-5-methyl-6H-thieno[2,3-b]pyrrol-2-yl)(6-methoxypyridin-3-yl)methanone (158)

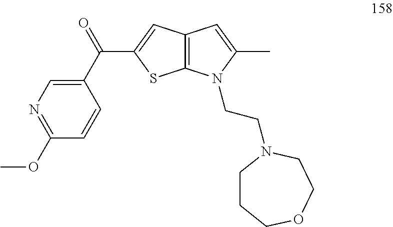 [SMILES: COc1ccc(C(=O)c2cc3cc(C)n(CCN4CCCOCC4)c3s2)cn1]

HNMR (CDCl₃/400 MHz) 8.72 (d, J=2.0 Hz, 1H), 8.07 (dd, J=8.8, 2.4 Hz, 1H), 7.54 (s, 1H), 6.85 (dd, J=8.4, 0.4 Hz, 1H), 6.19 (d, J=0.8 Hz, 1H), 4.06 (t, J=6.8 Hz, 2H), 4.03 (s, 3H), 3.79 (t, J=6.0 Hz, 2H), 3.72-3.70 (m, 2H), 2.94 (t, J=6.8 Hz, 2H), 2.81-2.76 (m, 4H), 2.40 (d, J=0.8 Hz, 3H), 1.90 (d, J=6.0 Hz, 2H); MS m/z: 400.22 (M+1).

### (6-(2-42-methoxyethyl)(methyl)amino)ethyl)-5-methyl-6H-thieno[2,3-b]pyrrol-2-yl)(6-methoxypyridin-3-yl)methanone (157)

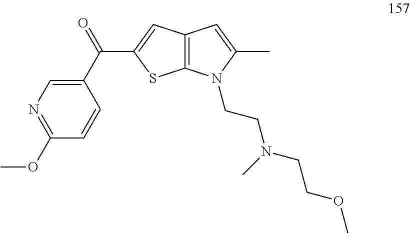

HNMR (CDCl₃/400 MHz) 8.71 (d, J=2.4 Hz, 1H), 8.07 (dd, J=8.8, 2.8 Hz, 1H), 7.53 (s, 1H), 6.84 (d, J=8.4 Hz, 1H), 6.18 (d, J=0.8 Hz, 1H), 4.07 (t, J=7.2 Hz, 2H), 4.03 (s, 3H), 3.44 (t, J=5.6 Hz, 2H), 3.33 (s, 3H), 2.84 (t, J=7.8 Hz, 2H), 2.66 (t, J=5.6 Hz, 2H), 2.39 (s, 3H), 2.38 (d, J=0.8 Hz, 3H); MS m/z: 388.21 (M+1).

### (6-(2-(4,4-difluoropiperidin-1-yl)ethyl)-5-methyl-6H-thieno[2,3-b]pyrrol-2-yl)(6-methoxypyridin-3-yl)methanone (152)

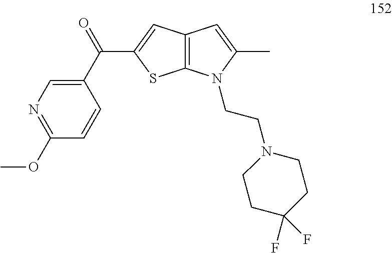 [SMILES: COc1ccc(C(=O)c2cc3cc(C)n(CCN4CCC(F)(F)CC4)c3s2)cn1]

HNMR (CDCl₃/400 MHz) 8.72 (dd, J=2.4, 0.4 Hz, 1H), 8.07 (dd, J=8.4, 2.4 Hz, 1H), 7.54 (s, 1H), 6.85 (dd, J=8.8, 0.8 Hz, 1H), 6.19 (d, J=0.8 Hz, 1H), 4.06 (t, J=6.4 Hz, 2H), 4.03 (s, 3H), 2.81 (t, J=6.4 Hz, 2H), 2.63-2.61 (m, 4H), 2.39 (d, J=0.8 Hz, 3H), 2.04-1.95 (m, 4H); MS m/z: 420.22 (M+1).

### (6-(2-(4-methoxypiperidin-1-yl)ethyl)-5-methyl-6H-thieno[2,3-b]pyrrol-2-yl)(6-methoxypyridin-3-yl)methanone (156)

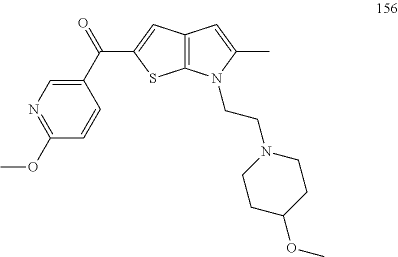

HNMR (CDCl₃/400 MHz) 8.67 (s, 1H), 8.02 (dd, J=8.8, 2.0 Hz, 1H), 7.49 (t, J=0.4 Hz, 1H), 6.80 (dd, J=4.8, 1.2 Hz, 1H), 6.13 (s, 1H), 4.02-3.99 (m, 2H), 3.98 (s, 3H), 3.30 (s, 3H), 3.21-3.17 (m, 1H), 2.73-2.67 (m, 4H), 2.34 (m, 3H), 2.23 (t, J=9.2 Hz, 2H), 1.87-1.85 (m, 2H), 1.61-1.53 (m, 2H); MS m/z: 414.26 (M+1).

### Methyl 2-(4-methoxybenzoyl)-5-methyl-6H-thieno[2,3-b]pyrrole-6-carbodithioate (155)

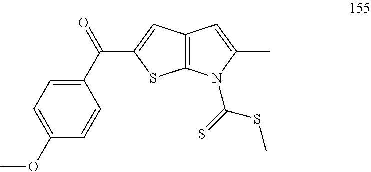

HNMR (CDCl₃/400 MHz) 7.90 (dd, J=7.2, 2.0 Hz, 2H), 7.52 (s, 1H), 7.00 (dd, J=6.4, 2.0 Hz, 2H), 6.50 (d, J=0.8 Hz, 1H), 3.91 (s, 3H), 2.90 (d, J=0.8 Hz, 3H), 2.84 (s, 3H); MS m/z: 360.04 (M+1).

### Example 6

### Synthesis of (6-(3-azidopropyl)-5-methyl-6H-thieno[2,3-b]pyrrol-2-yl)(4-methoxyphenyl)methanone

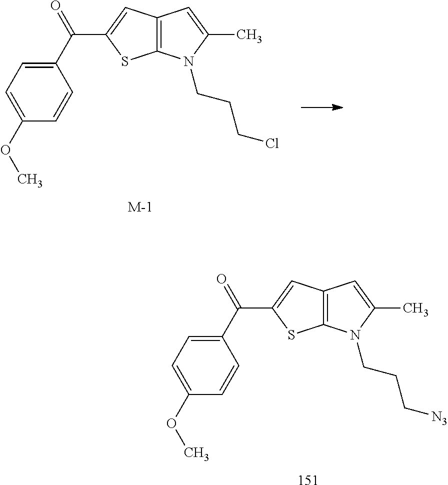 [SMILES: COc1ccc(C(=O)c2cc3cc(C)n(CCCCl)c3s2)cc1 ; "M-1" ; COc1ccc(C(=O)c2cc3cc(C)n(CCCN=[N+]=[N-])c3s2)cc1 ; "151"]

Sodium azide (243 mg, 3.74 mmol) was added to a solution of (6-(3-chloropropyl)-5-methyl-6H-thieno[2,3-b]pyrrol-2-yl)(4-methoxyphenyl)methanone (808 mg, 2.32 mmol) in 46 mL of DMSO. The reaction mixture was heated at 60° C. for 18 h. The reaction mixture was poured into H₂O (50 mL) and extracted with EtOAc (50 mL×2). The organic layers were combined and washed with H₂O (3×50 mL). The organic layer was dried with MgSO₄, concentrated in vacuo, and triturated with Et₂O to afford 799 mg of (6-(3-azidopropyl)-5-methyl-6H-thieno[2,3-b]pyrrol-2-yl)(4-methoxyphenyl)methanone (151, 97%) as a yellow solid that was used directly in the next step.

### Example 7

### Synthesis of (6-(3-aminopropyl)-5-methyl-6H-thieno[2,3-b]pyrrol-2-yl)(4-methoxyphenyl)methanone (151)

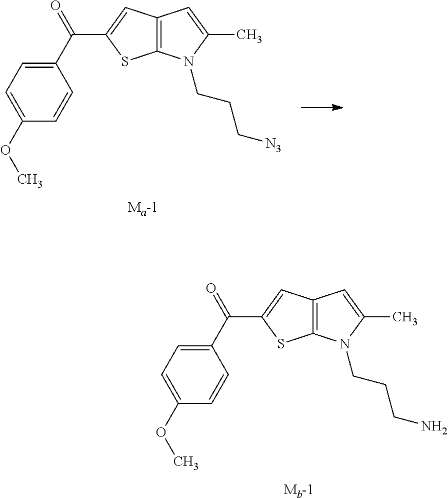 [SMILES: COc1ccc(C(=O)c2cc3cc(C)n(CCCN=[N+]=[N-])c3s2)cc1 ; "Ma-1" ; COc1ccc(C(=O)c2cc3cc(C)n(CCCN)c3s2)cc1 ; "Mb-1"]

A 0.15 M suspension of tin(IV) chloride dihydrate in a 1N aqueous solution of NaOH (4.5 ml, 0.669 mmol) maintained at 0° C. was added to a yellow suspension of (6-(3-azidopropyl)-5-methyl-6H-thieno[2,3-b]pyrrol-2-yl)(4-methoxyphenyl)methanone (158 mg, 0.446 mmol) in 3.1 mL of EtOH at 0° C. The mixture was stirred at rt for 24 h. The resulting reaction mixture was extracted with CHCl₃ (100 mL). The organic layer was dried with Na₂SO₄ and concentrated in vacuo to afford 137 mg (94%) of (6-(3-aminopropyl)-5-methyl-6H-thieno[2,3-b]pyrrol-2-yl)(4-methoxyphenyl)methanone as a yellow oil.

### Example 8

### Synthesis of 1,1,1-trifluoro-N-(3-(2-(4-methoxybenzoyl)-5-methyl-6H-thieno[2,3-b]pyrrol-6-yl)propyl)methanesulfonamide (75)

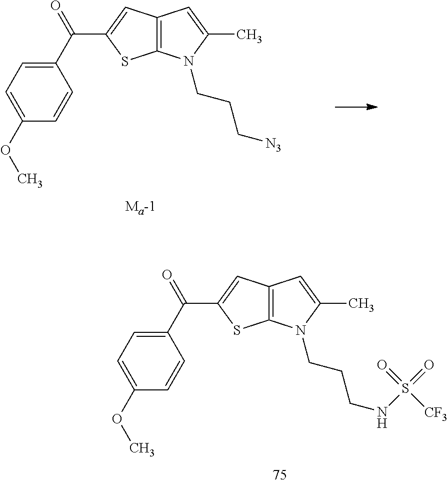 [SMILES: COc1ccc(C(=O)c2cc3cc(C)n(CCCN=[N+]=[N-])c3s2)cc1 ; COc1ccc(C(=O)c2cc3cc(C)n(CCCNS(=O)(=O)C(F)(F)F)c3s2)cc1]

Triethylphosphite (0.222 ml, 1.27 mmol) was added to a solution of (6-(3-azidopropyl)-5-methyl-6H-thieno[2,3-b]pyrrol-2-yl)(4-methoxyphenyl)methanone (150 mg, 0.423 mmol) in 4 mL of benzene. The reaction mixture was sealed in a vial and heated at 80° C. for 48 h. The mixture was cooled to rt. A 4.0 M of HCl in dioxane (0.317 ml, 1.27 mmol) was added to the reaction. The reaction mixture was heated at reflux for an additional 3 h. The reaction was cooled to rt and concentrated in vacuo to afford a red oil. The residue was dissolved in dichloromethane (4.0 mL) and triethylamine (1.18 ml, 8.46 mmol) and DMAP (5.17 mg, 0.042 mmol) were added to the mixture. The reaction was cooled to 0° C. Trifluoromethane sulfonic anhydride (0.072 mL, 0.423 mmol) was added to the reaction. The reaction mixture was allowed to warm to rt and stirred a total of 18 h. The mixture was diluted with dichloromethane (100 mL), washed with a saturated aqueous solution of ammonium chloride (50 mL×2), and washed with brine (10 mL). The organic layer was dried with MgSO₄ and concentrated in vacuo to afford an oil. The residue was purified by flash silica gel chromatography (40% EtOAc in hexanes) to afford 45.7 mg (23%) of 1,1,1-trifluoro-N-(3-(2-(4-methoxybenzoyl)-5-methyl-6H-thieno[2,3-b]pyrrol-6-yl)propyl)methanesulfonamide as a yellow foam; ¹H NMR (CDCl₃/400 MHz) δ 7.84-7.80 (m, 2H), 7.52 (s, 1H), 6.90-6.86 (m, 2H), 6.19 (d, J=0.8, 1H), 6.13 (s, 3H), 4.09 (t, J=7.2 Hz, 2H), 3.89 (s, 3H), 3.39 (t, J=7.2 Hz, 2H), 2.36 (d, J=0.8 Hz, 3H), 2.22-2.20 (m, 2H).

### Example 9

### Synthesis of N-(3-(2-(4-methoxybenzoyl)-5-methyl-6H-thieno[2,3-b]pyrrol-6-yl)propyl)benzenesulfonamide (81)

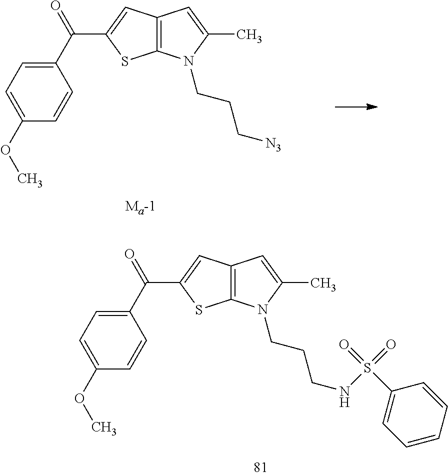 [SMILES: COc1ccc(C(=O)c2cc3cc(C)n(CCCN=[N+]=[N-])c3s2)cc1 ; COc1ccc(C(=O)c2cc3cc(C)n(CCCNS(=O)(=O)c4ccccc4)c3s2)cc1]

Triphenylphosphine (263 mg, 1.00 mmol) was added to a solution of (6-(3-azidopropyl)-5-methyl-6H-thieno[2,3-b]pyrrol-2-yl)(4-methoxyphenyl)methanone (118.6 mg, 0.335 mmol) in 4 mL of benzene. The reaction mixture was sealed in a vial and heated at 80° C. for 48 h. The mixture was cooled to rt. H₂O (0.018 mL, 1.004 mmol) and MeOH (2.000 mL) were added to the reaction. The mixture was heated at reflux for 3 h. The reaction mixture was cooled to rt and concentrated in vacuo. The residue was dissolved in dichloromethane. Triethylamine (0.933 mL, 6.69 mmol) and DMAP (4.09 mg, 0.033 mmol) were added to the mixture. The reaction turned from red to yellow. This mixture was cooled to 0° C. Benzenesulfonyl chloride (0.051 mL, 0.402 mmol) was added to the reaction. The resulting mixture stirred at rt for 16 h. The reaction was diluted with dichloromethane and washed with a saturated aqueous solution ammonium chloride (20 mL). The organic layer was dried with MgSO₄ and concentrated in vacuo to afford an oil. The residue was purified by flash silica gel chromatography (0% to 20% t-BuOMe in dichloromethane) to afford 54.2 mg (35%) of give N-(3-(2-(4-methoxybenzoyl)-5-methyl-6H-thieno[2,3-b]pyrrol-6-yl)propyl)benzenesulfonamide as a brown oil; ¹H NMR (CDCl₃/400 MHz) δ 7.86-7.83 (m, 2H), 7.82-7.81 (m, 2H), 7.52 (s, 1H), 7.53-7.45 (m, 3H), 7.01-6.95 (m, 2H), 6.17 (d, J=0.8 Hz, 1H), 4.63 (t, J=6.4 Hz, 1H), 4.03 (t, J=6.8 Hz, 2H), 3.89 (s, 3H), 2.98 (q, J=6.4 Hz, 2H), 2.34 (d, J=0.8 Hz, 3H), 2.07-2.05 (m, 2H).

### (4-methoxyphenyl)(5-methyl-6-(2-morpholinoethylsulfonyl)-6H-thieno[2,3-b]pyrrol-2-yl)methanone (139)

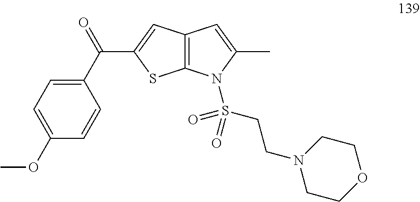 [SMILES: COc1ccc(C(=O)c2cc3cc(C)n(S(=O)(=O)CCN4CCOCC4)c3s2)cc1]

¹H NMR (CDCl₃/400 MHz) 7.87 (m, 2H), 7.49 (s, 1H), 7.00 (m, 2H), 6.35 (d, J=1.2 Hz, 1H), 3.90 (s, 3H), 3.51 (t, J=6.0 Hz, 2H), 3.39 (br s, 4H), 2.80 (t, J=6.0 Hz, 2H), 2.56 (d, J=1.2 Hz, 3H), 2.24 (br s, 4H); MS m/z: 449.08 (M+1).

The following compounds were prepared according to Scheme 1 or Scheme 2:

### (6-(2-(diethylamino)ethyl)-5-methyl-6H-thieno[2,3-b]pyrrol-2-yl)(6-methoxypyridin-3-yl)methanone (142)

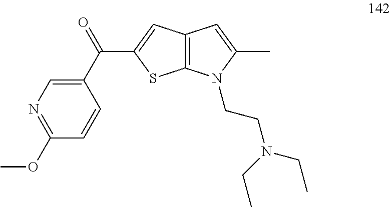 [SMILES: CCN(CC)CCn1c(C)cc2cc(C(=O)c3ccc(OC)nc3)sc21]

¹H NMR (CDCl₃/400 MHz) 8.68 (d, J=2.4 Hz, 1H), 8.04 (dd, J=8.4, 2.4 Hz, 1H), 7.51 (s, 1H), 6.82 (d, J=8.4 Hz, 1H), 6.15 (d, J=0.8 Hz, 1H), 4.00 (s, 3H), 3.98 (t, J=7.2 Hz, 2H), 2.78 (t, J=7.2 Hz, 2H), 2.55 (q, J=7.2 Hz, 4H), 2.37 (d, J=0.8 Hz, 3H), 0.99 (t, J=7.2 Hz, 6H); MS m/z: 372.21 (M+1).

### (6-methoxypyridin-3-yl)(5-methyl-6-(2-(4-(methylsulfonyl)piperazin-1-yl)ethyl)-6H-thieno[2,3-b]pyrrol-2-yl)methanone (143)

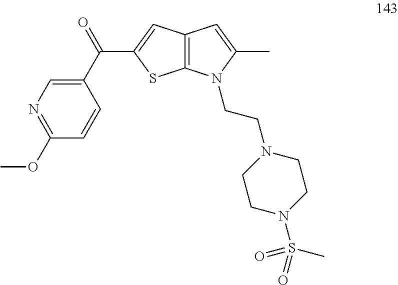

¹H NMR (CDCl₃/400 MHz) 8.70 (dd, J=2.4, 0.8 Hz, 1H), 8.05 (dd, J=8.8, 2.4 Hz, 1H), 7.52 (s, 1H), 6.83 (dd, J=8.8, 0.8 Hz, 1H), 6.18 (d, J=1.2 Hz, 1H), 4.05 (t, J=6.0 Hz, 2H), 4.02 (s, 3H), 3.24 (t, J=4.8 Hz, 4H), 2.80 (t, J=6.0 Hz, 2H), 2.78 (s, 3H), 2.61 (t, J=4.8 Hz, 4H), 2.37 (d, J=1.2 Hz, 3H); MS m/z: 463.19 (M+1).

### (6-methoxypyridin-3-yl)(5-methyl-6-((tetrahydro-2H-pyran-4-yl)methyl)-6H-thieno[2,3-b]pyrrol-2-yl)methanone (144)

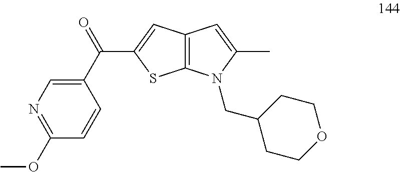 [SMILES: COc1ccc(C(=O)c2cc3cc(C)n(CC4CCOCC4)c3s2)cn1]

¹H NMR (CDCl₃/400 MHz) 8.71 (d, J=1.6 Hz, 1H), 8.06 (dd, J=8.4, 2.4 Hz, 1H), 7.54 (s, 1H), 6.84 (d, J=8.4 Hz, 1H), 6.19 (d, J=0.8 Hz, 1H), 4.02 (s, 3H), 3.98 (dd, J=11.6, 3.6 Hz, 2H), 3.83 (d, J=8.0 Hz, 2H), 3.36 (td, J=11.6, 2.0 Hz, 2H), 2.37 (d, J=0.8 Hz, 3H), 2.26-2.19 (m, 1H), 1.57-1.54 (m, 2H), 1.47-1.38 (m, 2H); MS m/z: 371.20 (M+1).

### (2-fluoro-4-methoxyphenyl)(5-methyl-6-((tetrahydro-2H-pyran-4-yl)methyl)-6H-thieno[2,3-b]pyrrol-2-yl)methanone (145)

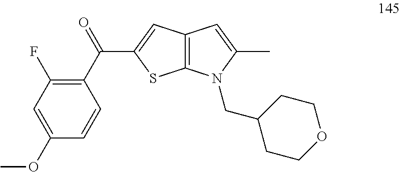 [SMILES: COc1ccc(C(=O)c2cc3cc(C)n(CC4CCOCC4)c3s2)c(F)c1]

¹H NMR (CDCl₃/400 MHz) 7.55 (t, J=8.4 Hz, 1H), 7.40 (d, J=2.4 Hz, 1H), 6.77 (dd, J=8.8, 2.4 Hz, 1H), 6.69 (dd, J=11.6, 2.4 Hz, 1H), 6.15 (d, J=1.2 Hz, 1H), 3.98 (dd, J=11.6, 3.6 Hz, 2H), 3.87 (s, 3H), 3.81 (d, J=3.6 Hz, 2H), 3.35 (td, J=12.0, 2.0 Hz, 2H), 2.38 (d, J=0.8 Hz, 3H), 2.25-2.19 (m, 1H), 1.56-1.53 (m, 2H), 1.47-1.38 (m, 2H); MS m/z: 288.17 (M+1).

### 1-(2-(2-(6-methoxynicotinoyl)-5-methyl-6H-thieno[2,3-b]pyrrol-6-yl)ethyl)pyrrolidine-2-carboxylic acid (146)

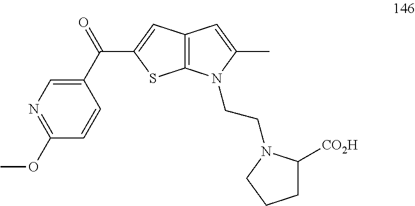 [SMILES: COc1ccc(C(=O)c2cc3cc(C)n(CCN4CCCC4C(=O)O)c3s2)cn1]

¹H NMR (CDCl₃/400 MHz) 8.66 (s, 1H), 8.02 (d, J=8.4 Hz, 1H), 7.48 (s, 1H), 6.82 (d, J=8.4 Hz, 1H), 6.17 (s, 1H), 5.54 (s, 1H), 4.31-4.13 (m, 2H), 4.01 (s, 3H), 3.64-3.43 (m, 2H), 3.37-3.07 (m, 2H), 2.72-2.56 (m, 1H), 2.35 (s, 3H), 2.31-2.03 (m, 1H), 2.00-1.80 (m, 2H); MS m/z: 414.20 (M+1).

### (6-methoxypyridin-3-yl)(6-(2-(3-methoxypyrrolidin-1-yl)ethyl)-5-methyl-6H-thieno[2,3-b]pyrrol-2-yl)methanone (147)

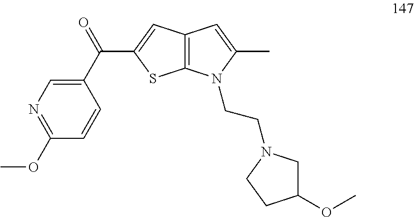

¹H NMR (CDCl₃/400 MHz) 8.70-8.68 (s, 1H), 8.05 (dd, J=8.4, 2.4 Hz, 1H), 7.51 (s, 1H), 6.83 (dd, J=8.4, 0.4 Hz, 1H), 6.16 (d, J=0.8 Hz, 1H), 4.11-4.05 (m, 2H), 4.01 (s, 3H), 3.95-3.87 (m, 1H), 3.28 (s, 3H), 2.90-2.84 (m, 2H), 2.73-2.65 (m, 2H), 2.53-2.45 (m, 1H), 2.36 (d, J=0.8 Hz, 3H), 2.12-2.01 (m, 1H), 1.88-1.78 (m, 2H); MS m/z: 400.22 (M+1).

### 4-(2-(2-(6-methoxynicotinoyl)-5-methyl-6H-thieno[2,3-b]pyrrol-6-yl)ethyl)morpholin-3-one (148)

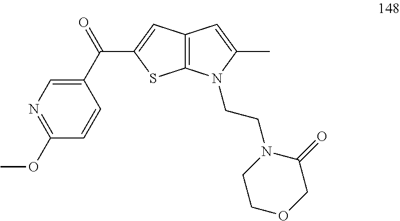 [SMILES: COc1ccc(C(=O)c2cc3cc(C)n(CCN4CCOCC4=O)c3s2)cn1]

¹H NMR (CDCl₃/400 MHz) 8.71-8.69 (s, 1H), 8.05 (dd, J=8.4, 2.0 Hz, 1H), 7.53 (s, 1H), 6.83 (dd, J=8.4, 0.8 Hz, 1H), 6.20 (d, J=0.8 Hz, 1H), 4.25 (t, J=5.6 Hz, 2H), 4.14 (s, 2H), 4.01 (s, 3H), 3.73 (t, J=5.6 Hz, 2H), 3.62-3.56 (m, 2H), 2.88-2.83 (m, 2H), 2.36 (d, J=0.8 Hz, 3H); MS m/z: 400.18 (M+1).

### (6-methoxypyridin-3-yl)(5-methyl-6-(2-((tetrahydro-2H-pyran-4-yl)methylamino)ethyl)-6H-thieno[2,3-b]pyrrol-2-yl)methanone (149)

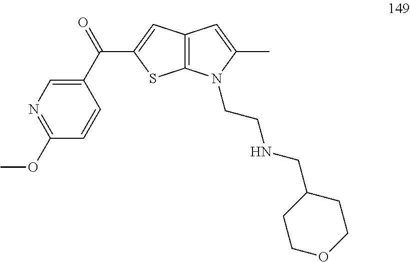 [SMILES: COc1ccc(C(=O)c2cc3cc(C)n(CCNCC4CCOCC4)c3s2)cn1]

¹H NMR (CDCl₃/400 MHz) 8.70 (d, J=2.4 Hz, 1H), 8.05 (dd, J=8.4, 2.4 Hz, 1H), 7.53 (s, 1H), 6.83 (d, J=8.4 Hz, 1H), 6.18 (s, 1H), 4.07 (t, J=6.0 Hz, 2H), 4.01 (s, 3H), 3.93 (dd, J=11.6, 3.6 Hz, 2H), 3.34 (td, J=11.6, 1.6 Hz, 2H), 3.05 (t, J=6.0 Hz, 2H), 2.49 (d, J=6.8 Hz, 2H), 2.38 (d, J=0.8 Hz, 3H), 1.68-1.53 (m, 3H), 1.32-1.08 (m, 3H); MS m/z: 414.23 (M+1).

The compounds below were prepared according to Scheme 3:

### N-(3-(2-(4-Methoxybenzoyl)-5-methyl-6H-thieno[2,3-b]pyrrol-6-yl)propyl)ethanesulfonamide (74)

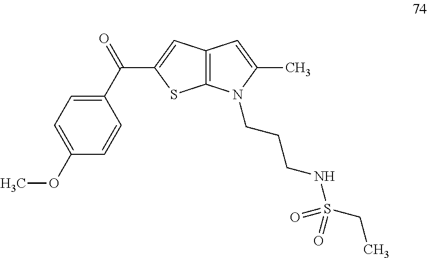 [SMILES: CCS(=O)(=O)NCCCn1c(C)cc2cc(C(=O)c3ccc(OC)cc3)sc21]

¹H NMR (CDCl₃/400 MHz) δ 7.84-7.80 (m, 2H), 7.51 (s, 1H), 6.99-6.95 (m, 2H), 6.17 (d, J=0.8, 1H), 4.70 (t, J=6.8 Hz, 1H), 4.07 (t, J=6.8 Hz, 2H), 3.88 (s, 3H), 3.18 (q, J=6.4 Hz, 2H), 3.04 (q, J=7.2 Hz, 2H), 2.36 (s, 3H), 2.14-2.12 (m, 2H), 1.34 (t, J=7.2 Hz, 3H).

### 1,1,1-Trifluoro-N-(3-(2-(4-methoxybenzoyl)-5-methyl-6H-thieno[2,3-b]pyrrol-6-yl)propyl)methanesulfonamide (75)

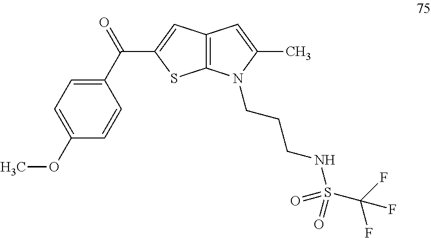 [SMILES: COc1ccc(C(=O)c2cc3cc(C)n(CCCNS(=O)(=O)C(F)(F)F)c3s2)cc1]

¹H NMR (CDCl₃/400 MHz) δ 7.84-7.80 (m, 2H), 7.52 (s, 1H), 6.90-6.86 (m, 2H), 6.19 (d, J=0.8, 1H), 6.13 (s, 3H), 4.09 (t, J=7.2 Hz, 2H), 3.89 (s, 3H), 3.39 (t, J=7.2 Hz, 2H), 2.36 (d, J=0.8 Hz, 3H), 2.22-2.20 (m, 2H).

### 2,2,2-Trifluoro-N-(3-(2-(4-methoxybenzoyl)-5-methyl-6H-thieno[2,3-b]pyrrol-6-yl)propyl)acetamide (76)

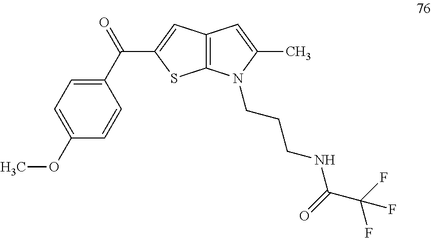 [SMILES: COc1ccc(C(=O)c2cc3cc(C)n(CCCNC(=O)C(F)(F)F)c3s2)cc1]

The title compound was prepared by the acylation of (6-(3-aminopropyl)-5-methyl-6H-thieno[2,3-b]pyrrol-2-yl)(4-methoxyphenyl)methanone.

¹H NMR (CDCl₃/400 MHz) δ 7.86-7.81 (m, 2H), 7.59 (s, 1H), 6.90-6.86 (m, 2H), 6.69 (s, 1H), 6.19 (s, J=0.8 Hz, 1H), 4.06 (t, J=6.8 Hz, 2H), 3.89 (s, 3H), 3.41 (t, J=6.8 Hz, 2H), 2.35 (d, J=0.8 Hz, 1H), 2.20-2.18 (m, 2H).

### N-(3-(2-(4-Methoxybenzoyl)-5-methyl-6H-thieno[2,3-b]pyrrol-6-yl)propyl)benzenesulfonamide (81)

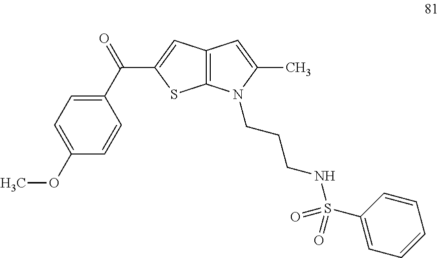 [SMILES: COc1ccc(C(=O)c2cc3cc(C)n(CCCNS(=O)(=O)c4ccccc4)c3s2)cc1]

¹H NMR (CDCl₃/400 MHz) δ 7.86-7.83 (m, 2H), 7.82-7.81 (m, 2H), 7.52 (s, 1H), 7.53-7.45 (m, 3H), 7.01-6.95 (m, 2H), 6.17 (d, J=0.8 Hz, 1H), 4.63 (t, J=6.4 Hz, 1H), 4.03 (t, J=6.8 Hz, 2H), 3.89 (s, 3H), 2.98 (q, J=6.4 Hz, 2H), 2.34 (d, J=0.8 Hz, 3H), 2.07-2.05 (m, 2H).

### N-(3-(2-(2-Fluoro-4-methoxybenzoyl)-5-methyl-6H-thieno[2,3-b]pyrrol-6-yl)propyl)methanesulfonamide (87)

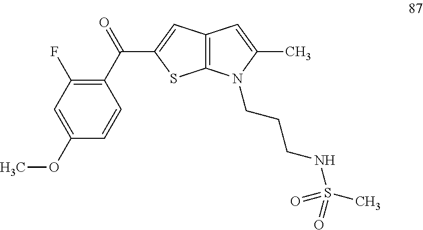

¹H NMR (CDCl₃/400 MHz) δ 7.55 (t, J=8.4 Hz, 1H), 7.41 (d, J=2.4 Hz, 1H), 6.77 (dd, J=8.4, 2.0 Hz, 1H), 6.69 (dd, J=12.0, 2.4 Hz, 1H), 6.17 (d, J=0.8 Hz, 1H), 4.25 (t, J=5.6 Hz, 1H), 4.09 (t, J=6.8 Hz, 2H), 3.87 (s, 3H), 3.16 (q, J=6.4 Hz, 2H), 2.94 (s, 3H), 2.37 (d, J=0.8 Hz, 3H), 2.18-2.15 (m, 2H).

### N-(3-(2-(2-Chloro-4-methoxybenzoyl)-5-methyl-6H-thieno[2,3-b]pyrrol-6-yl)propyl)methanesulfonamide (91)

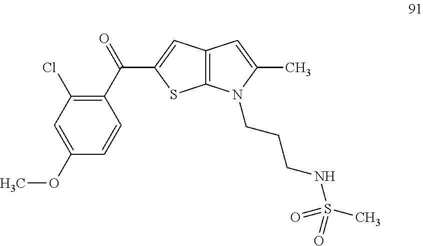

¹H NMR (CD₃OD/400 MHz) δ 7.44 (d, J=8.4 Hz, 1H), 7.28 (s, 1H), 7.10 (d, J=2.4 Hz, 1H), 6.99 (dd, J=8.8, 2.4 Hz, 1H), 6.20 (d, J=0.8 Hz, 1H), 4.12 (t, J=7.2 Hz, 2H), 3.88 (s, 3H), 3.11 (t, J=6.8 Hz, 2H), 2.93 (s, 3H), 2.39 (d, J=0.8 Hz, 3H), 2.10-2.06 (m, 2H); MS m/z: 441 (M+1).

### N-(3-(2-(4-Ethoxybenzoyl)-5-methyl-6H-thieno[2,3-b]pyrrol-6-yl)propyl)methanesulfonamide (94)

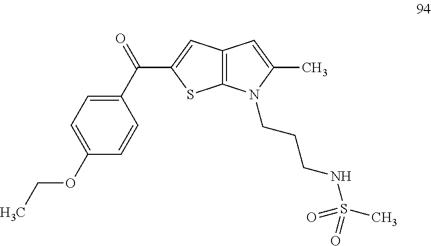 [SMILES: CCOc1ccc(C(=O)c2cc3cc(C)n(CCCNS(C)(=O)=O)c3s2)cc1]

¹H NMR (CDCl₃/400 MHz) δ 7.86-7.83 (m, 2H), 7.53 (s, 1H), 6.99-6.94 (m, 2H), 6.19 (d, J=0.8 Hz, 1H), 4.24-4.22 (m, 1H), 4.11 (q, J=7.2 Hz, 2H), 4.13 (t, J=6.8 Hz, 2H), 3.18 (q, J=6.8 Hz, 2H), 2.93 (s, 3H), 2.38 (d, J=0.8 Hz, 3H), 2.19-2.16 (m, 2H), 1.46 (t, J=7.2 Hz, 3H).

### N-(3-(2-(4-Ethoxy-2-fluorobenzoyl)-5-methyl-6H-thieno[2,3-b]pyrrol-6-yl)propyl)methanesulfonamide (95)

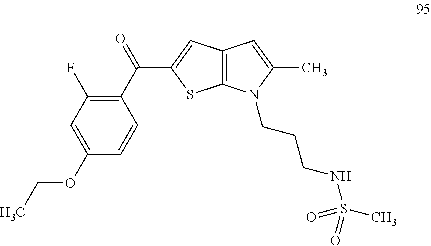 [SMILES: CCOc1ccc(C(=O)c2cc3cc(C)n(CCCNS(C)(=O)=O)c3s2)c(F)c1]

¹H NMR (CDCl₃/400 MHz) δ 7.54 (t, J=12.0 Hz, 1H), 7.41 (d, J=2.4 Hz, 1H), 6.75 (dd, J=8.4, 2.0 Hz, 2H), 6.67 (dd, J=12.0, 2.0 Hz, 1H), 6.17 (d, J=1.2 Hz, 1H), 4.35-4.34 (m, 1H), 4.09 (q, J=7.2 Hz, 2H), 4.08 (t, J=6.8 Hz, 2H), 3.17 (q, J=6.8 Hz, 2H), 2.94 (s, 3H), 2.37 (d, J=0.8 Hz, 3H), 2.16-2.14 (m, 2H), 1.45 (t, J=7.2 Hz, 3H).

### N-(3-(2-(4-Methoxy-2-methylbenzoyl)-5-methyl-6H-thieno[2,3-b]pyrrol-6-yl)propyl)methanesulfonamide (96)

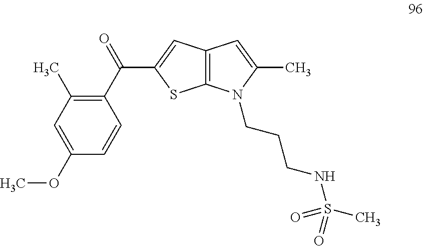

¹H NMR (CDCl₃/400 MHz) δ 7.45 (d, J=8.4 Hz, 1H), 7.28 (s, 1H), 6.81-6.80 (m, 1H), 6.78-6.73 (m, 1H), 6.15 (d, J=1.2 Hz, 1H), 4.28-4.24 (m, 1H), 4.09 (t, J=6.8 Hz, 2H), 3.85 (s, 3H), 3.18 (q, J=6.8 Hz, 2H), 2.94 (s, 3H), 2.41 (s, 3H), 2.37 (d, J=1.2 Hz, 3H), 2.17-2.15 (m, 2H), 1.57 (s, 3H); MS m/z: 421.50 (M+1).

### N-(3-(2-(2-Fluoro-4-methylbenzoyl)-5-methyl-6H-thieno[2,3-b]pyrrol-6-yl)propyl)methanesulfonamide (97)

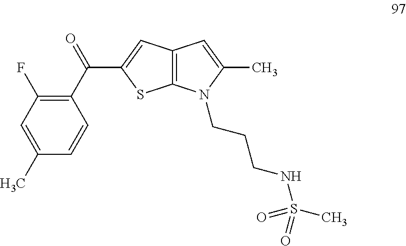 [SMILES: Cc1ccc(C(=O)c2cc3cc(C)n(CCCNS(C)(=O)=O)c3s2)c(F)c1]

¹H NMR (CDCl₃/400 MHz) δ 7.46 (t, J=7.6 Hz, 1H), 7.39-7.37 (m, 1H), 7.06-7.03 (m, 1H), 7.00-6.97 (m, 1H), 6.16 (d, J=1.2 Hz, 1H), 4.36-4.35 (m, 1H), 4.08 (t, J=6.8 Hz, 2H), 3.18 (q, J=6.8 Hz, 3H), 2.95 (s, 3H), 2.42 (s, 3H), 2.36 (d, J=1.2 Hz, 31-1), 2.16-2.13 (m, 2H); MS m/z: 409.26 (M+1).

### N-(4-(2-(4-Methoxybenzoyl)-5-methyl-6H-thieno[2,3-b]pyrrol-6-yl)butyl)methanesulfonamide (102)

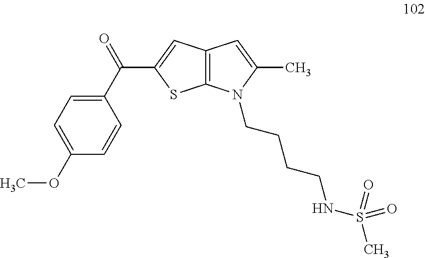 [SMILES: COc1ccc(C(=O)c2cc3cc(C)n(CCCCNS(C)(=O)=O)c3s2)cc1]

¹H NMR (CDCl₃/400 MHz) δ 7.84-7.79 (m, 2H), 7.50 (d, J=1.2 Hz, 1H), 7.15-6.95 (m, 2H), 6.16 (s, 1H), 4.29-3.91 (m, 2H), 3.88 (s, 3H), 3.13 (d, J=6.4 Hz, 2H), 2.91 (d, J=1.2 Hz, 3H), 2.34 (s, 3H), 1.93-1.91 (m, 2H), 1.62-1.60 (m, 2H).

### N-(3-(2-(6-Methoxynicotinoyl)-5-methyl-6H-thieno[2,3-b]pyrrol-6-yl)propyl)methanesulfonamide (109)

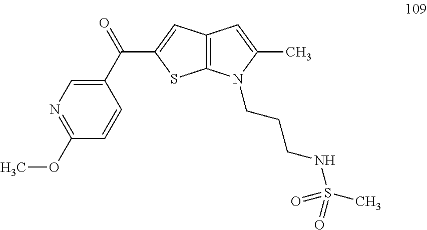

¹H NMR (CDCl₃/400 MHz) δ 8.67 (dd, J=2.8, 0.8 Hz, 1H), 8.03 (dd, J=8.4, 2.8 Hz, 1H), 7.51 (s, 1H), 6.82 (dd, J=8.4, 0.8 Hz, 1H), 6.17 (d, J=1.2 Hz, 1H), 5.05 (t, J=5.6 Hz, 1H), 4.07 (t, J=7.2 Hz, 2H), 4.00 (s, 3H), 3.22-3.15 (m, 2H), 2.95 (s, 3H), 2.35 (d, J=1.2 Hz, 3H), 2.18-2.09 (m, 2H).

### N-(2-(2-(4-Methoxybenzoyl)-5-methyl-6H-thieno[2,3-b]pyrrol-6-yl)ethyl)methanesulfonamide (71)

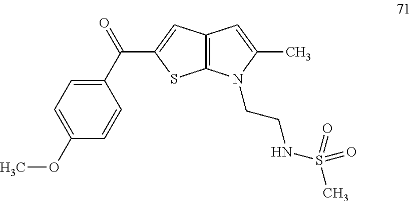 [SMILES: COc1ccc(C(=O)c2cc3cc(C)n(CCNS(C)(=O)=O)c3s2)cc1]

¹H NMR (CDCl₃/400 MHz) δ 7.85-7.80 (m, 2H), 7.49 (s, 1H), 7.00-6.95 (m, 2H), 6.17 (d, J=1.2 Hz, 1H), 5.13 (br s, 1H), 4.16 (t, J=6.0 Hz, 2H), 3.88 (s, 3H), 3.61-3.54 (m, 2H), 2.79 (s, 3H), 2.40 (d, J=0.4 Hz, 3H).

### (2,3-Dichlorophenyl)(5-methyl-6-(2-(pyrrolidin-1-yl)ethyl)-6H-thieno[2,3-b]pyrrol-2-yl)methanone (28)

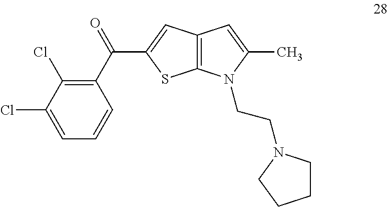 [SMILES: Cc1cc2cc(C(=O)c3cccc(Cl)c3Cl)sc2n1CCN1CCCC1]

¹H NMR (CDCl₃/400 MHz) δ 7.55 (dd, J=7.6, 2.0 Hz, 1H), 7.35 (dd, J=7.6, 2.0 Hz, 1H), 7.29 (t, J=7.6 Hz, 1H), 7.20 (br s, 1H), 6.12 (s, 1H), 4.08 (t, J=6.8 Hz, 2H), 2.88 (t, J=6.8 Hz, 2H), 2.59 (br s, 4H), 2.36 (s, 3H), 1.81 (br s, 4H).

### (4-Methoxyphenyl)(5-methyl-6-(3-morpholinopropyl)-6H-thieno[2,3-b]pyrrol-2-yl)methanone (107)

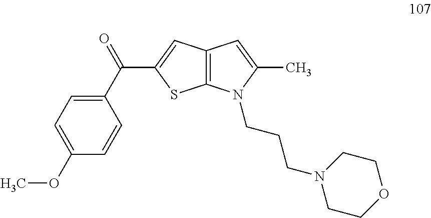 [SMILES: COc1ccc(C(=O)c2cc3cc(C)n(CCCN4CCOCC4)c3s2)cc1]

¹H NMR (CDCl₃/400 MHz) δ 7.88-7.82 (m, 2H), 7.51 (s, 1H), 7.02-6.95 (m, 2H), 6.16 (d, J=0.8 Hz, 1H), 4.05 (t, J=6.8 Hz, 2H), 3.89 (s, 3H), 3.71-3.73 (m, 4H), 2.46-2.39 (m, 4H), 2.38 (d, J=0.8 Hz, 3H), 2.33 (t, J=6.8 Hz, 2H), 2.03-2.00 (m, 2H); MS m/z: 399.22 (M+1).

### (2-Chloro-4-methoxyphenyl)(6-(3-chloropropyl)-5-methyl-6H-thieno[2,3-b]pyrrol-2-yl)methanone (85)

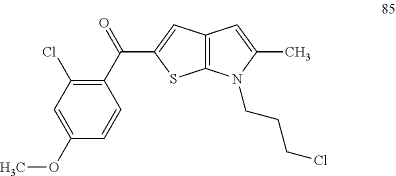 [SMILES: COc1ccc(C(=O)c2cc3cc(C)n(CCCCl)c3s2)c(Cl)c1]

¹H NMR (CDCl₃/400 MHz) δ 7.42 (d, J=8.4 Hz, 1H), 7.26 (d, J=1.2 Hz, 1H), 7.00 (d, J=2.4 Hz, 1H), 6.86 (dd, J=8.4, 2.4 Hz, 1H), 6.14 (d, J=0.8 Hz, 1H), 4.15 (t, J=6.4 Hz, 2H), 3.86 (s, 3H), 3.53 (t, J=6.0 Hz, 1H), 2.39 (d, J=1.2 Hz, 3H), 2.34-2.30 (m, 2H); MS m/z: 383 (M+1).

### (2,3-Dichlorophenyl)(5-methyl-6-(2-(4-methylpiperazin-1-yl)ethyl)-6H-thieno[2,3-b]pyrrol-2-yl)methanone (33)

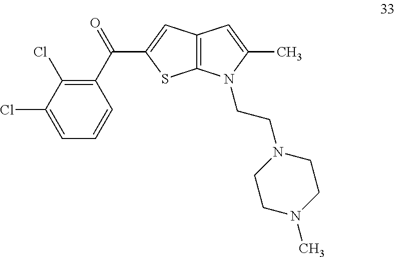 [SMILES: Cc1cc2cc(C(=O)c3cccc(Cl)c3Cl)sc2n1CCN1CCN(C)CC1]

¹H NMR (CDCl₃/400 MHz) δ 7.55 (dd, J=8.0, 1.2 Hz, 1H), 7.35 (dd, J=7.6, 1.2 Hz, 1H), 7.29 (t, J=8.0 Hz, 1H), 7.20 (br s, 1H), 6.12 (s, 1H), 4.04 (t, J=6.8 Hz, 2H), 2.74 (t, J=6.8 Hz, 2H), 2.70-2.30 (br m, 8H), 2.36 (s, 3H), 2.29 (s, 3H).

### (4-Methoxyphenyl)(5-methyl-6-(2-morpholinoethyl)-6H-thieno[2,3-b]pyrrol-2-yl)methanol (100)

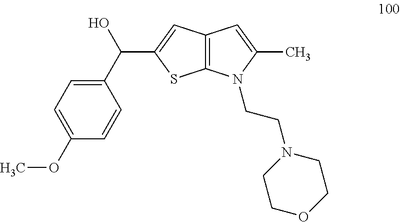 [SMILES: COc1ccc(C(O)c2cc3cc(C)n(CCN4CCOCC4)c3s2)cc1]

The title compound was prepared by lithium aluminum hydride reduction of compound 36. ¹H NMR (CDCl₃/400 MHz) δ 7.41-7.37 (m, 2H), 6.90-6.87 (m, 2H), 6.72 (d, J=0.8 Hz, 1H), 6.01 (d, J=0.8 Hz, 1H), 5.98 (s, 1H), 3.97 (t, J=6.8 Hz, 2H), 3.80 (s, 3H), 3.69-3.65 (m, 4H), 2.75 (s, 1H), 2.65 (t, J=7.2 Hz, 2H), 2.46-2.43 (m, 4H), 2.33 (s, 3H); MS m/z: 387.20 (M+1).

### (6-(3-(tert-Butylamino)propyl)-5-methyl-6H-thieno[2,3-b]pyrrol-2-yl)(4-methoxyphenyl)methanone (108)

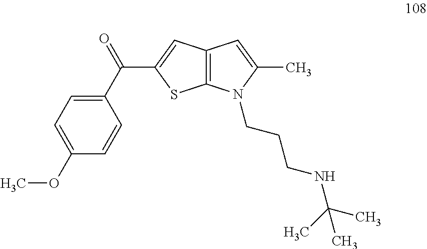 [SMILES: COc1ccc(C(=O)c2cc3cc(C)n(CCCNC(C)(C)C)c3s2)cc1]

¹H NMR (CDCl₃/400 MHz) δ 7.88-7.83 (m, 2H), 7.51 (s, 1H), 7.00-6.95 (m, 2H), 6.16 (d, J=0.8 Hz, 1H), 4.05 (t, J=7.2 Hz, 2H), 3.89 (s, 3H), 2.60 (t, J=7.2 Hz, 2H), 2.38 (d, J=0.8 Hz, 3H), 1.99-1.97 (m, 2H), 1.07 (s, 9H); MS m/z: 386.24 (M+1).

### N-(3-(2-(4-Methoxybenzoyl)-5-methyl-6H-thieno[2,3-b]pyrrol-6-yl)propyl)-N-methylmethanesulfonamide (69)

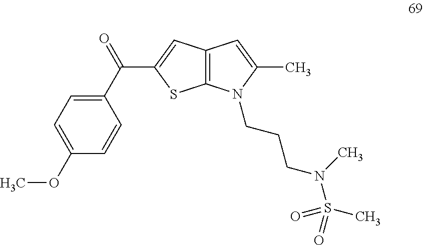

¹H NMR (CDCl₃/400 MHz) δ 7.87-7.82 (m, 2H), 7.52 (s, 1H), 7.00-6.95 (m, 2H), 6.17 (d, J=1.2 Hz, 1H), 4.05 (t, J=7.2 Hz, 2H), 3.88 (s, 3H), 3.21 (t, J=6.8 Hz, 2H), 2.86 (s, 3H), 2.79 (s, 3H), 2.37 (d, J=1.2 Hz, 3H), 2.20-2.11 (m, 2H).

### Example 10

### Synthesis of N-(3-(2-(4-methoxybenzoyl)-5-methyl-6H-thieno[2,3-b]pyrrol-6-yl)propyl)aminosulfonamide (105)

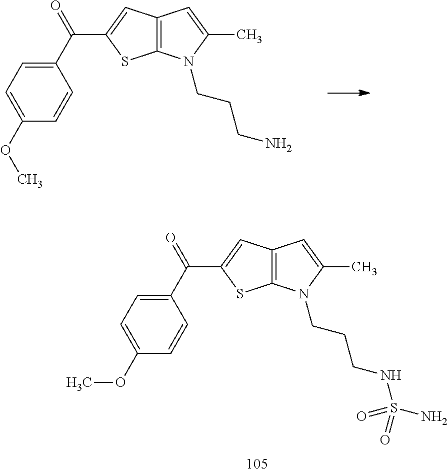 [SMILES: COc1ccc(C(=O)c2cc3cc(C)n(CCCN)c3s2)cc1 ; COc1ccc(C(=O)c2cc3cc(C)n(CCCNS(N)(=O)=O)c3s2)cc1]

A mixture of (6-(3-aminopropyl)-5-methyl-6H-thieno[2,3-b]pyrrol-2-yl)(4-methoxyphenyl)methanone (274 mg, 0.834 mmol) and sulfuric diamide (241 mg, 2.503 mmol) in 55.6 mL of dioxane was heated at reflux for 4 h. A precipitate was formed and removed via filtration. The yellow solution was diluted with EtOAc (50 mL) and washed with H₂O (20 mL×3). The organic layer was layer was dried with MgSO₄ and concentrated in vacuo to afford an oil. The residue was purified by flash silica gel chromatography (10% to 80% acetone in hexanes containing 2% triethylamine) to afford 169 mg (50%) of the title compound as a pale yellow solid; ¹H NMR (CDCl₃/400 MHz) δ 7.86-7.81 (m, 2H), 7.50 (s, 1H), 7.00-6.95 (m, 2H), 6.17 (d, J=1.2 Hz, 1H), 4.70 (br s, 2H), 4.08 (t, J=6.8 Hz, 2H), 3.88 (s, 3H), 3.18 (t, J=6.8 Hz, 2H), 2.61-2.50 (m, 1H), 2.38-2.35 (m, 3H), 1.05-1.03 (m, 2H); MS m/z: 408.51 (M+1).

### Example 11

### Synthesis of N-(3-(2-(4-methoxybenzoyl)-5-methyl-6H-thieno[2,3-b]pyrrol-6-yl)propyl)methanesulfonamide (44)

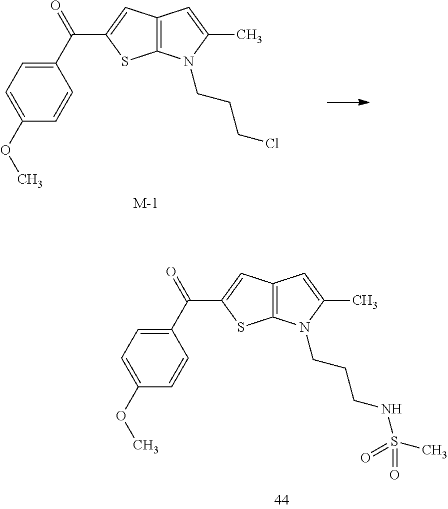 [SMILES: COc1ccc(C(=O)c2cc3cc(C)n(CCCCl)c3s2)cc1 ; "M-1" ; COc1ccc(C(=O)c2cc3cc(C)n(CCCNS(C)(=O)=O)c3s2)cc1]

A solution of methanesulfonamide (8.01 g, 84 mmol) in 40 mL of DMF was added to a suspension of 60% sodium hydride (2.020 g, 84 mmol) in 100 mL of DMF (100 mL). The mixture was heated at 60° C. for 1 h. The reaction was cooled to rt. Tetrabutylammonium idodide (1.727 g, 2.81 mmol) was added to the reaction mixture followed by a yellow solution of (6-(3-chloropropyl)-5-methyl-6H-thieno[2,3-b]pyrrol-2-yl)(4-methoxyphenyl)methanone (0.976 g, 2.81 mmol) in 50 mL of DMF. The mixture was heated at 55° C. for 40 h. The reaction was cooled to rt and the tetrabutylammonium salt was removed by filtration. The resulting solution was diluted with EtOAc (300 mL) and washed with H₂O (100 mL). The aqueous layer was extracted with EtOAc (200 mL). The organic layers were combined and further washed with H₂O (5×200 mL). The organic layer was dried with MgSO₄ and concentrated in vacuo to afford a solid. The residue was purified by flash silica gel chromatography (0% to 30% acetone in hexanes containing 2% triethylamine) to afford 846 mg (74%) of N-(3-(2-(4-methoxybenzoyl)-5-methyl-6H-thieno[2,3-b]pyrrol-6-yl)propyl) as a yellow solid. ¹H NMR (CDCl₃/400 MHz) δ 7.86-7.84 (m, 2H), 7.52 (s, 1H), 6.99-6.97 (m, 2H), 6.19 (s, 1H), 4.52 (t, J=5.6 Hz, 1H), 4.09 (t, J=6.8 Hz, 2H), 3.89 (s, 3H), 3.18 (m, 2H), 2.94 (s, 3H), 2.38 (s, 3H), 2.20-2.10 (m, 2H).

### Example 12

### Synthesis of (6-(2-chloroethyl)-5-methyl-6H-thieno[2,3-b]pyrrol-2-yl)(6-methoxypyridin-3-yl)

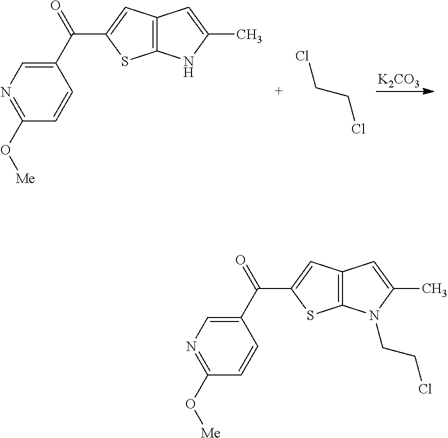

(6-methoxypyridin-3-yl)(5-methyl-6H-thieno[2,3-b]pyrrol-2-yl)methanone (510 mg, 1.87 mmol), 1,2-dichloroethane (4.41 mL, 56.2 mmol) and potassium carbonate (1.3 g, 9.36 mmol) was mixed in DMF (43 mL) a sealed vial (it was mixed and stood at rt for 3 hr prior to heating) and heated at 100° C. with stirring for 17.25 h. The reaction mixture became light brown. The reaction mixture was diluted with water (200 mL) and EtOAc (200 mL). The aqueous layer was extracted with EtOAc (2×200 mL). The combined organic layers were washed 2× with water (200 mL). The organic layer was then dried with MgSO4 and concentrated. The crude product was added to a 80 g ISCO silica gel column and was purified with a gradient of 0 to 40% ethyl acetate/hexane (Product eluted at ˜30% EtOAc). The title product was obtained as a light yellow crystalline solid (48% yield). HNMR (CDCl₃/400 MHz) 8.71 (dd, 1H), 8.06 (dd, 1H), 7.55 (s, 1H), 6.85 (dd, 1H), 6.21 (d, 1H), 4.39 (t, 2H), 4.02 (s, 3H), 3.86 (t, 2H), 2.41 (d, 3H);

### Example 13

### Synthesis of (6-methoxypyridin-3-yl)(5-methyl-6-(2-((tetrahydrofuran-3-yl)amino)ethyl)-6H-thieno[2,3-b]pyrrol-2-yl)methanone (164)

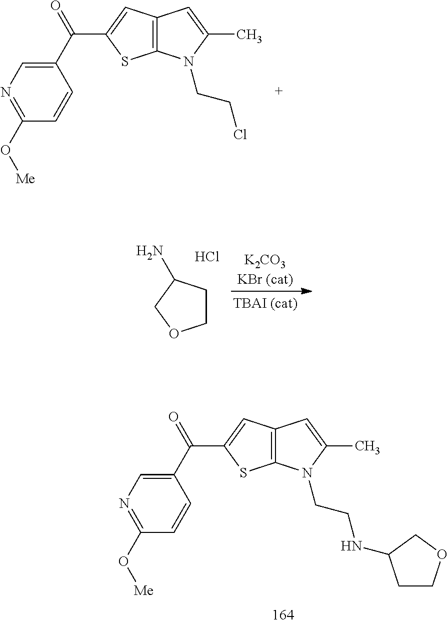 [SMILES: COc1ccc(C(=O)c2cc3cc(C)n(CCCl)c3s2)cn1 ; NC1CCOC1 ; Cl ; "K2CO3KBr (cat)" ; COc1ccc(C(=O)c2cc3cc(C)n(CCNC4CCOC4)c3s2)cn1]

To a solution of (6-(2-chloroethyl)-5-methyl-6H-thieno[2,3-b]pyrrol-2-yl)(6-methoxypyridin-3-yl)methanone (100.5 mg, 0.3 mmol) and tetrahydrofuran-3-amine hydrochloride (594 mg, 4.80 mmol) in acetonitrile (8 mL) was added KBr (cat) and TBAI (cat). The reaction mixture was heated in a sealed pressure tube at 80° C. for 48 h. The reaction mixture was diluted with water and extracted with DCM (3×100 mL). The organic layers were combined, dried with MgSO4, and concentrated in vacuo to afford a yellow residue. The residue was purified by reverse phase HPLC (5%-495% acetonitrile in H2O containing 0.1% TFA) to afford 44 mg (38%) of (6-methoxypyridin-3-yl)(5-methyl-6-(2-(tetrahydro-2H-pyran-4-ylamino)ethyl)-6H-thieno[2,3-b]pyrrol-2-yl)methanone (14.8 mg, 0.037 mmol, 12.58% yield) as a yellow oil. HNMR (CDCl₃/400 MHz) 8.71 (dd, J=2.4, 0.4 Hz, 1H), 8.07 (dd, J=8.8, 2.4 Hz, 1H), 7.54 (s, 1H), 6.84 (dd, J=8.8, 0.8 Hz, 1H), 6.19 (d, J=0.8 Hz, 1H), 4.08 (t, J=6.4 Hz, 2H), 4.02 (s, 3H), 3.88 (dd, J=11.2, 8.0 Hz, 1H), 3.79-3.71 (m, 2H), 3.54 (dd, J=9.2, 3.6 Hz, 1H), 3.41-3.37 (m, 1H), 3.12-3.01 (m, 2H), 2.39 (d, J=0.8 Hz, 3H), 2.12-2.04 (m, 1H), 1.70-1.63 (m, 1H); MS m/z: 386.20 (M+1).

### (6-methoxypyridin-3-yl)(5-methyl-6-(2-((tetrahydro-2H-pyran-4-yl)amino)ethyl)-6H-thieno[2,3-b]pyrrol-2-yl)methanone (163)

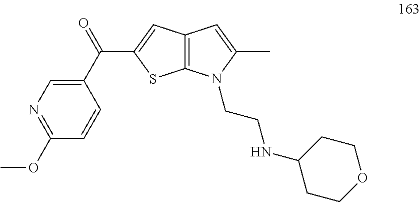 [SMILES: COc1ccc(C(=O)c2cc3cc(C)n(CCNC4CCOCC4)c3s2)cn1]

HNMR (CDCl₃/400 MHz) 8.71 (d, J=2.4 Hz, 1H), 8.07 (dd, J=8.8, 2.4 Hz, 1H), 7.54 (s, 1H), 6.85 (d, J=8.8 Hz, 1H), 6.19 (d, J=0.8 Hz, 1H), 4.08 (t, J=6.8 Hz, 2H), 4.03 (s, 3H), 3.97-3.93 (m, 2H), 3.37 (td, J=11.6, 2.0 Hz, 2H), 3.33-3.28 (m, 1H), 3.11 (t, J=6.8 Hz, 2H), 2.71-2.66 (m, 1H), 2.40 (d, J=0.8 Hz, 3H), 1.80 (dd, J=12.8, 2.0 Hz, 2H), 1.68-1.62 (m, 1H); MS m/z: 400.23 (M+1).

The compounds below were prepared according to Scheme 4:

### N-(3-(2-(2,3-Dichlorobenzoyl)-5-methyl-6H-thieno[2,3-b]pyrrol-6-yl)propyl)methanesulfonamide (37)

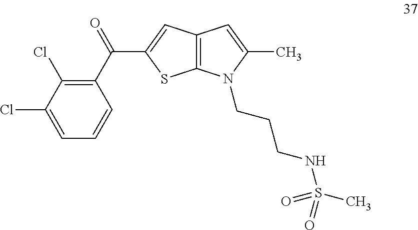 [SMILES: Cc1cc2cc(C(=O)c3cccc(Cl)c3Cl)sc2n1CCCNS(C)(=O)=O]

¹H NMR (CDCl₃/400 MHz) δ 7.56 (dd, J=7.6, 2.0 Hz, 1H), 7.34 (dd, J=7.6, 2.0 Hz, 1H), 7.30 (t, J=7.6 Hz, 1H), 7.21 (br s, 1H), 6.15 (d, J=0.8 Hz 1H), 4.64 (t, J=6.0 Hz 1H), 4.09 (t, J=7.0 Hz, 2H), 2.95 (s, 3H), 2.36 (d, J=0.8 Hz, 3H), 2.19-2.10 (m, 2H);

### N-(3-(2-(4-Methoxybenzoyl)-5-methyl-6H-thieno[2,3-b]pyrrol-6-yl)propyl)acetamide (63)

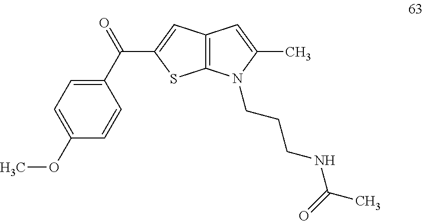 [SMILES: COc1ccc(C(=O)c2cc3cc(C)n(CCCNC(C)=O)c3s2)cc1]

This compound was prepared by the acylation of (6-(3-aminopropyl)-5-methyl-6H-thieno[2,3-b]pyrrol-2-yl)(4-methoxyphenyl)methanone (151). ¹H NMR (CDCl₃/400 MHz) δ 7.86-7.82 (m, 2H), 7.52 (s, 1H), 7.02-6.94 (m, 2H), 6.14 (d, J=0.8, 1H), 5.64 (s, 1H), 4.01 (t, J=6.8 Hz, 2H), 3.28 (q, J=6.8 Hz, 2H), 2.35 (d, J=0.8 Hz, 3H), 2.11-2.08 (m, 2H), 2.04 (s, 3H), 1.93 (s, 3H).

### Example 14

### Synthesis of (5-methyl-6-(2-morpholinoethyl)-6H-thieno[2,3-b]pyrrol-2-yl)(p-tolyl)methanimine (121)

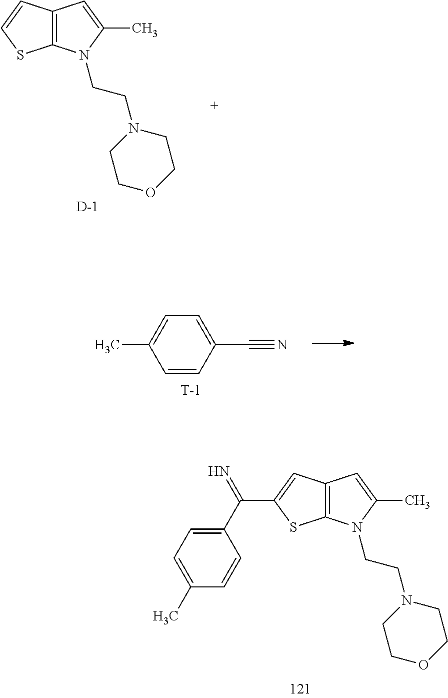

A 1.6 M solution of n-butyllithium (3.55 mL, 5.67 mmol) in hexanes was slowly added to a −20° C. solution of 4-(2-(5-methyl-6H-thieno[2,3-b]pyrrol-6-yl)ethyl)morpholine (1.235 g, 4.93 mmol) in 30 mL of THF. The reaction mixture stirred at −20° C. for 1 h. A solution of 4-methylbenzonitrile (0.722 g, 6.17 mmol) in 5 ml, of THF was added relatively quickly to the reaction. The reaction mixture turned from light yellow to orange. The bath was allowed to warm with stirring to 5° C. over 2 h and the mixture turned a deeper orange hue, before it was diluted with 5 mL of a 3.0 M aqueous solution of HCl (4.93 mL, 14.80 mmol) at rt. The reaction mixture became green and heterogeneous. After stirring at rt for 5 minutes (min), the THF was removed in vacuo. The green residue was triturated with Et₂O (2×) to remove unreacted 4-methylbenzonitrile. The green residue, containing the hydrochloride salt of the imine, was taken up in H₂O and washed with EtOAc. The aqueous layer was neutralized with a saturated aqueous solution of NaHCO₃ and extracted with EtOAc (3 times). The organic layers were combined, washed with brine, dried with Na₂SO₄, and concentrated in vacuo to afford 1.68 g (93%) of (5-methyl-6-(2-morpholinoethyl)-6H-thieno[2,3-b]pyrrol-2-yl)(p-tolyl)methanimine as a tan solid; ¹H NMR (wet CDCl₃/400 MHz) δ 7.44 (d, J=7.2 Hz, 2H), 7.24 (d, J=8.0 Hz, 2H), 7.04 (s, 1H), 6.06 (s, 1H), 4.05 (t, J=7.2 Hz, 2H), 3.71 (t, J=7.2 Hz, 4H), 2.74 (t, J=7.2 Hz, 2H), 2.51-2.49 (m, 4H), 2.42 (s, 3H), 2.36 (s, 3H).

### Synthesis of 1,1,1-trifluoro-N-(3-(2-(4-methoxybenzoyl)-5-methyl-6H-thieno[2,3-b]pyrrol-6-yl)propyl)methanesulfonamide (154)

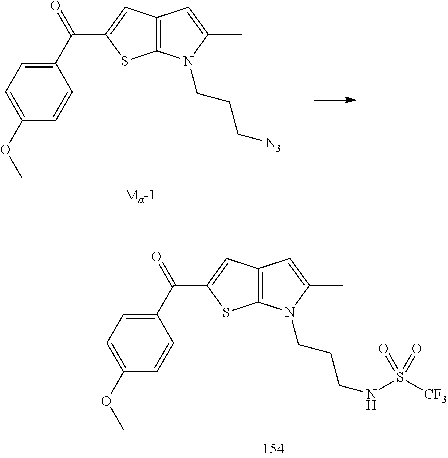 [SMILES: COc1ccc(C(=O)c2cc3cc(C)n(CCCN=[N+]=[N-])c3s2)cc1 ; "Ma-1" ; COc1ccc(C(=O)c2cc3cc(C)n(CCCNS(=O)(=O)C(F)(F)F)c3s2)cc1]

This title compound was produced from 151. Triethylphosphite (0.222 ml, 1.27 mmol) was added to a solution of (6-(3-azidopropyl)-5-methyl-6H-thieno[2,3-b]pyrrol-2-yl)(4-methoxyphenyl)methanone (150 mg, 0.423 mmol) in 4 mL of benzene. The reaction mixture was sealed in a vial and heated at 80° C. for 48 h. The mixture was cooled to rt. A 4.0 M of HCl in dioxane (0.317 ml, 1.27 mmol) was added to the reaction. The reaction mixture was heated at reflux for an additional 3 h. The reaction was cooled to rt and concentrated in vacuo to afford a red oil. The residue was dissolved in dichloromethane (4.0 mL) and triethylamine (1.18 ml, 8.46 mmol) and DMAP (5.17 mg, 0.042 mmol) were added to the mixture. The reaction was cooled to 0° C. Trifluoromethane sulfonic anhydride (0.072 mL, 0.423 mmol) was added to the reaction. The reaction mixture was allowed to warm to rt and stirred a total of 18 h. The mixture was diluted with dichloromethane (100 mL), washed with a saturated aqueous solution of ammonium chloride (50 mL×2), and washed with brine (10 mL). The organic layer was dried with MgSO₄ and concentrated in vacuo to afford an oil. The residue was purified by flash silica gel chromatography (40% EtOAc in hexanes) to afford 45.7 mg (23%) of 1,1,1-trifluoro-N-(3-(2-(4-methoxybenzoyl)-5-methyl-6H-thieno[2,3-b]pyrrol-6-yl)propyl)methanesulfonamide as a yellow foam; ¹H NMR (CDCl₃/400 MHz) δ 7.84-7.80 (m, 2H), 7.52 (s, 1H), 6.90-6.86 (m, 2H), 6.19 (d, J=0.8, 1H), 6.13 (s, 1H), 4.09 (t, J=7.2 Hz, 2H), 3.89 (s, 3H), 3.39 (t, J=7.2 Hz, 2H), 2.36 (d, J=0.8 Hz, 3H), 2.22-2.20 (m, 2H).

The following compounds were prepared according to Schemes 5 or 6:

### Synthesis of (5-Methyl-6-(2-morpholinoethyl)-6H-thieno[2,3-b]pyrrol-2-yl)(p-tolyl)methanimine bis hydrochloride salt (121*2HCl)

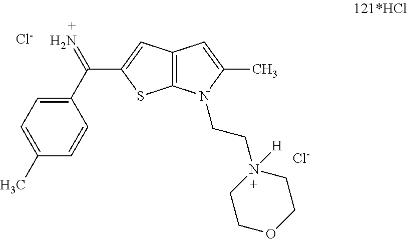

This salt was prepared from compound 121 by the following method: compound 121 was dissolved in MeOH. To the stirred solution were added 20 equivalents of HCl in MeOH. After 30 min of stirring at rt, all volatiles were evaporated off to afford the desired salt. ¹H NMR (D₂O/400 MHz) δ 7.61 (s, 1H), 7.43 (d, J=8.0 Hz, 2H), 7.28 (d, J=8.0 Hz, 2H), 6.24 (d, J=0.8 Hz, 1H), 4.28 (t, J=7.2 Hz, 2H), 3.77 (br s, 4H), 3.25 (t, J=6.8 Hz, 2H), 3.08 (br s, 4H), 2.28 (s, 3H), 2.25 (s, 3H).

### (4-Ethylphenyl)(5-methyl-6-(2-morpholinoethyl)-6H-thieno[2,3-b]pyrrol-2-yl)methanone (122)

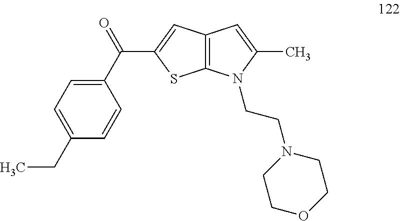 [SMILES: CCc1ccc(C(=O)c2cc3cc(C)n(CCN4CCOCC4)c3s2)cc1]

¹H NMR (CDCl₃/400 MHz) δ 7.79 (d, J=8.0 Hz, 2H), 7.52 (s, 1H), 7.31 (d, J=8.0 Hz, 2H), 6.17 (s, 1H), 4.02-4.00 (m, 2H), 3.75-3.66 (m, 4H), 2.74 (q, J=7.6 Hz, 2H), 2.54-2.46 (m, 4H), 2.38 (s, 3H), 1.28 (t, J=7.6 Hz, 3H).

### (6-Ethoxypyridin-3-yl)(5-methyl-6-(2-morpholinoethyl)-6H-thieno[2,3-b]pyrrol-2-yl)methanone (150)

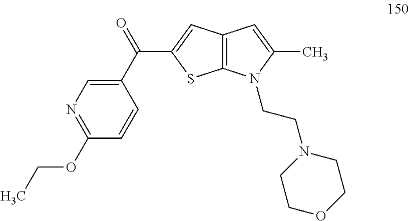 [SMILES: CCOc1ccc(C(=O)c2cc3cc(C)n(CCN4CCOCC4)c3s2)cn1]

¹H NMR (CDCl₃/400 MHz) δ 8.67-8.66 (m, 1H), 8.04 (dd, J=8.8, 2.4 Hz, 1H), 7.51 (s, 1H), 6.78 (dd, J=8.8, 0.8 Hz, 1H), 6.15 (d, J=1.2 Hz, 1H), 4.42 (q, J=6.8 Hz, 2H), 4.04 (t, J=6.8 Hz, 2H), 3.69-3.67 (m, 4H), 2.72 (q, J=6.8 Hz, 2H), 2.49-2.46 (m, 4H), 2.36 (d, J=1.2 Hz, 3H), 1.41 (t, J=6.8 Hz, 3H); MS m/z: 400.14 (M+1).

### N-(3-(2-(2,3-Dichlorobenzoyl)-5-methyl-6H-thieno[2,3-b]pyrrol-6-yl)propyl)methanesulfonamide (37)

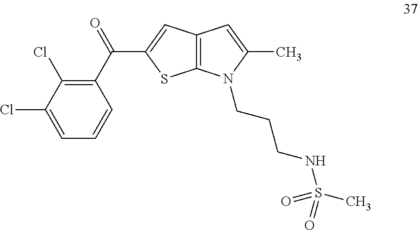 [SMILES: Cc1cc2cc(C(=O)c3cccc(Cl)c3Cl)sc2n1CCCNS(C)(=O)=O]

¹H NMR (CDCl₃/400 MHz) δ 7.56 (dd, J=7.6, 2.0 Hz, 1H), 7.34 (dd, J=7.6, 2.0 Hz, 1H), 7.30 (t, J=7.6 Hz, 1H), 7.21 (br s, 1H), 6.15 (d, J=0.8 Hz 1H), 4.64 (t, J=6.0 Hz 1H), 4.09 (t, J=7.0 Hz, 2H), 2.95 (s, 3H), 2.36 (d, J=0.8 Hz, 3H), 2.19-2.10 (m, 2H).

### Example 15

### Synthesis of (4-chloro-5-methyl-6-(2-morpholinoethyl)-6H-thieno[2,3-b]pyrrol-2-yl)(2,3-dichlorophenyl)methanone (27)

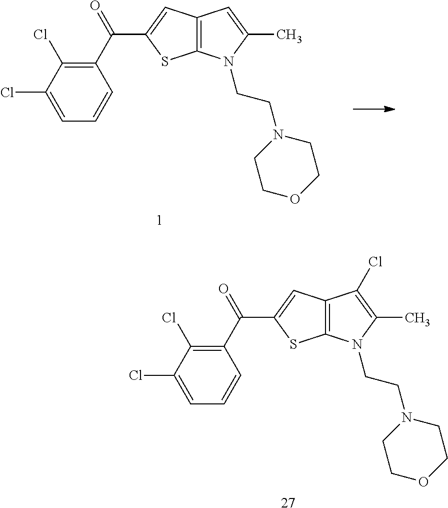 [SMILES: Cc1cc2cc(C(=O)c3cccc(Cl)c3Cl)sc2n1CCN1CCOCC1 ; "1" ; Cc1c(Cl)c2cc(C(=O)c3cccc(Cl)c3Cl)sc2n1CCN1CCOCC1 ; "27"]

N-Chlorosuccinimide (17.69 mg, 0.133 mmol) was added to a solution of (2,3-dichlorophenyl)(5-methyl-6-(2-morpholinoethyl)-6H-thieno[2,3-b]pyrrol-2-yl)methanone (37.4 mg, 0.088 mmol) in 2 mL of DMF. The yellow solution stirred at rt for 2 h and was heated at 55° C. for 2 h 40 min. The reaction was diluted with a saturated aqueous solution of NaHCO₃ and EtOAc. The aqueous layer was extracted with EtOAc (2 times) and the combined organic layers were washed with H₂O (2 times). The organic phase was dried with MgSO₄ and concentrated in vacuo. The residue was purified by flash silica gel chromatography (0% to 40% acetone in hexanes containing 2% triethylamine) to afford 28.7 mg (71%) of compound 27 as a yellow foam; ¹H NMR (CDCl₃/400 MHz) δ 7.58 (dd, J=7.6, 2.0 Hz, 1H), 7.35 (dd, J=7.6, 2.0 Hz, 1H), 7.31 (t, J=7.6 Hz, 1H), 7.18 (br, s 1H), 4.03 (t, J=6.4 Hz, 2H), 3.70 (t, J=4.4 Hz, 4H), 2.71 (t, J=6.4 Hz, 2H), 2.49 (t, J=4.4 Hz, 4H), 2.34 (s, 3H).

The following compound was prepared according to Example 15:

### (4-chloro-5-methyl-6-(2-morpholinoethyl)-6H-thieno[2,3-b]pyrrol-2-yl)(4-methoxyphenyl)methanone (140)

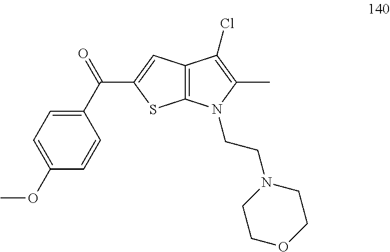 [SMILES: COc1ccc(C(=O)c2cc3c(Cl)c(C)n(CCN4CCOCC4)c3s2)cc1]

¹H NMR (CDCl₃/400 MHz) 7.85 (dd, J=7.2, 2.0 Hz, 2H), 7.47 (s, 1H), 6.98 (dd, J=6.8, 2.0 Hz, 2H), 4.03 (t, J=6.4 Hz, 2H), 3.88 (s, 3H), 3.69 (t, J=4.4 Hz, 4H), 2.71 (t, J=6.4 Hz, 2H), 2.48 (t, J=4.0 Hz, 4H), 2.34 (s, 3H); MS m/z: 419.17 (M+1).

### Example 16

### Synthesis of (2-Aminophenyl)(5-methyl-6-(2-morpholinoethyl)-6H-thieno[2,3-b]pyrrol-2-yl)methanone (48)

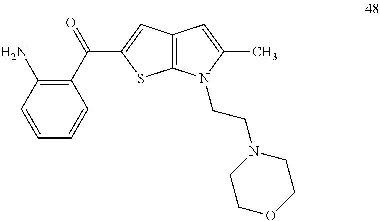 [SMILES: Cc1cc2cc(C(=O)c3ccccc3N)sc2n1CCN1CCOCC1]

2-(5-methyl-6-(2-morpholinoethyl)-6H-thieno[2,3-b]pyrrole-2-carbonyl)benzoic acid was prepared according to Scheme 1 and was further used without purification. To the crude product 2-(5-methyl-6-(2-morpholinoethyl)-6H-thieno[2,3-b]pyrrole-2-carbonyl)benzoic acid (382 mg, 0.959 mol) was added t-butyl alcohol (10 mL), triethylamine (267 μL, 1.98 mol), diphenyl phosphorazidate (528 mg, 1.92 mol) and was heated to 100° C. for 2 hours. The reaction was cooled, then diluted with a saturated aqueous solution of NaHCO₃ and EtOAc. The organic layer was separated, dried with MgSO₄ and concentrated in vacuo. The residue was purified by flash silica gel chromatography (50% to 100% ethyl acetate in hexanes) to afford 9 mg (2.5%) of the title compound. ¹H NMR (CDCl₃/400 MHz) δ 7.72 (m, 1H), 7.42 (m, 2H), 7.25 (m, 2H), 6.70 (m, 2H), 6.2 (s, 1H), 5.3 (bs, 2H), 4.05 (m, 2H), 3.65 (m, 4H), 2.75 (m, 2H), 2.50 (m, 2H), 2.42 (s, 3H). MS m/z: 370.2 (M+1).

### Example 17

### Synthesis of (4-methoxyphenyl)(5-methyl-6-(2-morpholinoethyl)-6H-thieno[2,3-b]pyrrol-2-yl)methanethione (104)

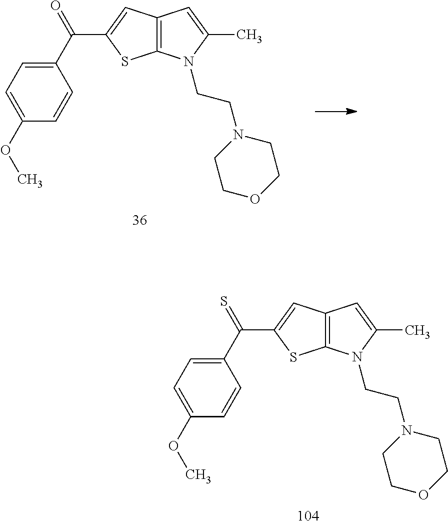 [SMILES: COc1ccc(C(=O)c2cc3cc(C)n(CCN4CCOCC4)c3s2)cc1 ; "36" ; COc1ccc(C(=S)c2cc3cc(C)n(CCN4CCOCC4)c3s2)cc1 ; "104"]

4-Methoxyphenylthiophosphoric cyclic di(thioanhydride), LR, 2,4-Bis(4-methoxyphenyl)-2,4-dithioxo-1,3,2,4-dithiadiphosphetane, 2,4-Bis-(4-methoxyphenyl)-1,3-dithia-2,4-diphosphetane 2,4-disulfide (Lawesson's reagent) (63.1 mg, 0.156 mmol) and NaHCO₃ (43.7 mg, 0.520 mmol) were added to a microwave vial containing (4-methoxyphenyl)(5-methyl-6-(2-morpholinoethyl)-6H-thieno[2,3-b]pyrrol-2-yl)methanone (100 mg, 0.260 mmol) in 2 mL of THF. The reaction mixture was heated at 100° C. in the microwave for 40 min. The reaction mixture was concentrated in vacuo. The residue was purified by flash silica gel chromatography (30% to 100% EtOAc in hexanes) to afford an impure residue which was subjected to a second purification via flash silica gel chromatography (0% to 10% A:B [A: acetonitrile/MeOH-7/1, B: dichloromethane) to afford 41.5 mg (40%) of compound 104 as a brown oil; ¹H NMR (CDCl₃/400 MHz) δ 7.69 (d, J=8.8 Hz, 2H), 7.26 (s, 1H), 6.90 (d, J=8.8 Hz, 2H), 6.13 (d, J=1.2 Hz, 1H), 4.03 (t, J=6.8 Hz, 2H), 3.87 (s, 3H), 3.71 (t, J=4.8 Hz, 4H), 2.75 (t, J=6.8 Hz, 2H), 2.51 (t, J=4.8 Hz, 4H), 2.35 (d, J=1.2 Hz, 3H); MS m/z: 401(M+1).

### Example 18

### Synthesis of 1-(4-methoxyphenyl)-1-(5-methyl-6-(2-morpholinoethyl)-6H-thieno[2,3-b]pyrrol-2-yl)ethanol (101)

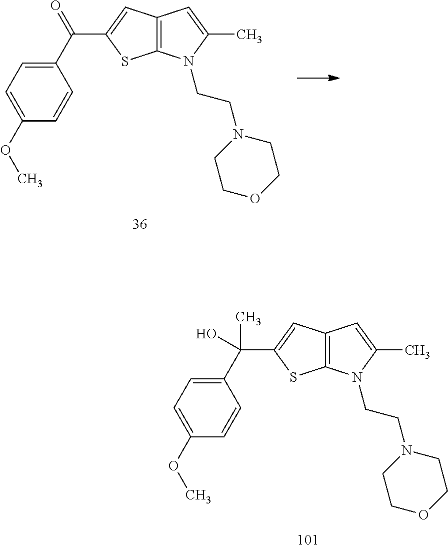 [SMILES: COc1ccc(C(=O)c2cc3cc(C)n(CCN4CCOCC4)c3s2)cc1 ; COc1ccc(C(C)(O)c2cc3cc(C)n(CCN4CCOCC4)c3s2)cc1]

Methylmagnesium bromide (780 μL, 0.780 mmol) was added to a solution of (4-methoxyphenyl)(5-methyl-6-(2-morpholinoethyl)-6H-thieno[2,3-b]pyrrol-2-yl)methanone (100 mg, 0.260 mmol) in 1.3 mL of THF. The reaction was heated at reflux for 16 h. An additional amount of methylmagnesium bromide (780 μL, 0.780 mmol) was added to the reaction mixture and heating was resumed at reflux for an additional 48 h. The reaction mixture was quenched by the addition of H₂O (10 mL) and extracted with dichloromethane (100 mL). The organic phase was dried with MgSO₄ and concentrated in vacuo to afford a red oil. The residue was purified by flash silica gel chromatography (0% to 50% EtOAc in hexanes to 0% to 30% acetone in hexanes containing 2% triethylamine) to afford 29.3 mg (22%) of compound 101 as a brown oil; MS m/z: 401.5 (M+1).

The following compound was prepared following the procedure in Example 18:

### 2,2,2-trifluoro-1-(4-methoxyphenyl)-1-(5-methyl-6-(2-morpholinoethyl)-6H-thieno[2,3-b]pyrrol-2-yl)ethanol (138)

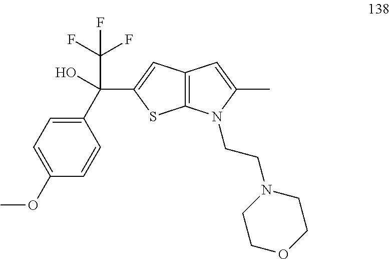 [SMILES: COc1ccc(C(O)(c2cc3cc(C)n(CCN4CCOCC4)c3s2)C(F)(F)F)cc1]

¹H NMR (CDCl₃/400 MHz) 7.66-7.53 (m, 2H), 7.07 (s, 1H), 6.95-6.86 (m, 2H), 6.07 (s, 1H), 4.01-3.94 (m, 2H), 3.84 (s, 3H), 2.69-2.60 (m, 2H), 2.49-2.40 (m, 4H), 2.35 (s, 3H); MS m/z: 455.21 (M+1).

### 4-(2-(5-methyl-2-(2,2,2-trifluoro-1-(4-methoxyphenyl)ethyl)-6H-thieno[2,3-b]pyrrol-6-yl)ethyl)morpholine (141)

This compound was obtained from the de-hydroxylation of compound 138:

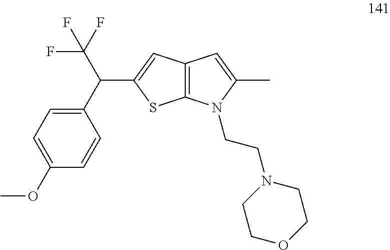 [SMILES: COc1ccc(C(c2cc3cc(C)n(CCN4CCOCC4)c3s2)C(F)(F)F)cc1]

¹H NMR (CDCl₃/400 MHz) 7.40-7.34 (m, 2H), 6.93-6.86 (m, 2H), 6.89 (d, J=9.2 Hz, 1H), 6.05 (d, J=0.8 Hz, 1H), 4.78 (q, J=9.2 Hz, 1H), 3.97 (t, J=5.8 Hz, 2H), 3.81 (s, 3H), 3.71-2.64 (m, 4H), 2.66 (t, J=5.8 Hz, 2H), 2.51-2.43 (m, 4H), 2.34 (d, J=0.8 Hz, 3H); MS m/z: 439.16 (M+1).

### Example 19

### Synthesis of (4-methoxyphenyl)(6-(2-morpholinoethyl)-6H-thieno[2,3-b]pyrrol-2-yl)methanone (160)

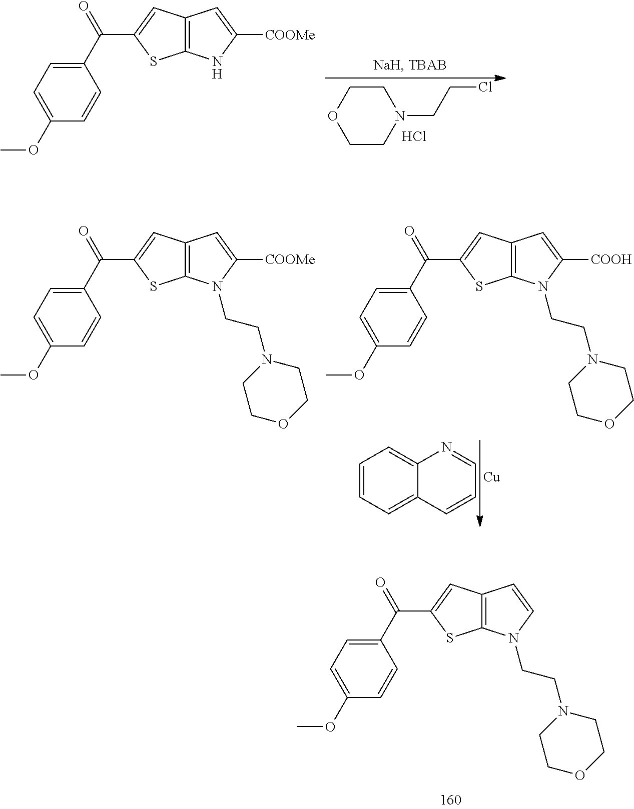

The mixture was stirred at room temperature for 15 min. To this mixture, was added 4-(2-chloroethyl)morpholine hydrochloride (64.9 mg, 0.349 mmol) in DMF (2 ml). The mixture was heated to 80° C. for 2 h. To the reaction mixture was added 4 mL H2O. the mixture was frozen and was put on the lyophilizer overnight. To the solid obtained was added DCM (10 mL) and a few drops of glacial HOAc. The mixture was stirred at it for 20 min. Then the solvent was removed (including the acid) under reduced pressure. the obtained crude product was dissolved in ACN and was loaded to a pre-packed (25 g) silica gel cartridge. The ISCO solvent was ACN (solvent A) and H2O (solvent B), 5% to 25%.

The ester was obtained at fraction #07. LC/MS 429.21 (M+1), yellow solid. ¹H NMR (CD3OD/400 MHz) 7.81 (d, J=8.8 Hz, 2H), 7.71 (s, 1H), 7.28 (s, 1H), 7.08 (d, J=9.2 Hz, 2H), 4.60 (t, J=6.4 Hz, 2H), 3.90 (s, 3H), 3.86 (s, 3H), 3.64 (t, J=4.8 Hz, 4H), 2.79 (t, J=6.4 Hz, 2H), 2.52 (t, J=4.4 Hz, 4H); MS m/z: 429.21 (M+1).

The acid was obtained at fraction #37. LC/MS 415.18 (M+1), yellow solid ¹H NMR (CD3OD/400 MHz): 7.85 (d, J=8.8 Hz, 2H), 7.72 (s, 1H), 7.21 (s, 1H), 7.08 (d, J=8.8 Hz, 2H), 4.73 (t, J=6.0 Hz, 2H), 3.90 (s, 3H), 3.78 (t, J=4.4 Hz, 4H), 3.24-3.20 (br s, 2H), 2.93-2.88 (br s, 4H); MS m/z: 415.18 (M+1).

To a solution of 2-(4-methoxybenzoyl)-6-(2-morpholinoethyl)-6H-thieno[2,3-b]pyrrole-5-carboxylic acid (45 mg, 0.109 mmol) in quinoline (20 ml, 0.109 mmol) was added copper (6.90 mg, 0.109 mmol). The reaction was heated to reflux at 160° C. for 2 h. The mixture was filtered over celite plug. The filtrated was poured on ice and was extracted with EtOAc (3×100 mL). LC/MS showed complete completion after 2 h. The organic layers were combined, dried over MgSO4, filtered and concentrated under vacuum to give a dark brown oil crude product. The crude product was purified by column chromatography by dry loading the sample and eluting with a gradient of 5% to 10% (ACN/MeOH 6:1) in DCM. ¹H NMR (CDCl3/400 MHz) 7.80 (d, J=8.8 Hz, 2H), 7.51 (s, 1H), 6.93-6.90 (m, 3H), 6.36 (d, J=2.8 Hz, 1H), 4.05 (t, J=6.4 Hz, 2H), 3.83 (s, 3H), 3.65 (t, J=4.4 Hz, 4H), 2.75 (t, J=6.4 Hz, 2H), 2.44 (br s, 4H); MS mh: 371.16 (M+1).

### (5,6-dimethyl-6H-thieno[2,3-b]pyrrol-2-yl)(6-methoxypyridin-3-yl)methanone (174)

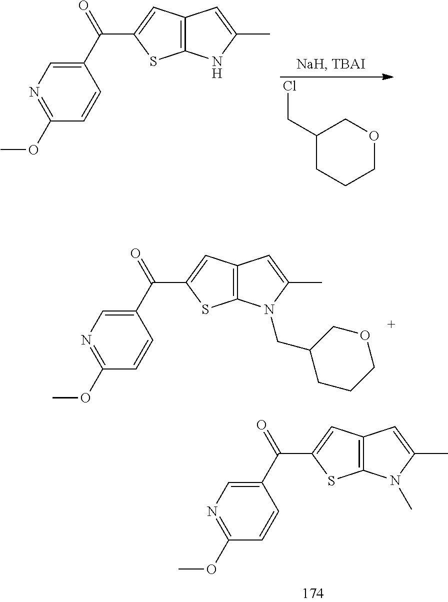

To (6-methoxypyridin-3-yl)(5-methyl-6H-thieno[2,3-b]pyrrol-2-3/1)methanone (200 mg, 0.734 mmol) and NaH (88 mg, 2.203 mmol) was added DMF (25 ml) in a round bottom flask at room temperature. The reaction was exothermic. To the mixture was added 3-(chloromethyl)tetrahydro-2H-pyran (99 mg, 0.734 mmol). Meanwhile tetra-n-butylammonium bromide (cat) was added. The reaction was warmed to 80° C. for 2 h. Saw two new peaks: LC/MS 371.24 (M+1) and LC/MS 287.14 (M+1). The reaction was poured onto water (100 mL) and extracted with DCM (3×40 mL). The organic layers were combined, dried over MgSO4, filtered and concentrated under vacuum to give a dark brown oil crude product. The crude product was purified by column chromatography by dry loading the sample and eluting with a gradient of 5% to 20% DCM/(ACN/MeOH 6:1). (5,6-dimethyl-6H-thieno[2,3-b]pyrrol-2-yl)(6-methoxypyridin-3-yl)methanone was obtained after the column chromatography. 1H NMR (CDCl3/400 MHz): 8.70 (d, J=2.4 Hz, 1H), 8.06 (dd, J=8.4, 2.4 Hz, 1H), 7.53 (s, 1H), 6.84 (d, J=8.8 Hz, 1H), 6.19 (d, J=1.2 Hz, 1H), 4.02 (s, 3H), 3.65 (s, 3H), 2.36 (s, 3H); MS m/z: 287.14 (M+1).

### Biological Activity

### CB1 and CB2 Clones

cDNA expression clones for human CB1 (hCB1, Genbank Accession No. AY225225) and human CB2 (hCB2, Genbank Accession No. AY242132) expressed in vector pcDNA3.1+ were purchased from UMR cDNA Resource Center, Rolla, Mo. (Clone ID CNR01L000 for hCB1; CNR0200000 for hCB2).

### Stable and Transient Transfection

Stable, HEK-293-derived cell lines that recombinantly express hCBI or hCB2 were established. In brief, the clone hCB1 (CNR IL) or hCB2 (CNR2) was transfected into human embryonic kidney cells (HEK-293) using Lipofectamine 2000 (Gibco, Cat#11668-019) according to the manufacturer's protocol. Transfected clones were isolated by single colony purification and clones were screened for receptor expression using a whole cell, ³H—CP 55,940 radioligand binding assay. HEK-293 stable cells were maintained in Dulbecco's modified Eagles medium (DMEM) containing 10% fetal bovine serum, 2 mM L-glutamine and 0.5 mg/mL G-418.

### Human CB1 and CB2 Cannabinoid Receptor Radioligand Binding Assays

Membranes were isolated from transfected cells as follows. Monolayers of cultured cells were washed twice with phosphate-buffered saline (PBS). Cells were scraped into 20 mM HEPES, pH 7.4, 10 mM EDTA containing complete cocktail protease inhibitors (Roche, Catalog #11 697 498 001) and were homogenized by an electric-powered mechanical probe homogenizer (Omni GLH; probe G7-195S) for 40 seconds at 7000 rpm. Homogenates were centrifuged 10 minutes at 1000×g at 4° C. The supernatant was collected and was centrifuged for 1 hour at 40,000×g. The supernatant was then decanted and the resulting pellet was re-suspended in 20 mM HEPES, pH 7.4, 5 mM MgCl₂, 1 mM EDTA, 10% sucrose with complete cocktail protease inhibitors. Protein concentration of membrane suspensions were measured by Bradford Protein Assay using bovine serum albumin as the standard (BioRad catalog #500-0006). Protein concentrations of membrane suspensions were adjusted with the final buffer in the range of 5 to 10 mg/mL and were stored at −80° C. until further use.

### Cannabinoid Receptor Radioligand Binding Assays

Radioligand binding assays were performed by incubating membranes (2-10 μg protein) prepared from HEK-293 cells expressing recombinant human cannabinoid receptors, CB1 or CB2, at room temperature with 0.5 nM cannabinoid receptor agonist, [³H]-CP 55,940 (Perkin Elmer, catalog #NET1051) in 0.2 mL of binding buffer (50 mM Tris-HCl, pH 7.5, 5 mM MgCl₂, 2.5 mM EDTA) and 0.1% fatty acid free bovine serum albumin (Sigma Cat. #A0821) for 90 minutes. A rapid filtration technique using Millipore FB filter plates (Catalog #MADVNOB) and filtration apparatus (Millipore system Catalog MAVM0960R) with vacuum aspiration was used to harvest and rinse labeled membranes (8 times with 0.2 mL of chilled binding buffer). The radioactivity bound to the filters was counted with 0.05 mL of liquid scintillant (UltraGold MV, PerkinElmer catalog #6013159) in a scintillation counter (Perkin Elmer Microbeta instrument). Nonspecific binding was determined in the presence of unlabeled 1 μM CP 55,940 (Sigma Aldrich, catalog #C1112). Binding data were analyzed using GraphPad Prism (GraphPad Software, Inc. San Diego, Calif.).

### Human and Rodent CB1 and CB2 Receptor Functional Assays

Functional assays which monitor G-protein coupled receptor or downstream cellular responses can be used to characterize potential CB1 receptor and/or CB2 receptor agonist or antagonist activities. Direct activation (or inhibition of activation) can be monitored using a GTPγS assay (membrane-based assay) or cAMP assay (whole cell-based assay).

GTPγS assay

³⁵S GTPγS binding assays were performed by incubating recombinant cell membranes prepared above (5 μg) in the presence of scintillation proximity assay beads (SPA beads, Catalog #RPNQ0252 GE Healthcare, Buckinghamshire, England) in GTPγS binding buffer [50 mM HEPES (pH 7.4), 100 mM NaCl, 5 mM MgCl₂, 0.001% saponin (Sigma catalog #S4521)] supplemented with 20 uM GDP (Sigma catalog #G7127) in the presence or absence of test compound. The reaction was carried out in 96-well microplates with 0.1 nM [³⁵S]GTPγS (specific activity=1250 Ci/mmol; Perkin Elmer catalog #NEG030X250UC) in a final volume of 100 uL. After a 90 min room temperature incubation, the reaction was analyzed using a scintillation counter (Perkin Elmer Microbeta instrument). Binding data were analyzed using GraphPad Prism.

cAMP Assay

cAMP assays were performed in HEK-293 cells stably expressing human CB1 or CB2 receptors. For cannabinoid receptor functional assays measuring agonist effects on cellular cAMP levels, monolayers of cultured cells were harvested with enzyme-free PBS-based cell dissociation buffer (Gibco, Cat#13151-04). Cells suspensions were centrifuged and the cells were washed once with PBS, were centrifuged again, and the cells were re-suspended in HBSS (Hank's Balanced Salt Solution, Cellgro, Cat #21-022-CV) solution containing 10 mM HEPES and 0.1% fatty acid free BSA (Sigma, Cat #A0281). Cell suspensions were prepared at 1,500,000 cells per ml. Stock solutions of test substances (10 mM) in DMSO were diluted to 1 mM using 30% DMSO as diluent. Test substances of solutions (1 mM) were further diluted down to 3× of final assay concentrations in the above HBSS buffer containing 0.1% BSA in the presence of 90 uM forskolin (Sigma Cat#F6886). To perform the assay, 20 uL of cell suspension (1,500,000 cells/mL) were added to each well in 96 well plate and treated with 10 uL test substance solution diluted as described above. Cells and compounds were incubated at 37° C. for 30 minutes. Cells were lysed and cAMP concentration was measured using DiscoveRx −XS+ cAMP assay kit (DiscoveRx Corporation Ltd., Fremont, Calif., USA, Cat #90-0075-03), following the manufacturer's protocol. GraphPad Prism software was used to calculate EC₅₀ values using sigmoidal dose response curve fitting. The maximal amount of cAMP produced by forskolin was defined as 100%. CB1 or CB2 agonists reduced forskolin-stimulated cAMP signaling. The EC₅₀ value of an agonist compound was defined as the concentration at which 50% of the forskolin-stimulated cAMP synthesis was inhibited.

Animal Models

1. Animal Model for Assessing Anti-Inflammatory Activity: Complete Freund's Adjuvant (CFA) Induced Inflammatory Pain

In this rat model of inflammatory pain, 100 uL of CFA diluted 1:1 with phosphate buffered saline was injected into the subplantar region of the right hind paw on Day 1. On day 3, test compounds were administered orally and rats were assessed for their reaction to a mechanical stimuli applied via an Analgesy® meter. Injection of CFA increased the rats' reactivity to painful stimuli and this was reflected in a decrease in the amount of pressure they could tolerate prior to withdrawing their paw from the apparatus (hyperalgesia). An anti-hyperalgesic activity of the test compound was denoted by an increase in the amount of pressure they can tolerate prior to withdrawing their paw from the apparatus. The mean±SEM for each treatment group was determined and a Dunnett test was applied for comparison between vehicle and treated groups. Differences were considered significant at P<0.05. (see Bertorelli et al. 1999 Brit Journ Pharmacol 128:1252).

2. Animal Model for Assessing Analgesic Activity: Phenylbenzoquinone-Induced (PBC)) Writhing Model

This model is described by Siegmund et al. (1957 Proc Soc Exp Bio Med 95:729). Briefly, one hour after oral (PO) or intraperitoneal (IP) dosing with a test compound, morphine or vehicle, 0.02% phenylbenzoquinone (PBQ) solution (12.5 mL/kg) was injected by intraperitoneal route into the mouse. The number of stretches and writhings were recorded from the 5th to the 10th minutes after PBQ injection, and were also counted between the 35^{th} and 40^{th} minutes and between the 60^{th} and 65^{th} minutes to provide a kinetic assessment. The results were expressed as the number of stretches and writhings (mean±SEM) and the percentage of variation of the nociceptive threshold calculated from the mean value of the vehicle-treated group. The statistical significance of any differences between the treated groups and the control group was determined by a Dunnett's test using the residual variance after a one-way analysis of variance (P_{<}0.05) using SigmaStat Software.

Data for compounds of the invention are summarized in Table 1, Table 2 and Table 3

TABLE 1
hCB2 and hCB1 Activity

| | | | | | hCB2 | hCB1 |
|---|---|---|---|---|---|---|
| | hCB2 | hCB1 | hCB2 | hCB1 | IC50 | IC50 |
| | EC50 | EC50 | EC50 | EC50 | (CP | (CP |
| Comp | (GTPyS | (GTPyS | (cAMP | (cAMP | displ, | displ, |
| # | ag, nM) | ag, nM) | ag, nM) | ag, nM | nM) | nM) |
| 1 | A | NS | A | D | A | B |
| 2 | NS | NS | C | NS | C | D |
| 3 | A | NS | A | NS | B | C |
| 4 | A | NS | A | NS | B | C |
| 5 | A | NS | A | B | A | B |
| 6 | A | NS | A | NS | B | C |
| 7 | B | NS | A | C | B | C |
| 8 | B | NS | A | NS | B | C |
| 9 | C | NS | B | NS | B | C |
| 10 | A | NS | A | B | A | B |
| 11 | A | NS | A | B | B | B |
| 12 | A | NS | A | B | A | C |
| 13 | A | NS | A | B | A | B |
| 14 | ND | NS | B | NS | C | D |
| 15 | A | B | A | B | A | A |
| 16 | NS | NS | B | NS | C | D |
| 17 | B | NS | A | NS | B | D |
| 18 | A | NS | A | B | A | B |
| 19 | B | NS | A | NS | B | D |
| 20 | B | NS | ND | NS | C | D |
| 21 | A | NS | A | C | B | B |
| 22 | ND | NS | B | B | C | D |
| 23 | A | NS | A | NS | A | B |
| 24 | A | NS | A | NS | A | B |
| 25 | A | NS | A | NS | A | C |
| 26 | A | NS | A | B | A | B |
| 27 | A | B | A | B | A | A |
| 28 | A | NS | A | NS | A | B |
| 29 | B | NS | B | NS | C | D |
| 30 | A | B | A | NS | A | B |
| 31 | A | NS | A | NS | A | B |
| 32 | A | NS | A | C | A | C |
| 33 | NS | A | NS | NS | C | D |
| 34 | NS | NS | C | NS | D | D |
| 35 | A | B | A | NS | A | B |
| 36 | A | NS | A | C | A | C |
| 37 | A | NS | A | NS | A | D |
| 38 | A | NS | A | NS | A | C |
| 40 | ND | NS | B | NS | C | D |
| 41 | A | NS | A | C | A | B |
| 42 | A | NS | A | NS | B | C |
| 43 | A | NS | A | NS | B | D |
| 44 | A | NS | A | NS | A | C |
| 45 | A | B | A | NS | A | A |
| 46 | A | A | A | A | A | A |
| 47 | B | NS | A | NS | C | D |
| 48 | A | NS | A | C | A | D |
| 49 | B | NS | B | NS | C | D |
| 50 | C | NS | A | NS | C | D |
| 51 | A | NS | A | NS | A | C |
| 53 | A | B | A | A | A | A |
| 54 | A | NS | A | NS | B | C |
| 55 | A | NS | A | C | B | C |
| 56 | A | NS | A | NS | B | D |
| 57 | A | A | A | A | A | A |
| 58 | A | NS | A | NS | B | D |
| 59 | B | NS | B | NS | C | D |
| 60 | C | NS | A | NS | C | D |
| 61 | NS | NS | NS | NS | NS | NS |
| 62 | A | B | A | B | A | C |
| 63 | NS | NS | NS | NS | NS | NS |
| 64 | A | NS | A | B | A | C |
| 65 | B | NS | B | NS | B | C |
| 66 | NS | NS | ND | NS | D | D |
| 67 | B | NS | ND | NS | C | D |
| 68 | A | NS | A | C | A | C |
| 69 | B | NS | ND | NS | C | D |
| 70 | A | NS | A | B | A | B |
| 71 | B | NS | B | NS | C | D |
| 72 | B | NS | B | NS | C | D |
| 73 | NS | NS | NS | NS | D | D |
| 74 | ND | NS | B | NS | C | D |
| 75 | NS | NS | NS | NS | NS | NS |
| 76 | B | A | B | NS | B | C |
| 77 | B | A | B | C | C | C |
| 78 | A | A | A | C | A | C |
| 79 | NS | NS | B | NS | D | D |
| 80 | A | NS | A | NS | B | C |
| 81 | NS | NS | NS | NS | C | D |
| 82 | A | B | A | B | A | B |
| 83 | B | NS | A | C | B | C |
| 84 | B | NS | A | NS | B | C |
| 85 | A | NS | A | C | A | C |
| 86 | A | C | A | C | A | B |
| 87 | A | NS | A | NS | B | D |
| 88 | A | NS | A | B | A | B |
| 89 | A | NS | A | C | B | C |
| 90 | A | NS | A | B | A | B |
| 91 | A | NS | A | NS | A | D |
| 92 | A | NS | A | C | B | C |
| 93 | A | NS | A | C | A | B |
| 94 | A | NS | B | NS | B | ND |
| 95 | B | NS | A | NS | B | ND |
| 96 | A | NS | A | NS | A | C |
| 97 | A | NS | A | NS | B | ND |
| 98 | A | NS | A | NS | A | C |
| 99 | A | NS | A | NS | B | C |
| 100 | A | NS | A | NS | B | C |
| 101 | A | NS | A | NS | B | C |
| 102 | A | NS | A | NS | A | C |
| 103 | B | NS | B | NS | B | C |
| 104 | B | NS | B | NS | C | C |
| 105 | B | NS | C | NS | C | D |
| 106 | A | NS | A | NS | B | C |
| 107 | A | B | A | B | A | B |
| 108 | B | NS | C | NS | D | D |
| 109 | A | NS | A | NS | B | D |
| 110 | A | NS | A | C | A | C |
| 111 | B | NS | A | NS | B | C |
| 112 | B | NS | B | NS | B | C |
| 113 | A | NS | A | NS | A | C |
| 114 | A | NS | C | C | A | C |
| 115 | B | NS | B | NS | B | C |
| 116 | A | NS | A | NS | B | C |
| 117 | A | NS | A | NS | B | C |
| 118 | B | NS | B | NS | NS | NS |
| 119 | A | B | A | B | A | C |
| 120 | A | B | A | B | A | B |
| 121 | A | NS | A | NS | B | C |
| 122 | A | NS | A | C | A | C |
| 123 | B | NS | A | NS | B | C |
| 124 | A | NS | A | C | B | C |
| 125 | B | NS | B | NS | D | D |
| 126 | A | C | A | C | A | B |
| 127 | A | NS | A | C | A | C |
| 128 | A | NS | B | NS | B | C |
| 129 | A | NS | B | NS | B | C |
| 130 | B | NS | B | NS | C | D |
| 131 | A | NS | A | NS | B | C |
| 132 | B | NS | B | NS | C | C |
| 133 | A | NS | A | B | A | B |
| 134 | B | NS | A | NS | B | C |
| 135 | B | NS | A | NS | B | C |
| 136 | A | NS | A | NS | A | C |
| 137 | A | C | A | B | A | C |
| 138 | A | NS | A | NS | B | C |
| 139 | A | B | A | B | A | B |
| 140 | A | NS | A | NS | B | C |
| 141 | B | NS | A | NS | B | C |
| 142 | B | NS | B | NS | C | D |
| 143 | NS | NS | NS | NS | D | D |
| 144 | A | NS | A | NS | B | C |
| 145 | A | NS | A | C | A | C |
| 146 | NS | NS | B | NS | B | D |
| 147 | B | NS | B | NS | C | D |
| 148 | NS | NS | ND | NS | D | D |
| 149 | NS | NS | B | NS | D | D |
| 150 | B | NS | A | NS | C | D |
| 151 | ND | ND | ND | ND | ND | ND |
| 181 | B | C | NS | ND | ND | NS |
| 180 | A | C | A | ND | ND | B |
| 179 | D | D | ND | NS | NS | NS |
| 178 | D | D | NS | NS | NS | NS |
| 177 | C | D | C | NS | NS | ND |
| 176 | D | D | A | NS | NS | NS |
| 175 | A | B | A | ND | C | A |
| 174 | D | D | ND | NS | NS | ND |
| 173 | B | C | A | ND | ND | A |
| 172 | D | D | NS | NS | NS | NS |
| 171 | D | D | ND | NS | NS | ND |
| 170 | A | A | A | A | B | A |
| 169 | C | D | ND | NS | NS | B |
| 168 | D | D | ND | NS | NS | ND |
| 167 | C | D | A | NS | NS | A |
| 166 | D | D | NS | NS | NS | NS |
| 165 | B | D | A | B | NS | B |
| 164 | C | D | A | NS | NS | C |
| 163 | C | D | B | NS | NS | ND |
| 162 | D | D | B | NS | NS | NS |
| 161 | C | D | B | NS | NS | B |
| 160 | C | D | B | ND | NS | NS |
| 159 | D | D | C | NS | NS | NS |
| 158 | B | D | A | NS | NS | B |
| 157 | C | D | A | NS | NS | B |
| 152 | A | C | A | ND | ND | A |
| 156 | D | D | ND | NS | NS | NS |
| 155 | B | D | ND | NS | NS | NS |
| 154 | C | D | B | ND | NS | 158 |

Less than 100 nM = A, between 100 nM and 1 μM = B, between 1 μM and 10 μM = C greater than 10 μM = D.
NS means “Not Significant,” which means less than 30% agonist activity when compared to the positive control.
ND means “Not Determined.”

TABLE 2
PBQ writhing model:

| Compound | | | Number of | Statistical |
|---|---|---|---|---|
| Number | Dose | Route | Writhes | Significance |
| I-25 | 0 | IP | 22.3 ± 1.35 | |
| | 30 | | 5.9 ± 2.46 | P < 0.01 |
| I-1 | 0 | IP | 22.3 ± 1.35 | |
| | 30 | | 0.4 ± 0.31 | P < 0.001 |
| I-16 | 0 | IP | 14.1 ± 2.38 | |
| | 30 | | 10.0 ± 2.76 | NS |
| I-34 | 0 | IP | 15.6 ± 2.21 | |
| | 30 | | 3.4 ± 0.97 | P < 0.001 |
| I-58 | 0 | IP | 11.7 ± 2.48 | |
| | 30 | | 1.1 ± 0.38 | P < 0.01 |
| I-37 | 0 | PO | 18.0 ± 1.98 | |
| | 30 | | 13.0 ± 1.65 | NS |
| I-43 | 0 | PO | 18.0 ± 1.98 | |
| | 30 | | 13.8 ± 1.88 | NS |
| I-6 | 0 | PO | 16.5 ± 2.2 | |
| | 30 | | 3.7 ± 0.7 | P < 0.001 |
| I-10 | 0 | PO | 16.5 ± 2.2 | |
| | 30 | | 3.7 ± 0.7 | P < 0.05 |
| I-44 | 0 | PO | 16.5 ± 2.2 | |
| | 30 | | 8.25 ± 2.54 | P < 0.05 |
| I-1 | 0 | PO | 23.2 ± 1.47 | |
| | 30 | | 7.5 ± 1.91 | P < 0.001 |
| I-44 | 0 | PO | 15.3 ± 1.4 | |
| | 3 | | 13.9 ± 1.4 | |
| | 10 | | 9.4 ± 2.6 | |
| | 30 | | 9.7 ± 1.7 | P < 0.01 |
| I-41 | 0 | IP | 20.0 ± 2.8 | |
| | 30 | | 5.91 ± 2.04 | P < 0.001 |
| I-68 | 0 | PO | 14.46 ± 3.7 | |
| | 3 | | 9.6 ± 2.2 | |
| | 10 | | 5.92 ± 1.55 | P < 0.01 |
| | 30 | | 2.2 ± 0.89 | P < 0.01 |
| I-68 | 0 | PO | 14.6 ± 3.5 | |
| | 30 | | 6.4 ± 1.48 | P < 0.05 |
| I-70 | 0 | PO | 14.6 ± 3.5 | |
| | 30 | | 3.2 ± 1.57 | P < 0.01 |
| I-36 | 0 | PO | 17.0 ± 2.1 | |
| | 3 | | 14.5 ± 1.59 | |
| | 10 | | 12.1 ± 2.8 | |
| | 30 | | 7.3 ± 2.5 | P < 0.01 |
| I-87 | 0 | PO | 19.1 ± 2.5 | |
| | 30 | | 17.2 ± 2.5 | NS |
| I-58 | 0 | PO | 19.1 ± 2.5 | |
| | 30 | | 15.9 ± 2.7 | NS |
| I-144 | 0 | PO | 12.8 ± 2.52 | |
| | 3 | | 5.04 ± 1.8 | P < 0.01 |
| | 10 | | 3.24 ± 1.08 | P < 0.01 |
| | 30 | | 1.8 ± 0.72 | P < 0.001 |
| I-109 | 0 | PO | 18.2 ± 1.44 | |
| | 3 | | 13.3 ± 2.3 | |
| | 10 | | 8.93 ± 2.28 | P < 0.01 |
| | 30 | | 5.78 ± 1.75 | P < 0.001 |

TABLE 3
CFA model

| Com- | | | Pre CFA | Post CFA | Statistical |
|---|---|---|---|---|---|
| pound | Dose | | PWT | PWT | Signif- |
| Number | (mg/kg) | Route | (grams) | (grams) | icance^{a} |
| I-44 | 0 | PO | 202.2 ± 10.1 | 137 ± 10.5 | |
| | 10 | | 210 ± 16.1 | 164.4 ± 8.4 | |
| | 30 | | 205 ± 10.2 | 153.7 ± 10.7 | |
| | 56 | | 200 ± 16.7 | 150 ± 13.8 | |
| I-87 | 0 | PO | 204 ± 17.2 | 97.7 ± 12.4 | |
| | 60 | | 204 ± 15.8 | 100.7 ± 11.3 | |
| I-68 | 0 | PO | 204 ± 17.2 | 97.7 ± 12.4 | |
| | 60 | | 204 ± 13.6 | 178.7 ± 24.4 | P < 0.05 |
| I-109 | 0 | PO | 204.0 ± 12.6 | 89.3 ± 11.1 | |
| | 60 | | 204 ± 12.5 | 132 ± 12.3 | P < 0.05 |
| I-58 | 0 | PO | 204 ± 12.6 | 89.3 ± 11.1 | |
| | 60 | | 204.3 ± 12.51 | 170.3 ± 19.7 | P < 0.01 |
| I-95 | 0 | PO | 211.7 ± 14.6 | 115.7 ± 20.6 | |
| | 60 | | 211.3 ± 13.8 | 111.7 ± 20.5 | |
| I-89 | 0 | PO | 211.7 ± 14.6 | 115.7 ± 20.6 | |
| | 60 | | 211.3 ± 13.7 | 178.7 ± 19.6 | P < 0.05 |
| I-70 | 0 | PO | 199 ± 18.9 | 111.7 ± 19.7 | |
| | 60 | | 200.7 ± 16.9 | 195.7 ± 16.4 | P < 0.01 |
| I-111 | 0 | PO | 207 ± 14.7 | 106.7 ± 10.4 | |
| | 60 | | 207.66 ± 13.3 | 140.7 ± 13.7 | |
| I-78 | 0 | PO | 207 ± 14.7 | 106.7 ± 10.4 | |
| | 60 | | 207.3 ± 14.6 | 181.7 ± 22.3 | P < 0.05 |
| I-124 | 0 | PO | 207 ± 14.7 | 106.7 ± 10.4 | |
| | 60 | | 207.3 ± 14.4 | 196.7 ± 27.3 | P < 0.01 |
| I-36 | 0 | PO | 209.3 ± 8.5 | 102.8 ± 9.3 | |
| | 3 | | 219 ± 12.0 | 114.3 ± 11.8 | |
| | 10 | | 203 ± 12.7 | 90.7 ± 10.4 | |
| | 30 | | 219 ± 11.9 | 157.3 ± 11.4 | P < 0.05 |
| | 56 | | 205 ± 13.1 | 191 ± 12.1 | P < 0.01 |

While a number of embodiments of this invention have been described, it is apparent that the examples may be altered to provide other embodiments of this invention. Therefore, it will be appreciated that the scope of this invention is to be defined by the appended claims rather than by the specific embodiments which have been represented by way of example.

1. A compound of formula I 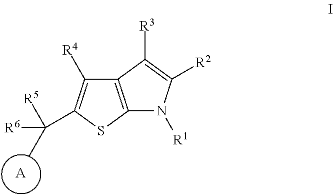
wherein
R¹ is V—R⁸;
V is a covalent bond between R⁸ and the nitrogen to which V is bonded, or is a divalent linker between R⁸ and the nitrogen to which V is bonded, wherein said linker is a saturated or unsaturated C₁₋₆ aliphatic which is optionally substituted with up to 6 instances of halogen, a C₁₋₄ aliphatic, —OR¹⁴, —CN, —SR¹⁴, —CO₂R¹⁴, —OC(O)R¹⁴, —C(O)N(R¹⁴)₂, —N(R)C(O)R¹⁴, —N(R)C(O)OR¹⁴, —OC(O)N(R¹⁴H)₂, —N(R)C(O)NR¹⁴ or —N(R¹⁴H)₂; wherein said C₁₋₄ aliphatic is optionally substituted with up to 6 instances of halogen, —OR¹⁴, —CN or —N(R¹⁴H)₂; and wherein up to two saturated carbons of said C₁₋₆ aliphatic are replaced by —O—, —C(O)—, —C(S)—, —C(O)N(R)—, —N(R)C(O)—, —C(O)O—, —OC(O)—, —C(═N—N(R¹⁴H)₂)—, —C(═N—OR¹⁴H)—, —N(R)—, —N(R)S(O)₂—, —S(O)₂N(R)—, —N(R)S(O)₂N(R)—, —N(R)C(O)O—, —OC(O)N(R)—, —N(R)C(O)N(R)—, —OC(O)N(R)—, —S—, —S(O)—, —S(O)₂—, —C(O)S—, —SC(O)—, —C(S)S—, —SC(S)—, —OC(S)—, —C(S)O—, —C(S)N(R)—, —N(R)C(S)—, —N(R)C(S)S—, —SC(S)N(R)—, —N(R)C(S)O—, —N(R)C(O)S—, —OC(S)N(R)— or —SC(O)N(R)—;
each occurrence of R is independently selected from hydrogen, a C₁₋₄ aliphatic or a C₁₋₄ haloaliphatic;
each occurrence of R¹⁴ is independently selected from hydrogen, a C₁₋₄ aliphatic, a C₁₋₄ haloaliphatic, a C₃₋₇ cycloaliphatic or a 3-7 membered heterocyclyl; or two R¹⁴ attached to the same or different atom(s), together with the atom(s) to which they are attached, form a C₃₋₇ cycloaliphatic or 3-7 membered heterocycle; wherein said cycloaliphatic and heterocyclyl are optionally and independently substituted by up to 6 instances of halogen, —CN, oxo, C₁₋₄ alkyl, C₁₋₄ haloalkyl, C₁₋₄ alkoxy or C₁₋₄ haloalkoxy;
R⁸ is hydrogen, halogen, —NO₂, —CN, a C₁-C₆ aliphatic, a 5-14 membered aryl group, a 5-14 membered heteroaryl group, a C₃-C₁₂ cycloaliphatic group, or a 3-14 membered heterocyclyl group; wherein said aliphatic, aryl, heteroaryl, cycloaliphatic and heterocyclyl group are optionally and independently substituted with up to 6 instances of R¹⁵;
each occurrence of R¹⁵ is independently selected from halogen, oxo, —NO₂, —CN, —OR¹⁷, —SR¹⁷, —S(O)₂R¹⁷, —SO₂N(R¹⁷)₂, —S(O)R¹⁷, —N(R¹⁷)₂, —C(O)OR¹⁷, —C(O)R¹⁷, —C(O)C(O)R¹⁷, —C(═N—N(R¹⁷)₂)R¹⁷, —N(R′)C(O)R¹⁷, —N(R′)C(O)OR¹⁷, —N(R′)S(O)₂R¹⁷, —N(R′)S(O)₂N(R′)R¹⁷, —N(R′)C(O)N(R′)R¹⁷, —N(R′)N(R′)R¹⁷, —C(O)NOR¹⁷, —C(O)N(R¹⁷)₂, —OC(O)R¹⁷, —OC(O)N(R¹⁷)₂, —C(O)SR¹⁷, —SC(O)R¹⁷, —C(S)SR¹⁷, —SC(S)R¹⁷, —OC(S)R¹⁷, —C(S)OR¹⁷, —C(S)N(R′)R¹⁷, —N(R′)C(S)R¹⁷, —N(R′)C(S)SR¹⁷, —SC(S)N(R′)R¹⁷, —N(R′)C(S)OR¹⁷, —N(R′)C(O)SR¹⁷, —OC(S)N(R′)R¹⁷, —SC(O)N(R′)R¹⁷, or a C₁₋₄ aliphatic, wherein said aliphatic is optionally substituted with up to 6 instances of —R¹⁶;
each occurrence of R′ is independently selected from hydrogen, C₁₋₄ aliphatic or C₁₋₄ haloaliphatic;
each occurrence of R¹⁶ is independently selected from halogen, oxo, —OR¹⁸, —CN, —CO₂R¹⁸, —C(O)N(R¹⁸)₂, —N(R″)C(O)R¹⁸, —OC(O)N(R¹⁸)₂, —N(R″)C(O)OR¹⁸, —N(R¹⁸)C(O)N(R¹⁸)₂, —N(R¹⁸)₂, a C₃₋₇ cycloaliphatic or a 3-7 membered heterocycle; wherein said cycloaliphatic and heterocycle can be optionally and independently substituted by up to 6 instances of halogen, —CN, oxo, C₁₋₄ alkyl, C₁₋₄ haloalkyl, C₁₋₄ alkoxy or C₁₋₄ haloalkoxy;
each occurrence of R¹⁷ is independently selected from hydrogen, a C₁-C₄ aliphatic, a C₁-C₄ haloaliphatic, a C₃₋₇ cycloaliphatic or a 3-7 membered heterocyclyl; or two R¹⁷ attached to the same or different atom(s), together with the atom(s) to which they are attached, form a 3-7 membered heterocycle; wherein said cycloaliphatic and heterocyclyl are optionally and independently substituted by up to 6 instances of halogen, —CN, oxo, C₁₋₄ alkyl, C₁₋₄ haloalkyl, C₁₋₄ alkoxy or C₁₋₄ haloalkoxy;
each occurrence of R″ is independently selected from hydrogen, C₁₋₄ aliphatic or C₁₋₄ haloaliphatic;
each occurrence of R¹⁸ is independently selected from hydrogen, alkyl aryl, a C₁₋₄ aliphatic or a C₁-C₄ haloaliphatic; or two R¹⁸ attached to the same or different atom(s), together with the atom(s) to which they are attached, form a 3-7 membered heterocycle; wherein said 3-7 membered heterocyclyl ring is optionally substituted by up to 6 instances of halogen, —CN, oxo, C₁₋₄ alkyl, C₁₋₄ haloalkyl, C₁₋₄ alkoxy or C₁₋₄ haloalkoxy;
R² is hydrogen, halogen, —CN, —NO₂, a C₁₋₄ aliphatic or a C₃₋₆ cycloaliphatic, wherein said aliphatic and cycloaliphatic are optionally and independently substituted with up to 6 instances of halogen, —CN, —OH, —O(C₁₋₂ alkyl), —O(C₁₋₂ haloalkyl), C₁₋₂ alkyl or C₁₋₂ haloalkyl;
R³ is hydrogen, halogen, —CN, —NO₂, —C(O)NR^{X}, —C(O)OR^{X}, a C₁₋₄ aliphatic or a C₃₋₆ cycloaliphatic, wherein said aliphatic and cycloaliphatic are optionally and independently substituted with up to 6 instances of halogen, —CN, —OH, —O(C₁₋₂ alkyl), —O(C₁₋₂ haloalkyl), C₁₋₂ alkyl or C₁₋₂ haloalkyl;
R⁴ is hydrogen, halogen, —CN, —NO₂, —C(O)NR^{X}, a C₁₋₄ aliphatic or a C₃₋₆ cycloaliphatic, wherein said aliphatic and cycloaliphatic are independently and optionally substituted with up to 6 instances of halogen, —CN, —OH, —O(C₁₋₂ alkyl), —O(C₁₋₂ haloalkyl), —C₁₋₂ alkyl or —C₁₋₂ haloalkyl.
R⁵ is chosen from hydrogen, halogen, —CN, —OH, —C(O)OR^{X}, C₁₋₄ aliphatic, a 3-7 membered heterocyclyl, C₃₋₆ cycloaliphatic, —O—(C₁₋₄ aliphatic) or —O—(C₃₋₆ cycloaliphatic); wherein said aliphatic, cycloaliphatic, —O—(C₁₋₄ aliphatic) and —O—(C₃₋₆ cycloaliphatic) are optionally and independently substituted with up to three instances of halogen;
R⁶ is chosen from hydrogen, halogen, C₁₋₄ aliphatic, C₁₋₄ haloaliphatic, —O(C₁₋₄ aliphatic) or —O—(C₁₋₄ haloaliphatic); or
R⁵ and R⁶ are taken together to form ═O, ═S, ═NR^{W} or a 3-6 membered cycloaliphatic or heterocyclyl, wherein said cycloaliphatic or heterocyclyl is optionally and independently substituted with up to 6 instances of halogen, —CN, —OH, —O(C₁₋₄ aliphatic), —NO₂, —N(R^{Y})₂ or a C₁₋₄ aliphatic; wherein said aliphatic is optionally and independently substituted with up to 6 instances of —OR^{Y} or halogen;
R^{W} is selected from hydrogen, C₁₋₄ alkyl, C₁₋₄ haloalkyl, C₃₋₆ cycloalkyl, —O(C₁₋₄ alkyl), —O(C₁₋₄ haloalkyl), —N(C₁₋₄ alkyl)₂ haloalkyl)₂ or —O(aryl); wherein said aryl is optionally substituted by up to 6 instances of halogen, —NO₂, C₁₋₄ alkyl, C₁₋₄ haloalkyl, —O(C₁₋₄ alkyl) or —O(C₁₋₄ haloalkyl);
each occurrence of R^{X} and R^{Y} are independently selected from hydrogen or a C₁₋₄ aliphatic, wherein said aliphatic is optionally and independently substituted with up to three instances of halogen, OR^{iv} or N(R^{iv})₂;
each occurrence of R^{iv} is independently selected from hydrogen, C₁₋₄ aliphatic or C₁₋₄ haloaliphatic;
ring A is phenyl or a 5-6 membered monocyclic heteroaryl having 1-3 heteroatoms independently selected from N, O or S; wherein said phenyl and heteroaryl are optionally and independently substituted with up to five instances of R¹²;
each occurrence of R¹² is independently selected from halogen, —NO₂, —CN, a C₁₋₄ aliphatic optionally substituted with up to four instances of R¹⁹, —OR¹³, —SR¹³, —S(O)₂R¹³, —SO₂N(R¹³)₂, —S(O)R¹³, —N(R¹³)₂, —C(O)OR¹³, —C(O)R¹³, —C(O)C(O)R¹³, —C(═N—N(R¹³)₂)R¹³, —N(R¹³)C(O)R¹³, —N(R¹³)C(O)OR¹³, —N(R¹³)C(S)R¹³, —N(R¹³)C(S)OR¹³, —N(R¹³)S(O)₂R¹³, —N(R¹³)S(O)₂N(R¹³)R¹³, —N(R¹³)C(O)N(R¹³)R¹³, —N(R¹³)C(S)N(R¹³)R¹³, —N(R¹³)N(R¹³)R¹³, —C(O)NOR¹³, —C(O)N(R¹³)₂, —C(O)N(R¹³)N(R¹³)₂, —C(S)N(R¹³)₂, —C(S)(R¹³), —C(S)OR¹³, —C(S)N(R¹³)N(R¹³)₂, —OC(O)R¹³ or —OC(O)N(R¹³)₂; or two R¹² attached to different atom(s), together with the atom(s) to which they are attached, form a 3-7 membered heterocycle or C₃₋₇ cycloaliphatic; wherein said cycloaliphatic and heterocycle are independently and optionally substituted with up to 6 instances of halogen, —CN, —OH, C₁₋₂ alkyl, C₁₋₂ haloalkyl, C₁₋₂ alkoxy or C₁₋₂ haloalkoxy;
each R¹³ is independently selected from hydrogen, a C₁-C₄ aliphatic, a C₃₋₇ cycloaliphatic or a 3-7 membered heterocyclyl, wherein said aliphatic, cycloaliphatic and heterocyclyl are independently and optionally substituted with up to 6 instances of halogen; or two R¹³ attached to the same or different atom(s), together with the atom(s) to which they are attached, form a 3-7 membered heterocycle; wherein said heterocycle is optionally substituted with up to 6 instances of halogen, —CN, —OH, C₁₋₂ alkyl, C₁₋₂ haloalkyl, C₁₋₂ alkoxy or C₁₋₂ haloalkoxy;
each occurrence of R¹⁹ is independently selected from halogen, —OR²⁰, —NO₂, —CN, —CO₂R²⁰, —C(O)N(R²⁰)₂, —N(R²⁰)C(O)R²⁰, —N(R²⁰C(O)OR²⁰, —N(R²⁰)₂, a C₃₋₇ cycloaliphatic, a 3-7 membered heterocyclyl, a C₁₋₄ aliphatic or a C₁₋₄ haloaliphatic; wherein said cycloaliphatic and heterocyclyl are optionally and independently substituted with up to 6 instances of halogen, —CN, —OH, C₁₋₂ alkyl, C₁₋₂ haloalkyl, C₁₋₂ alkoxy or C₁₋₂ haloalkoxy; and
each occurrence of R²⁰ is independently selected from hydrogen, a C₁₋₄ aliphatic or a C₁₋₄ haloaliphatic; or two R²⁰ attached to the same or different atom(s), together with the atom(s) to which they are attached, form a 3-7 membered heterocycle, wherein said heterocycle is optionally substituted with up to 6 instances of halogen, —CN, —OH, C₁₋₂ alkyl, C₁₋₂ haloalkyl, C₁₋₂ alkoxy or C₁₋₂ haloalkoxy.

2. The compound according to claim 1, wherein V is a bond or a C₁₋₆ aliphatic, wherein up to two saturated carbons of said C₁₋₆ aliphatic are replaced by —O—, —C(O)—, —C(O)N(R)—, —OC(O)—, —C(O)O—, —N(R)—, —N(R)C(O)—, —N(R)S(O)₂—, —N(R)S(O)₂N(R)—, —S(O)₂— or —S(O)₂N(R)—.

3. The compound according to claim 2, wherein V is a bond, methylene, ethylene, propylene, butylene or pentylene, wherein up to two carbons of said said methylene, ethylene, propylene, butylene or pentylene are replaced by —O—, —C(O)—, —C(O)N(R)—, —C(O)O—, —N(R)—, —N(R)C(O)—, —N(R)S(O)₂—, —N(R)S(O)₂N(R)—, —S(O)₂— or —S(O)₂N(R)—.

4. The compound according to claim 3, wherein V is a bond.

5. The compound according to claim 3, wherein V is methylene, ethylene, propylene, butylene or pentylene.

6. The compound according to claim 3, wherein —V—R⁸ is selected from the group consisting of: 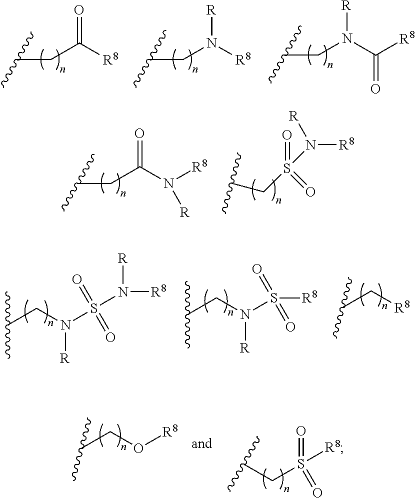
wherein n is an integer selected from 0, 1, 2, 3 or 4.

7. The compound according to claim 1, wherein R⁸ is independently selected from hydrogen, halogen, —NO₂, —CN, a C₁-C₆ aliphatic, a 5-14 membered aryl group, a 5-14 membered heteroaryl group, a C₃₋₁₂ cycloaliphatic group, or a 3-14 membered heterocyclyl group, wherein said C₁-C₆ aliphatic or said aryl, heteroaryl, cycloaliphatic or heterocyclyl is optionally and independently substituted with up to four instances of halogen, oxo, —NO₂, —CN, C₁₋₄ aliphatic, —OR¹⁷, —C(O)R¹⁷, —C(O)OR¹⁷ or —C(O)N(R′)R¹⁷; wherein said C₁₋₄ aliphatic is optionally substituted with up to four instances of —R¹⁶.

8. The compound according to claim 7, wherein R⁸ is independently selected from hydrogen, halogen, —NO₂, —CN or a C₁-C₆ aliphatic, wherein said C₁-C₆ aliphatic is optionally and independently substituted with up to four instances of halogen, oxo, —CN or C₁₋₄ aliphatic, C₁₋₄ haloaliphatic, C₁₋₄ alkoxy, C₁₋₄ haloalkoxy or —C(O)OR¹⁷.

9. The compound according to claim 8, wherein R⁸ is independently selected from hydrogen, fluoride, chloride, methyl, ethyl, propyl, butyl, vinyl, isopropyl, t-butyl, methoxymethyl, methoxy, isopropoxy, ethoxy, —C(OH)(CH₃)₂, trifluoromethyl, trifluoromethoxy or —CO₂H.

10. The compound according to claim 7, wherein R⁸ is independently selected from a 5-14 membered aryl group, or a 5-14 membered heteroaryl group, wherein said aryl or heteroaryl is optionally and independently substituted with up to four instances of halogen, oxo, —NO₂, —CN, —OR¹⁷, —C(O)OR¹⁷ or a C₁₋₆ alkyl, said alkyl being optionally substituted with up to 6 instances of —R¹⁶.

11. The compound according to claim 10, wherein R⁸ is independently selected from the group consisting of:

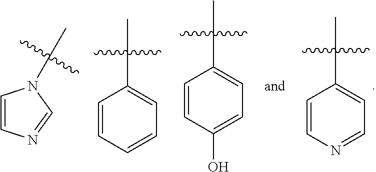

12. The compound according to claim 7, wherein R⁸ is independently selected from a 3-12 membered cycloaliphatic group or a 3-14 membered heterocyclyl group, wherein each of said cycloaliphatic or heterocyclic ring is optionally and independently substituted with up to four instances of halogen, oxo, —CN, a C₁₋₄ aliphatic, a C₁₋₄ haloaliphatic, C₁₋₄ alkoxy or C₁₋₄ haloalkoxy.

13. The compound according to claim 12, wherein R⁸ is independently selected from the group consisting of: 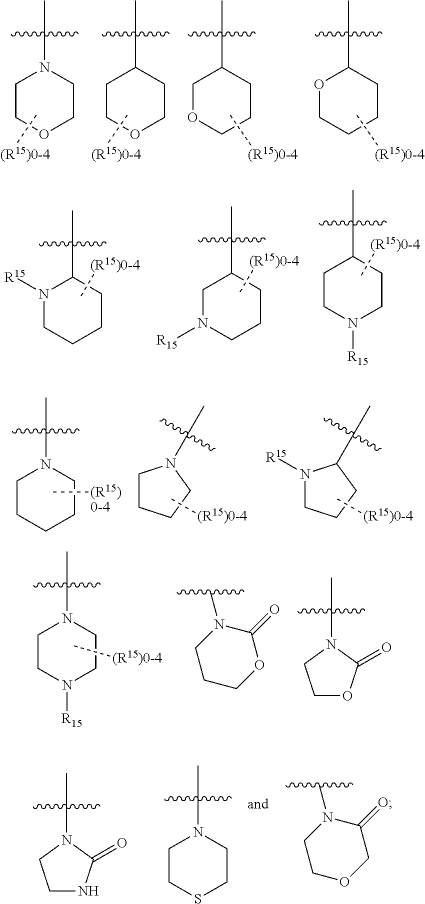
wherein R¹⁵ on a carbon atom is an optional substituent selected from halogen, —CN, C₁₋₄ alkyl, C₁₋₄ haloalkyl, C₁₋₄ alkoxy, C₁₋₄ haloalkoxy, —SO₂(C₁₋₄ alkyl) or —C(O)OR¹⁷; and
R¹⁵ on a nitrogen atom is an optional substituent selected from C₁₋₄ alkyl, C₁₋₄ haloalkyl, C₁₋₄ alkoxy, C₁₋₄ haloalkoxy, —SO₂(C₁₋₄ alkyl) or —C(O)OR¹⁷.

14. The compound according to claim 13, wherein R⁸ is independently selected from the group consisting of: 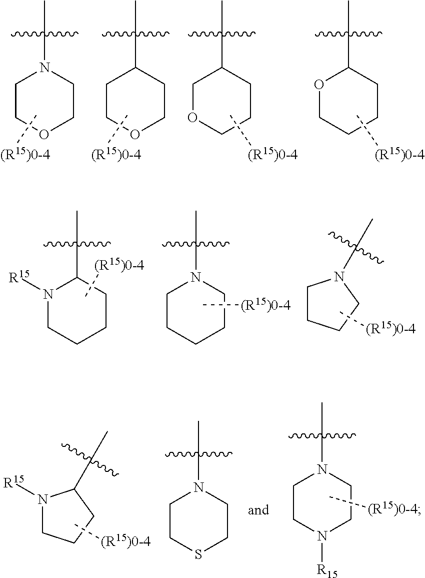
wherein R¹⁵ on a carbon atom is an optional substituent selected from halogen, C₁₋₄ alkyl, C₁₋₄ haloalkyl, C₁₋₄ alkoxy, C₁₋₄ haloalkoxy, —SO₂(C₁₋₄ alkyl) or —C(O)OR¹⁷; and
R¹⁵ on a nitrogen atom is an optional substituent selected from C₁₋₄ alkyl, C₁₋₄ haloalkyl, C₁₋₄ alkoxy, C₁₋₄ haloalkoxy, —SO₂(C₁₋₄ alkyl) or —C(O)OR¹⁷.

15. The compound according to claim 14, wherein R⁸ is independently selected from the group consisting of: 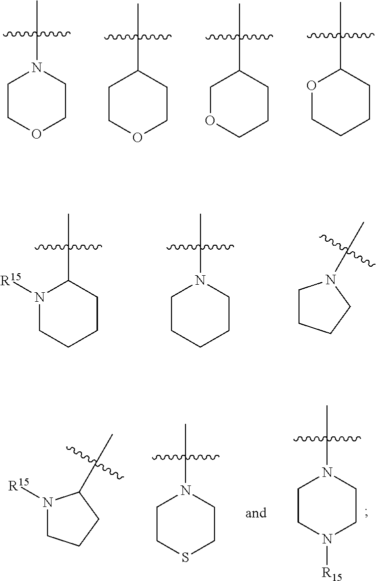
wherein R¹⁵ on a N atom is an optional substituent selected from C₁₋₄-alkyl or C₁₋₄ haloalkyl.

16. The compound according to claim 1, wherein R² is hydrogen, halogen or a C₁₋₄ aliphatic; wherein said C₁₋₄ aliphatic is optionally substituted with up to six instances of halogen, —O(C₁₋₂ alkyl), —O(C₁₋₂ haloalkyl), C₁₋₂ alkyl or C₁₋₂ haloalkyl.

17. The compound according to claim 16, wherein R² is a C₁₋₄ aliphatic.

18. The compound according to claim 17 wherein R² is methyl.

19. The compound according to claim 1, wherein R³ is hydrogen, halogen, —C(O)OR^{X} or a C₁₋₄ aliphatic; said aliphatic being optionally substituted with up to six instances of halogen, —O(C₁₋₂ alkyl), —O(C₁₋₂ haloalkyl), C₁₋₂ alkyl or C₁₋₂ haloalkyl.

20. The compound according to claim 19, wherein R³ is hydrogen or halogen.

21. The compound according to claim 20, wherein R³ is chloro or fluoro.

22. The compound according to claim 21, wherein R³ is chloro.

23. The compound according to claim 20, wherein R³ is hydrogen.

24. The compound according to claim 1, wherein R⁴ is hydrogen, halogen or a C₁₋₄ aliphatic optionally substituted with up to six instances of halogen, —O(C₁₋₂ alkyl), —O(C₁₋₂ haloalkyl), C₁₋₂ alkyl or C₁₋₂ haloalkyl.

25. The compound according to claim 24, wherein R⁴ is hydrogen or halogen.

26. The compound according to claim 25, wherein R⁴ is hydrogen.

27. The compound according to claim 1, wherein ring A is phenyl or a 5-6 membered monocyclic heteroaryl having 1-3 heteroatoms independently selected from N, O or S, wherein said phenyl or heteroaryl is optionally substituted with up to five instances of R¹².

28. The compound according to claim 1, wherein ring A is phenyl or a 6-membered heteroaryl ring containing up to two nitrogen atoms, wherein said phenyl or said heteroaryl ring is optionally substituted with up to three instances of chloro, fluoro, —CN, —NO₂, C₁₋₄ alkyl, C₁₋₄ haloalkyl, C₁₋₄ haloalkoxy, C₁₋₄ alkoxy, C₁₋₄ alkylthio, hydroxy, or amino; or wherein said phenyl or heteroaryl ring is fused with a 5 membered heterocycle or cycloaliphatic.

29. The compound according to claim 28, wherein ring A is benzo[d][1,3]dioxole-5-yl, 2,3-dihydrobenzofuran-7-yl or phenyl, wherein the phenyl ring is optionally substituted with up to 3 instances of chloro, fluoro, hydroxy, methyl, ethyl, methoxy, ethoxy, trifluoromethyl, trifluoromethoxy, thiomethoxy, isopropoxy, —N(C₁₋₄ alkyl)₂, or amino.

30. The compound according to claim 29, wherein ring A is phenyl, optionally substituted with up to 2 instances of chloro, fluoro, hydroxy, methyl, ethyl, methoxy, ethoxy, trifluoromethyl, trifluoromethoxy, thiomethoxy, isopropoxy, —N(C₁₋₄ alkyl)₂, or amino.

31. The compound according to claim 1, wherein R⁵ is chosen from hydrogen, halogen, —CN, —OH, —O(C₁₋₄ aliphatic), a C₁₋₄ aliphatic or a 3-7 membered heterocyclyl, wherein said C₁₋₄ aliphatic and said —O(C₁₋₄ aliphatic) of R⁵ are optionally and independently substituted with up to three instances of halogen; and R⁶ is chosen from hydrogen, halogen, C₁₋₄ aliphatic or —O(C₁₋₄ aliphatic), wherein said C₁₋₄ aliphatic of R⁶ is optionally substituted with up to three instances of halogen.

32. The compound according to claim 31, wherein R⁵ is chosen from hydrogen, halogen, —OH, —O(C₁₋₄ aliphatic) or —O(C₁₋₄ haloaliphatic); and R⁶ is chosen from hydrogen, halogen or a C₁₋₄ aliphatic, optionally substituted with up to three instances of halogen.

33. The compound according to claim 32, wherein R⁵ is hydrogen or fluoro and R⁶ is hydrogen or fluoro.

34. The compound according to claim 32, wherein R⁵ is —OH and R⁶ is hydrogen.

35. The compound according to claim 32, wherein R⁵ is hydrogen, —OH, —O(C₁₋₄ alkyl) or —O(C₁₋₄ haloalkyl) and R⁶ is C₁₋₄ alkyl or C₁₋₄ haloalkyl.

36. The compound according to claim 1, wherein said compound is of formula I-A, or is a pharmaceutically acceptable salt thereof: 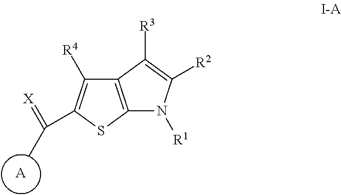
wherein X is O, S or NH; and Ring A, R¹, R², R³ and R⁴ are defined as in claim 1.

37. The compound according to claim 36, wherein X is O.

38. The compound according to claim 37, wherein said compound is of formula I-B, or is a pharmaceutically acceptable salt thereof: 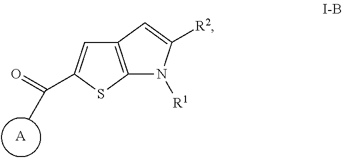
wherein Ring A, R¹ and R² are defined as in claim 1.

39. The compound according to claim 38, wherein said compound is of formula I-C, or is a pharmaceutically acceptable salt thereof: 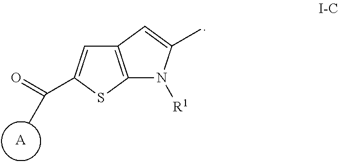
wherein Ring A and R¹ are defined as in claim 1.

40. The compound according to claim 36, wherein said compound is of formula I-D: 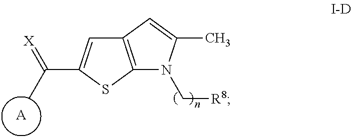
or is a pharmaceutically acceptable salt thereof, wherein
n is selected from 0, 1, 2 or 3; and R⁸ is defined as in claim 1.

41. The compound according to claim 40, wherein n is selected from 2 or 3.

42. The compound according to claim 40, wherein X is O.

43. The compound according to claim 40, wherein R⁸ is a 5-8 membered heterocyclyl, optionally substituted with up to 6 instances of R¹⁵.

44. The compound according to claim 43, wherein R⁸ is tetrahydropyran, morpholinyl, thiomorpholinyl, piperidinyl, pyrrolidinyl, piperazinyl or oxazolidinyl; wherein each is optionally substituted with up to 6 instances of oxo, C₁₋₂ alkyl or C₁₋₂ haloalkyl.

45. The compound according to claim 43, wherein R⁸ is selected from the group consisting of: 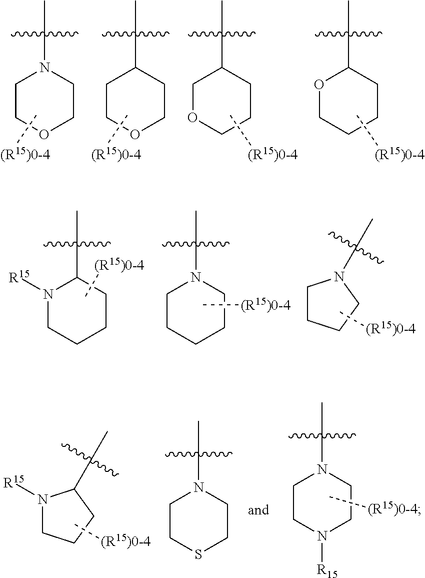
wherein R¹⁵ on a carbon atom is an optional substituent selected from halogen, C₁₋₄ alkyl, C₁₋₄ haloalkyl, C₁₋₄ alkoxy, C₁₋₄ haloalkoxy, —SO₂(C₁₋₄ alkyl) or —C(O)OR¹⁷; and
R¹⁵ on a nitrogen atom is an optional substituent selected from C₁₋₄ alkyl, C₁₋₄ haloalkyl, C₁₋₄ alkoxy, C₁₋₄ haloalkoxy, —SO₂(C₁₋₄ alkyl) or —C(O)OR¹⁷.

46. The compound according to claim 45, wherein R⁸ is selected from the group consisting of: 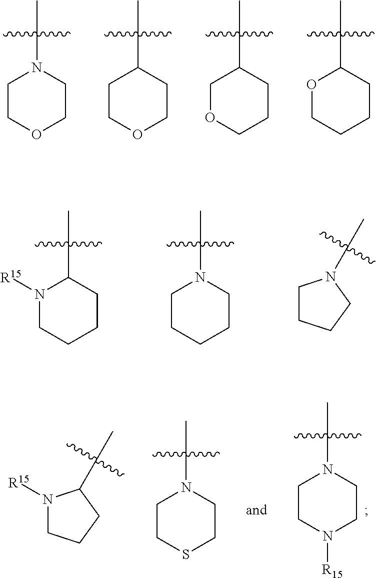
wherein R¹⁵ is C₁₋₄-alkyl or C₁₋₄ haloalkyl.

47. The compound according to claim 40, wherein R⁸ is C₁-C₆ aliphatic, wherein said aliphatic is optionally and independently substituted with up to four instances of R¹⁵.

48. The compound according to claim 1, wherein the compound is selected from the group consisting of:

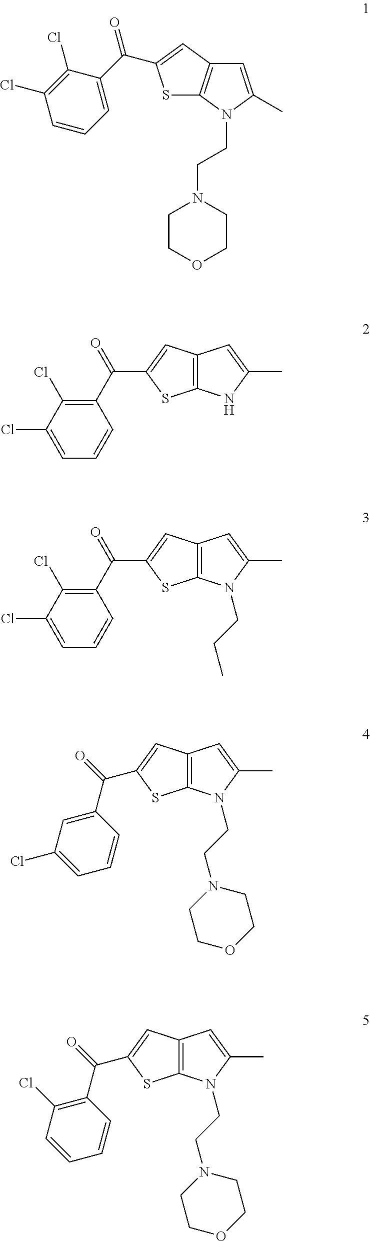 [SMILES: Cc1cc2cc(C(=O)c3cccc(Cl)c3Cl)sc2n1CCN1CCOCC1 ; Cc1cc2cc(C(=O)c3cccc(Cl)c3Cl)sc2[nH]1 ; CCCn1c(C)cc2cc(C(=O)c3cccc(Cl)c3Cl)sc21 ; Cc1cc2cc(C(=O)c3cccc(Cl)c3)sc2n1CCN1CCOCC1 ; Cc1cc2cc(C(=O)c3ccccc3Cl)sc2n1CCN1CCOCC1]

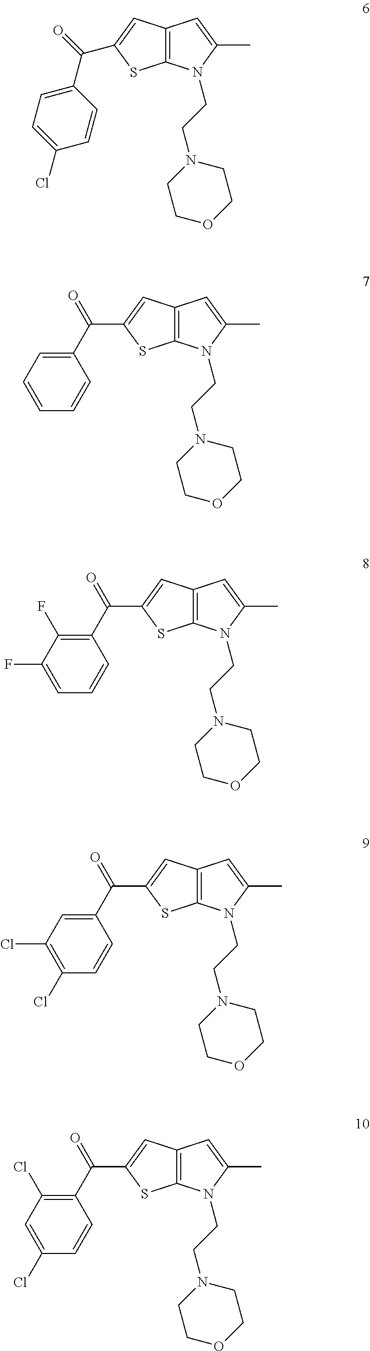 [SMILES: Cc1cc2cc(C(=O)c3ccc(Cl)cc3)sc2n1CCN1CCOCC1 ; Cc1cc2cc(C(=O)c3ccccc3)sc2n1CCN1CCOCC1 ; Cc1cc2cc(C(=O)c3cccc(F)c3F)sc2n1CCN1CCOCC1 ; Cc1cc2cc(C(=O)c3ccc(Cl)c(Cl)c3)sc2n1CCN1CCOCC1 ; Cc1cc2cc(C(=O)c3ccc(Cl)cc3Cl)sc2n1CCN1CCOCC1]

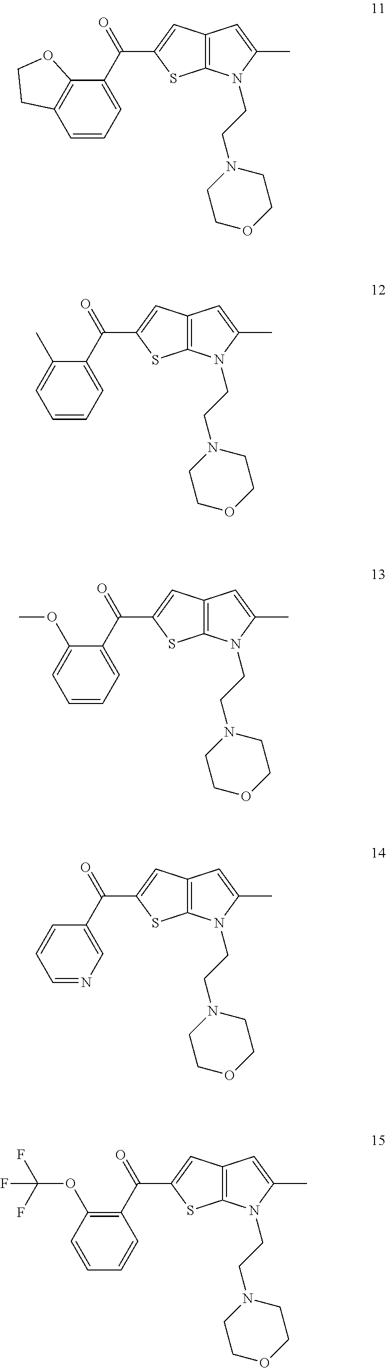

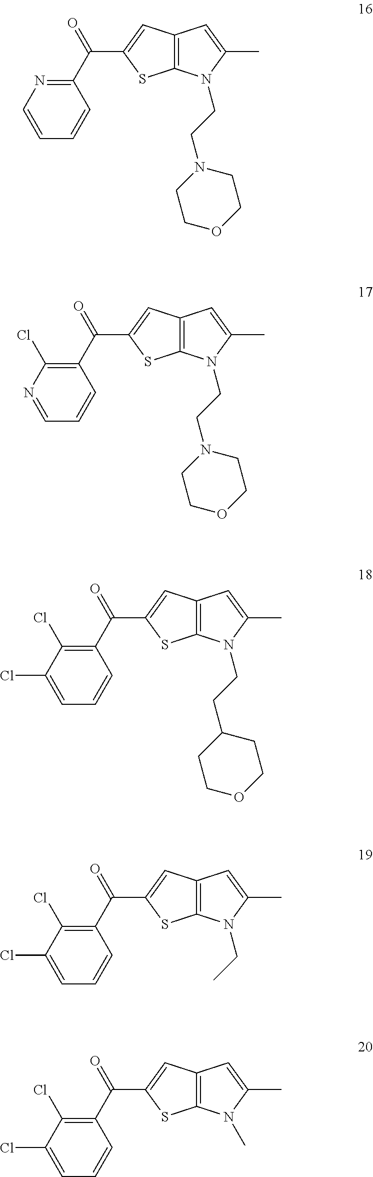 [SMILES: Cc1cc2cc(C(=O)c3ccccn3)sc2n1CCN1CCOCC1 ; Cc1cc2cc(C(=O)c3cccnc3Cl)sc2n1CCN1CCOCC1 ; Cc1cc2cc(C(=O)c3cccc(Cl)c3Cl)sc2n1CCC1CCOCC1 ; CCn1c(C)cc2cc(C(=O)c3cccc(Cl)c3Cl)sc21 ; Cc1cc2cc(C(=O)c3cccc(Cl)c3Cl)sc2n1C]

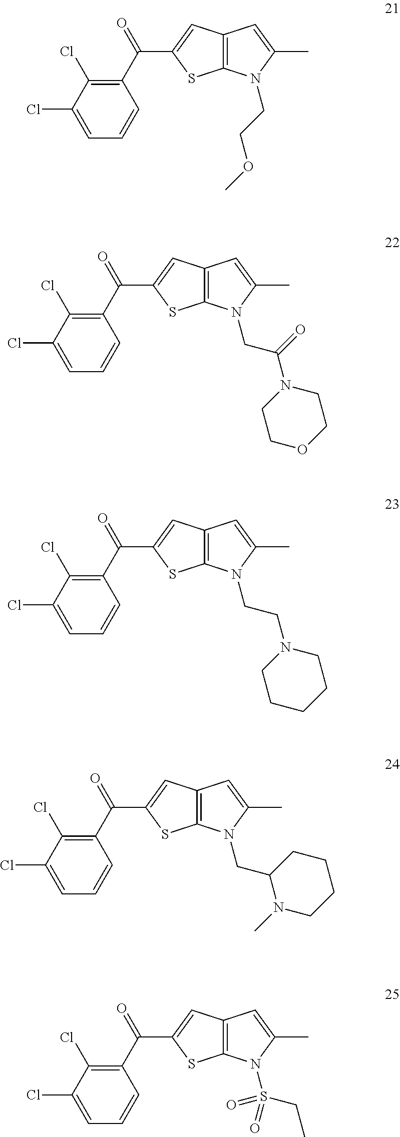 [SMILES: COCCn1c(C)cc2cc(C(=O)c3cccc(Cl)c3Cl)sc21 ; Cc1cc2cc(C(=O)c3cccc(Cl)c3Cl)sc2n1CC(=O)N1CCOCC1 ; Cc1cc2cc(C(=O)c3cccc(Cl)c3Cl)sc2n1CCN1CCCCC1 ; Cc1cc2cc(C(=O)c3cccc(Cl)c3Cl)sc2n1CC1CCCCN1C ; CCS(=O)(=O)n1c(C)cc2cc(C(=O)c3cccc(Cl)c3Cl)sc21]

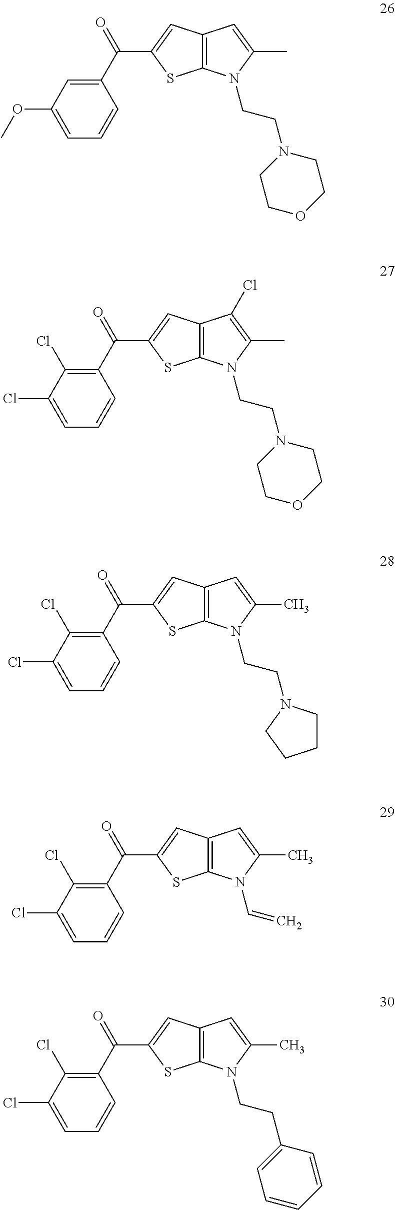 [SMILES: COc1cccc(C(=O)c2cc3cc(C)n(CCN4CCOCC4)c3s2)c1 ; Cc1c(Cl)c2cc(C(=O)c3cccc(Cl)c3Cl)sc2n1CCN1CCOCC1 ; Cc1cc2cc(C(=O)c3cccc(Cl)c3Cl)sc2n1CCN1CCCC1 ; C=Cn1c(C)cc2cc(C(=O)c3cccc(Cl)c3Cl)sc21 ; Cc1cc2cc(C(=O)c3cccc(Cl)c3Cl)sc2n1CCc1ccccc1]

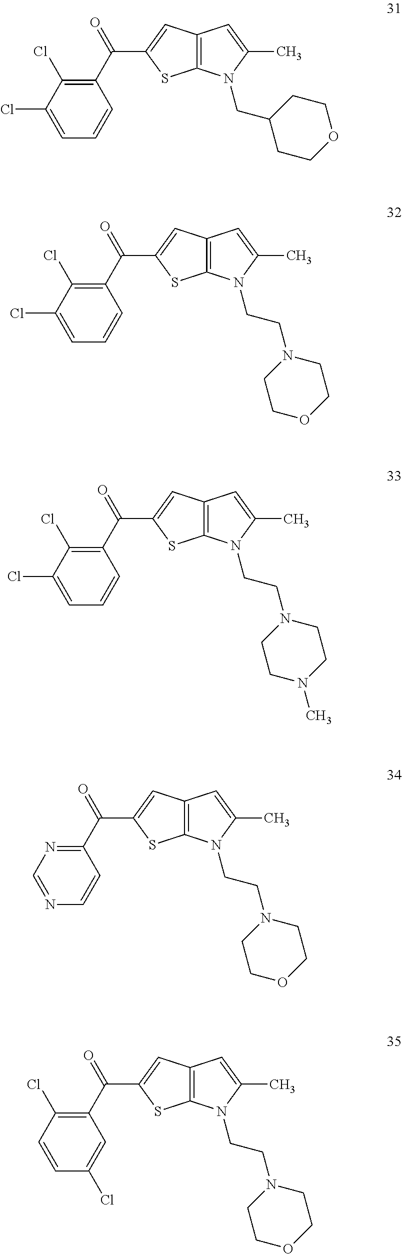 [SMILES: Cc1cc2cc(C(=O)c3cccc(Cl)c3Cl)sc2n1CC1CCOCC1 ; Cc1cc2cc(C(=O)c3cccc(Cl)c3Cl)sc2n1CCN1CCOCC1 ; Cc1cc2cc(C(=O)c3cccc(Cl)c3Cl)sc2n1CCN1CCN(C)CC1 ; Cc1cc2cc(C(=O)c3ccncn3)sc2n1CCN1CCOCC1 ; Cc1cc2cc(C(=O)c3cc(Cl)ccc3Cl)sc2n1CCN1CCOCC1]

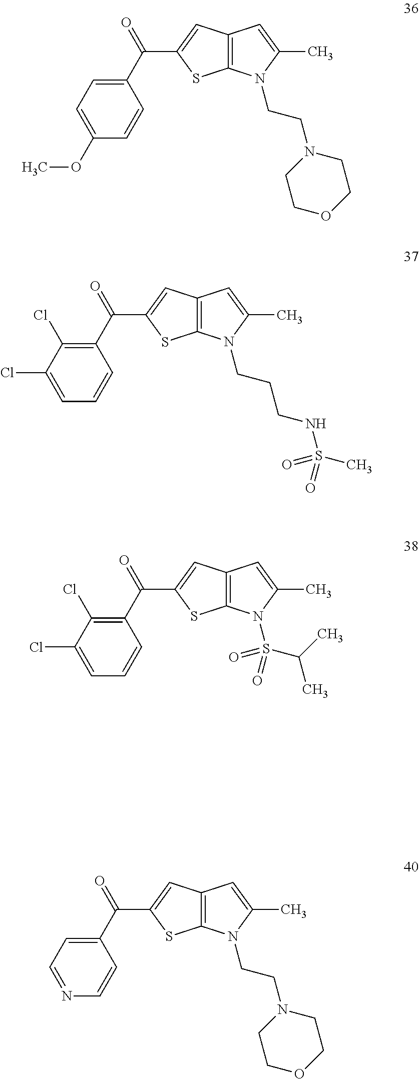 [SMILES: COc1ccc(C(=O)c2cc3cc(C)n(CCN4CCOCC4)c3s2)cc1 ; Cc1cc2cc(C(=O)c3cccc(Cl)c3Cl)sc2n1CCCNS(C)(=O)=O ; Cc1cc2cc(C(=O)c3cccc(Cl)c3Cl)sc2n1S(=O)(=O)C(C)C ; Cc1cc2cc(C(=O)c3ccncc3)sc2n1CCN1CCOCC1]

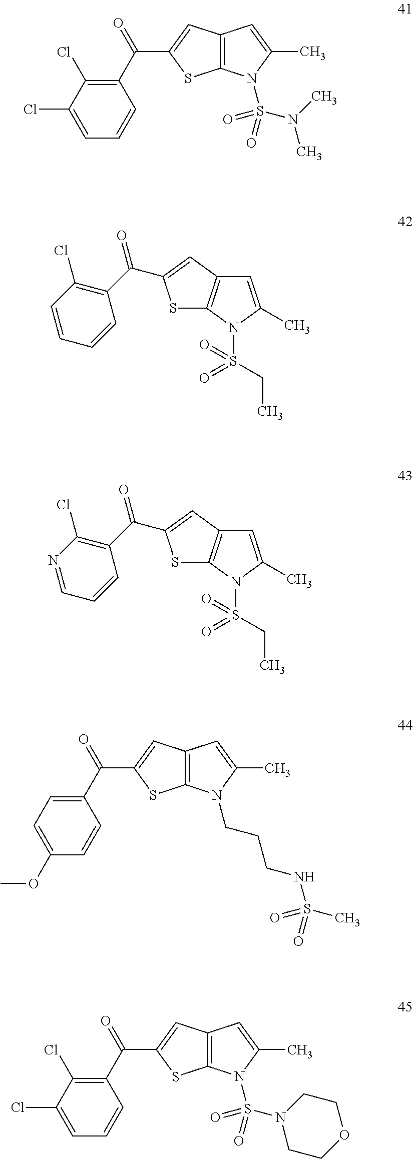

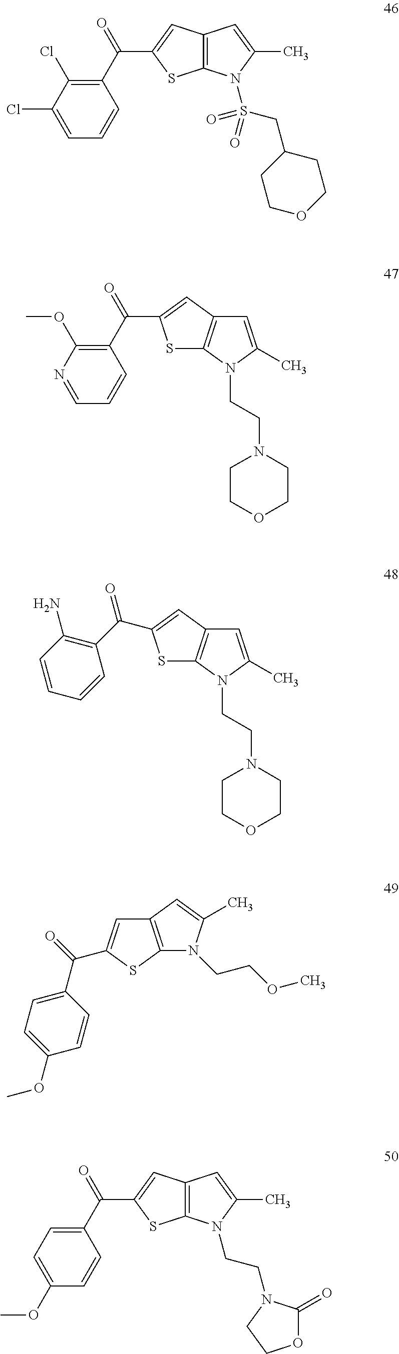

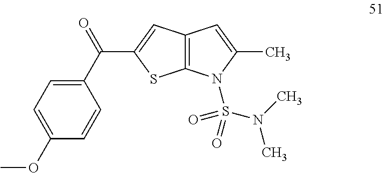

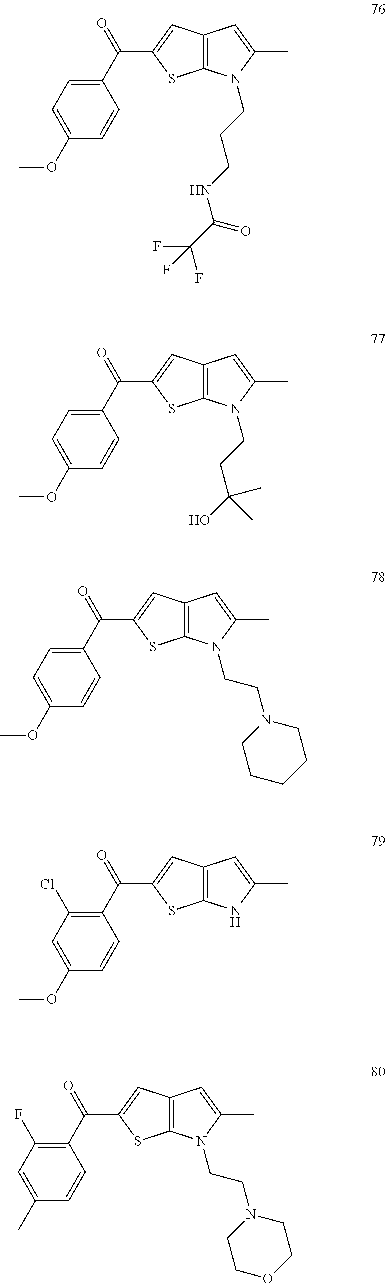

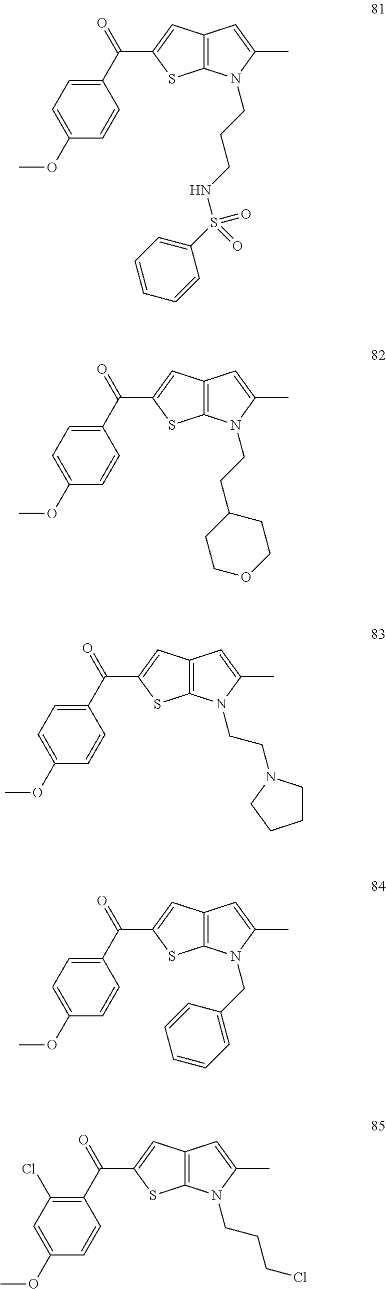

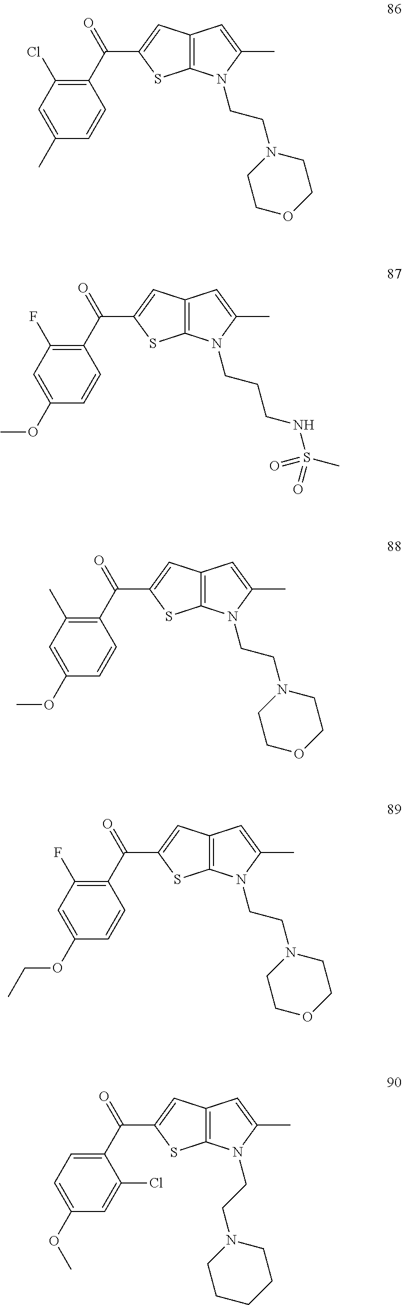

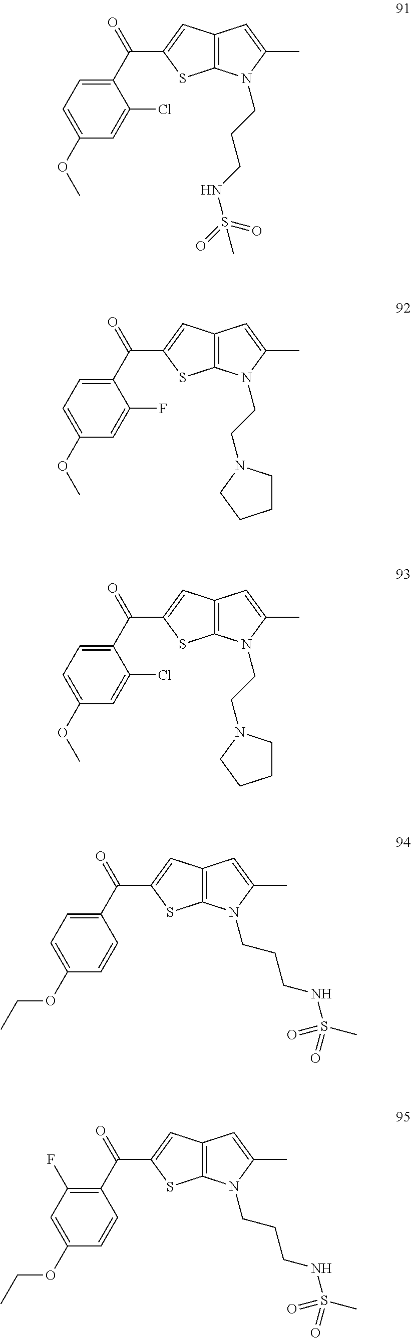

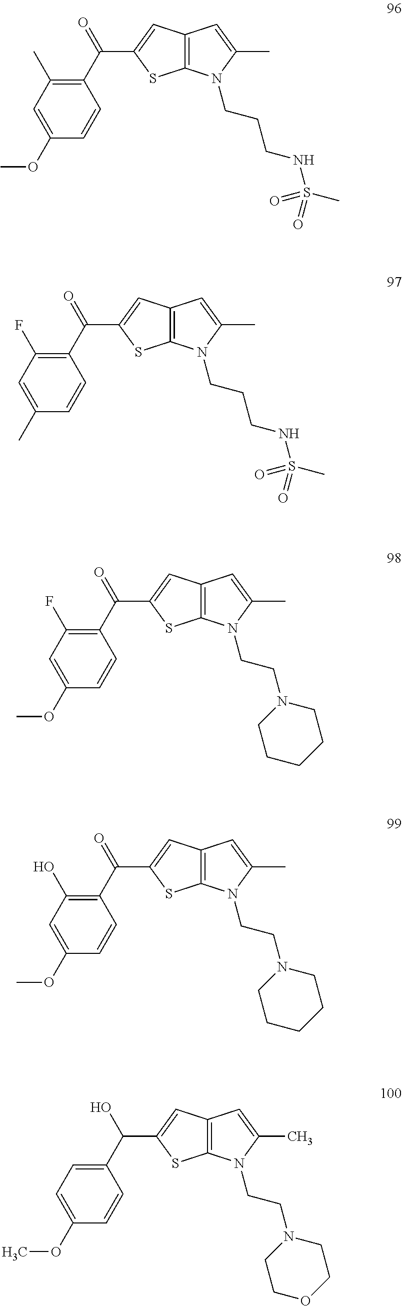

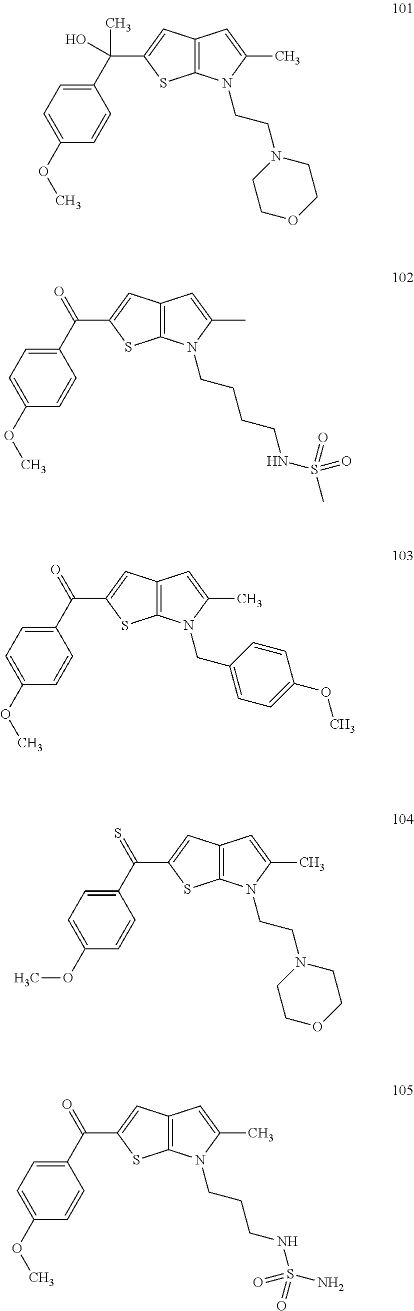

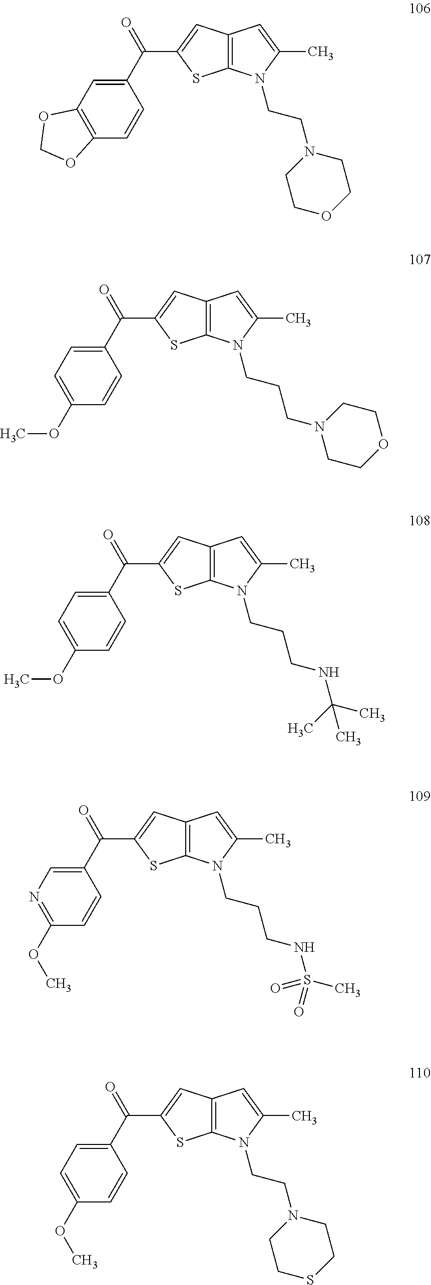 [SMILES: Cc1cc2cc(C(=O)c3ccc4c(c3)OCO4)sc2n1CCN1CCOCC1 ; COc1ccc(C(=O)c2cc3cc(C)n(CCCN4CCOCC4)c3s2)cc1 ; COc1ccc(C(=O)c2cc3cc(C)n(CCCNC(C)(C)C)c3s2)cc1 ; COc1ccc(C(=O)c2cc3cc(C)n(CCCNS(C)(=O)=O)c3s2)cn1 ; COc1ccc(C(=O)c2cc3cc(C)n(CCN4CCSCC4)c3s2)cc1]

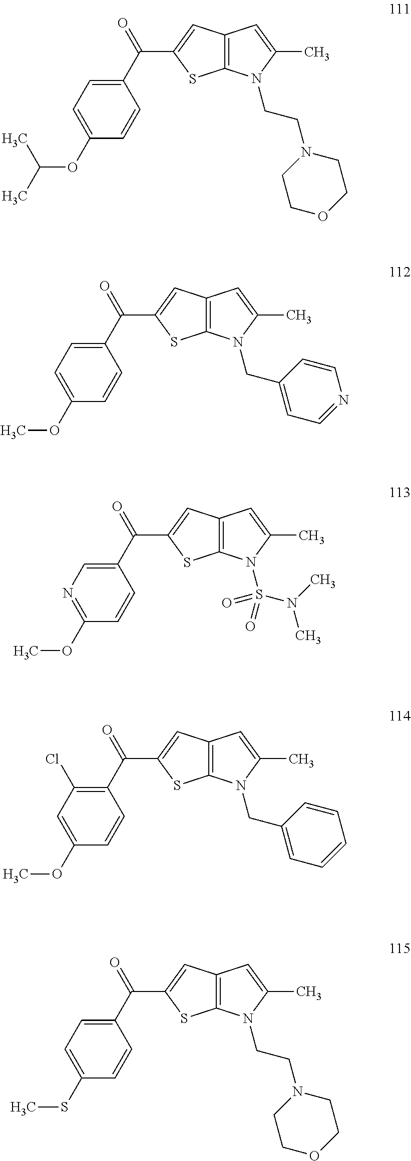

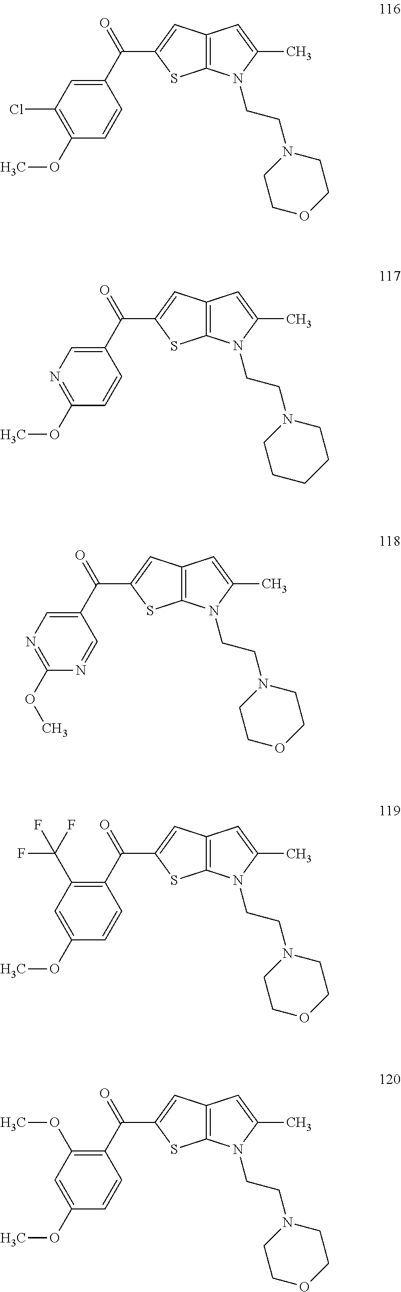 [SMILES: COc1ccc(C(=O)c2cc3cc(C)n(CCN4CCOCC4)c3s2)cc1Cl ; COc1ccc(C(=O)c2cc3cc(C)n(CCN4CCCCC4)c3s2)cn1 ; COc1ncc(C(=O)c2cc3cc(C)n(CCN4CCOCC4)c3s2)cn1 ; COc1ccc(C(=O)c2cc3cc(C)n(CCN4CCOCC4)c3s2)c(C(F)(F)F)c1 ; COc1ccc(C(=O)c2cc3cc(C)n(CCN4CCOCC4)c3s2)c(OC)c1]

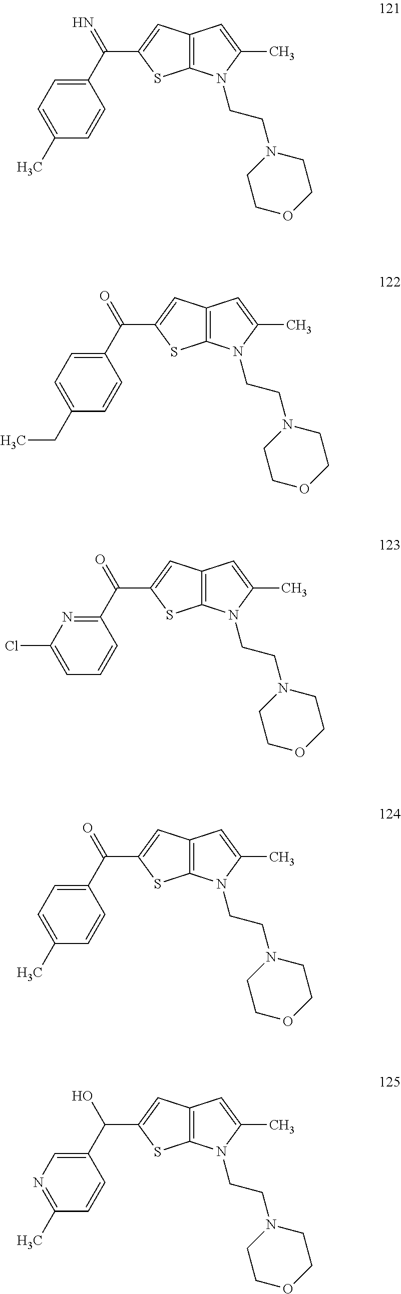 [SMILES: Cc1ccc(C(=N)c2cc3cc(C)n(CCN4CCOCC4)c3s2)cc1 ; CCc1ccc(C(=O)c2cc3cc(C)n(CCN4CCOCC4)c3s2)cc1 ; Cc1cc2cc(C(=O)c3cccc(Cl)n3)sc2n1CCN1CCOCC1 ; Cc1ccc(C(=O)c2cc3cc(C)n(CCN4CCOCC4)c3s2)cc1 ; Cc1ccc(C(O)c2cc3cc(C)n(CCN4CCOCC4)c3s2)cn1]

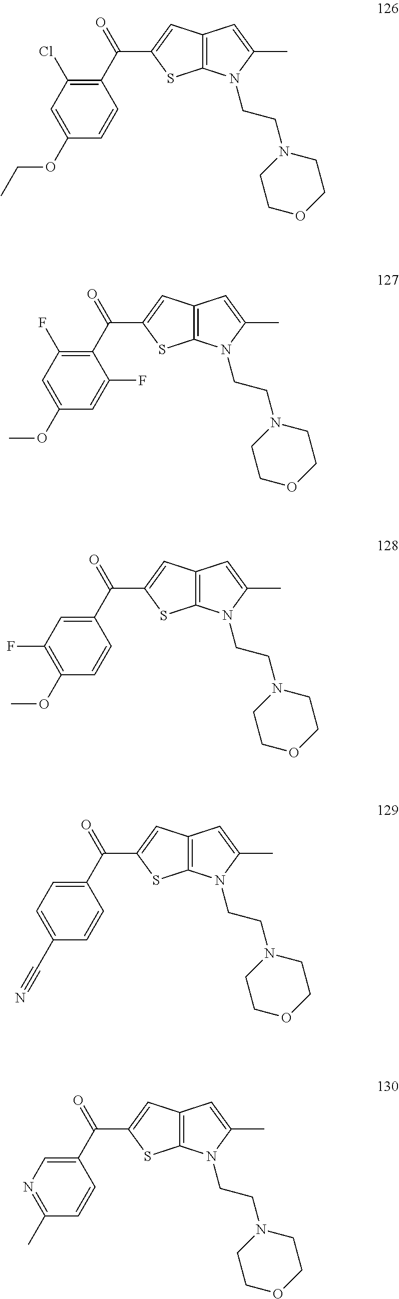 [SMILES: CCOc1ccc(C(=O)c2cc3cc(C)n(CCN4CCOCC4)c3s2)c(Cl)c1 ; COc1cc(F)c(C(=O)c2cc3cc(C)n(CCN4CCOCC4)c3s2)c(F)c1 ; COc1ccc(C(=O)c2cc3cc(C)n(CCN4CCOCC4)c3s2)cc1F ; Cc1cc2cc(C(=O)c3ccc(C#N)cc3)sc2n1CCN1CCOCC1 ; Cc1ccc(C(=O)c2cc3cc(C)n(CCN4CCOCC4)c3s2)cn1]

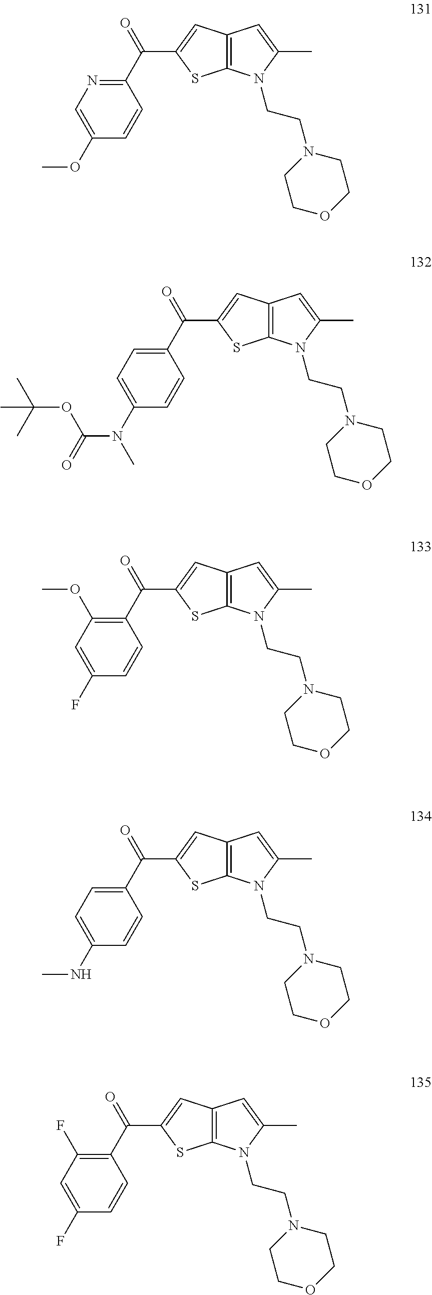 [SMILES: COc1ccc(C(=O)c2cc3cc(C)n(CCN4CCOCC4)c3s2)nc1 ; Cc1cc2cc(C(=O)c3ccc(N(C)C(=O)OC(C)(C)C)cc3)sc2n1CCN1CCOCC1 ; COc1cc(F)ccc1C(=O)c1cc2cc(C)n(CCN3CCOCC3)c2s1 ; CNc1ccc(C(=O)c2cc3cc(C)n(CCN4CCOCC4)c3s2)cc1 ; Cc1cc2cc(C(=O)c3ccc(F)cc3F)sc2n1CCN1CCOCC1]

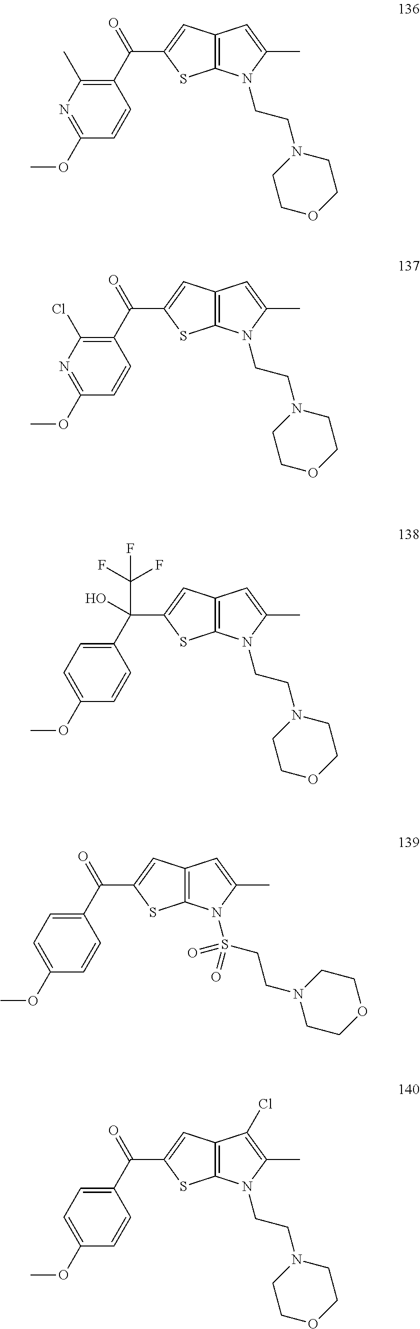 [SMILES: COc1ccc(C(=O)c2cc3cc(C)n(CCN4CCOCC4)c3s2)c(C)n1 ; COc1ccc(C(=O)c2cc3cc(C)n(CCN4CCOCC4)c3s2)c(Cl)n1 ; COc1ccc(C(O)(c2cc3cc(C)n(CCN4CCOCC4)c3s2)C(F)(F)F)cc1 ; COc1ccc(C(=O)c2cc3cc(C)n(S(=O)(=O)CCN4CCOCC4)c3s2)cc1 ; COc1ccc(C(=O)c2cc3c(Cl)c(C)n(CCN4CCOCC4)c3s2)cc1]

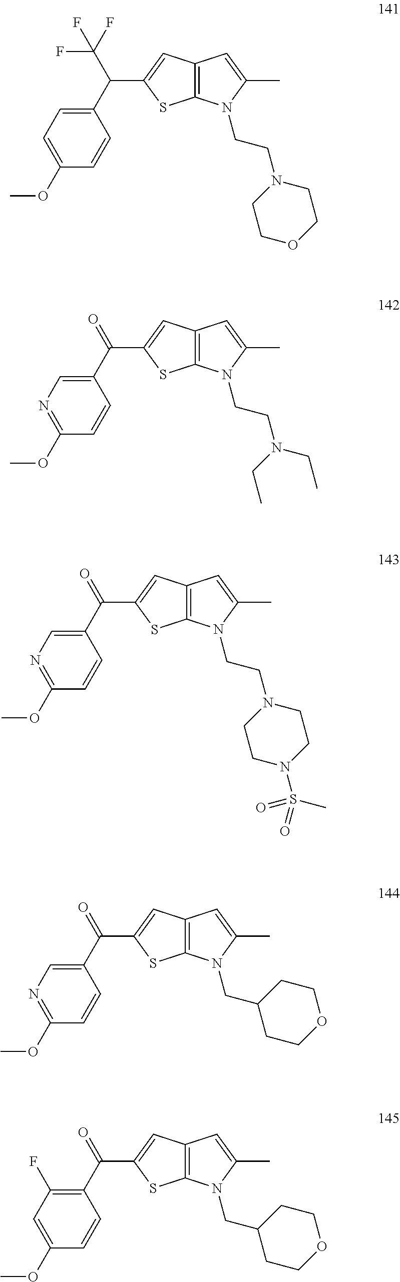

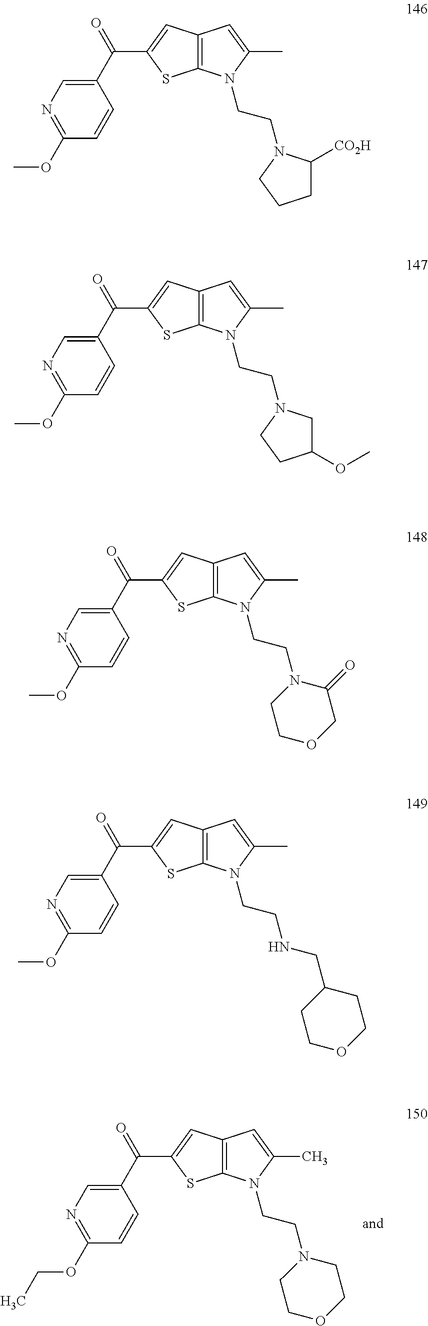

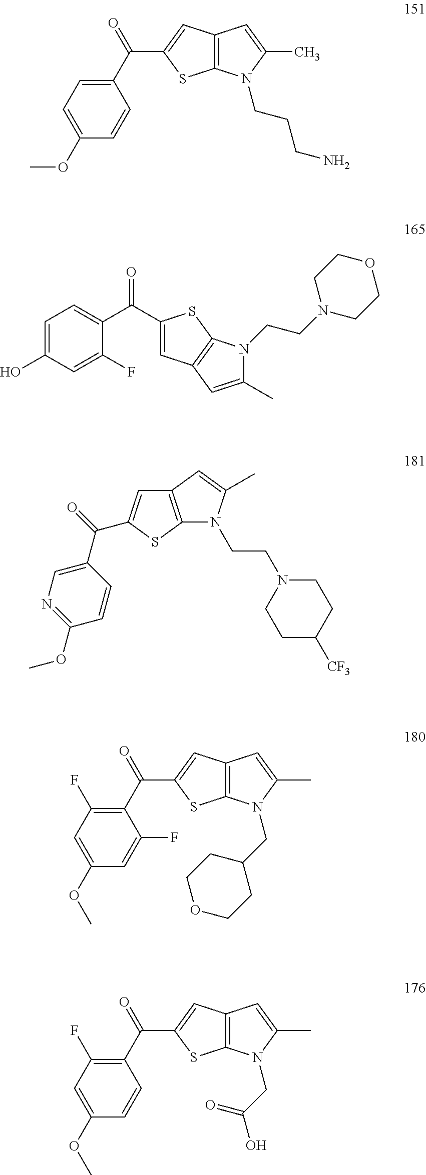 [SMILES: COc1ccc(C(=O)c2cc3cc(C)n(CCCN)c3s2)cc1 ; Cc1cc2cc(C(=O)c3ccc(O)cc3F)sc2n1CCN1CCOCC1 ; COc1ccc(C(=O)c2cc3cc(C)n(CCN4CCC(C(F)(F)F)CC4)c3s2)cn1 ; COc1cc(F)c(C(=O)c2cc3cc(C)n(CC4CCOCC4)c3s2)c(F)c1 ; COc1ccc(C(=O)c2cc3cc(C)n(CC(=O)O)c3s2)c(F)c1]

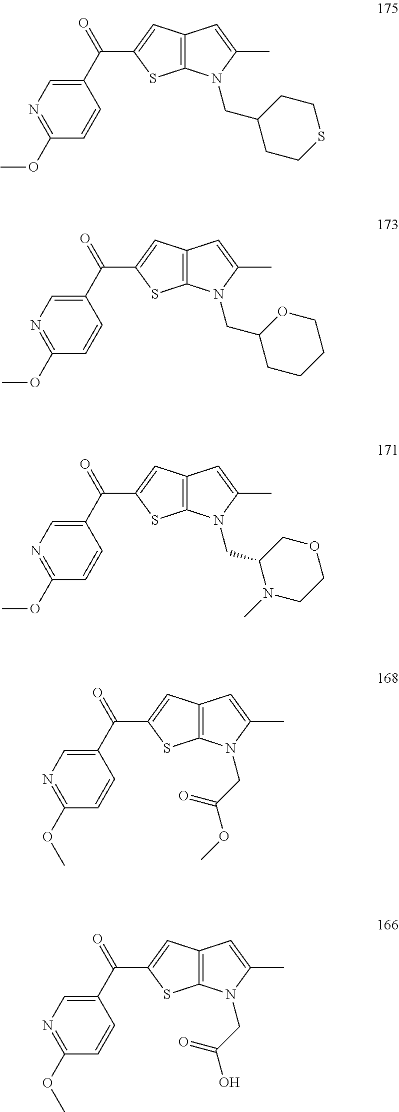 [SMILES: COc1ccc(C(=O)c2cc3cc(C)n(CC4CCSCC4)c3s2)cn1 ; COc1ccc(C(=O)c2cc3cc(C)n(CC4CCCCO4)c3s2)cn1 ; COc1ccc(C(=O)c2cc3cc(C)n(C[C@@H]4COCCN4C)c3s2)cn1 ; COC(=O)Cn1c(C)cc2cc(C(=O)c3ccc(OC)nc3)sc21 ; COc1ccc(C(=O)c2cc3cc(C)n(CC(=O)O)c3s2)cn1]

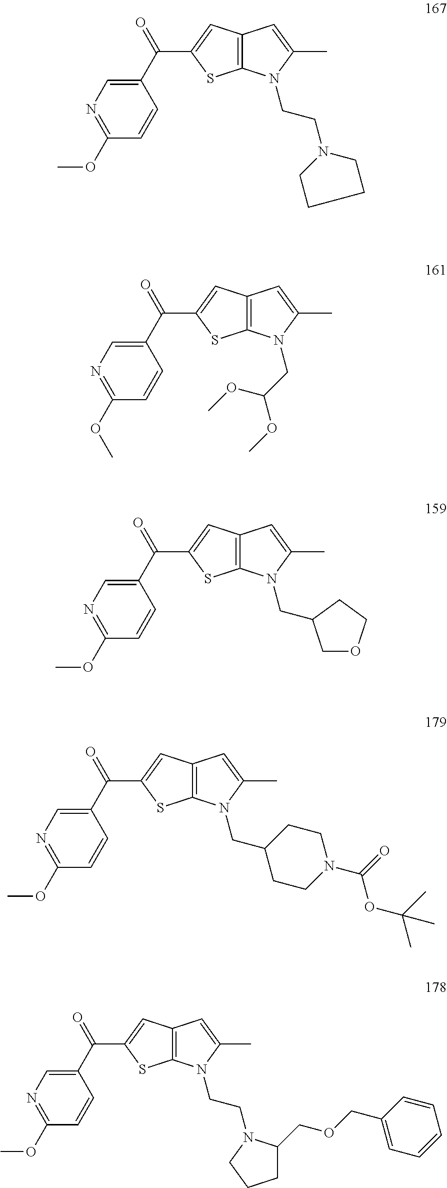

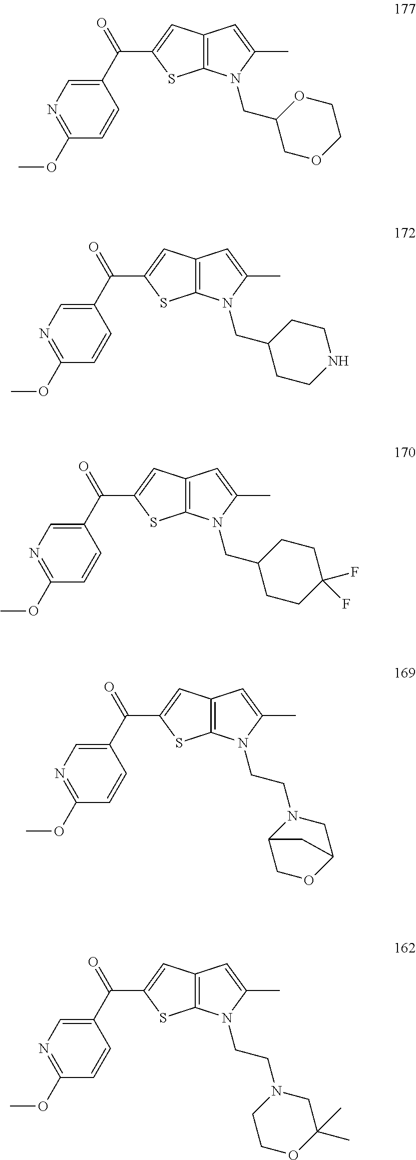

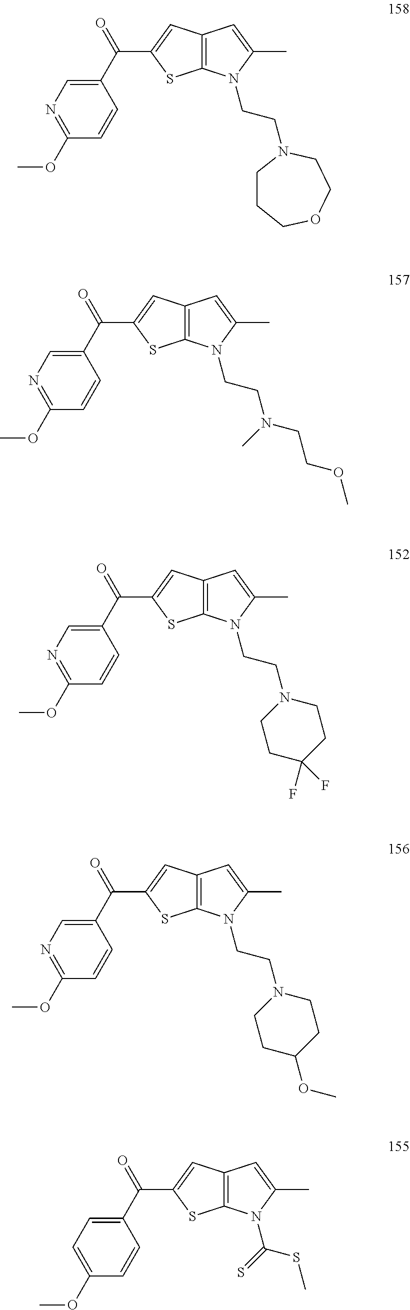

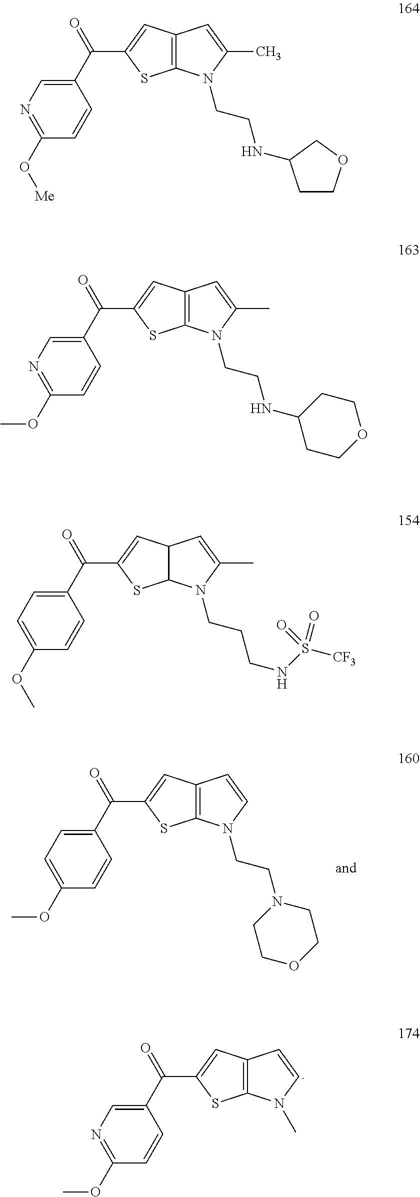 [SMILES: COc1ccc(C(=O)c2cc3cc(C)n(CCNC4CCOC4)c3s2)cn1 ; COc1ccc(C(=O)c2cc3cc(C)n(CCNC4CCOCC4)c3s2)cn1 ; COc1ccc(C(=O)C2=CC3C=C(C)N(CCCNS(=O)(=O)C(F)(F)F)C3S2)cc1 ; COc1ccc(C(=O)c2cc3ccn(CCN4CCOCC4)c3s2)cc1 ; COc1ccc(C(=O)c2cc3ccn(C)c3s2)cn1]

49. A pharmaceutical composition comprising a compound according to claim 1, or a pharmaceutically acceptable salt thereof, and a pharmaceutically acceptable carrier, vehicle or adjuvant.

50. The pharmaceutical composition of claim 49, further comprising at least one additional therapeutic agent.

51. The pharmaceutical composition of claim 50, wherein the additional therapeutic agent is chosen from the group consisting of pain relieving agents, non-steroidal anti-inflammatory drugs (NSAIDs), cannabinoid receptor agonists, opiate receptor agonists, sodium channel blockers, N-type calcium channel blockers, local anesthetics, VR1 agonists and antagonists, agents used for migraine, anti-inflammatory and/or immunosuppressive agents, agents designed to treat tobacco abuse (e.g., nicotine receptor partial agonists and nicotine replacement therapies), ADD/ADHD agents, agents to treat alcoholism, such as opioid antagonists, agents for reducing alcohol withdrawal symptoms such as benzodiazepines and beta-blockers, antihypertensive agents such as ACE inhibitors and Angiotensin II Receptor blockers, Renin inhibitors, vasodilators, agents used to treat glaucoma such as direct-acting Miotics (cholinergic agonists), indirect acting Miotics (cholinesterase inhibitors), Carbonic anhydrase inhibitors, selective adrenergic agonists, Osmotic diuretics, antidepressants such as SSRIs, tricyclic antidepressants, and dopaminergic antidepressants, cognitive improvement agents, acetylcholinesterase inhibitors, anti-emetic agents (e.g., 5HT3 antagonists), neuroprotective agents, neuroprotective agents currently under investigation, antipsychotic medications, agents used for multiple sclerosis, disease-modifying antirheumatic drugs (DMARDS), biological response modifiers (BRMs), COX-2 selective inhibitors, COX-1 inhibitors, immunosuppressives, PDE4 inhibitors, corticosteroids, histamine H1 receptor antagonists, histamine H2 receptor antagonists, proton pump inhibitors, leukotriene antagonists, 5-lipoxygenase inhibitors, nicotinic acetylcholine receptor agonists, P2X3 receptor antagonists, NGF agonists and antagonists, NK1 and NK2 antagonists, NMDA antagonist, potassium channel modulators, GABA modulators, and serotonergic and noradrenergic modulators.

52. A method for the treatment or prevention of pain comprising administering, alone or in combination therapy, to a patient in need thereof a therapeutically or prophylactically acceptable dose of a pharmaceutical composition according to claim 49.

53. The method according to claim 52, wherein the pain is chronic pain, acute pain, perioperative pain (e.g., associated with surgery), postoperative pain, visceral pain, inflammatory pain, cancer pain, headache pain, neuropathic pain, dental pain (such as odontalgia), bone pain, joint pain (e.g., osteoarthritis or rheumatoid arthritis), myofascial pain (e.g., muscular injury, fibromyalgia), labor pain, pain associated with injuries, pain resulting from trauma, pain resulting from allergies, pain resulting from dermatitis, pain resulting from immunodeficiency, pain resulting from Hodgkin's disease, pain resulting from Myasthenia gravis, pain resulting from nephrotic syndrome, pain resulting from scleroderma, pain resulting from thyroiditis, central and peripheral pathway mediated pain, or pain associated with or the result of injury or age.

54. A method for the treatment or prevention of autoimmune disorders comprising administering, alone or in combination therapy, to a patient in need thereof a therapeutically or prophylactically acceptable dose of a pharmaceutical composition according to claim 49.

55. The method according to claim 54, wherein the autoimmune disorder is selected from the group consisting of alopecia areata (also known as systemic sclerosis (SS)), amyloses, amyotrophic lateral sclerosis, ankylosing spondylarthritis, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, connective tissue diseases, crest syndrome, Crohn's disease, Degos' disease, dermatomyositis-juvenile, discoid lupus, essential mixed cryoglobulinemia, fibromyalgia-fibromyositis, graft vs. host disease, transplantation rejection, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, lupus erythematosus, Meniere's disease, multiple sclerosis, myasthenia gravis, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, reactional arthritis, Reiter's syndrome, rheumatic fever, rheumatoid arthritis, sarcoidosis, scleroderma (progressive systemic sclerosis (PSS), Sjogren's syndrome, stiff-man syndrome, systemic lupus erythematosus, Takayasu arteritis, temporal arteritis/giant cell arteritis, ulcerative colitis, undifferentiated spondylarthritis, uveitis, vitiligo, and Wegener's granulomatosis.

56. A method for the treatment or prevention of disease-states or indications that are accompanied by inflammatory processes comprising administering, alone or in combination therapy, to a patient in need thereof a therapeutically or prophylactically acceptable dose of a pharmaceutical composition according to claim 49.

57. The method according to claim 56, wherein the disease-states or indications that are accompanied by inflammatory processes are chosen from the group consisting of:
lung diseases such as asthma, bronchitis, allergic rhinitis, emphysema, adult respiratory distress syndrome (ARDS), pigeon fancier's disease, farmer's lung, chronic obstructive pulmonary disease (COPD), asthma including allergic asthma (atopic or non-atopic) as well as exercise-induced bronchoconstriction, occupational asthma, viral- or bacterial exacerbation of asthma, other non-allergic asthmas and “wheezy-infant syndrome”, pneumoconiosis, including aluminosis, anthracosis, asbestosis, chalicosis, ptilosis, siderosis, silicosis, tabacosis and byssinosis;
rheumatic diseases or autoimmune diseases or musculoskeletal diseases such as all forms of rheumatic diseases, especially rheumatoid arthritis, acute rheumatic fever, and polymyalgia rheumatica; reactive arthritis; rheumatic soft tissue diseases; inflammatory soft tissue diseases of other genesis; arthritic symptoms in degenerative joint diseases (arthroses); tendinitis, bursitis, osteoarthritis, traumatic arthritis, gout (metabolic arthritis); collagenoses of any genesis, e.g., systemic lupus erythematosus, scleroderma, polymyositis, dermatomyositis, Sjogren syndrome, Still disease, Felty syndrome; and osteoporosis and other bone resorption diseases;
allergic diseases including all forms of allergic reactions, e.g., allergic rhinitis, allergic conjunctivitis infectious parasitic, angioneurotic edema, hay fever, insect bites, allergic reactions to drugs, blood derivatives, contrast agents, etc., anaphylactic shock (anaphylaxis), urticaria, angioneurotic edema, delayed or immediate hypersensitivity, and contact dermatitis;
vascular diseases such as panarteritis nodosa, polyarteritis nodosa, periarteritis nodosa, arteritis temporalis, Wegner granulomatosis, giant cell arthritis, atherosclerosis, reperfusion injury and erythema nodosum;
dermatological diseases such as dermatitis, psoriasis, sunburn, burns, and eczema;
renal, urinary and pancreatic diseases such as nephrotic syndrome and all types of nephritis (such as glomerulonephritis); pancreatitis; bladder hyperrelexia following bladder inflammation;
hepatic diseases such as acute liver cell disintegration; acute hepatitis of various genesis (such as viral, toxic, drug-induced) and chronically aggressive and/or chronically intermittent hepatitis, liver fibrosis associated with liver injury or disease, including fibrosis caused or exacerbated by alcoholic liver cirrhosis, chronic viral hepatitis, non alcoholic steatohepatitis and primary liver cancer;
gastrointestinal diseases such as inflammatory bowel diseases, irritable bowel syndrome, regional enteritis (Crohns disease), colitis ulcerosa, gastritis, aphthous ulcer, celiac disease, regional ileitis, and gastroesophageal reflux disease;
neurodegenerative diseases such as in the treatment of neurodegeneration following stroke, cardiac arrest, pulmonary bypass, traumatic brain injury, spinal cord injury or the like;
eye diseases such as allergic keratitis, uveitis, or iritis, conjunctivitis, blepharitis, neuritis nervi optici, choroiditis, glaucoma and sympathetic ophthalmia;
diseases of the ear, nose, and throat (ENT) area such as tinnitus, allergic rhinitis or hay fever, otitis externa, caused by contact eczema, infection, etc., and otitis media;
neurological diseases such as brain edema, particularly tumor-related brain edema, multiple sclerosis, acute encephalomyelitis, meningitis, acute spinal cord injury, trauma, dementia, particularly degenerative dementia (including senile dementia, Alzheimer's disease, Parkinson's disease and Creutzfeldt-Jacob disease, Huntington's chorea, Pick's disease, motor neuron disease), vascular dementia (including multi-infarct dementia and dementia associated with intracranial space occupying lesions, infections and related conditions such as HIV infection), Guillain-Barre syndrome, myasthenia gravis, stroke, and various forms of seizures (such as nodding spasms);
blood diseases such as acquired hemolytic anemia, aplastic anemia, and idiopathic thrombocytopenia;
tumor diseases such as acute lymphatic leukemia, Hodgkin's disease, malignant lymphoma, lymphogranulomatoses, lymphosarcoma, solid malignant tumors, and extensive metastases;
endocrine diseases such as endocrine opthalmopathy, endocrine orbitopathia, thyrotoxic crisis, Thyroiditis de Quervain, Hashimoto thyroiditis, Morbus Basedow, granulomatous thyroiditis, struma lymphomatosa, Graves disease, type I diabetes (such as insulin-dependent diabetes); Organ and tissue transplantations and graft-versus-host diseases; and
severe states of shock such as septic shock, anaphylactic shock, and systemic inflammatory response syndrome (SIRS); and various other disease-states or conditions including, restenosis following percutaneous transluminal coronary angioplasty, acute and chronic pain, atherosclerosis, reperfusion injury, congestive heart failure, myocardial infarction, thermal injury, multiple organ injury secondary to trauma, necrotizing enterocolitis and syndromes associated with hemodialysis, leukopheresis, granulocyte transfusion, sarcoidosis, gingivitis, pyrexia. Edema resulting from trauma associated with burns, sprains or fracture, cerebral edema and angioedema, and diabetes (such as diabetic vasculopathy, diabetic neuropathy, diabetic retinopathy, post capillary resistance and diabetic symptoms associated with insulitis (e.g. Hyperglycemia, diuresis, proteinuria and increased nitrite and kallikrein urinary excretion)).

58. A method for the treatment or prevention of substance abuse related syndromes, disorders, diseases or withdrawal symptoms comprising administering, alone or in combination therapy, to a patient in need thereof a therapeutically or prophylactically acceptable dose of a pharmaceutical composition according to claim 49.

59. The method according to claim 58, wherein the substance abuse related syndromes, disorders, diseases or withdrawal symptoms are chosen from the group consisting of drug abuse and drug withdrawal, wherein the abused substances include alcohol, amphetamines, amphetamine like substances, caffeine, cannabis, cocaine, hallucinogens, inhalants, opioids, nicotine (and/or tobacco products), heroin abuse, barbiturates, phencyclidine (or phencyclidine-like compounds), sedative-hypnotics, benzodiazepines, or combinations of any of the foregoing; and the withdrawal symptoms include tobacco craving or nicotine dependency, addiction, or withdrawal.

60. A method for the treatment or prevention of psychiatric disorders comprising administering, alone or in combination therapy, to a patient in need thereof a therapeutically or prophylactically acceptable dose of a pharmaceutical composition according to claim 49.

61. The method according to claim 60, wherein the psychiatric disorders are chosen from the group consisting of depressions (including major depressive disorder, bipolar depression, unipolar depression, single or recurrent major depressive episodes (e.g., with or without psychotic features, catatonic features, and/or melancholic features), postpartum onset, seasonal affective disorder, dysthymic disorders (e.g., with early or late onset and with or without atypical features), neurotic depression and social phobia, depression accompanying dementia, anxiety, psychosis, social affective disorders, and/or cognitive disorders), manic-depressive psychoses, bipolar disorders, extreme psychotic states (such as mania, schizophrenia, and excessive mood swings where behavioral stabilization is desired), attention disorders such as ADHD (attention deficit hyperactivity disorders), autism, anxiety states, generalized anxiety, agoraphobia, as well as those behavioral states characterized by social withdrawal.

62. A method for the treatment or prevention of neurological or neurodegenerative disorders comprising administering, alone or in combination therapy, to a patient in need thereof a therapeutically or prophylactically acceptable dose of a pharmaceutical composition according to claim 49.

63. The method according to claim 63, wherein the neurological or neurodegenerative disorders are chosen from the group consisting of dementia, particularly degenerative dementia (including senile dementia, Alzheimer's disease, Pick's disease, Huntington's chorea, Parkinson's disease and Creutzfeldt-Jakob disease, motor neuron disease); vascular dementia (including multi-infarct dementia); as well as dementia associated with intracranial space occupying lesions; trauma; infections and related conditions (including HIV infection); dementia in Parkinson's disease, metabolism; toxins; anoxia and vitamin deficiency; and mild cognitive impairment associated with ageing, particularly Age Associated Memory Impairment; amyotrophic lateral sclerosis (ALS), multiple sclerosis, epilepsy, ischemia, traumatic head or brain injury, brain inflammation, eye injury, stroke and neuroinflammation.

64. A method for the treatment or prevention of ocular disorders comprising administering, alone or in combination therapy, to a patient in need thereof a therapeutically or prophylactically acceptable dose of a pharmaceutical composition according to claim 49.

65. The method according to claim 64, wherein the ocular disorders are chosen from the group consisting of glaucoma (such as normal tension glaucoma), glaucoma-associated intraocular pressure retinitis, retinopathies, uveitis, acute injury to the eye tissue (e.g. conjunctivitis), high intraocular pressure, family history of glaucoma, glaucoma in the contralateral eye and high myopia.

66. The method according to claim 52, wherein the patient is a human.

67. The method according to claim 52, wherein the patient is a companion animal, exotic animal or a farm animal such as a dog, cat, mouse, rat, hamster, gerbil, guinea pig, rabbit, horse, pig or cow.

68. A method of increasing CB receptor activity in a biological sample, comprising contacting said biological sample with a composition according to claim 49.
